(19) United States
(12) Patent Application Publication
(10) Pub. No.: US 2022/0131081 A1
(43) Pub. Date: Apr. 28, 2022

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Doo-Hyeon MOON, Gyeonggi-do (KR); Su-Hyun LEE, Gyeonggi-do (KR); Du-Yong PARK, Gyeonggi-do (KR); Sang-Hee CHO, Gyeonggi-do (KR); Dong-Hyung LEE, Gyeonggi-do (KR)

(21) Appl. No.: 17/425,681

(22) PCT Filed: Jan. 22, 2020
(86) PCT No.: PCT/KR2020/001050

§ 371 Date: Jul. 23, 2021

(30) Foreign Application Priority Data
Jan. 25, 2019 (KR) 10-2019-0009730
Jan. 17, 2020 (KR) 10-2020-0006475

(51) Int. Cl. H01L 51/00; C09K 11/02; C07D 409/10; C09K 11/06; C07D 405/10; C07D 405/14; H01L 51/50

(52) U.S. Cl. CPC H01L 51/0067; C09K 11/02; C07D 409/10; H01L 51/0074; C09K 11/06; H01L 51/0073; C09K 2211/185; C07D 405/14; H01L 2251/558; H01L 51/5016; C09K 2211/1029; H01L 51/5096; C09K 2211/1007; C07D 405/10

(57) ABSTRACT

The present disclosure relates to an organic electroluminescent compound represented by formula 1 and an organic electroluminescent device comprising the same. By comprising the organic electroluminescent compound of the present disclosure, it is possible to provide an organic electroluminescent device having long lifetime and/or high luminous efficiency properties.

### TECHNICAL FIELD

The present disclosure relates to an organic electroluminescent compound and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic electroluminescent device was developed by Eastman Kodak in 1987, by using small aromatic diamine molecules and aluminum complexes as materials for forming a light-emitting layer (see Appl. Phys. Lett. 51, 913, 1987).

An organic electroluminescent device (OLED) changes electric energy into light by applying electricity to an organic electroluminescent material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc., if necessary. The materials used in the organic layer can be classified into a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc., depending on their functions. In the OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from an energy when the organic light-emitting compound returns to the ground state from the excited state.

The most important factor determining luminescent efficiency in an OLED is light-emitting materials. The light-emitting materials are required to have the following features: high quantum efficiency, high mobility of an electron and a hole, and uniformity and stability of the formed light-emitting material layer. The light-emitting material is classified into blue, green, and red light-emitting materials according to the light-emitting color, and further includes yellow or orange light-emitting materials. Furthermore, the light-emitting material is classified into a host material and a dopant material in a functional aspect. Recently, an urgent task is the development of an OLED having high efficiency and long lifetime. In particular, the development of highly excellent light-emitting material over conventional materials is urgently required, considering the EL properties necessary for medium- and large-sized OLED panels.

Meanwhile, Korean Patent Appl. Laid-Open No. 2017-0096769 and Korean Patent No. 1814875 disclose a heterocyclic compound and an organic electroluminescent device comprising the same. However, the development for improving performances of an OLED is still required.

### DISCLOSURE OF INVENTION

### Technical Problem

The objective of the present disclosure is to provide an organic electroluminescent compound effective for producing an organic electroluminescent device having long lifetime and/or high luminous efficiency properties.

### Solution to Problem

The present inventors have found that the above objective can be achieved by an organic electroluminescent compound represented by the following formula 1:

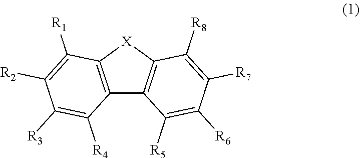

wherein

X represents O or S;

R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino; or may be linked to an adjacent substituent(s) to form a ring(s); and

at least one group of group R₅ and R₆, group R₆ and R₇, and group R₇ and R₈ are fused to the following formula 2 to form a ring(s):

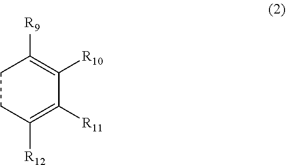

wherein

R₅ to R₈, which do not form a ring, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino;

R₉ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino, or *-L-ETU; with the proviso that at least one of R₉ to R₁₂ represents *-L-ETU;

L represents a single bond, a substituted or unsubstituted (C6-C30)arylene, or a substituted or unsubstituted (3- to 30-membered)heteroarylene; and

ETU represents a substituted or unsubstituted triazinyl, a substituted or unsubstituted quinazolinyl, a substituted or unsubstituted quinoxalinyl, a substituted or unsubstituted benzoquinoxalinyl, a substituted or unsubstituted dibenzoquinoxalinyl, a substituted or unsubstituted benzoquinazolinyl, a substituted or unsubstituted dibenzoquinazolinyl, a substituted or unsubstituted benzofuropyrazinyl, a substituted or unsubstituted benzothiopyrazinyl, a substituted or unsubstituted benzofuropyrimidinyl, or a substituted or unsubstituted benzothiopyrimidinyl.

### Advantageous Effects of Invention

The organic electroluminescent compound according to the present disclosure can provide an organic electroluminescent device having long lifetime and/or high luminous efficiency properties.

### MODE FOR THE INVENTION

Hereinafter, the present disclosure will be described in detail. However, the following description is intended to explain the disclosure, and is not meant in any way to restrict the scope of the disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, an electron injection material, etc.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, preferably 6 to 25 ring backbone carbon atoms, and more preferably 6 to 18 ring backbone carbon atoms. The above aryl or arylene may be partially saturated, and may comprise a Spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzofluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-Pert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, etc.

The term “(3- to 30-membered)heteroaryl(ene)” is an aryl(ene) having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinalyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-teat-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent. In the present disclosure, the substituents of the substituted alkyl, the substituted aryl(ene), the substituted heteroaryl(ene), the substituted silyl, the substituted amino, the substituted pyrimidinyl, the substituted triazinyl, the substituted quinazolinyl, the substituted quinoxalinyl, the substituted benzoquinoxalinyl, the substituted dibenzoquinoxalinyl, the substituted benzoquinazolinyl, the substituted dibenzoquinazolinyl, the substituted benzofuropyrazinyl, the substituted benzothiopyrazinyl, the substituted benzofuropyrimidinyl, and the substituted benzothiopyrimidinyl, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl, a (C1-C30)alkoxy; a (C1-C30)alkylthio; a (C3-C30)cycloalkyl; a (C3-C30)cycloalkenyl; a (3- to 7-membered)heterocycloalkyl, a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl, a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of a (C1-C20)alkyl; a (C6-C25)aryl, a (5- to 25-membered)heteroaryl unsubstituted or substituted with a (C6-C25)aryl(s); and a (C1-C10)alkyl(C6-C25)aryl. According to another embodiment of the present disclosure, the substituents, each independently, are at least one selected from the group consisting of a (C1-C10)alkyl; a (C6-C25)aryl; a (5- to 20-membered)heteroaryl unsubstituted or substituted with a (C6-C18)aryl(s); and a (C1-C5)alkyl(C6-C25)aryl. For example, the substituents, each independently, may be at least one selected from the group consisting of a methyl, a phenyl, a naphthyl, a biphenyl, a phenanthrenyl, a terphenyl, a triphenylenyl, a dimethylfluorenyl, a diphenylfluorenyl, a spirobifluorenyl, a carbazolyl substituted with a phenyl(s), a dibenzothiophenyl, a dibenzofuranyl, a benzonaphthothiophenyl, and a benzonaphthofuranyl.

In the formulas of the present disclosure, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; and preferably, a substituted or unsubstituted mono- or polycyclic (3- to 26-membered) alicyclic or aromatic ring, or the combination thereof. In addition, the ring may contain at least one heteroatom selected from B, N, O, S, Si, and P, preferably at least one heteroatom selected from N, O, and S. For example, the ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, a substituted or unsubstituted carbazole ring, etc.

Herein, the heteroaryl(ene) and the heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

In formula 1, R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino; or may be linked to an adjacent substituent(s) to form a ring(s). According to one embodiment of the present disclosure, R₁ to R₄, each independently, represent hydrogen, deuterium, a substituted or unsubstituted (C1-C20)alkyl, a substituted or unsubstituted (C6-C25)aryl, or a substituted or unsubstituted (5- to 30-membered)heteroaryl; or at least one group of group R₁ and R₂, group R₂ and R₃, and group R₃ and R₄ may be linked to each other to form a ring(s). According to another embodiment of the present disclosure, R₁ to R₄, each independently, represent hydrogen, deuterium, an unsubstituted (C6-C18)aryl, or a (5- to 25-membered)heteroaryl unsubstituted or substituted with a (C6-C18)aryl(s). For example, R₁ to R₄, each independently, represent hydrogen, a phenyl, a naphthyl, a biphenyl, a phenanthrenyl, a carbazolyl substituted with a phenyl(s), a dibenzothiophenyl, or a dibenzofuranyl.

In formula 1, at least one group of group R₅ and R₆, group R₆ and R₇, and group R₇ and R₈ are fused to the following formula 2 to form a ring(s). According to one embodiment of the present disclosure, R₅ and R₅, or R₆ and R₇, or R₇ and R₈ are fused to the following formula 2 to form a ring(s).

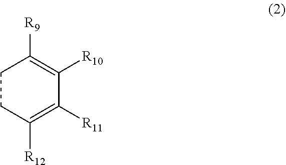

In formula 1, R₅ to R₈, which do not form a ring, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino. According to one embodiment of the present disclosure, R₅ to R₈, each independently, represent hydrogen, deuterium, a substituted or unsubstituted (C1-C10)alkyl, a substituted or unsubstituted (C6-C18)aryl, or a substituted or unsubstituted (5- to 20-membered)heteroaryl. According to another embodiment of the present disclosure, R₅ to R₈, each independently, represent hydrogen, deuterium, or an unsubstituted (C6-C18)aryl. For example, R₅ to R₈, each independently, may represent hydrogen, a phenyl, a naphthyl, or a biphenyl.

In formula 1, R₉ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino, or *-L-ETU. At least one of R₉ to R₁₂ represents *-L-ETU. According to one embodiment of the present disclosure, any one of R₉ to R₁₂ represents *-L-ETU. According to another embodiment of the present disclosure, R₉ to R₁₂, each independently, represent hydrogen, deuterium, or *-L-ETU; with the proviso that any one of R₉ to R₁₂ represents *-L-ETU.

L represents a single bond, a substituted or unsubstituted (C6-C30)arylene, or a substituted or unsubstituted (3- to 30-membered)heteroarylene. According to one embodiment of the present disclosure, L represents a single bond, a substituted or unsubstituted (C6-C25)arylene, or a substituted or unsubstituted (5- to 25-membered)heteroarylene. According to another embodiment of the present disclosure, L represents a single bond, an unsubstituted (C6-C18)arylene, or an unsubstituted (5- to 20-membered)heteroarylene. For example, L may represent a single bond, a phenylene, a naphthylene, a biphenylene, or a pyridylene.

ETU represents a substituted or unsubstituted triazinyl, a substituted or unsubstituted quinazolinyl, a substituted or unsubstituted quinoxalinyl, a substituted or unsubstituted benzoquinoxalinyl, a substituted or unsubstituted dibenzoquinoxalinyl, a substituted or unsubstituted benzoquinazolinyl, a substituted or unsubstituted dibenzoquinazolinyl, a substituted or unsubstituted benzofuropyrazinyl, a substituted or unsubstituted benzothiopyrazinyl, a substituted or unsubstituted benzofuropyrimidinyl, or a substituted or unsubstituted benzothiopyrimidinyl. According to one embodiment of the present disclosure, ETU represents a substituted triazinyl, a substituted quinazolinyl, a substituted quinoxalinyl, a substituted benzoquinoxalinyl, a substituted dibenzoquinoxalinyl, a substituted benzoquinazolinyl, a substituted benzofuropyrimidinyl, or a substituted benzothiopyrimidinyl. The substituent of the substituted triazinyl, the substituted quinazolinyl, the substituted quinoxalinyl, the substituted benzoquinoxalinyl, the substituted dibenzoquinoxalinyl, the substituted benzoquinazolinyl, the substituted benzofuropyrimidinyl, and the substituted benzothiopyrimidinyl, each independently, may be at least one selected from the group consisting of a substituted or unsubstituted (C6-C25)aryl, and a substituted or unsubstituted (5- to 30-membered)heteroaryl, and preferably, at least one selected from the group consisting of a phenyl, a naphthyl, a biphenyl, a phenanthrenyl, a terphenyl, a triphenylenyl, a dimethylfluorenyl, a diphenylfluorenyl, a spirobifluorenyl, a carbazolyl substituted with a phenyl(s), a dibenzothiophenyl, a dibenzofuranyl, a benzonaphthothiophenyl, and a benzonaphthofuranyl. For example, ETU may be represented by any one of the following.

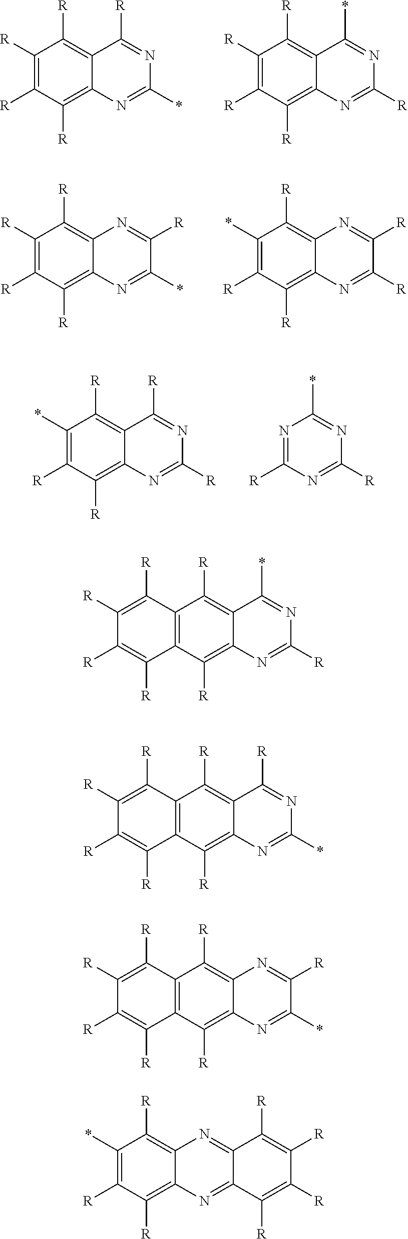

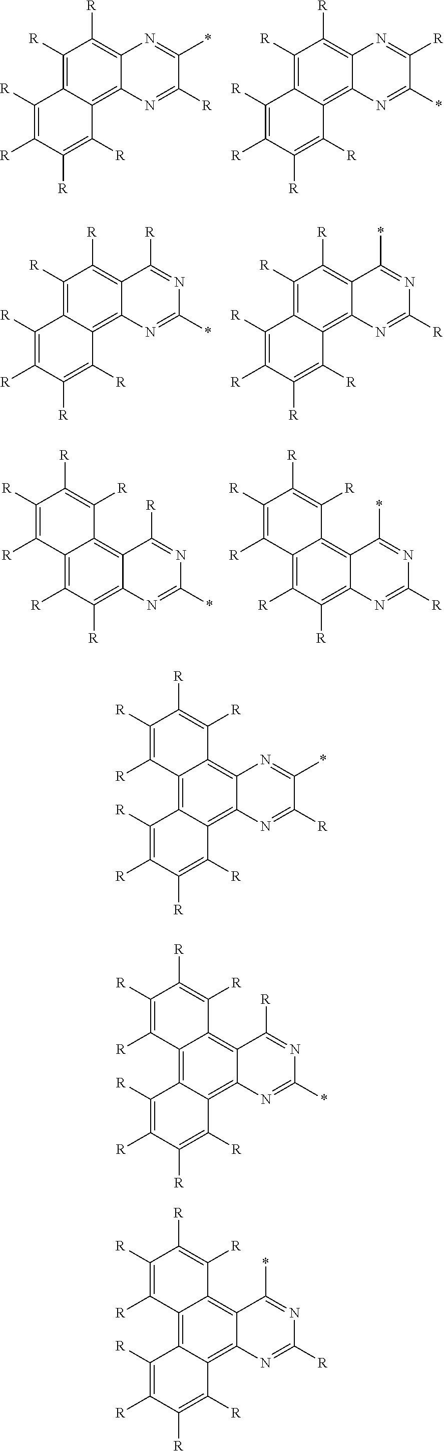

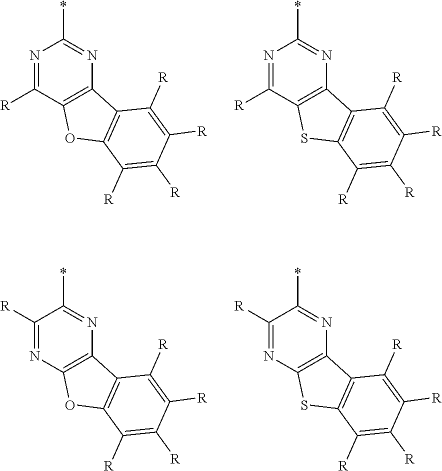

Herein, R, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted silyl, or a substituted or unsubstituted amino. According to one embodiment of the present disclosure, R, each independently, represents hydrogen, deuterium, a substituted or unsubstituted (C1-C10)alkyl, a substituted or unsubstituted (C6-C25)aryl, or a substituted or unsubstituted (5- to 30-membered)heteroaryl. According to another embodiment of the present disclosure, R, each independently, represents hydrogen, deuterium, a (C6-C25)aryl unsubstituted or substituted with a (C1-C10)alkyl(s) and/or a (C6-C18)aryl(s), or a (5- to 30-membered)heteroaryl unsubstituted or substituted with a (C6-C18)aryl(s). For example, R, each independently, may represent hydrogen, a phenyl, a naphthyl, a biphenyl, a phenanthrenyl, a terphenyl, a triphenylenyl, a dimethylfluorenyl, a diphenylfluorenyl, a spirobifluorenyl, a carbazolyl substituted with a phenyl(s), a dibenzothiophenyl, a dibenzofuranyl, a benzonaphthothiophenyl, or a benzonaphthofuranyl.

The compound represented by formula 1 may be represented by any one of the following formulas 1-1 to 1-3.

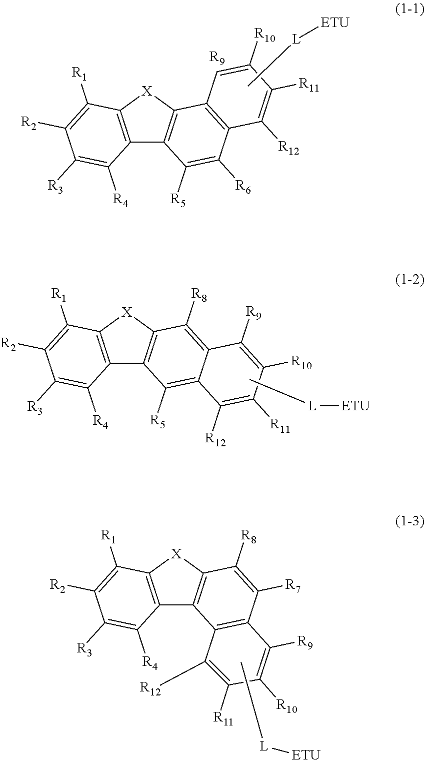

In formulas 1-1 to 1-3, R₅ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino; and R₁ to R₄, L, ETU, and X are as defined in formula 1 above. In addition, preferred embodiments and specific examples of R₁ to R₁₂, L, ETU, and X in formulas 1-1 to 1-3 are as mentioned in formula 1 above.

The compound represented by formula 1 may be any one selected from the group consisting of the following compounds, but is not limited thereto.

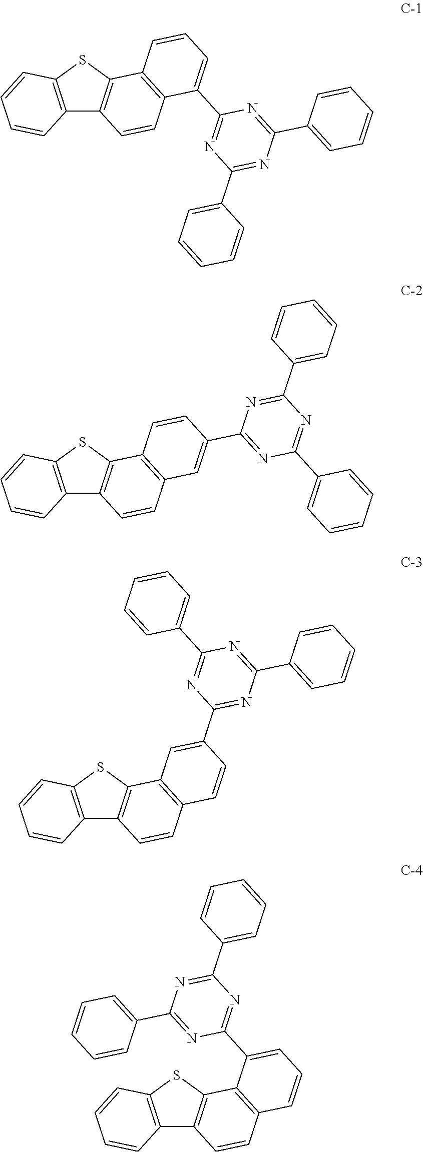 [SMILES: c1ccc(-c2nc(-c3ccccc3)nc(-c3cccc4c3ccc3c5ccccc5sc43)n2)cc1 ; c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1]

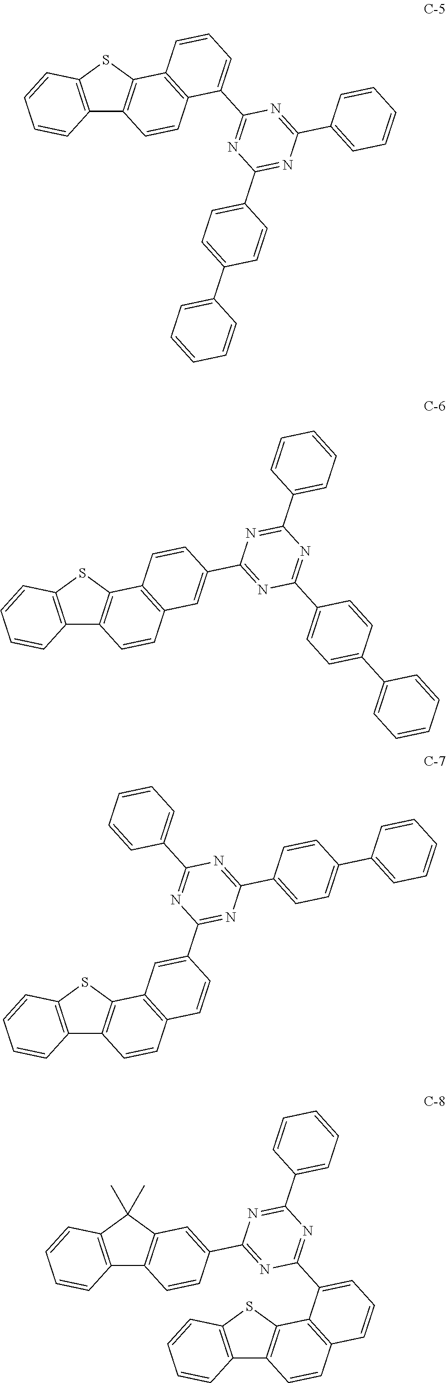 [SMILES: c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1 ; c1ccc(-c2ccc(-c3nc(-c4ccccc4)nc(-c4ccc5ccc6c7ccccc7sc6c5c4)n3)cc2)cc1 ; CC1(C)c2ccccc2-c2ccc(-c3nc(-c4ccccc4)nc(-c4cccc5ccc6c7ccccc7sc6c45)n3)cc21]

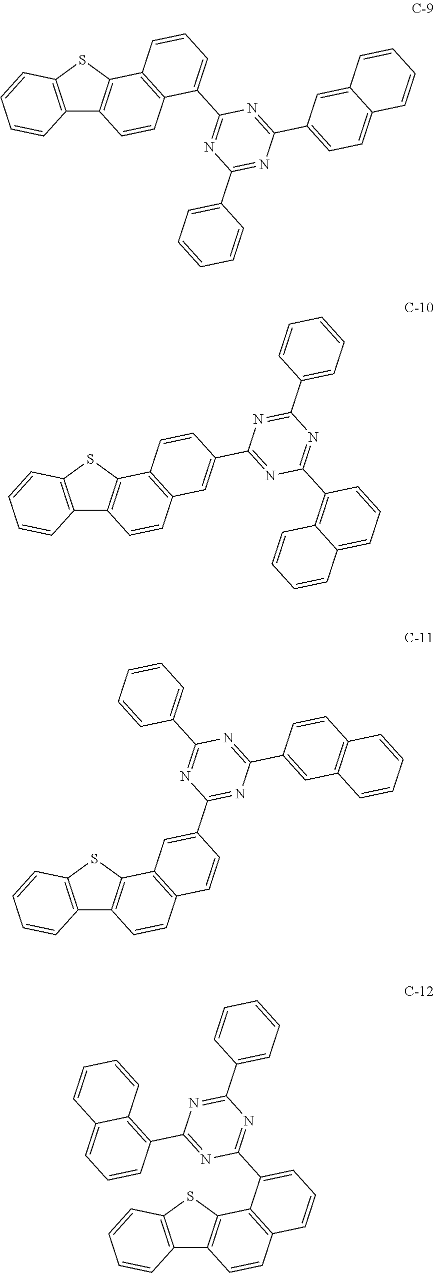 [SMILES: c1ccc(-c2nc(-c3ccc4ccccc4c3)nc(-c3cccc4c3ccc3c5ccccc5sc43)n2)cc1 ; c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1 ; c1ccc(-c2nc(-c3cccc4ccccc34)nc(-c3cccc4ccc5c6ccccc6sc5c34)n2)cc1]

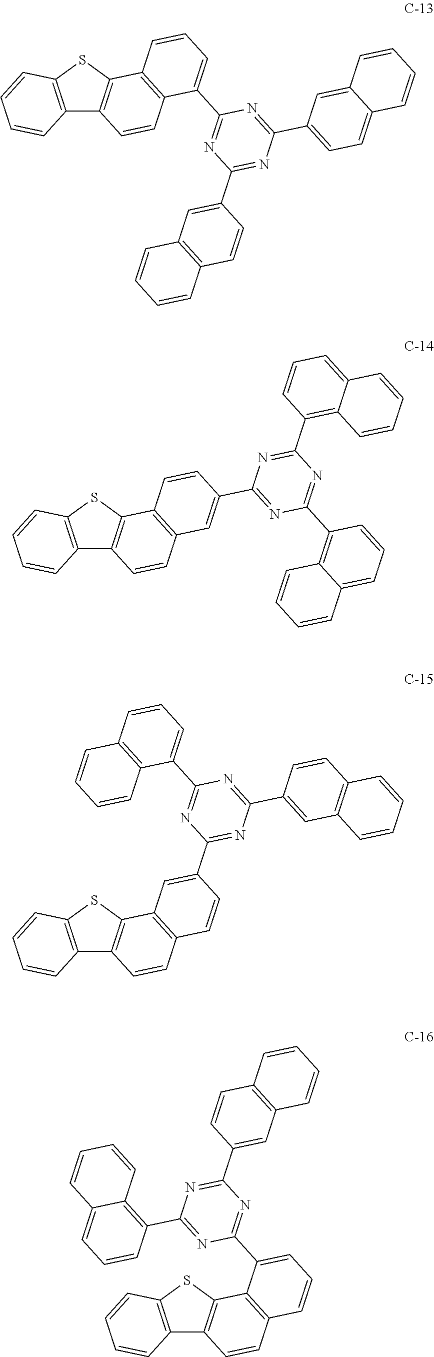 [SMILES: c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1]

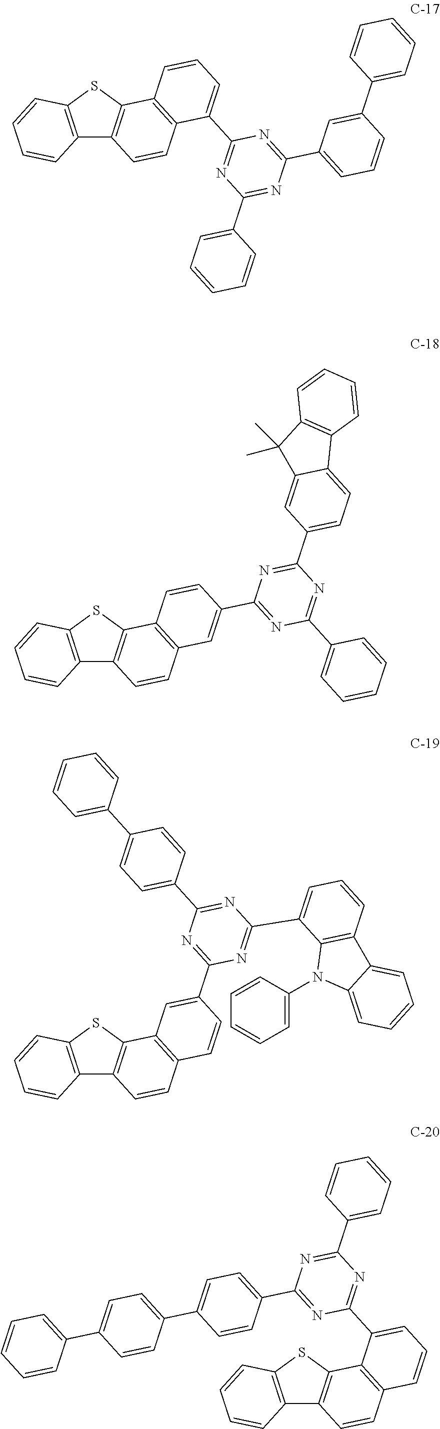 [SMILES: c1ccc(-c2cccc(-c3nc(-c4ccccc4)nc(-c4cccc5c4ccc4c6ccccc6sc54)n3)c2)cc1 ; CC1(C)c2ccccc2-c2ccc(-c3nc(-c4ccccc4)nc(-c4ccc5c(ccc6c7ccccc7sc56)c4)n3)cc21 ; c1ccc(-c2ccc(-c3nc(-c4ccc5ccc6c7ccccc7sc6c5c4)nc(-c4cccc5c6ccccc6n(-c6ccccc6)c45)n3)cc2)cc1 ; c1ccc(-c2ccc(-c3ccc(-c4nc(-c5ccccc5)nc(-c5cccc6ccc7c8ccccc8sc7c56)n4)cc3)cc2)cc1]

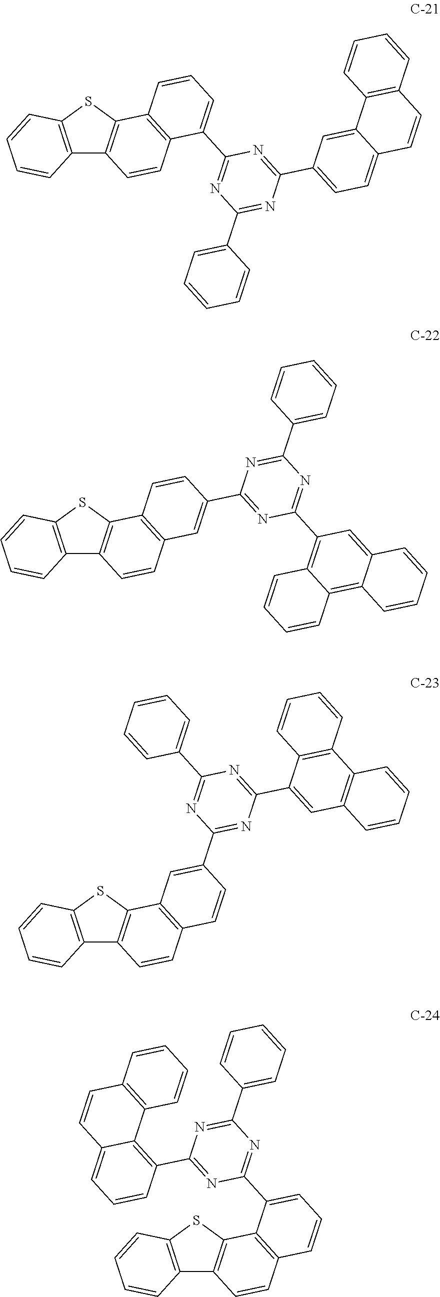 [SMILES: c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1 ; c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1]

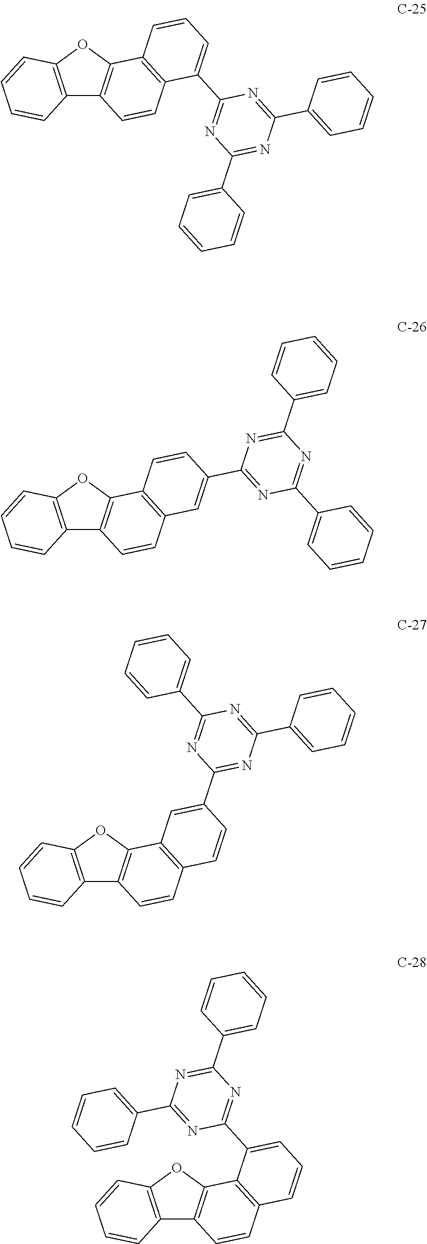 [SMILES: c1ccc(-c2nc(-c3ccccc3)nc(-c3cccc4c3ccc3c5ccccc5oc43)n2)cc1 ; c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1 ; c1ccc(-c2nc(-c3ccccc3)nc(-c3cccc4ccc5c6ccccc6oc5c34)n2)cc1]

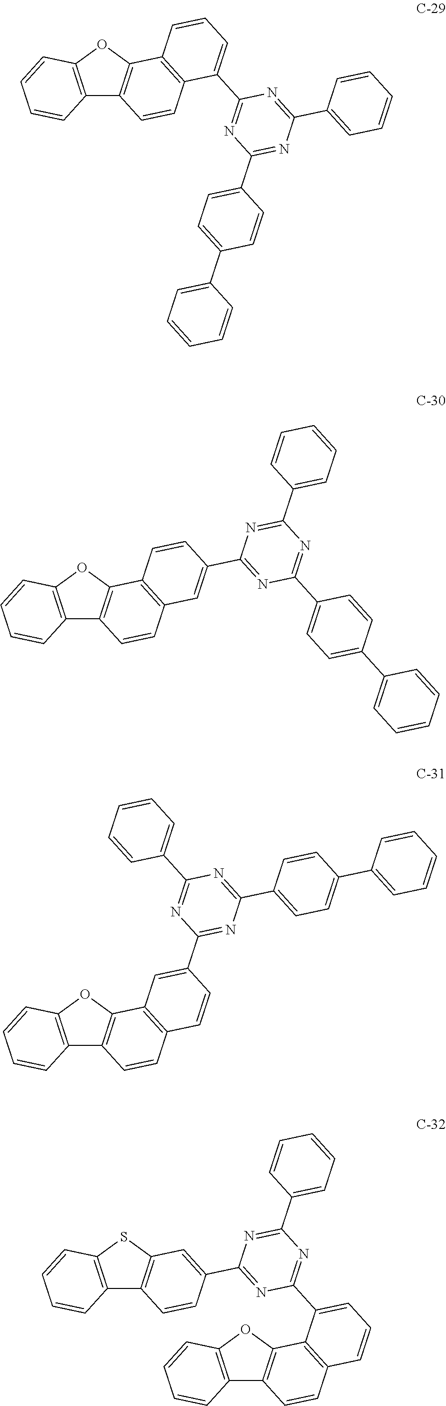 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1]

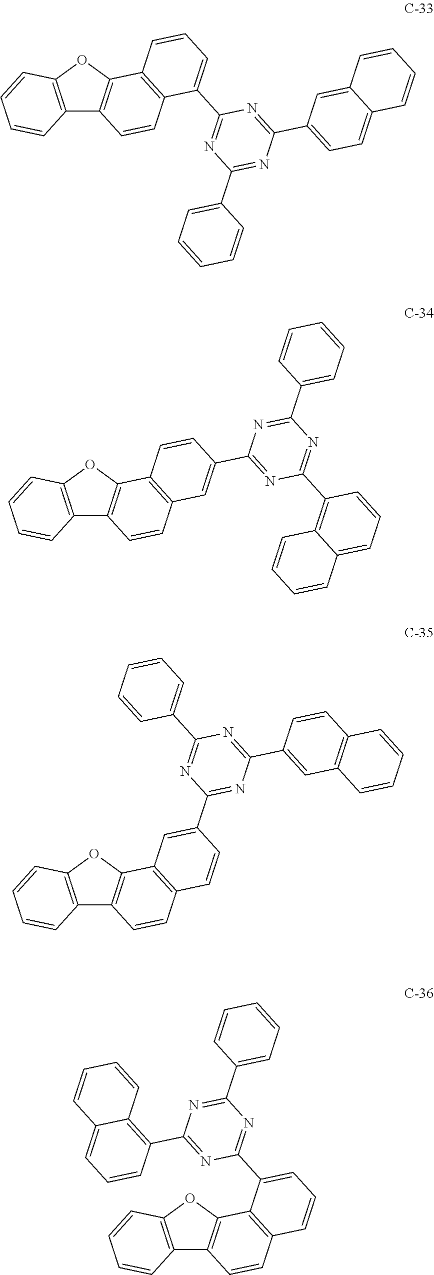 [SMILES: c1ccc(-c2nc(-c3ccc4ccccc4c3)nc(-c3cccc4c3ccc3c5ccccc5oc43)n2)cc1 ; c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1]

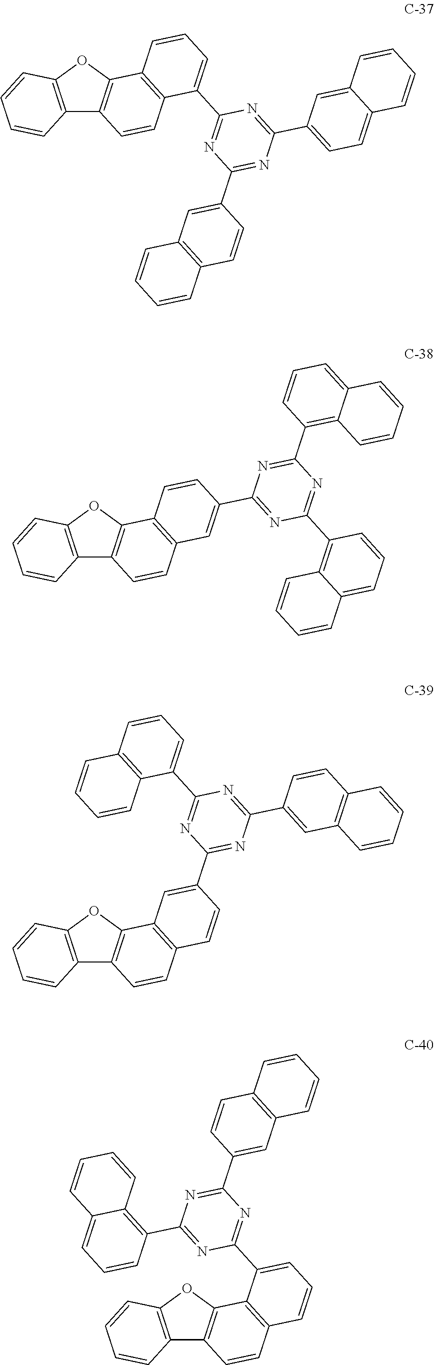 [SMILES: c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1 ; c1ccc2cc(-c3nc(-c4ccc5ccc6c7ccccc7oc6c5c4)nc(-c4cccc5ccccc45)n3)ccc2c1 ; c1ccc2cc(-c3nc(-c4cccc5ccccc45)nc(-c4cccc5ccc6c7ccccc7oc6c45)n3)ccc2c1]

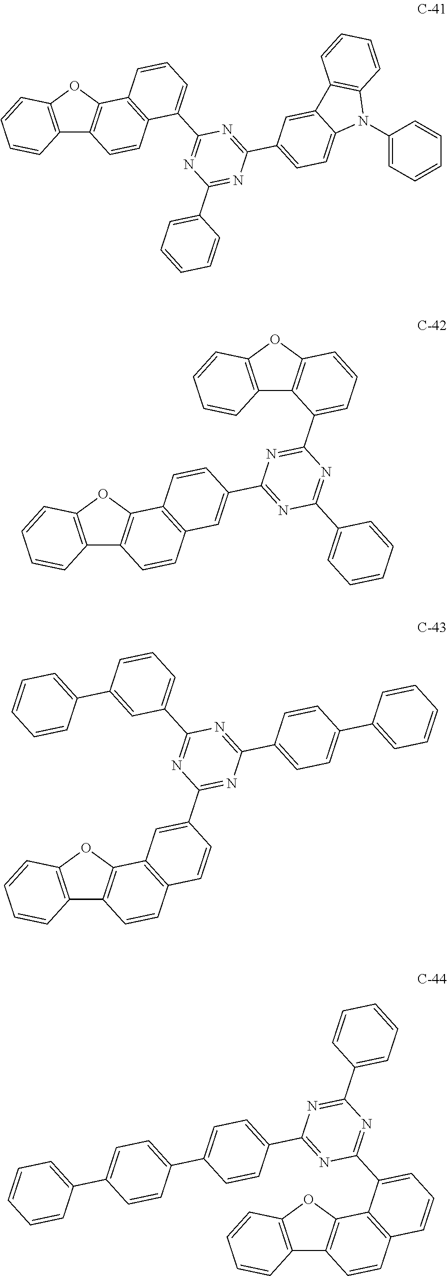 [SMILES: c1ccc(-c2nc(-c3ccc4c(c3)c3ccccc3n4-c3ccccc3)nc(-c3cccc4c3ccc3c5ccccc5oc43)n2)cc1 ; c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

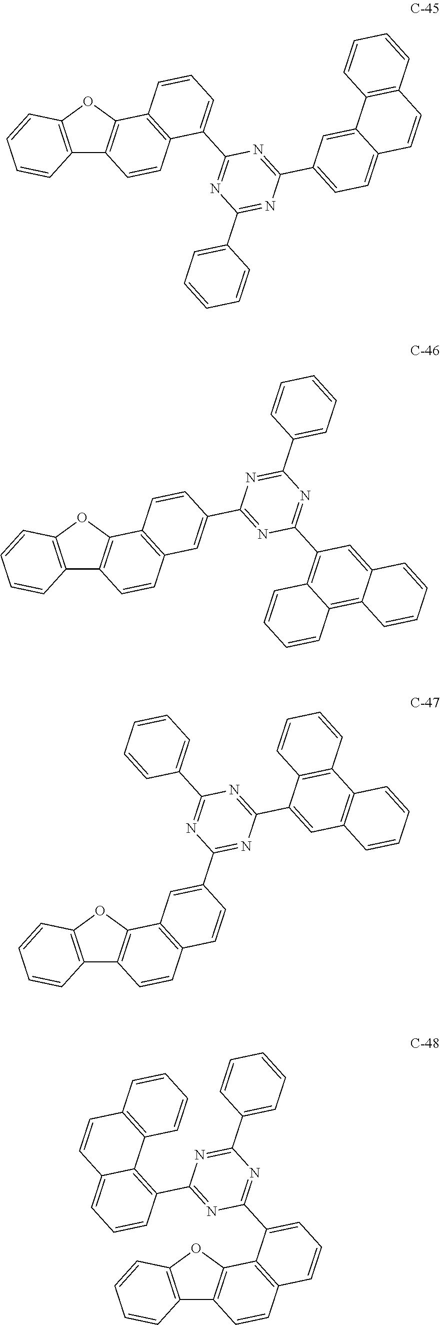 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3ccc4ccc5c6ccccc6oc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1]

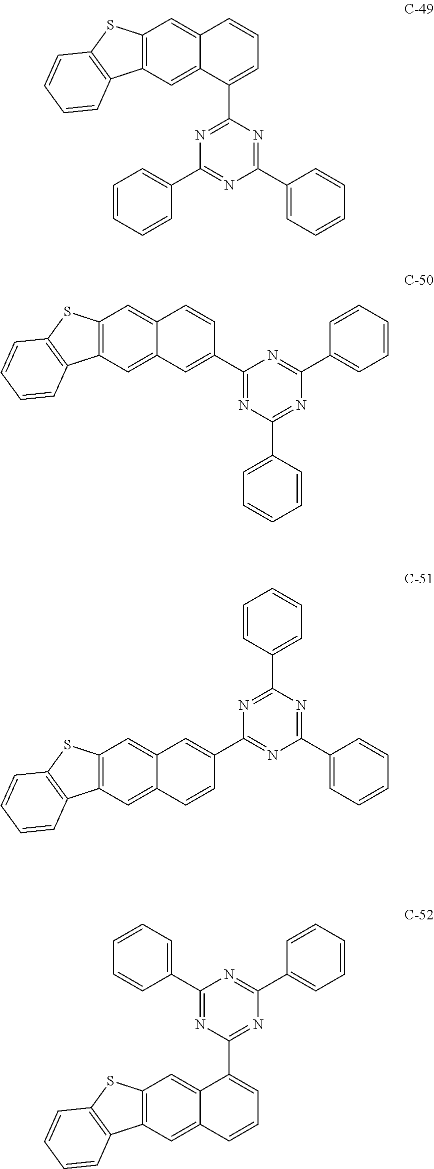 [SMILES: c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1 ; c1ccc(-c2nc(-c3ccccc3)nc(-c3ccc4cc5c(cc4c3)sc3ccccc35)n2)cc1 ; c1ccc(-c2nc(-c3ccccc3)nc(-c3cccc4cc5c(cc34)sc3ccccc35)n2)cc1]

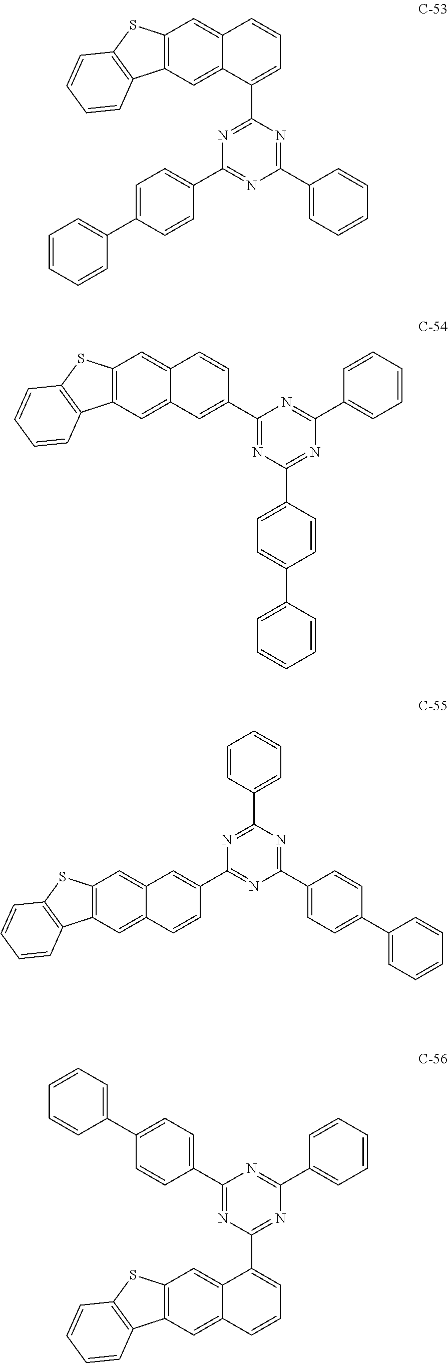 [SMILES: c1ccc(-c2ccc(-c3nc(-c4ccccc4)nc(-c4cccc5cc6sc7ccccc7c6cc45)n3)cc2)cc1 ; c1ccc(-c2ccc(-c3nc(-c4ccccc4)nc(-c4ccc5cc6sc7ccccc7c6cc5c4)n3)cc2)cc1 ; c1ccc(-c2ccc(-c3nc(-c4ccccc4)nc(-c4ccc5cc6c(cc5c4)sc4ccccc46)n3)cc2)cc1 ; c1ccc(-c2ccc(-c3nc(-c4ccccc4)nc(-c4cccc5cc6c(cc45)sc4ccccc46)n3)cc2)cc1]

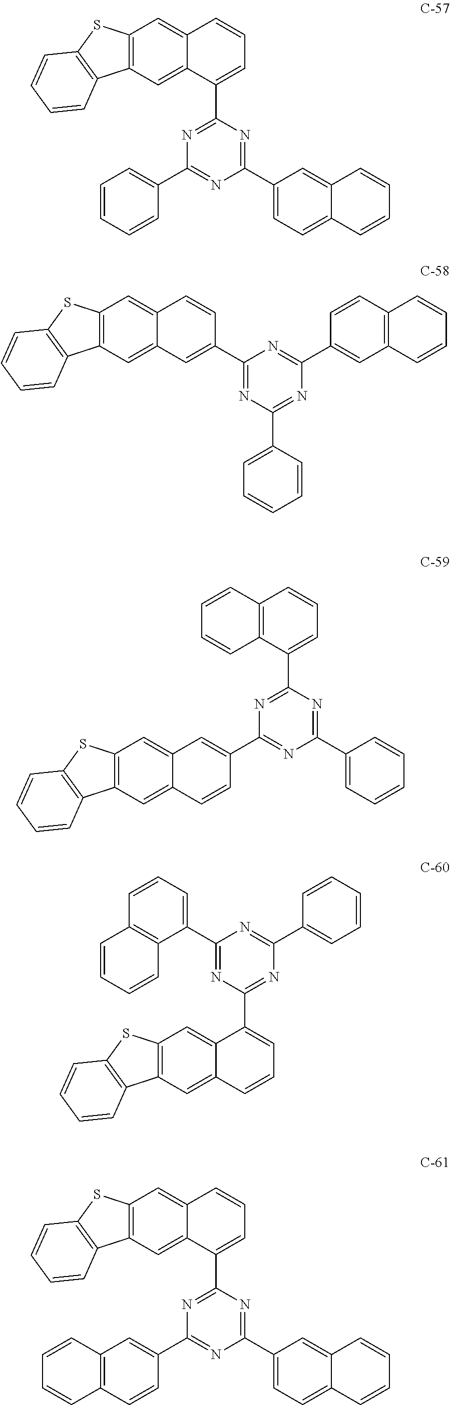 [SMILES: c1ccc(-c2nc(-c3ccc4ccccc4c3)nc(-c3cccc4cc5sc6ccccc6c5cc34)n2)cc1 ; c1ccc(-c2nc(-c3ccc4ccccc4c3)nc(-c3ccc4cc5sc6ccccc6c5cc4c3)n2)cc1 ; c1ccc(-c2nc(-c3ccc4cc5c(cc4c3)sc3ccccc35)nc(-c3cccc4ccccc34)n2)cc1 ; c1ccc(-c2nc(-c3cccc4ccccc34)nc(-c3cccc4cc5c(cc34)sc3ccccc35)n2)cc1 ; c1ccc2cc(-c3nc(-c4ccc5ccccc5c4)nc(-c4cccc5cc6sc7ccccc7c6cc45)n3)ccc2c1]

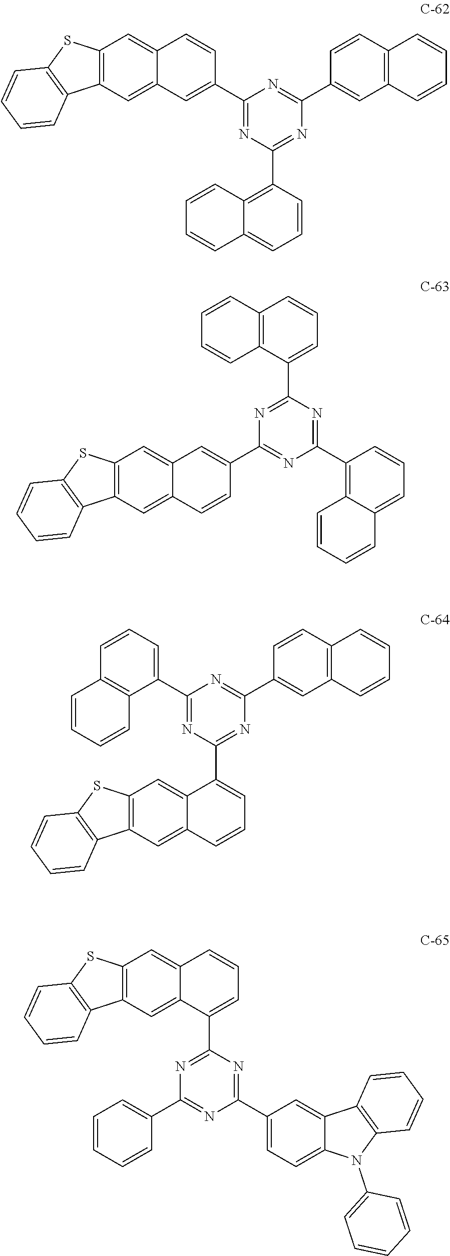 [SMILES: c1ccc2cc(-c3nc(-c4ccc5cc6sc7ccccc7c6cc5c4)nc(-c4cccc5ccccc45)n3)ccc2c1 ; c1ccc2c(-c3nc(-c4ccc5cc6c(cc5c4)sc4ccccc46)nc(-c4cccc5ccccc45)n3)cccc2c1 ; c1ccc2cc(-c3nc(-c4cccc5ccccc45)nc(-c4cccc5cc6c(cc45)sc4ccccc46)n3)ccc2c1 ; c1ccc(-c2nc(-c3ccc4c(c3)c3ccccc3n4-c3ccccc3)nc(-c3cccc4cc5sc6ccccc6c5cc34)n2)cc1]

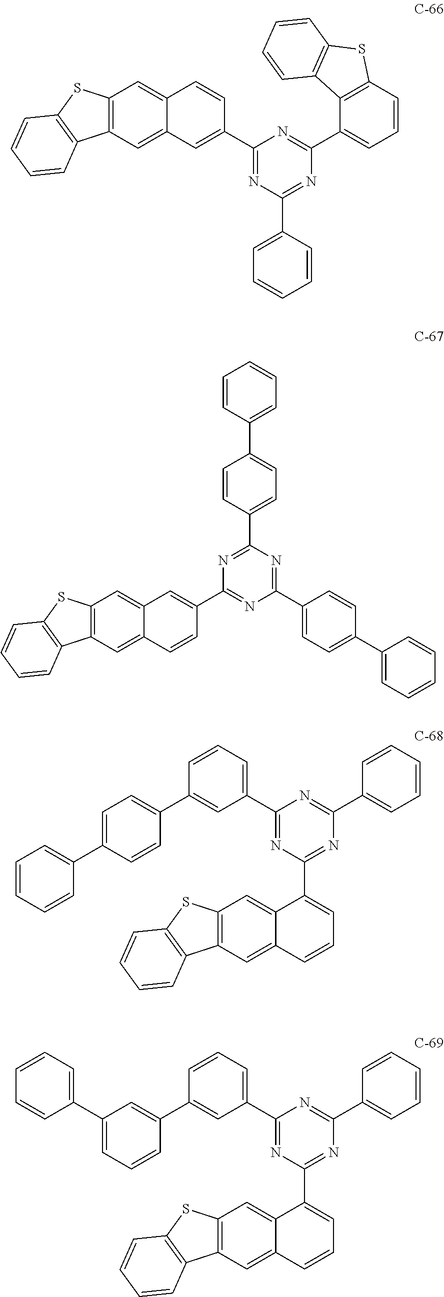 [SMILES: c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1 ; c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1]

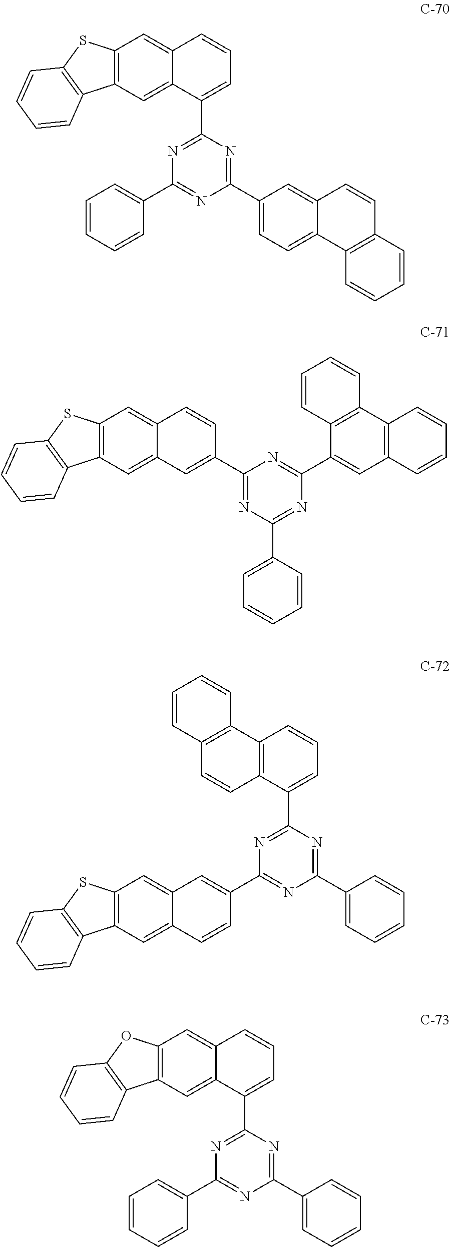 [SMILES: c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1 ; c1ccc(-c2nc(-c3ccccc3)nc(-c3cccc4cc5oc6ccccc6c5cc34)n2)cc1]

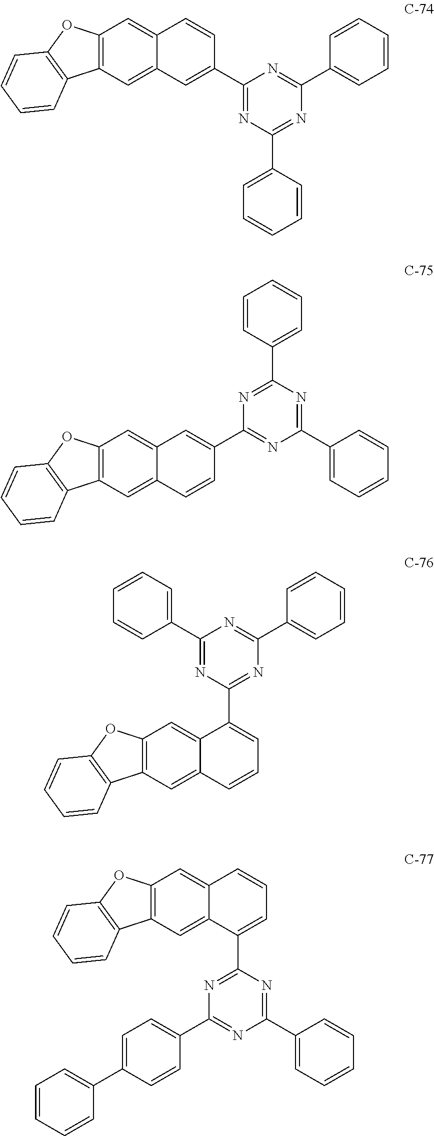

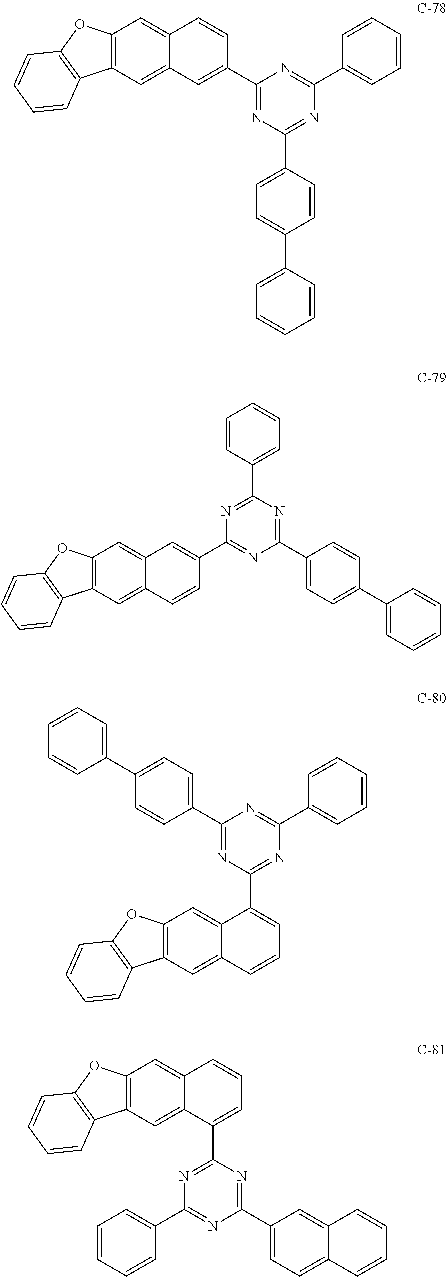 [SMILES: c1ccc(-c2ccc(-c3nc(-c4ccccc4)nc(-c4ccc5cc6oc7ccccc7c6cc5c4)n3)cc2)cc1 ; c1ccc(-c2ccc(-c3nc(-c4ccccc4)nc(-c4ccc5cc6c(cc5c4)oc4ccccc46)n3)cc2)cc1 ; c1ccc(-c2ccc(-c3nc(-c4ccccc4)nc(-c4cccc5cc6c(cc45)oc4ccccc46)n3)cc2)cc1 ; c1ccc(-c2nc(-c3ccc4ccccc4c3)nc(-c3cccc4cc5oc6ccccc6c5cc34)n2)cc1]

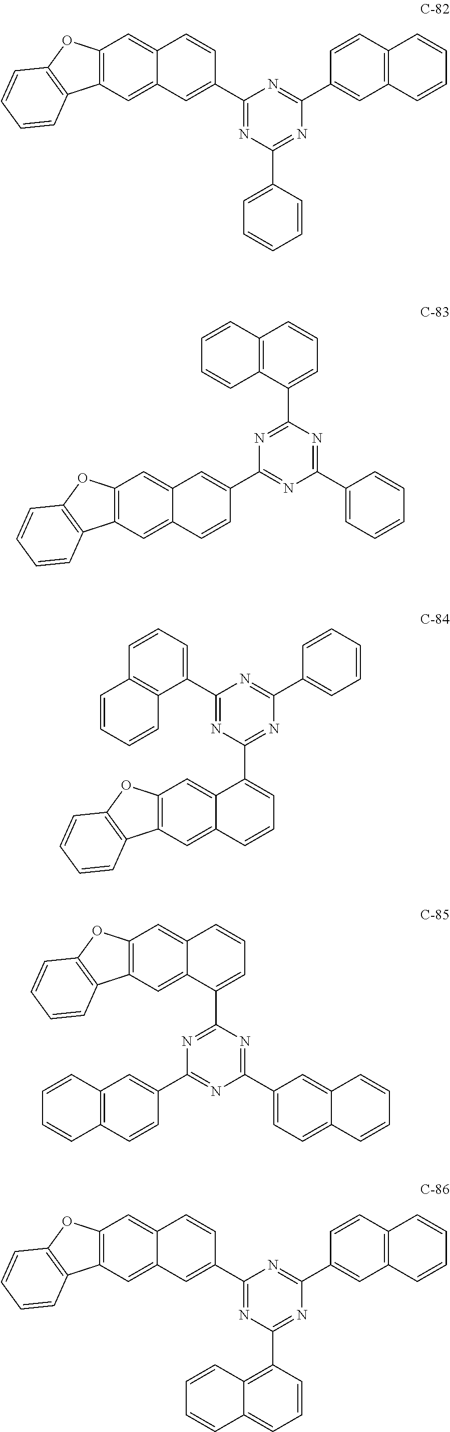 [SMILES: c1ccc(-c2nc(-c3ccc4ccccc4c3)nc(-c3ccc4cc5oc6ccccc6c5cc4c3)n2)cc1 ; c1ccc(-c2nc(-c3ccc4cc5c(cc4c3)oc3ccccc35)nc(-c3cccc4ccccc34)n2)cc1 ; c1ccc(-c2nc(-c3cccc4ccccc34)nc(-c3cccc4cc5c(cc34)oc3ccccc35)n2)cc1 ; c1ccc2cc(-c3nc(-c4ccc5ccccc5c4)nc(-c4cccc5cc6oc7ccccc7c6cc45)n3)ccc2c1 ; c1ccc2cc(-c3nc(-c4ccc5cc6oc7ccccc7c6cc5c4)nc(-c4cccc5ccccc45)n3)ccc2c1]

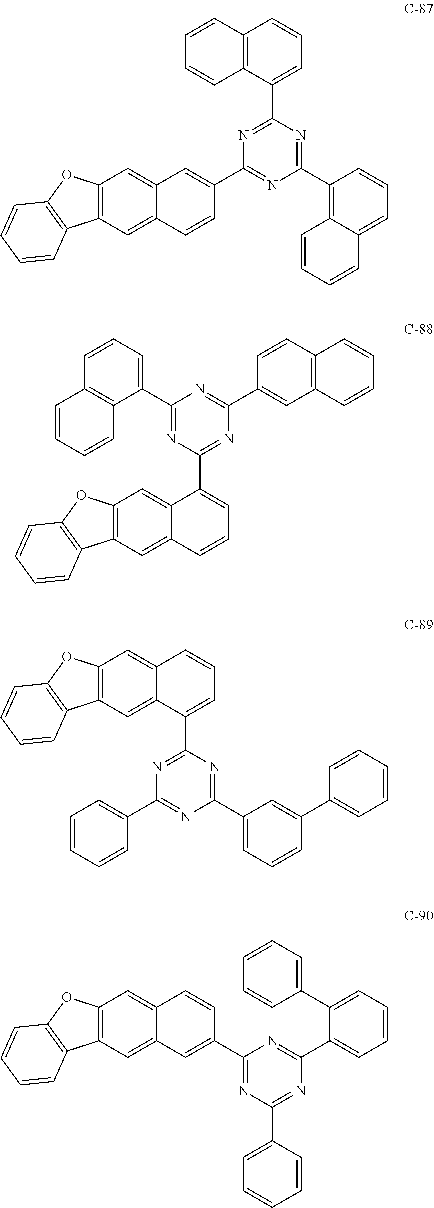 [SMILES: c1ccc2c(-c3nc(-c4ccc5cc6c(cc5c4)oc4ccccc46)nc(-c4cccc5ccccc45)n3)cccc2c1 ; c1ccc2cc(-c3nc(-c4cccc5ccccc45)nc(-c4cccc5cc6c(cc45)oc4ccccc46)n3)ccc2c1 ; c1ccc(-c2cccc(-c3nc(-c4ccccc4)nc(-c4cccc5cc6oc7ccccc7c6cc45)n3)c2)cc1 ; c1ccc(-c2nc(-c3ccc4cc5oc6ccccc6c5cc4c3)nc(-c3ccccc3-c3ccccc3)n2)cc1]

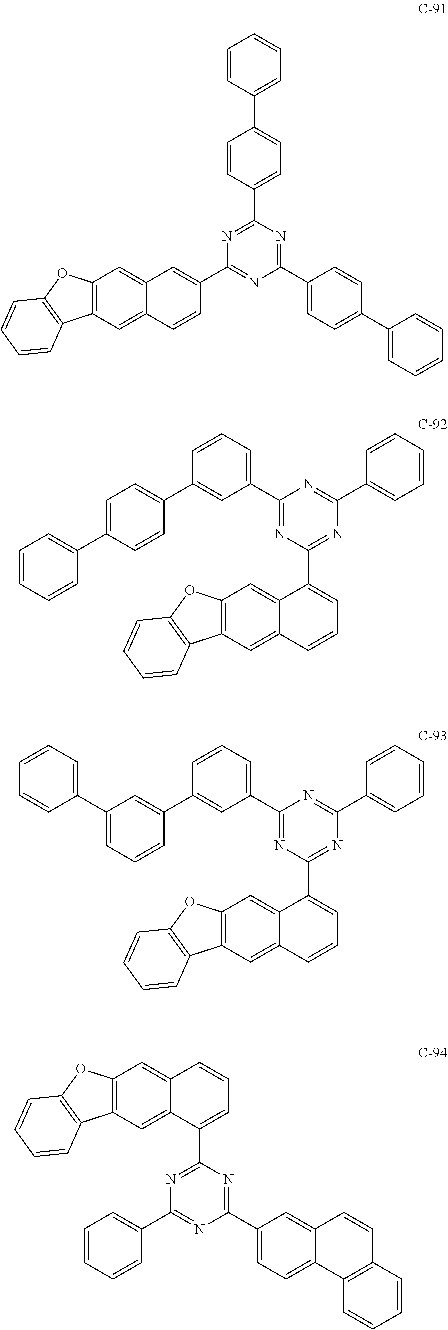 [SMILES: c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1 ; c1ccc(-c2nc(-c3ccc4c(ccc5ccccc54)c3)nc(-c3cccc4cc5oc6ccccc6c5cc34)n2)cc1]

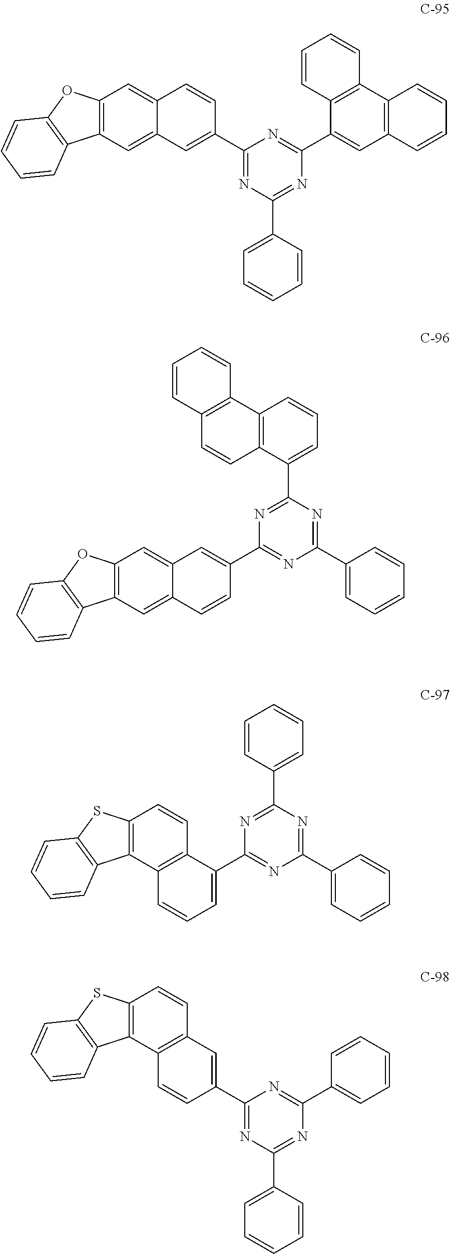 [SMILES: c1ccc(-c2nc(-c3ccc4cc5oc6ccccc6c5cc4c3)nc(-c3cc4ccccc4c4ccccc34)n2)cc1 ; c1ccc(-c2nc(-c3ccc4cc5c(cc4c3)oc3ccccc35)nc(-c3cccc4c3ccc3ccccc34)n2)cc1 ; c1ccc(-c2nc(-c3ccccc3)nc(-c3cccc4c3ccc3sc5ccccc5c34)n2)cc1 ; c1ccc(-c2nc(-c3ccccc3)nc(-c3ccc4c(ccc5sc6ccccc6c54)c3)n2)cc1]

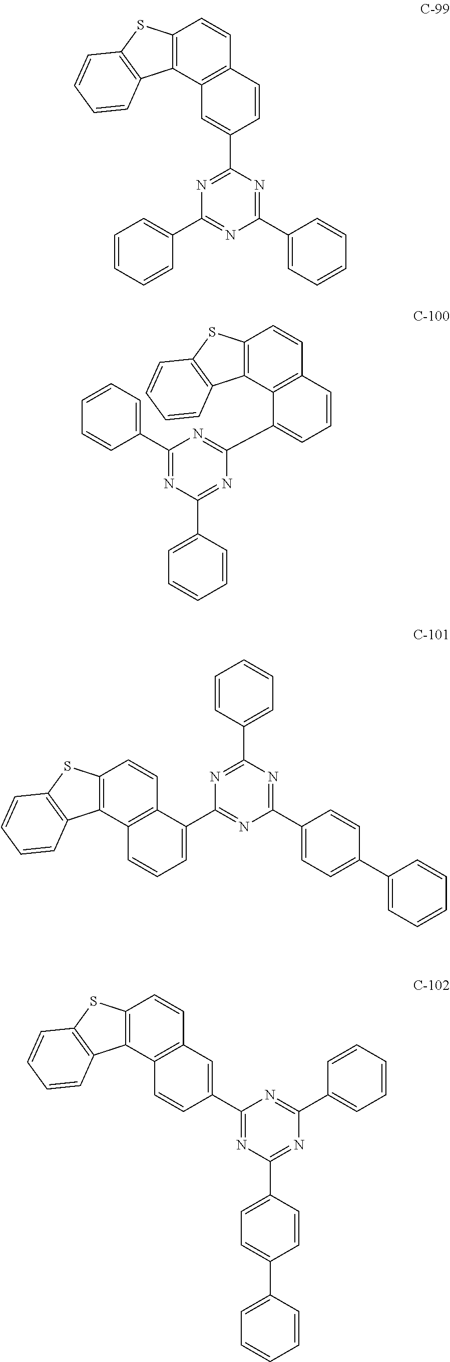 [SMILES: c1ccc(-c2nc(-c3ccccc3)nc(-c3ccc4ccc5sc6ccccc6c5c4c3)n2)cc1 ; c1ccc(-c2nc(-c3ccccc3)nc(-c3cccc4ccc5sc6ccccc6c5c34)n2)cc1 ; c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1]

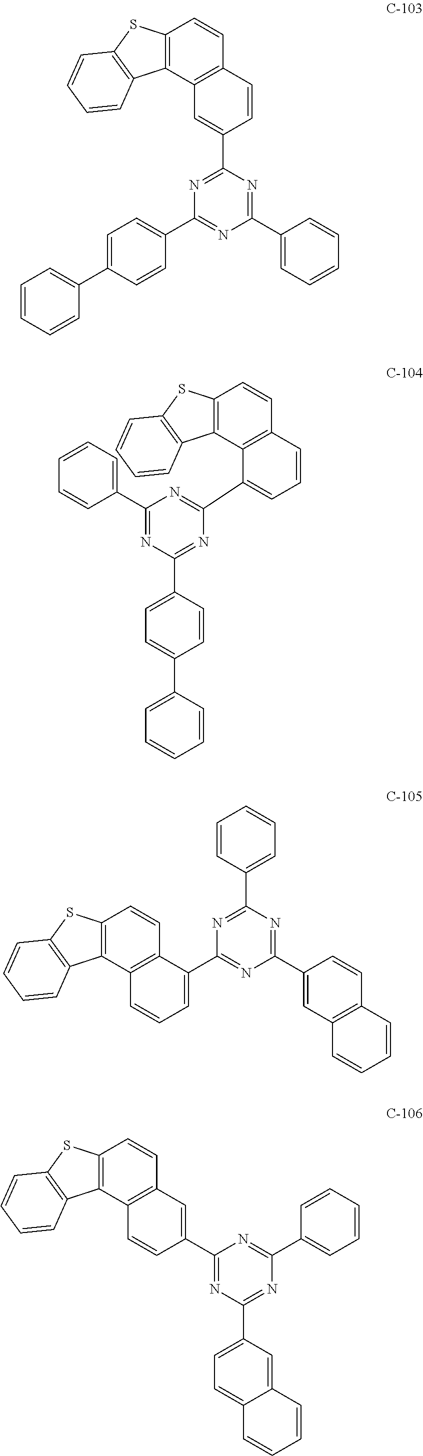 [SMILES: c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1 ; c1ccc(-c2nc(-c3ccc4ccccc4c3)nc(-c3ccc4c(ccc5sc6ccccc6c54)c3)n2)cc1]

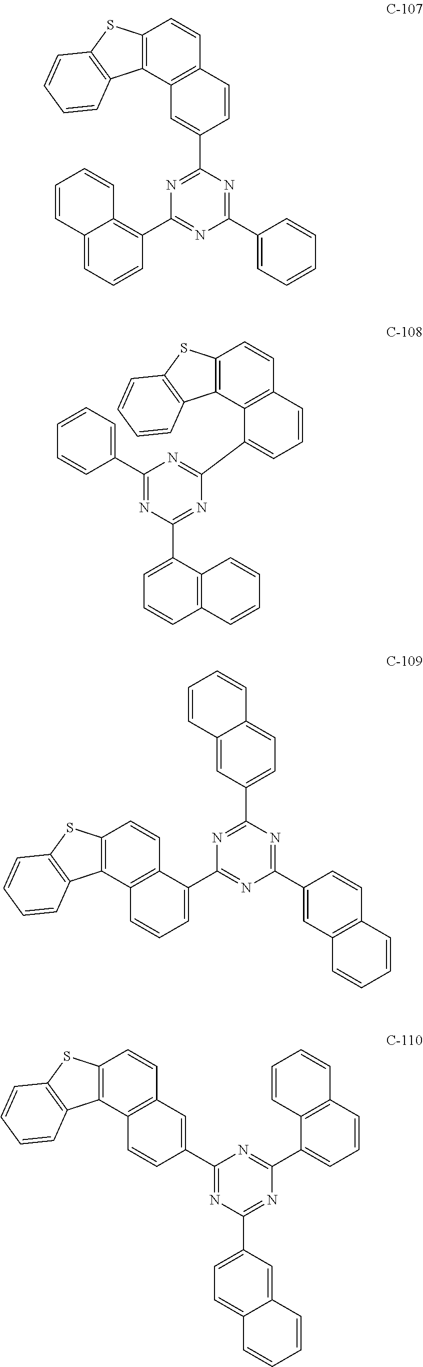 [SMILES: c1ccc(-c2nc(-c3ccc4ccc5sc6ccccc6c5c4c3)nc(-c3cccc4ccccc34)n2)cc1 ; c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1 ; c1ccc2cc(-c3nc(-c4ccc5c(ccc6sc7ccccc7c65)c4)nc(-c4cccc5ccccc45)n3)ccc2c1]

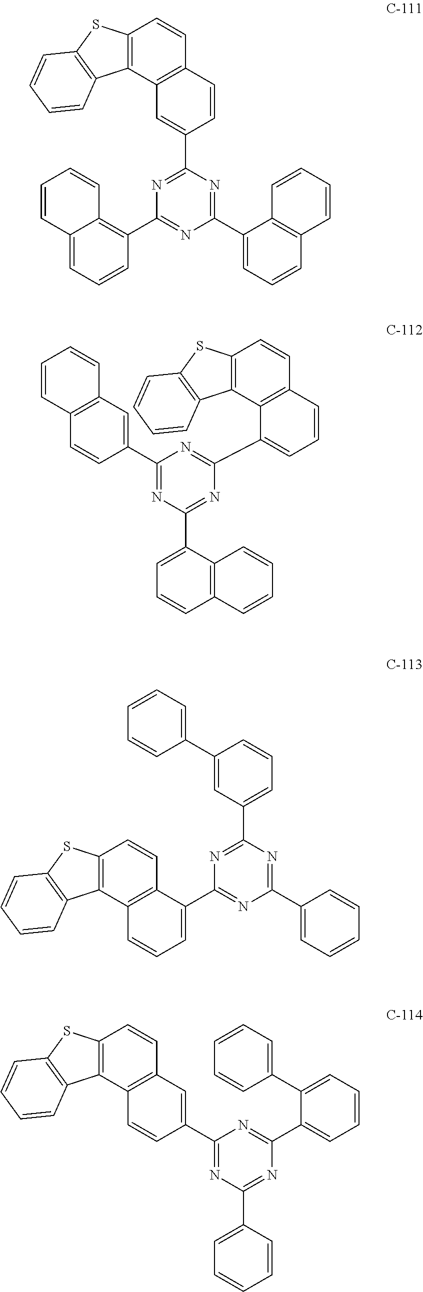 [SMILES: c1ccc2c(-c3nc(-c4ccc5ccc6sc7ccccc7c6c5c4)nc(-c4cccc5ccccc45)n3)cccc2c1 ; c1ccc2cc(-c3nc(-c4cccc5ccccc45)nc(-c4cccc5ccc6sc7ccccc7c6c45)n3)ccc2c1 ; c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1]

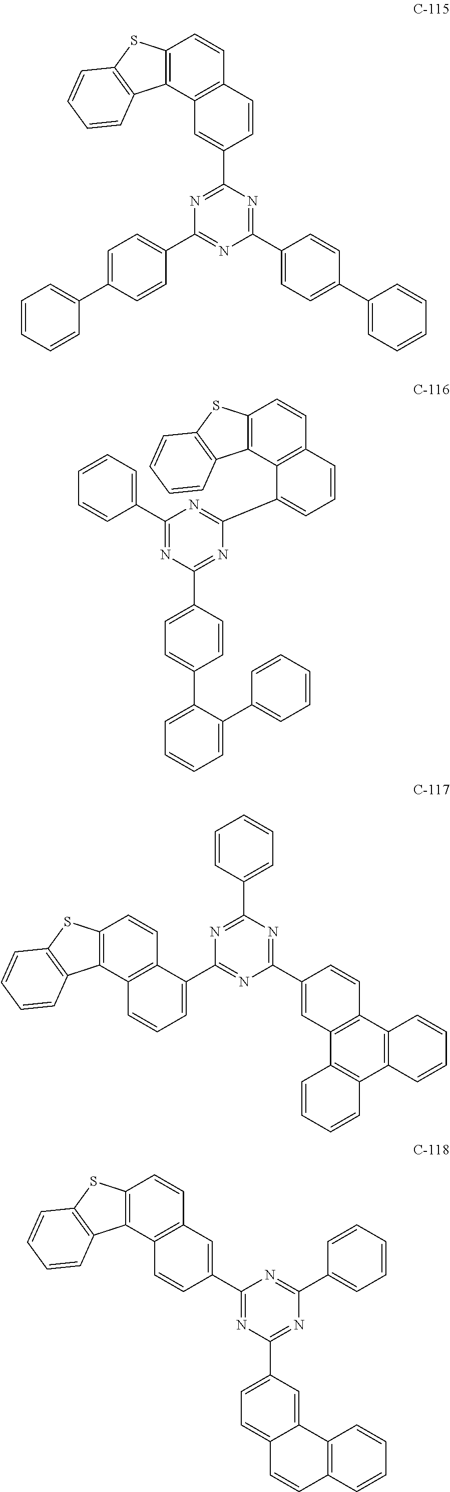 [SMILES: c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1 ; c1ccc(-c2nc(-c3ccc4c(ccc5sc6ccccc6c54)c3)nc(-c3ccc4ccc5ccccc5c4c3)n2)cc1]

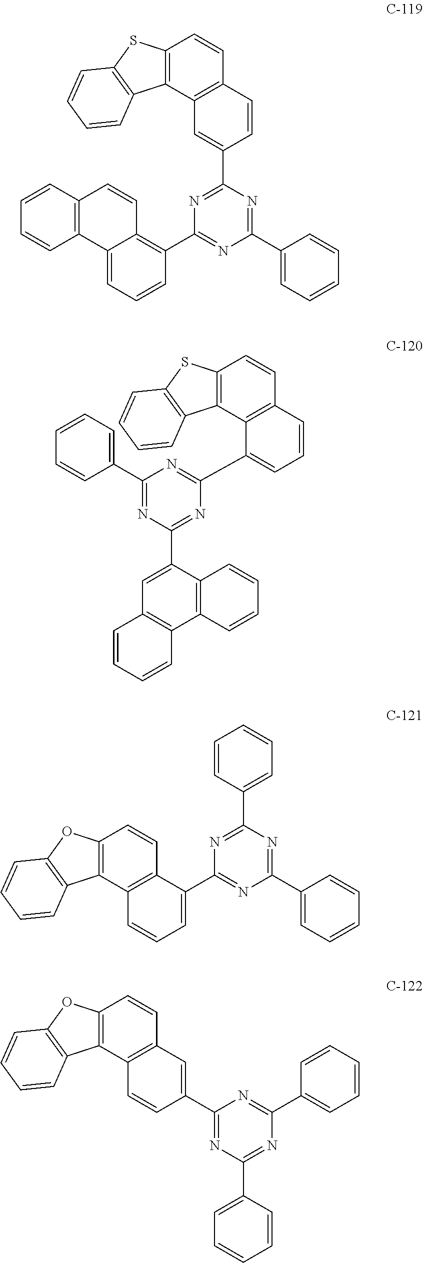 [SMILES: c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1 ; c1ccc(-c2nc(-c3ccccc3)nc(-c3cccc4c3ccc3oc5ccccc5c34)n2)cc1 ; c1ccc(-c2nc(-c3ccccc3)nc(-c3ccc4c(ccc5oc6ccccc6c54)c3)n2)cc1]

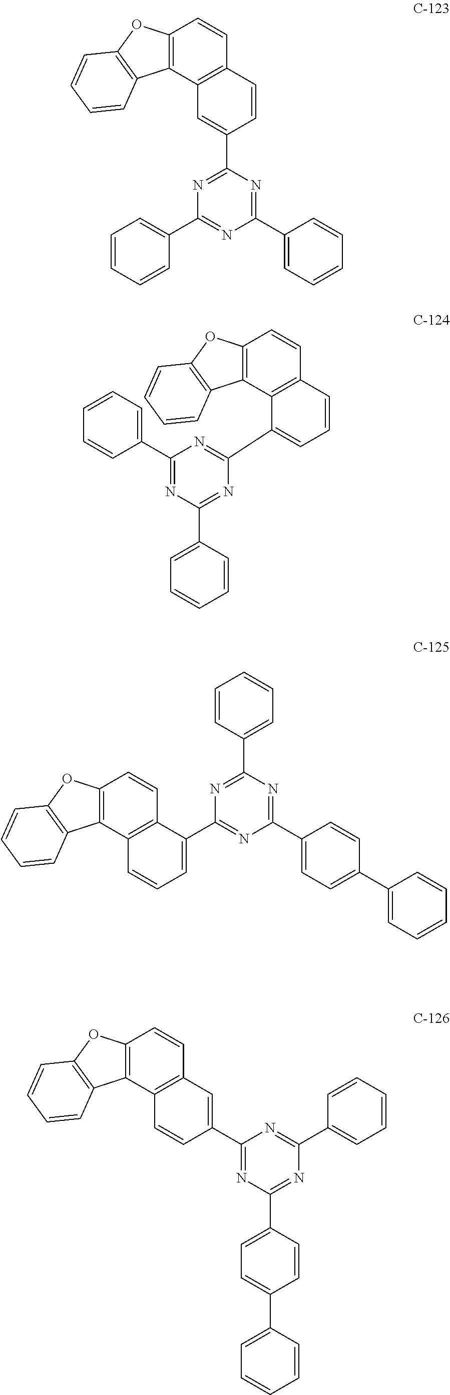 [SMILES: c1ccc(-c2nc(-c3ccccc3)nc(-c3ccc4ccc5oc6ccccc6c5c4c3)n2)cc1 ; c1ccc(-c2nc(-c3ccccc3)nc(-c3cccc4ccc5oc6ccccc6c5c34)n2)cc1 ; c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1]

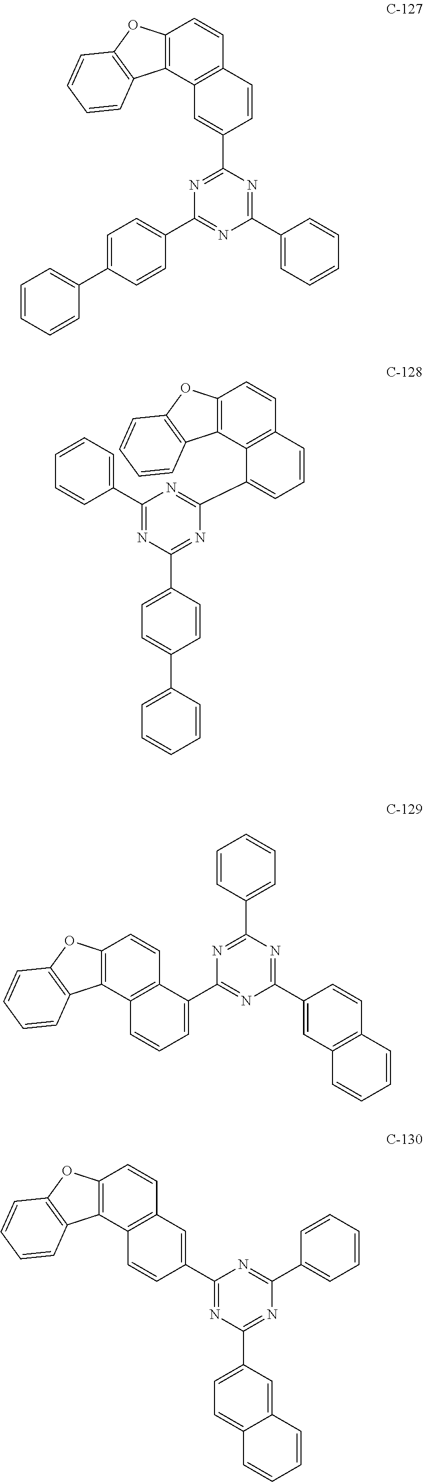 [SMILES: c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1 ; c1ccc(-c2nc(-c3ccc4ccccc4c3)nc(-c3cccc4c3ccc3oc5ccccc5c34)n2)cc1 ; c1ccc(-c2nc(-c3ccc4ccccc4c3)nc(-c3ccc4c(ccc5oc6ccccc6c54)c3)n2)cc1]

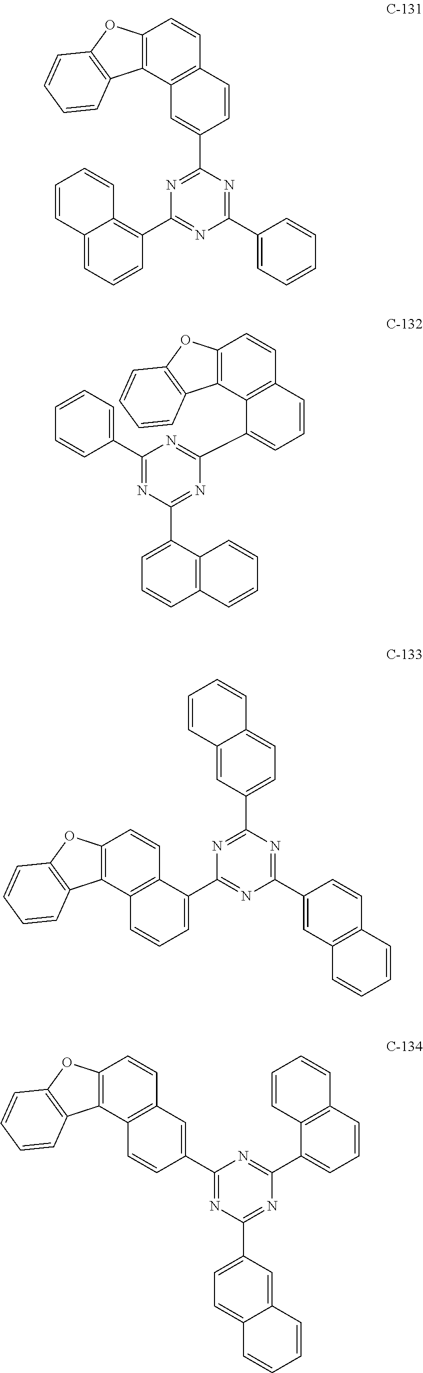 [SMILES: c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1 ; c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1]

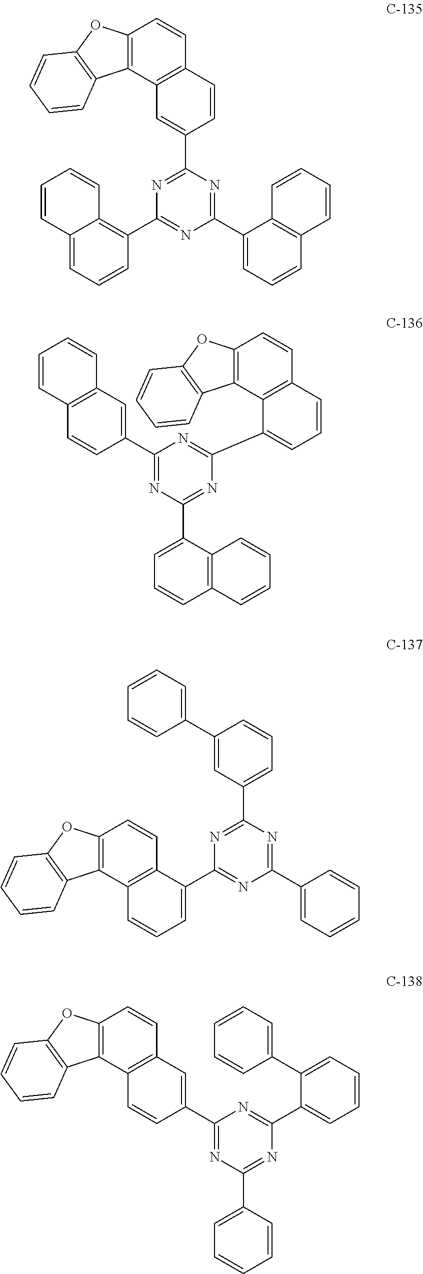 [SMILES: c1ccc2c(-c3nc(-c4ccc5ccc6oc7ccccc7c6c5c4)nc(-c4cccc5ccccc45)n3)cccc2c1 ; c1ccc2cc(-c3nc(-c4cccc5ccccc45)nc(-c4cccc5ccc6oc7ccccc7c6c45)n3)ccc2c1 ; c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1]

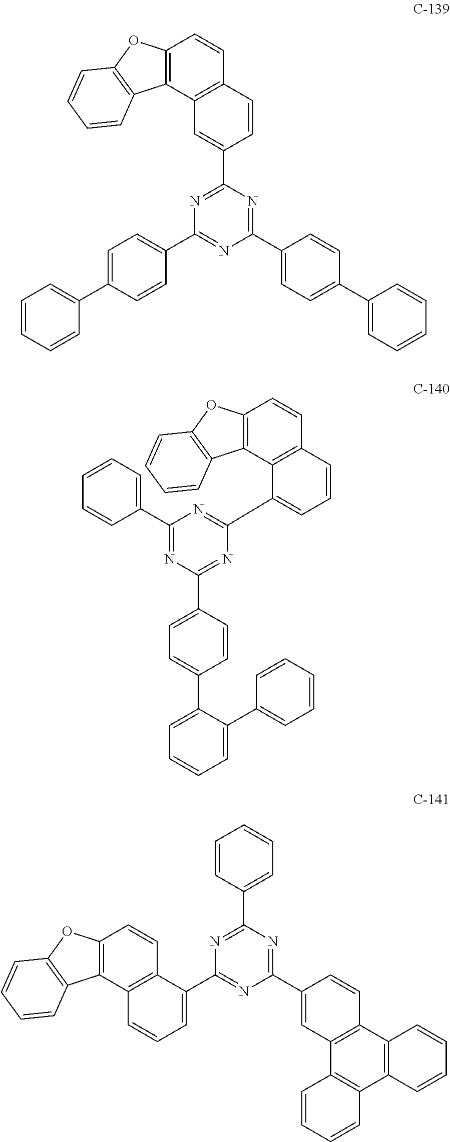 [SMILES: c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1 ; c1ccc(-c2nc(-c3ccc4c5ccccc5c5ccccc5c4c3)nc(-c3cccc4c3ccc3oc5ccccc5c34)n2)cc1]

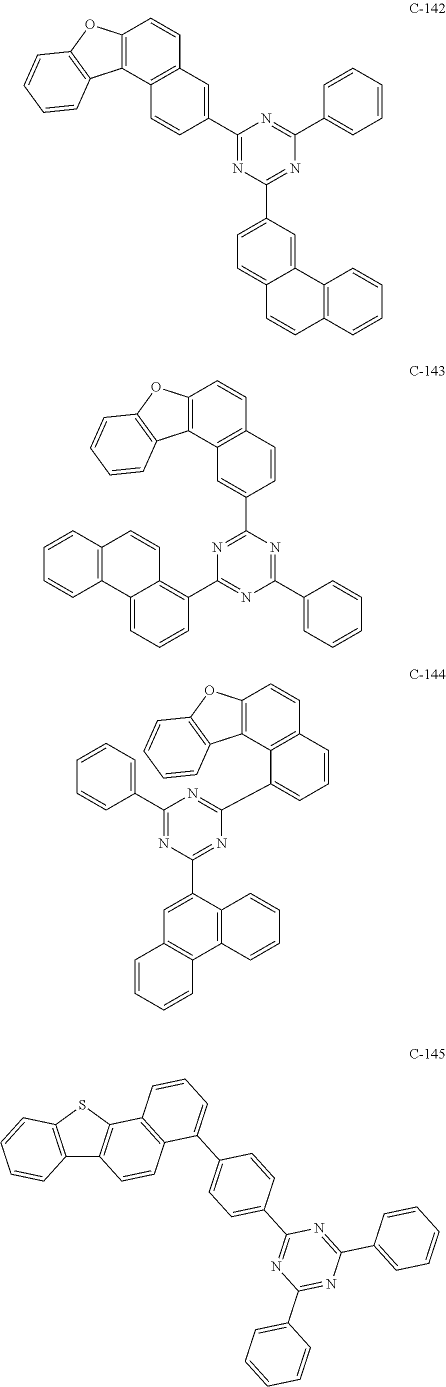 [SMILES: c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1 ; c1ccc(-c2nc(-c3ccccc3)nc(-c3ccc(-c4cccc5c4ccc4c6ccccc6sc54)cc3)n2)cc1]

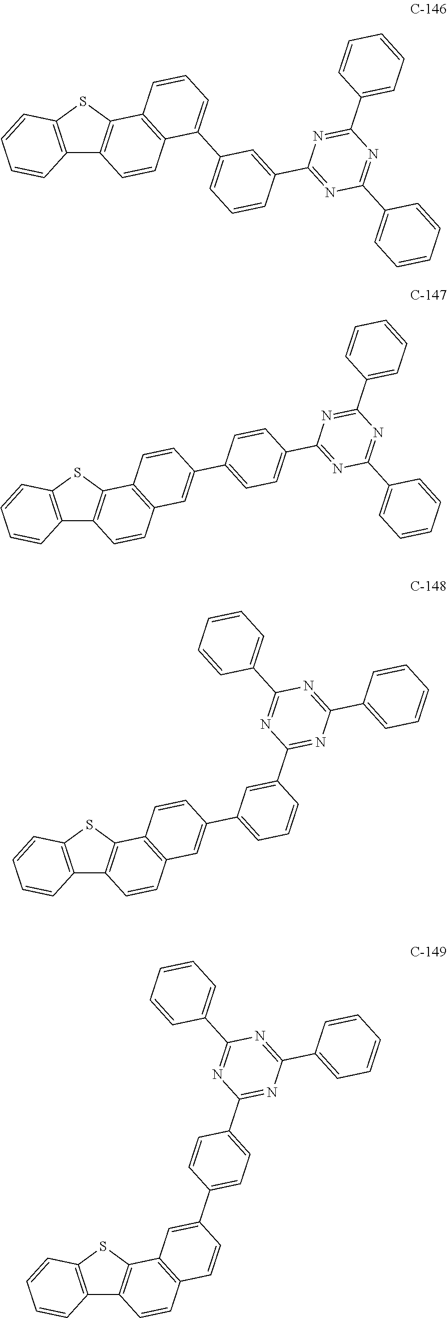 [SMILES: c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1]

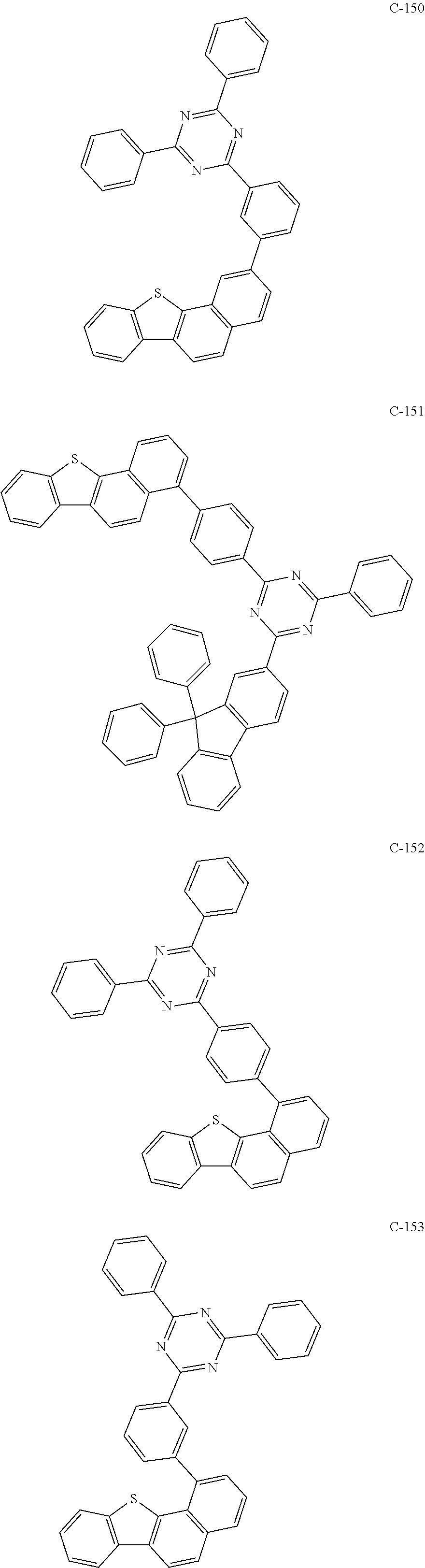 [SMILES: c1ccc(-c2nc(-c3ccccc3)nc(-c3cccc(-c4ccc5ccc6c7ccccc7sc6c5c4)c3)n2)cc1 ; c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1 ; c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc(-c4cccc5ccc6c7ccccc7sc6c45)c3)n2)cc1]

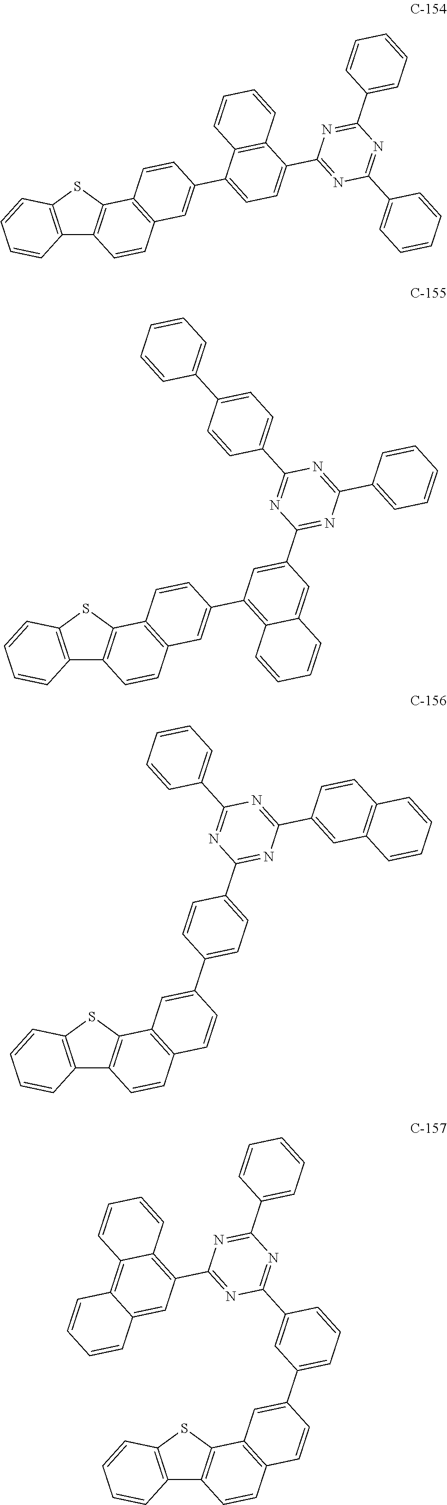 [SMILES: c1ccc(-c2nc(-c3ccccc3)nc(-c3ccc(-c4ccc5c(ccc6c7ccccc7sc56)c4)c4ccccc34)n2)cc1 ; c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1]

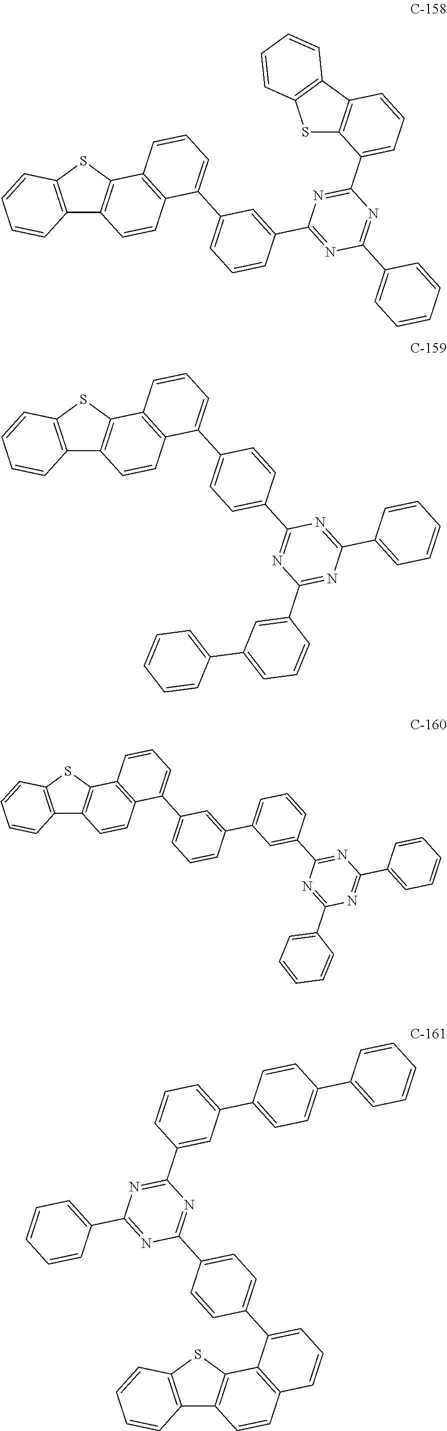 [SMILES: c1ccc(-c2nc(-c3cccc(-c4cccc5c4ccc4c6ccccc6sc54)c3)nc(-c3cccc4c3sc3ccccc34)n2)cc1 ; c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1]

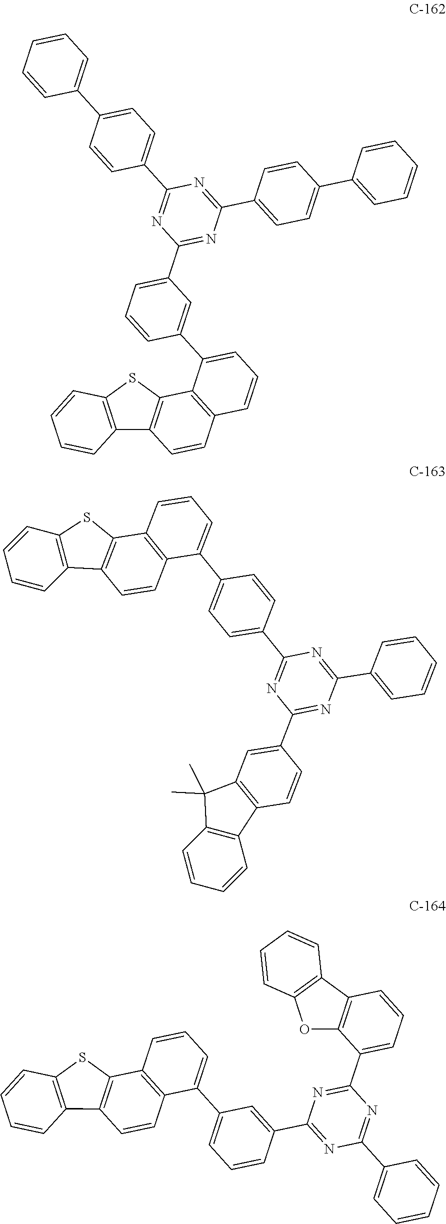 [SMILES: c1ccc(-c2ccc(-c3nc(-c4ccc(-c5ccccc5)cc4)nc(-c4cccc(-c5cccc6ccc7c8ccccc8sc7c56)c4)n3)cc2)cc1 ; CC1(C)c2ccccc2-c2ccc(-c3nc(-c4ccccc4)nc(-c4ccc(-c5cccc6c5ccc5c7ccccc7sc65)cc4)n3)cc21 ; c1ccc(-c2nc(-c3cccc(-c4cccc5c4ccc4c6ccccc6sc54)c3)nc(-c3cccc4c3oc3ccccc34)n2)cc1]

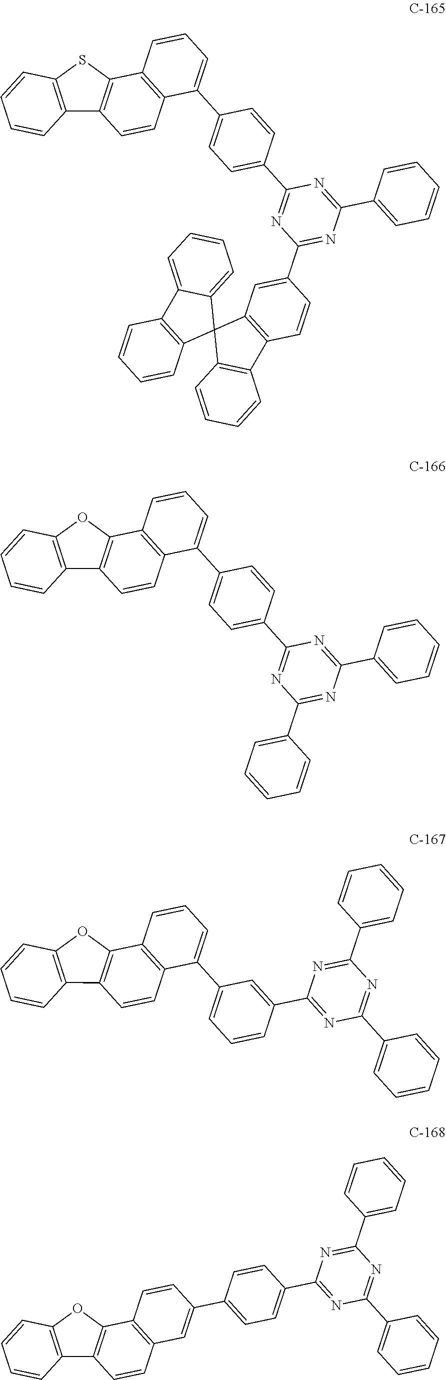 [SMILES: c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1 ; c1ccc(-c2nc(-c3ccccc3)nc(-c3ccc(-c4ccc5c(ccc6c7ccccc7oc56)c4)cc3)n2)cc1]

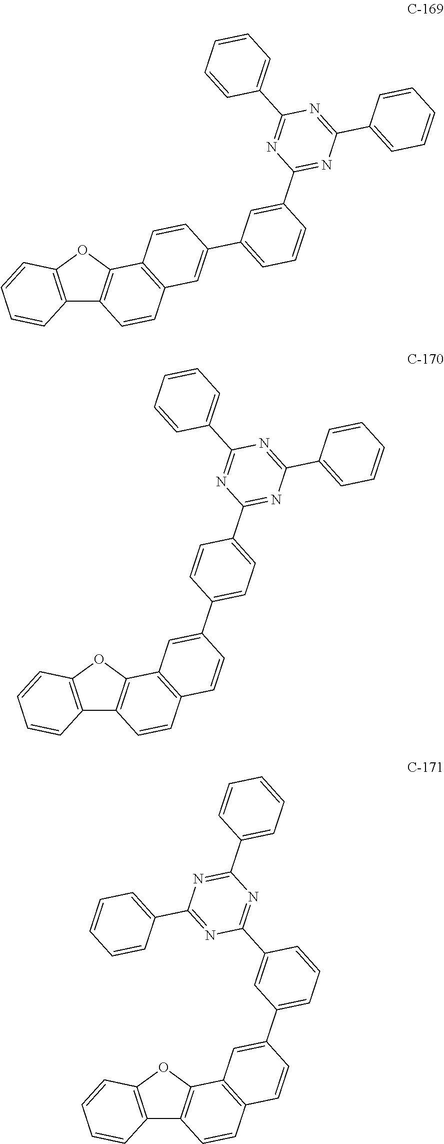 [SMILES: c1ccc(-c2nc(-c3ccccc3)nc(-c3cccc(-c4ccc5c(ccc6c7ccccc7oc56)c4)c3)n2)cc1 ; c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1]

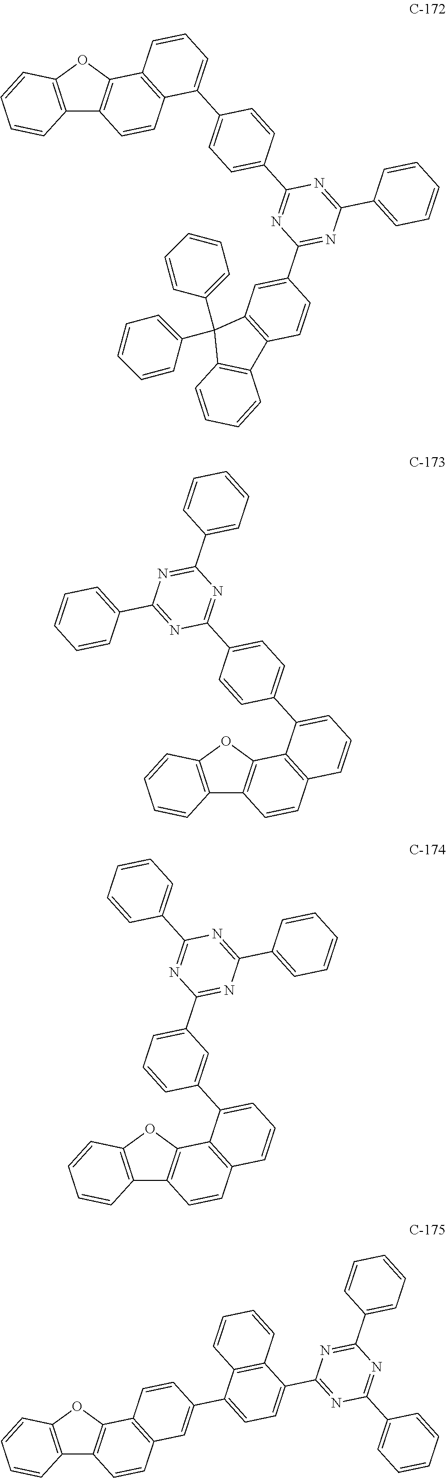

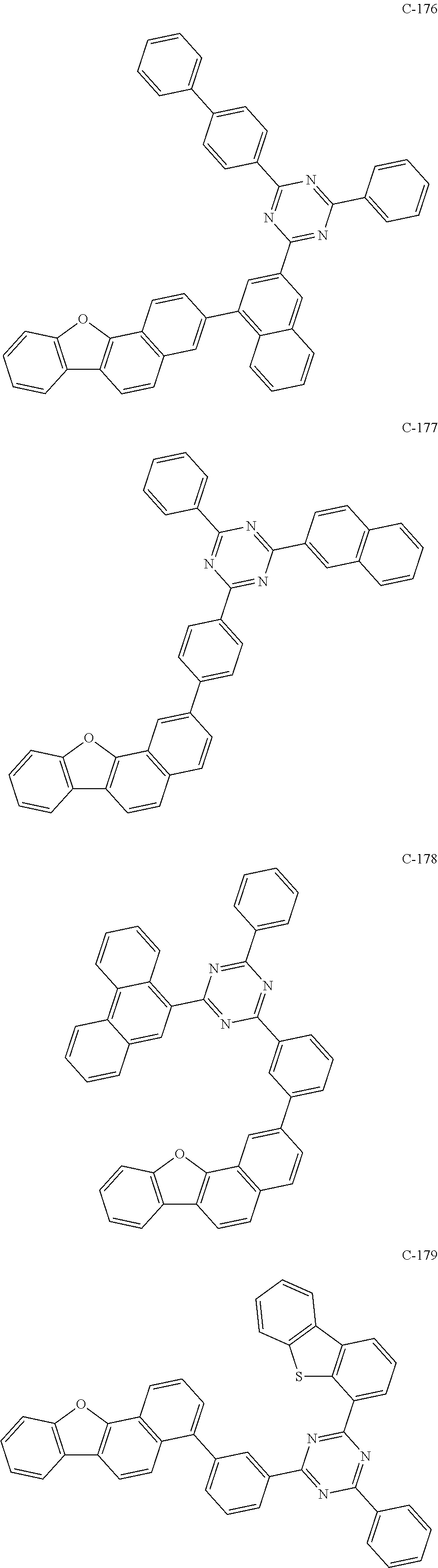 [SMILES: c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1 ; c1ccc(-c2nc(-c3cccc(-c4ccc5ccc6c7ccccc7oc6c5c4)c3)nc(-c3cc4ccccc4c4ccccc34)n2)cc1 ; c1ccc(-c2nc(-c3cccc(-c4cccc5c4ccc4c6ccccc6oc54)c3)nc(-c3cccc4c3sc3ccccc34)n2)cc1]

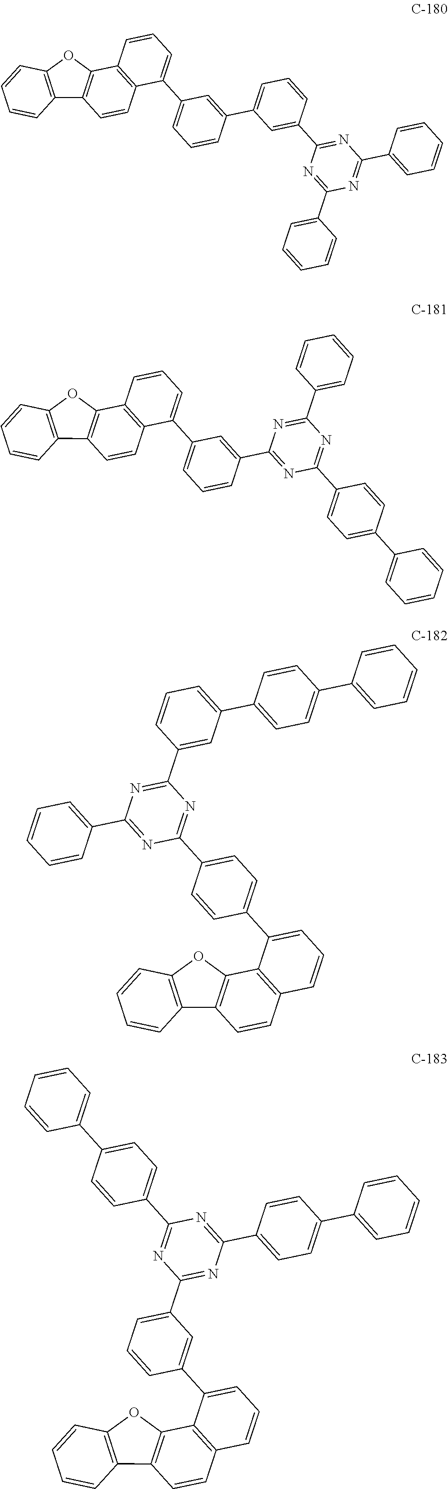

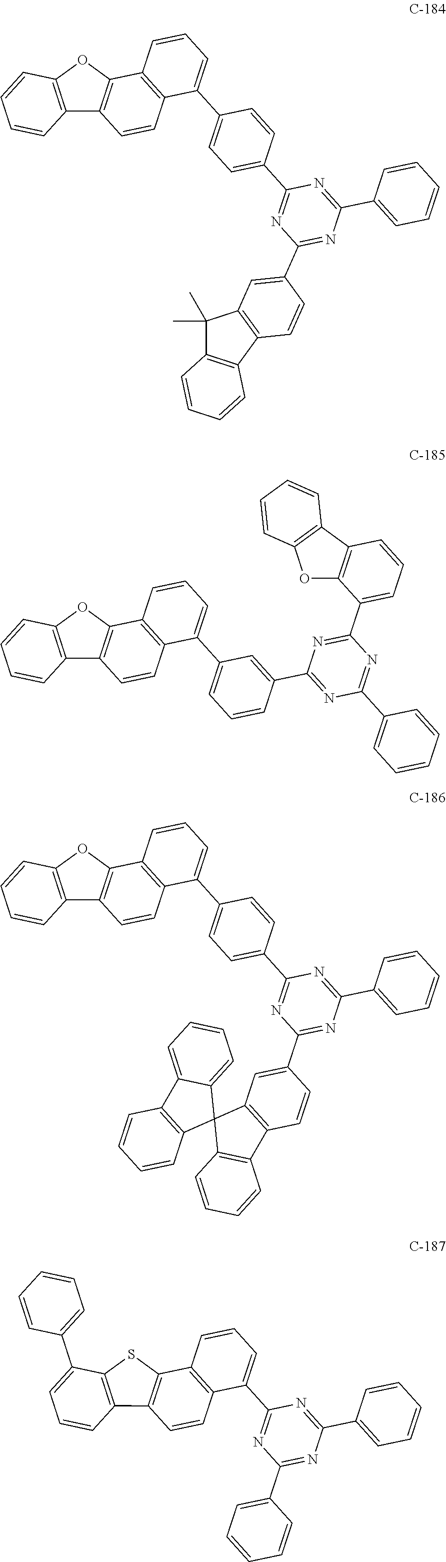 [SMILES: CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1 ; c1ccc(-c2nc(-c3ccccc3)nc(-c3cccc4c3ccc3c5cccc(-c6ccccc6)c5sc43)n2)cc1]

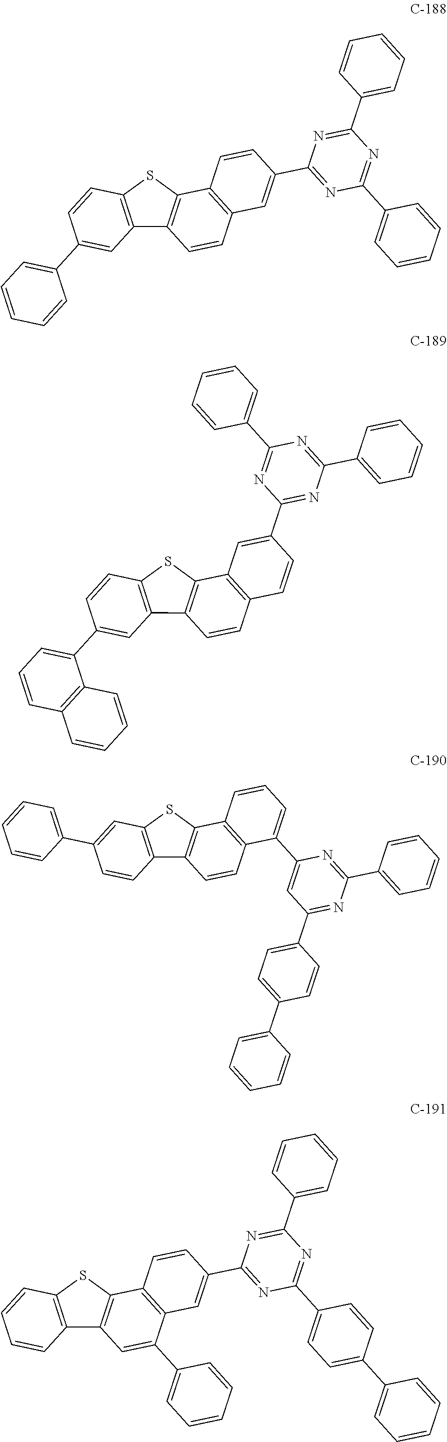 [SMILES: c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1 ; c1ccc(-c2ccc(-c3cc(-c4cccc5c4ccc4c6ccc(-c7ccccc7)cc6sc54)nc(-c4ccccc4)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1]

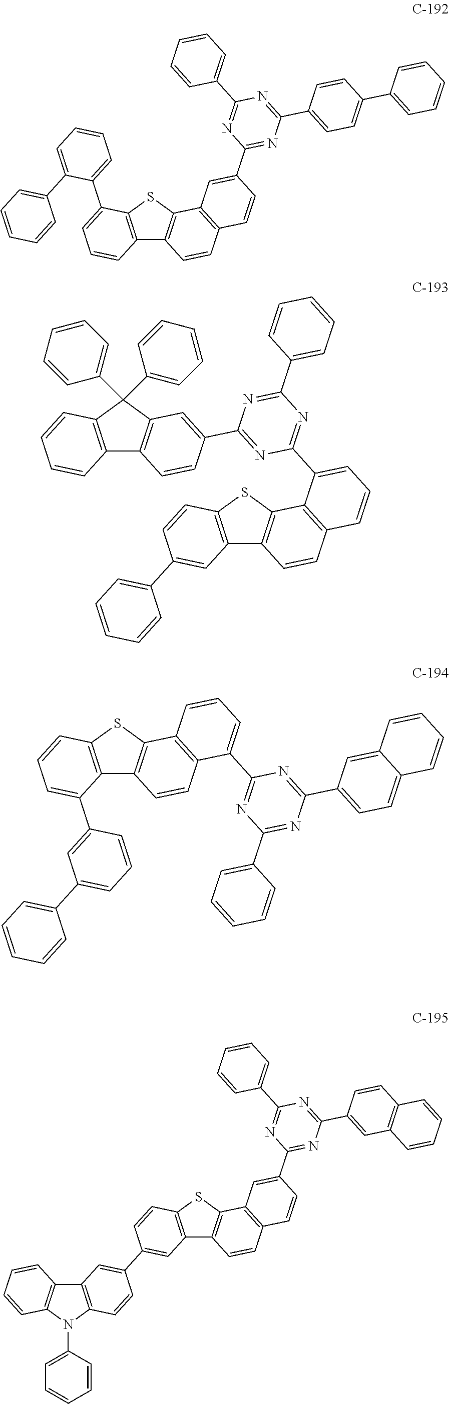 [SMILES: c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4sc5c6cccc(-c7nc(-c8ccccc8)nc(-c8ccc9ccccc9c8)n7)c6ccc5c34)c2)cc1 ; c1ccc(-c2nc(-c3ccc4ccccc4c3)nc(-c3ccc4ccc5c6cc(-c7ccc8c(c7)c7ccccc7n8-c7ccccc7)ccc6sc5c4c3)n2)cc1]

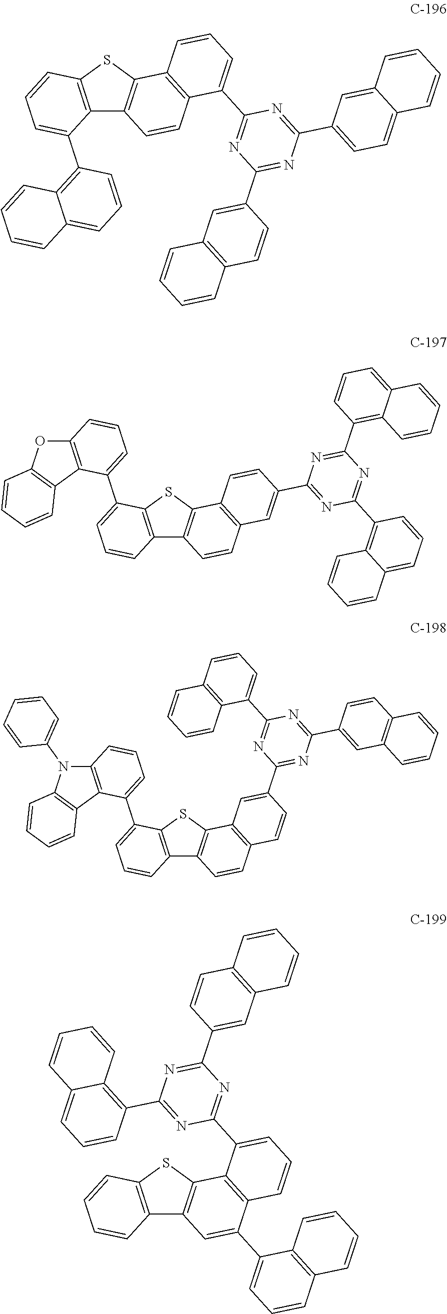 [SMILES: c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1 ; c1ccc(-n2c3ccccc3c3c(-c4cccc5c4sc4c6cc(-c7nc(-c8ccc9ccccc9c8)nc(-c8cccc9ccccc89)n7)ccc6ccc54)cccc32)cc1 ; c1ccc2cc(-c3nc(-c4cccc5ccccc45)nc(-c4cccc5c(-c6cccc7ccccc67)cc6c7ccccc7sc6c45)n3)ccc2c1]

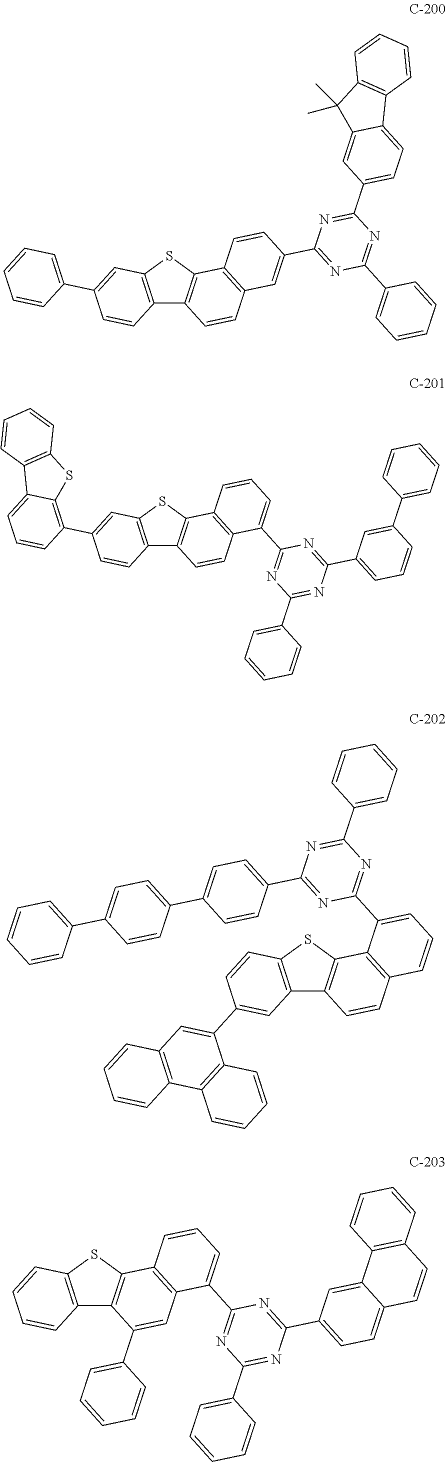 [SMILES: CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1]

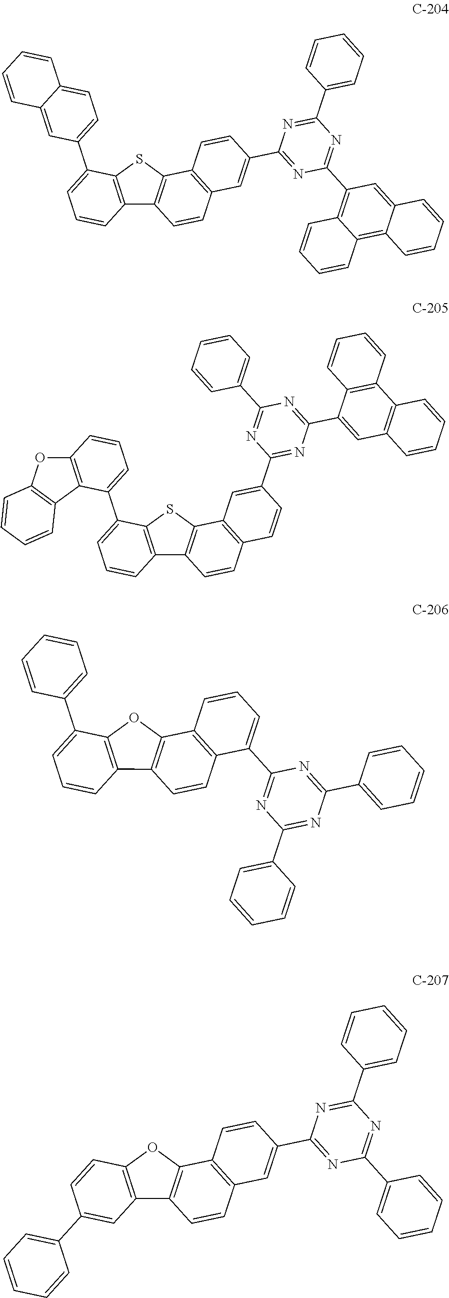 [SMILES: c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1 ; c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1]

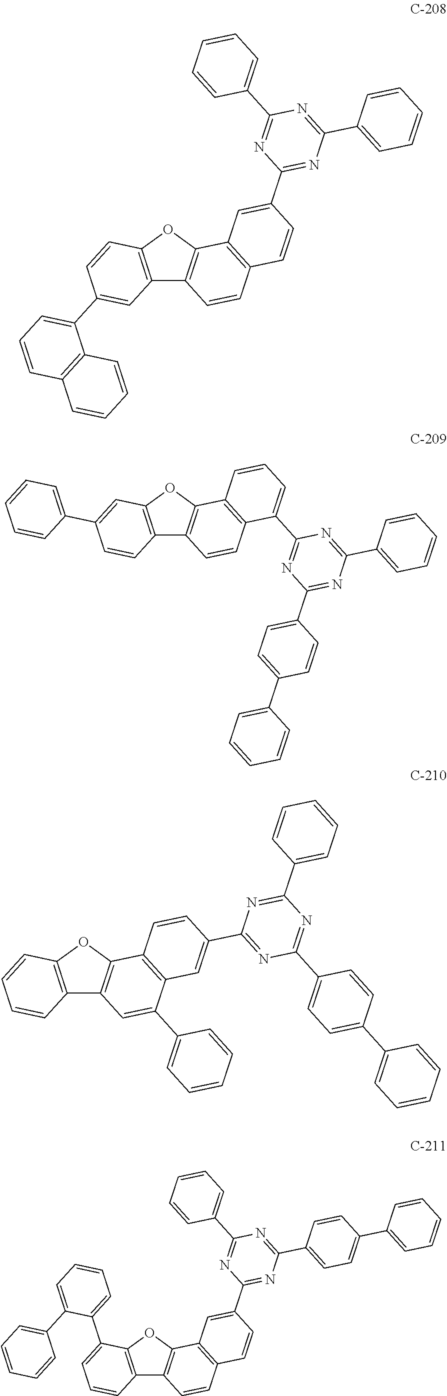 [SMILES: c1ccc(-c2nc(-c3ccccc3)nc(-c3ccc4ccc5c6cc(-c7cccc8ccccc78)ccc6oc5c4c3)n2)cc1 ; c1ccc(-c2ccc(-c3nc(-c4ccccc4)nc(-c4cccc5c4ccc4c6ccc(-c7ccccc7)cc6oc54)n3)cc2)cc1 ; c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1]

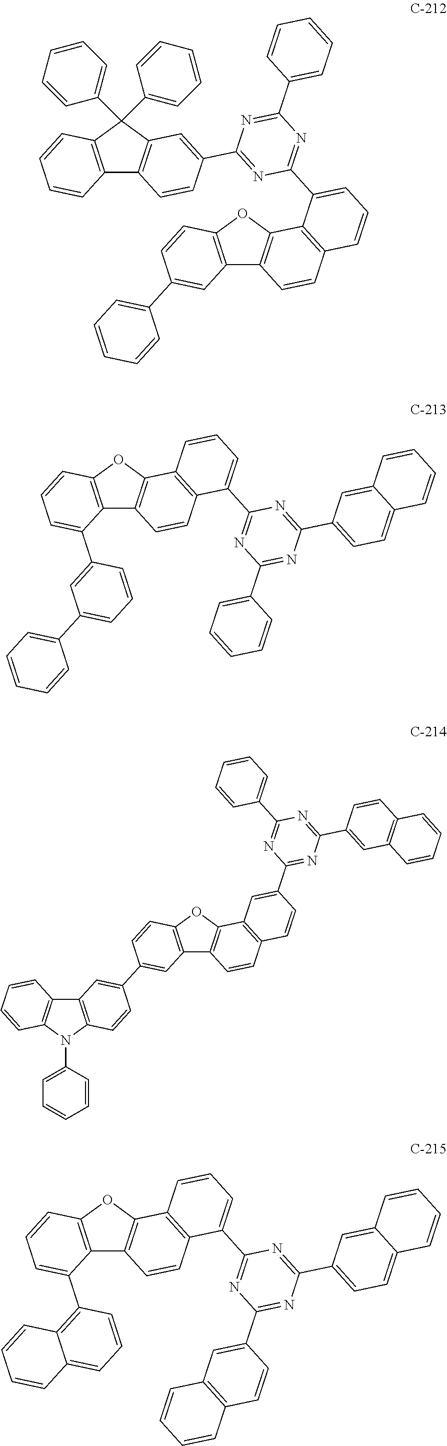 [SMILES: c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4oc5c6cccc(-c7nc(-c8ccccc8)nc(-c8ccc9ccccc9c8)n7)c6ccc5c34)c2)cc1 ; c1ccc(-c2nc(-c3ccc4ccccc4c3)nc(-c3ccc4ccc5c6cc(-c7ccc8c(c7)c7ccccc7n8-c7ccccc7)ccc6oc5c4c3)n2)cc1 ; c1ccc2cc(-c3nc(-c4ccc5ccccc5c4)nc(-c4cccc5c4ccc4c5oc5cccc(-c6cccc7ccccc67)c54)n3)ccc2c1]

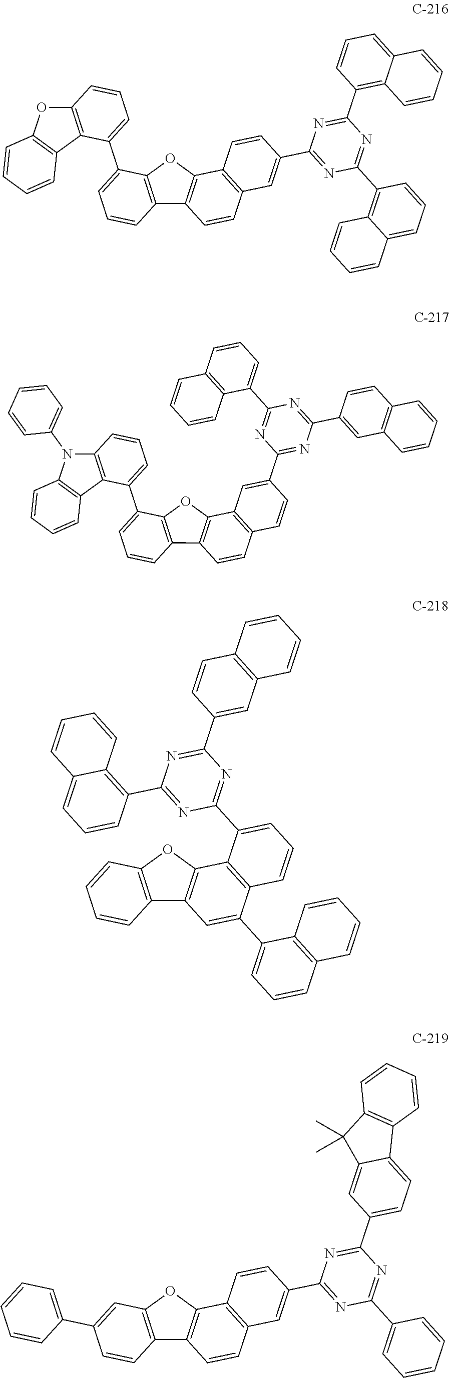 [SMILES: c1ccc2c(-c3nc(-c4ccc5c(ccc6c7cccc(-c8cccc9oc%10ccccc%10c89)c7oc56)c4)nc(-c4cccc5ccccc45)n3)cccc2c1 ; c1ccc(-n2c3ccccc3c3c(-c4cccc5c4oc4c6cc(-c7nc(-c8ccc9ccccc9c8)nc(-c8cccc9ccccc89)n7)ccc6ccc54)cccc32)cc1 ; c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21]

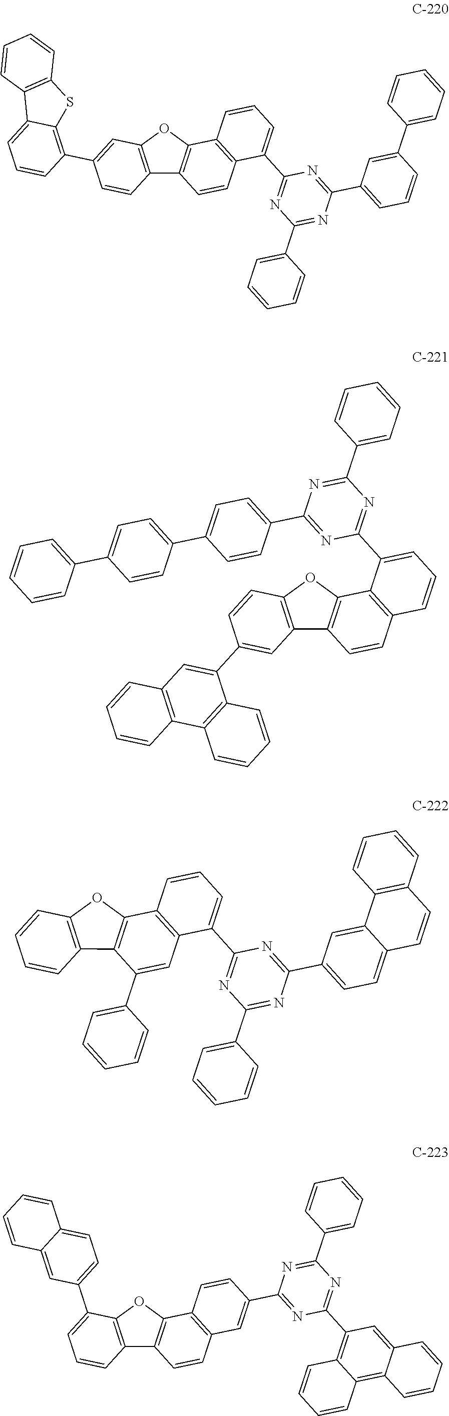 [SMILES: c1ccc(-c2cccc(-c3nc(-c4ccccc4)nc(-c4cccc5c4ccc4c6ccc(-c7cccc8c7sc7ccccc78)cc6oc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1]

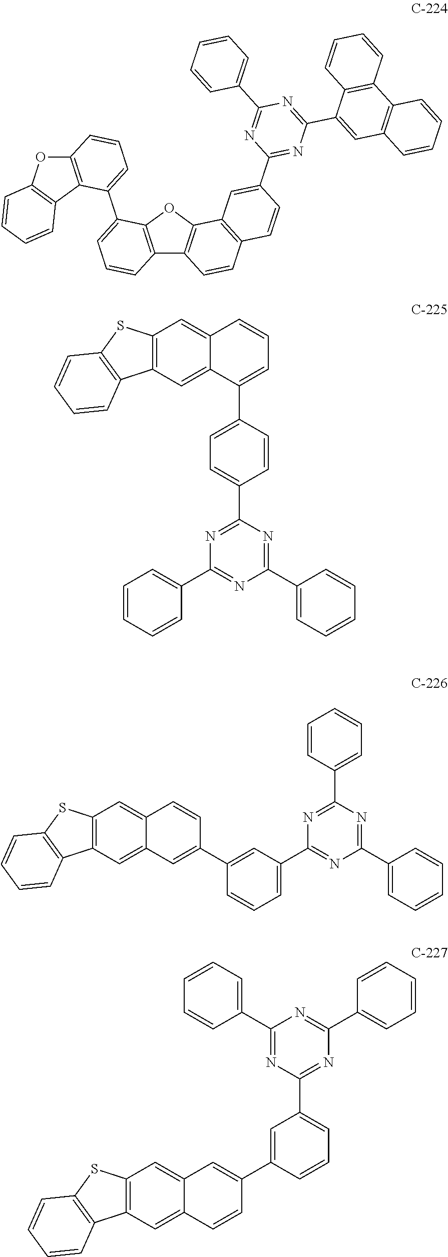 [SMILES: c1ccc(-c2nc(-c3ccc4ccc5c6cccc(-c7cccc8oc9ccccc9c78)c6oc5c4c3)nc(-c3cc4ccccc4c4ccccc34)n2)cc1 ; c1ccc(-c2nc(-c3ccccc3)nc(-c3ccc(-c4cccc5cc6sc7ccccc7c6cc45)cc3)n2)cc1 ; c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1]

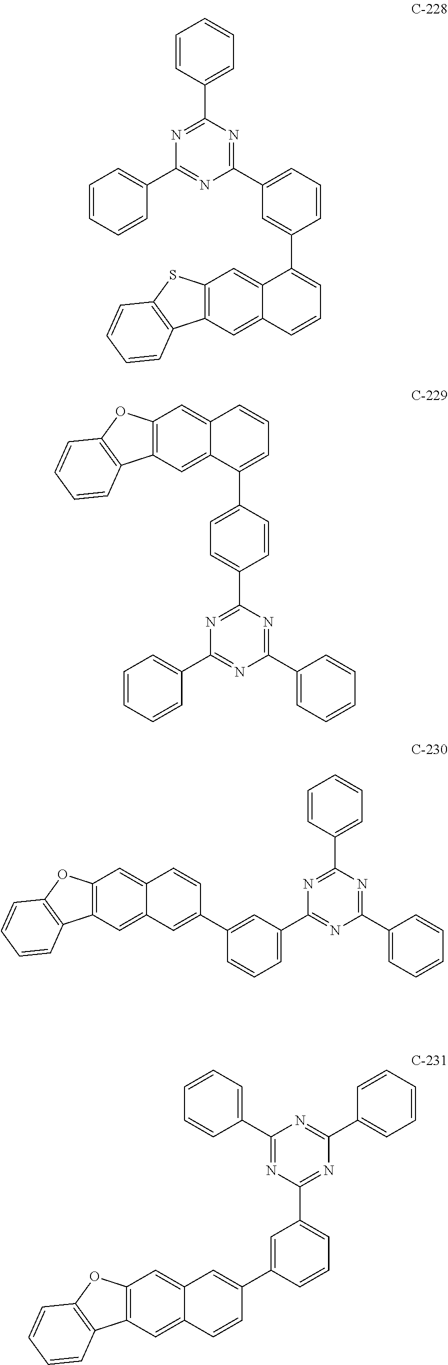 [SMILES: c1ccc(-c2nc(-c3ccccc3)nc(-c3cccc(-c4cccc5cc6c(cc45)sc4ccccc46)c3)n2)cc1 ; c1ccc(-c2nc(-c3ccccc3)nc(-c3ccc(-c4cccc5cc6oc7ccccc7c6cc45)cc3)n2)cc1 ; c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1]

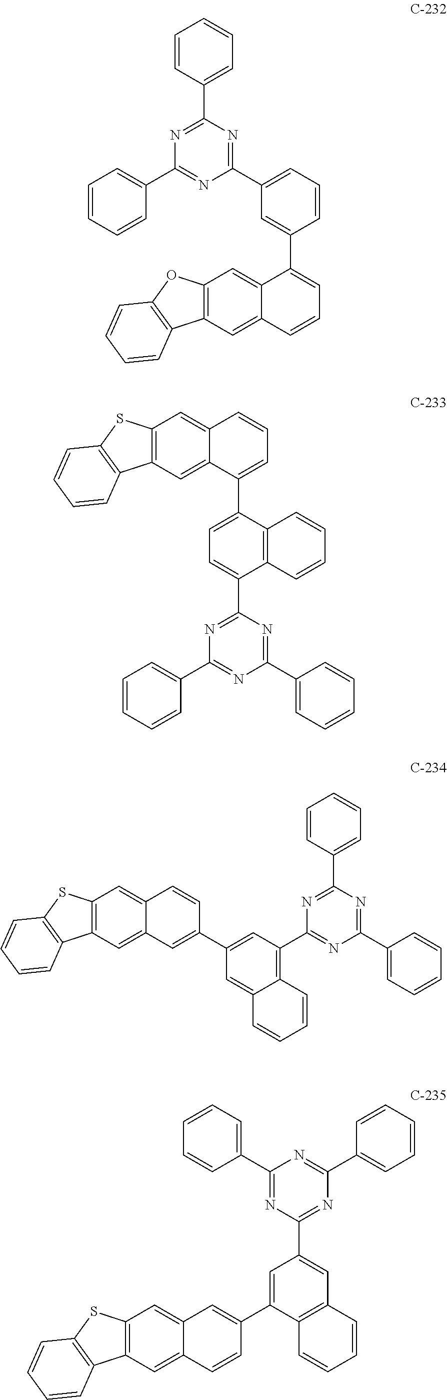 [SMILES: c1ccc(-c2nc(-c3ccccc3)nc(-c3cccc(-c4cccc5cc6c(cc45)oc4ccccc46)c3)n2)cc1 ; c1ccc(-c2nc(-c3ccccc3)nc(-c3ccc(-c4cccc5cc6sc7ccccc7c6cc45)c4ccccc34)n2)cc1 ; c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1]

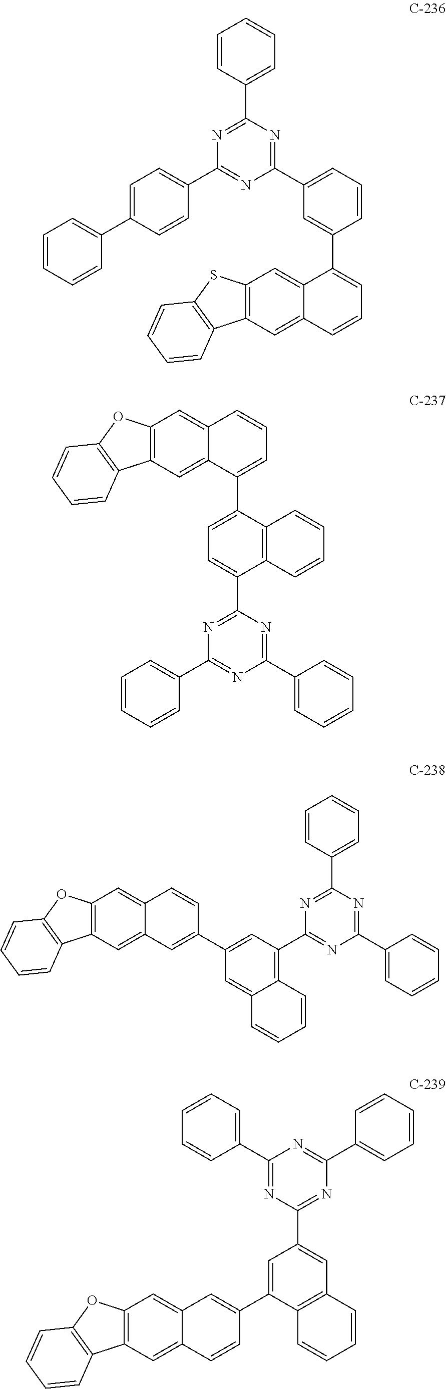 [SMILES: c1ccc(-c2ccc(-c3nc(-c4ccccc4)nc(-c4cccc(-c5cccc6cc7c(cc56)sc5ccccc57)c4)n3)cc2)cc1 ; c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1]

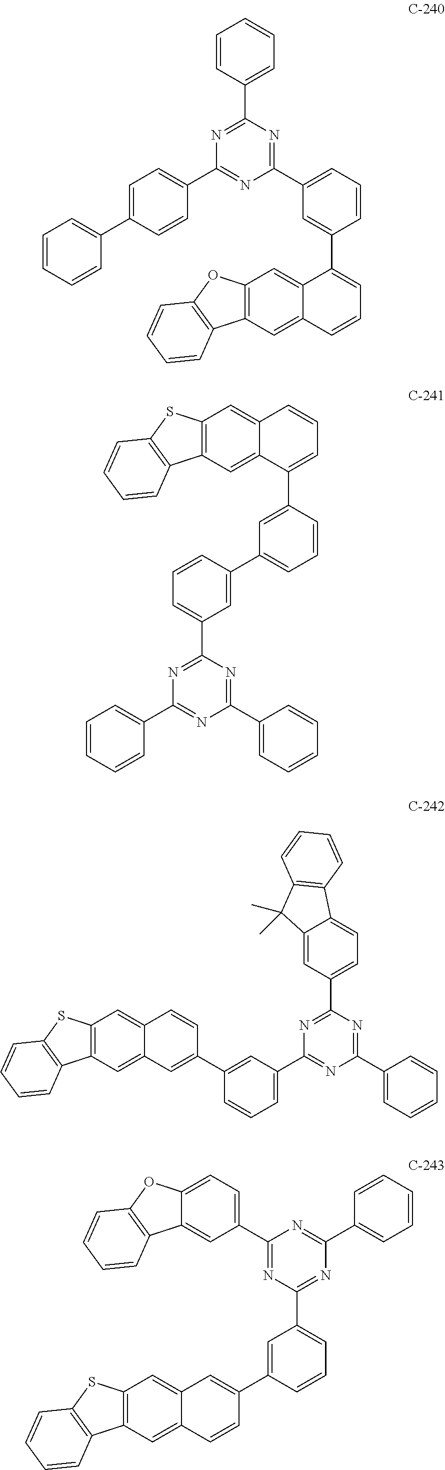 [SMILES: c1ccc(-c2ccc(-c3nc(-c4ccccc4)nc(-c4cccc(-c5cccc6cc7c(cc56)oc5ccccc57)c4)n3)cc2)cc1 ; c1ccc(-c2nc(-c3ccccc3)nc(-c3cccc(-c4cccc(-c5cccc6cc7sc8ccccc8c7cc56)c4)c3)n2)cc1 ; CC1(C)c2ccccc2-c2ccc(-c3nc(-c4ccccc4)nc(-c4cccc(-c5ccc6cc7sc8ccccc8c7cc6c5)c4)n3)cc21 ; c1ccc(-c2nc(-c3cccc(-c4ccc5cc6c(cc5c4)sc4ccccc46)c3)nc(-c3ccc4oc5ccccc5c4c3)n2)cc1]

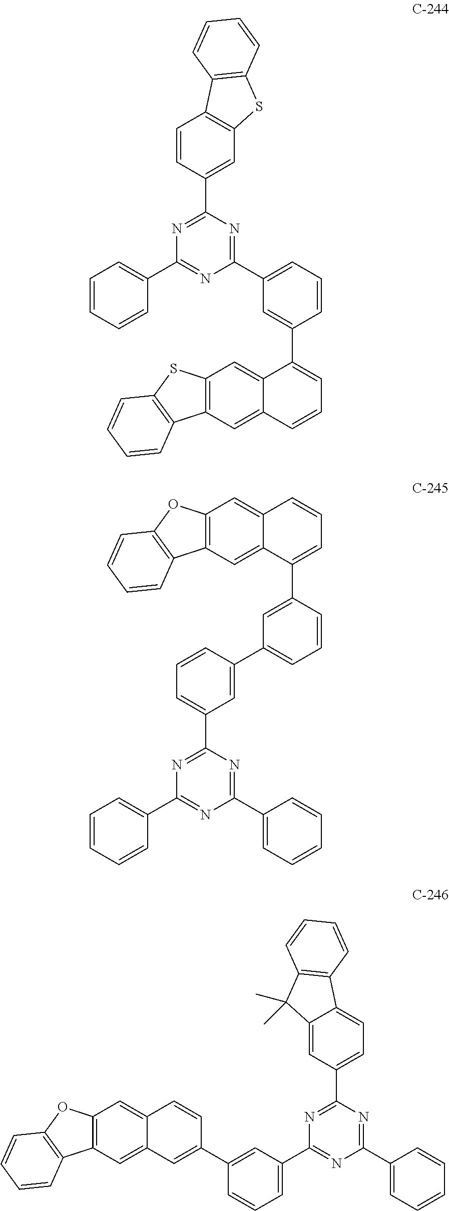 [SMILES: c1ccc(-c2nc(-c3cccc(-c4cccc5cc6c(cc45)sc4ccccc46)c3)nc(-c3ccc4c(c3)sc3ccccc34)n2)cc1 ; c1ccc(-c2nc(-c3ccccc3)nc(-c3cccc(-c4cccc(-c5cccc6cc7oc8ccccc8c7cc56)c4)c3)n2)cc1 ; CC1(C)c2ccccc2-c2ccc(-c3nc(-c4ccccc4)nc(-c4cccc(-c5ccc6cc7oc8ccccc8c7cc6c5)c4)n3)cc21]

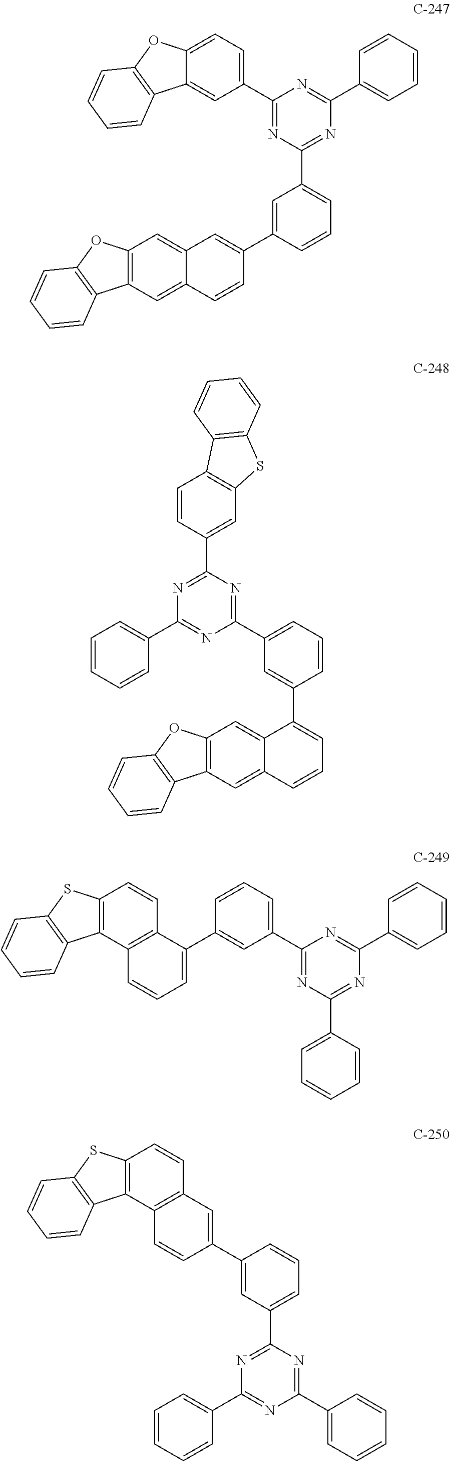 [SMILES: c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1 ; c1ccc(-c2nc(-c3ccccc3)nc(-c3cccc(-c4ccc5c(ccc6sc7ccccc7c65)c4)c3)n2)cc1]

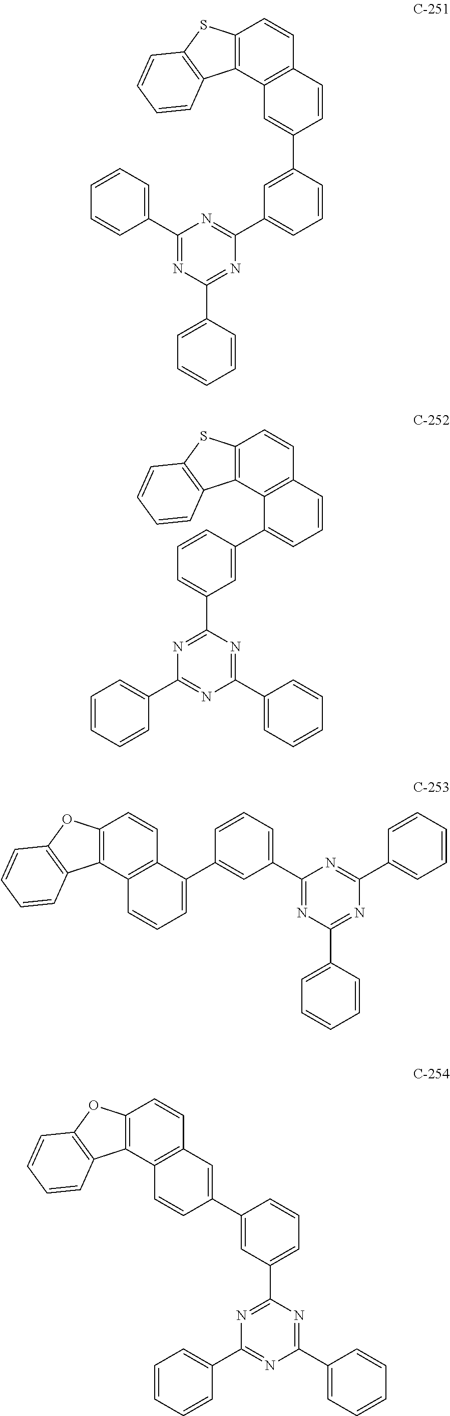 [SMILES: c1ccc(-c2nc(-c3ccccc3)nc(-c3cccc(-c4ccc5ccc6sc7ccccc7c6c5c4)c3)n2)cc1 ; c1ccc(-c2nc(-c3ccccc3)nc(-c3cccc(-c4cccc5ccc6sc7ccccc7c6c45)c3)n2)cc1 ; c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1]

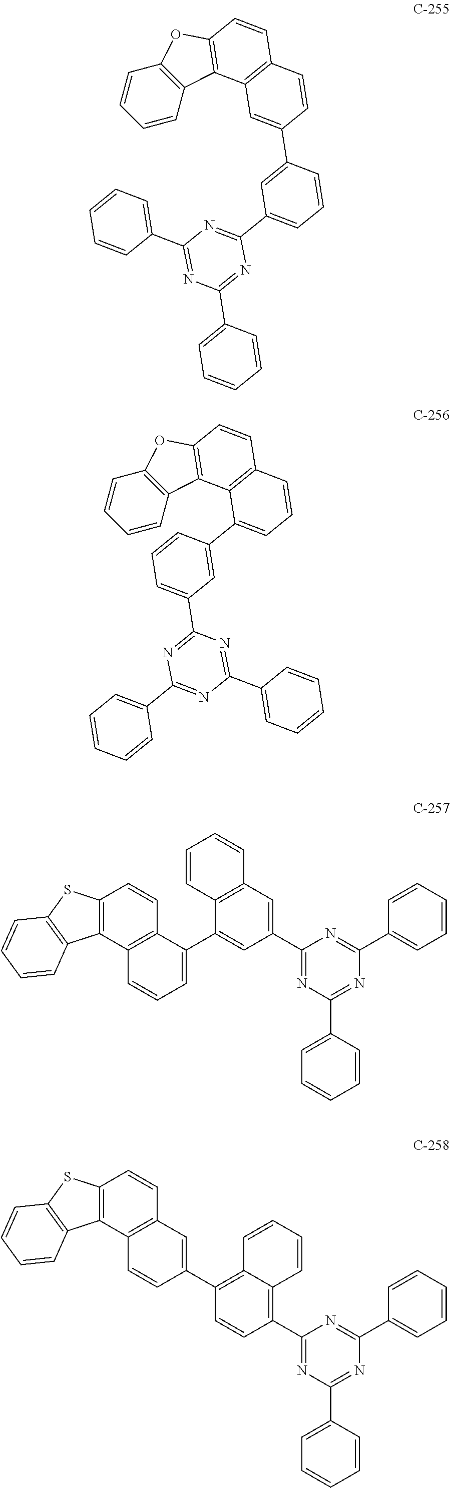

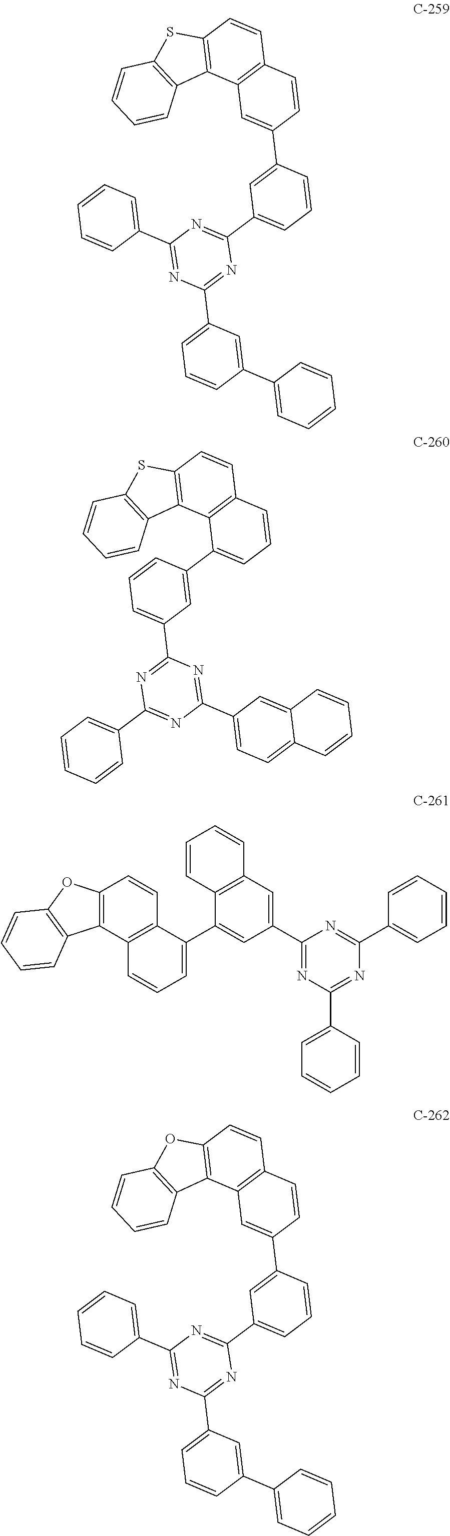 [SMILES: c1ccc(-c2cccc(-c3nc(-c4ccccc4)nc(-c4cccc(-c5ccc6ccc7sc8ccccc8c7c6c5)c4)n3)c2)cc1 ; c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1 ; c1ccc(-c2cccc(-c3nc(-c4ccccc4)nc(-c4cccc(-c5ccc6ccc7oc8ccccc8c7c6c5)c4)n3)c2)cc1]

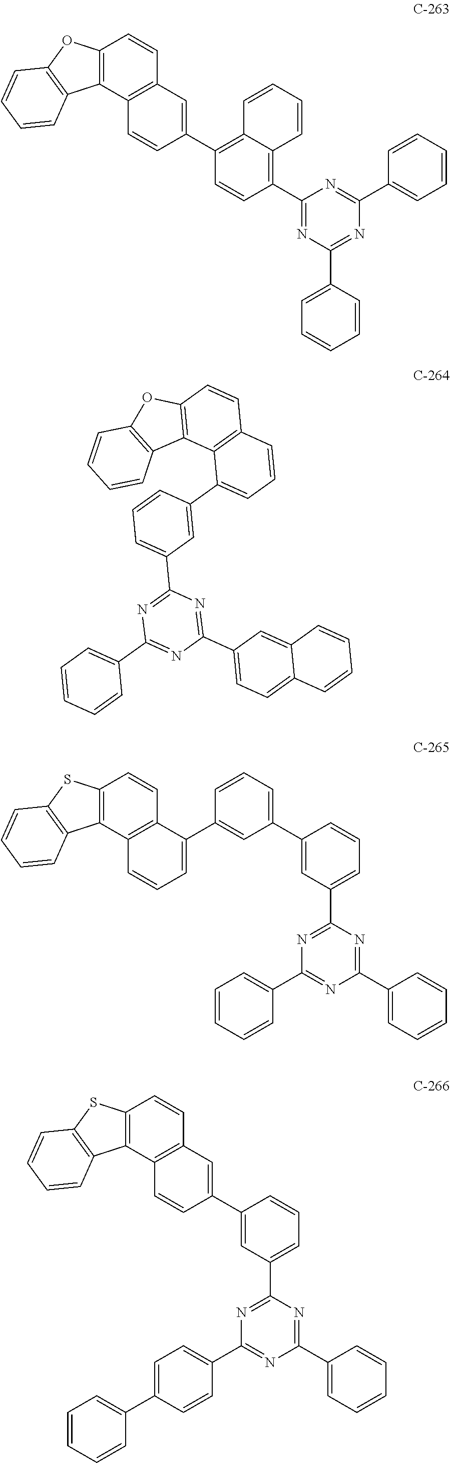 [SMILES: c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1 ; c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1]

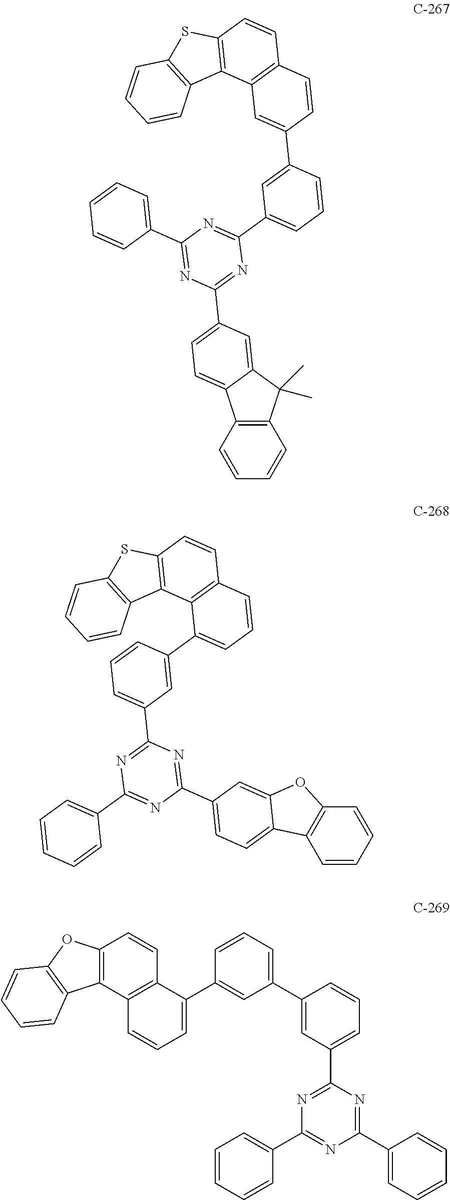 [SMILES: CC1(C)c2ccccc2-c2ccc(-c3nc(-c4ccccc4)nc(-c4cccc(-c5ccc6ccc7sc8ccccc8c7c6c5)c4)n3)cc21 ; c1ccc(-c2nc(-c3cccc(-c4cccc5ccc6sc7ccccc7c6c45)c3)nc(-c3ccc4c(c3)oc3ccccc34)n2)cc1 ; c1ccc(-c2nc(-c3ccccc3)nc(-c3cccc(-c4cccc(-c5cccc6c5ccc5oc7ccccc7c56)c4)c3)n2)cc1]

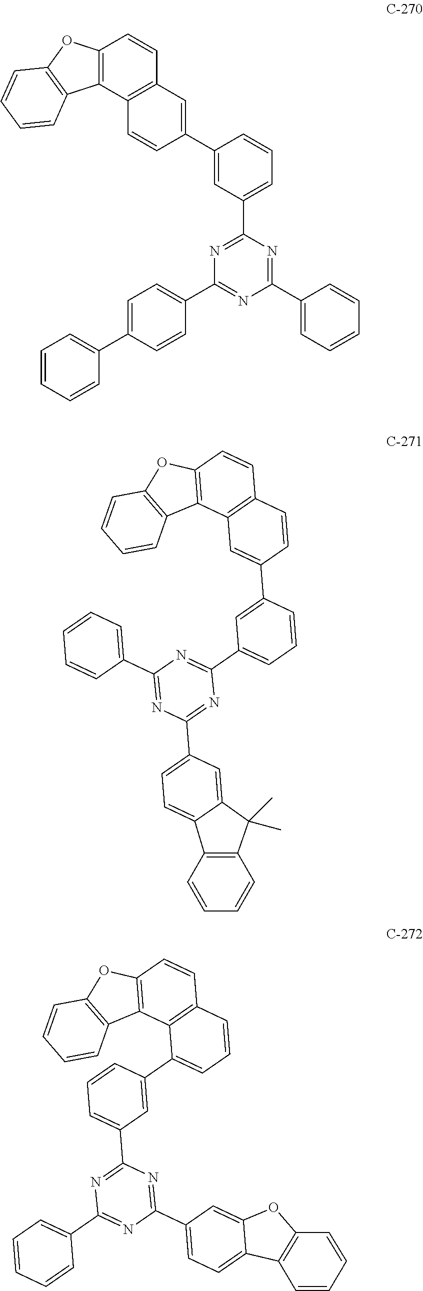 [SMILES: c1ccc(-c2ccc(-c3nc(-c4ccccc4)nc(-c4cccc(-c5ccc6c(ccc7oc8ccccc8c76)c5)c4)n3)cc2)cc1 ; CC1(C)c2ccccc2-c2ccc(-c3nc(-c4ccccc4)nc(-c4cccc(-c5ccc6ccc7oc8ccccc8c7c6c5)c4)n3)cc21 ; c1ccc(-c2nc(-c3cccc(-c4cccc5ccc6oc7ccccc7c6c45)c3)nc(-c3ccc4c(c3)oc3ccccc34)n2)cc1]

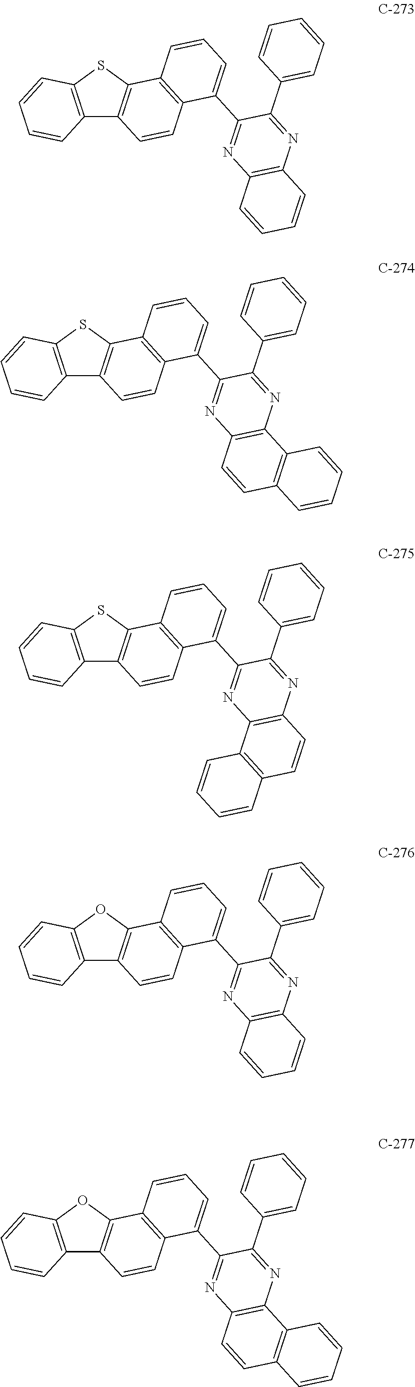 [SMILES: c1ccc(-c2nc3ccccc3nc2-c2cccc3c2ccc2c4ccccc4sc32)cc1 ; c1ccc(-c2nc3c(ccc4ccccc43)nc2-c2cccc3c2ccc2c4ccccc4sc32)cc1 ; c1ccc(-c2nc3ccc4ccccc4c3nc2-c2cccc3c2ccc2c4ccccc4sc32)cc1 ; c1ccc(-c2nc3ccccc3nc2-c2cccc3c2ccc2c4ccccc4oc32)cc1 ; c1ccc(-c2nc3c(ccc4ccccc43)nc2-c2cccc3c2ccc2c4ccccc4oc32)cc1]

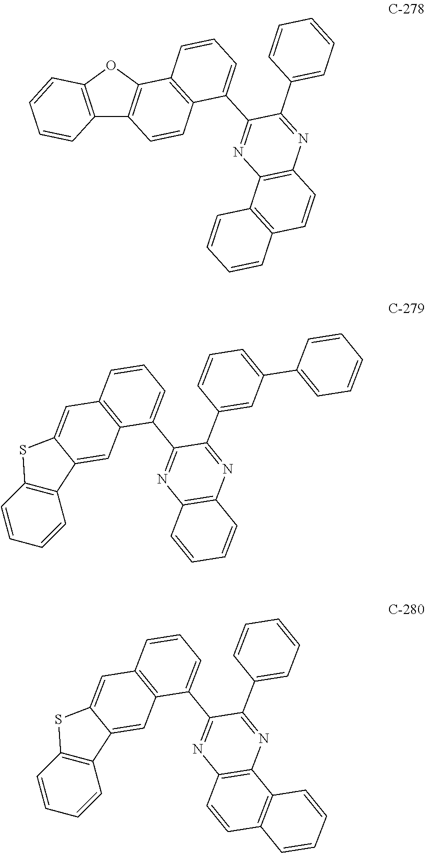 [SMILES: c1ccc(-c2nc3ccc4ccccc4c3nc2-c2cccc3c2ccc2c4ccccc4oc32)cc1 ; c1ccc(-c2cccc(-c3nc4ccccc4nc3-c3cccc4cc5sc6ccccc6c5cc34)c2)cc1 ; c1ccc(-c2nc3c(ccc4ccccc43)nc2-c2cccc3cc4sc5ccccc5c4cc23)cc1]

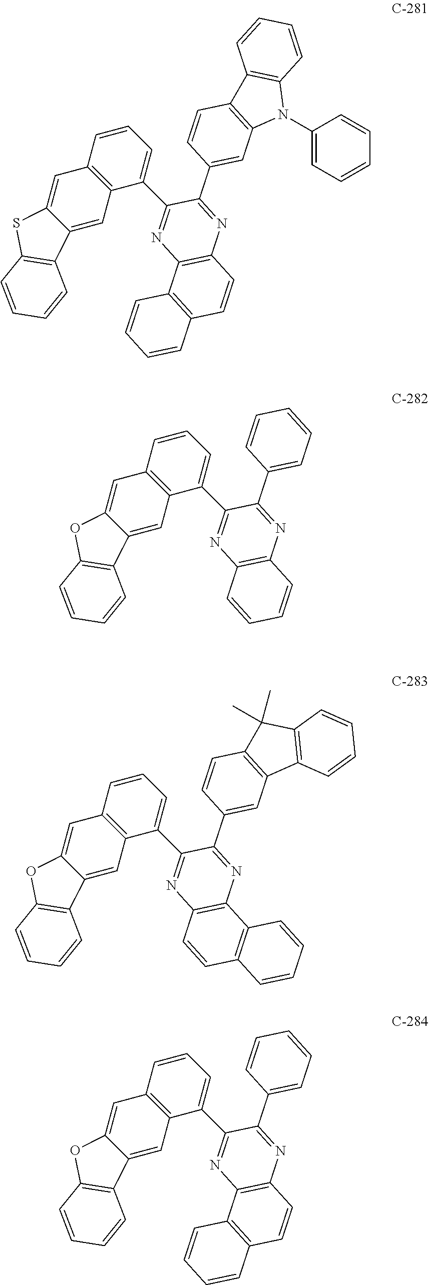 [SMILES: c1ccc(-n2c3ccccc3c3ccc(-c4nc5ccc6ccccc6c5nc4-c4cccc5cc6sc7ccccc7c6cc45)cc32)cc1 ; c1ccc(-c2nc3ccccc3nc2-c2cccc3cc4oc5ccccc5c4cc23)cc1 ; CC1(C)c2ccccc2-c2cc(-c3nc4c(ccc5ccccc54)nc3-c3cccc4cc5oc6ccccc6c5cc34)ccc21 ; c1ccc(-c2nc3ccc4ccccc4c3nc2-c2cccc3cc4oc5ccccc5c4cc23)cc1]

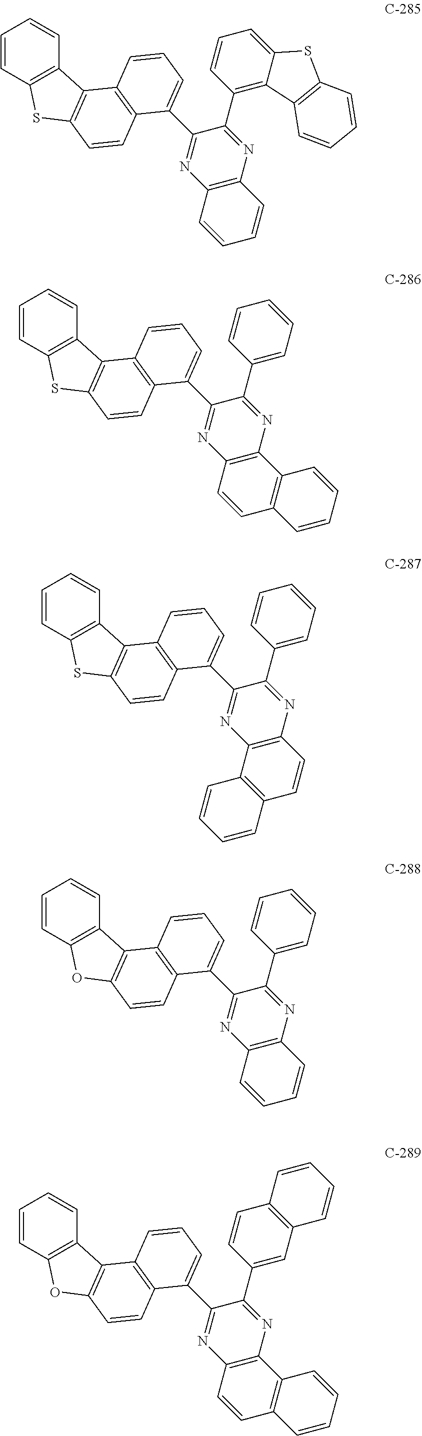 [SMILES: c1ccc2nc(-c3cccc4sc5ccccc5c34)c(-c3cccc4c3ccc3sc5ccccc5c34)nc2c1 ; c1ccc(-c2nc3c(ccc4ccccc43)nc2-c2cccc3c2ccc2sc4ccccc4c23)cc1 ; c1ccc(-c2nc3ccc4ccccc4c3nc2-c2cccc3c2ccc2sc4ccccc4c23)cc1 ; c1ccc(-c2nc3ccccc3nc2-c2cccc3c2ccc2oc4ccccc4c23)cc1 ; c1ccc2cc(-c3nc4c(ccc5ccccc54)nc3-c3cccc4c3ccc3oc5ccccc5c34)ccc2c1]

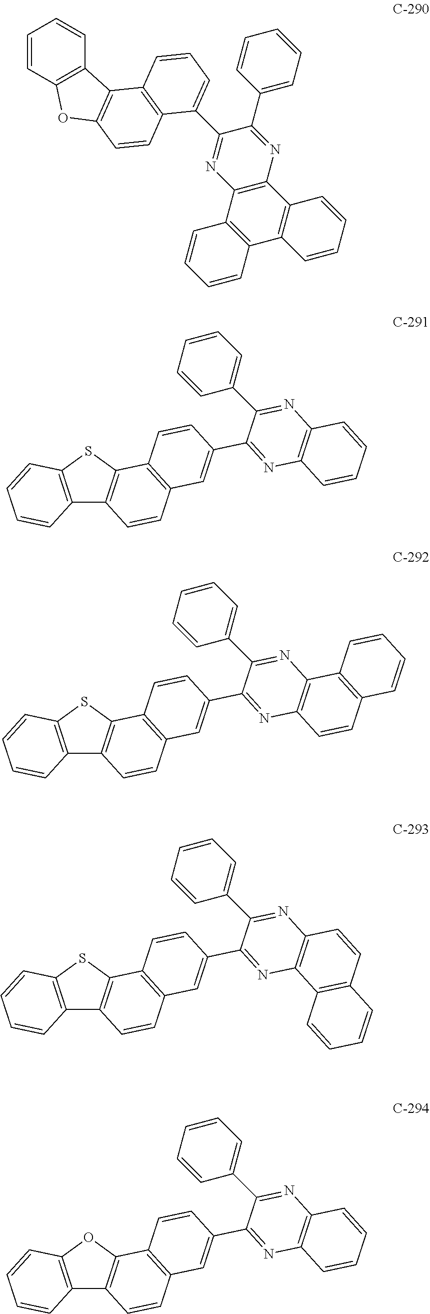

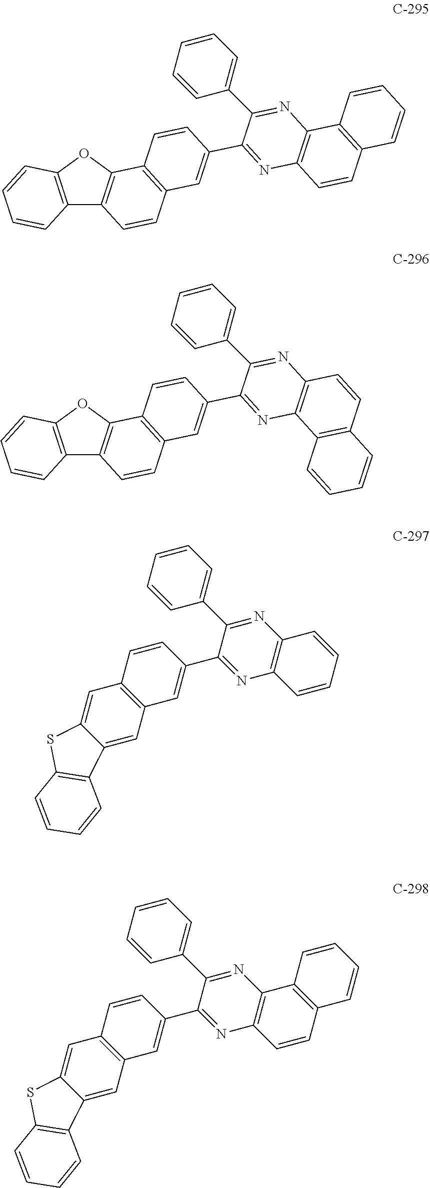 [SMILES: c1ccc(-c2nc3c(ccc4ccccc43)nc2-c2ccc3c(ccc4c5ccccc5oc34)c2)cc1 ; c1ccc(-c2nc3ccc4ccccc4c3nc2-c2ccc3c(ccc4c5ccccc5oc34)c2)cc1 ; c1ccc(-c2nc3ccccc3nc2-c2ccc3cc4sc5ccccc5c4cc3c2)cc1 ; c1ccc(-c2nc3c(ccc4ccccc43)nc2-c2ccc3cc4sc5ccccc5c4cc3c2)cc1]

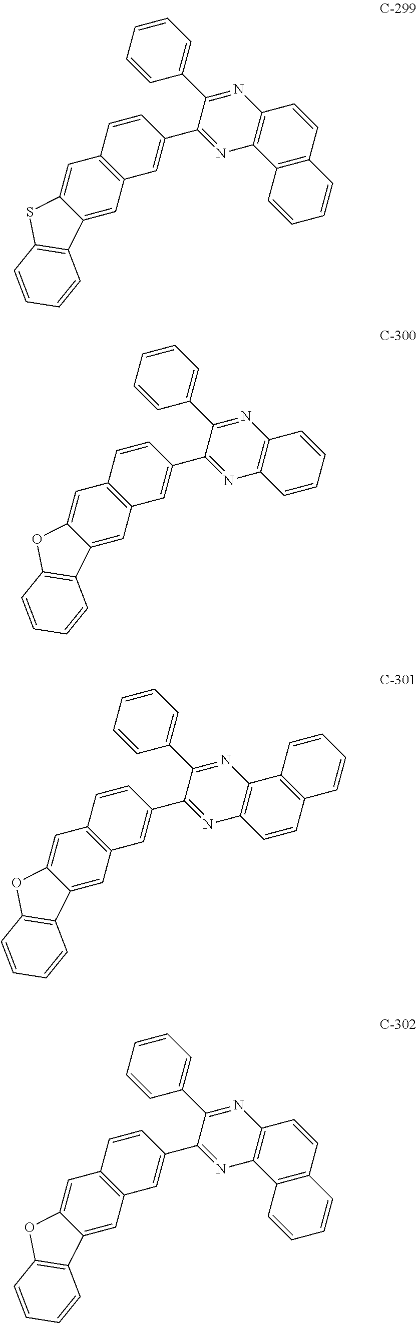 [SMILES: c1ccc(-c2nc3ccc4ccccc4c3nc2-c2ccc3cc4sc5ccccc5c4cc3c2)cc1 ; c1ccc(-c2nc3ccccc3nc2-c2ccc3cc4oc5ccccc5c4cc3c2)cc1 ; c1ccc(-c2nc3c(ccc4ccccc43)nc2-c2ccc3cc4oc5ccccc5c4cc3c2)cc1 ; c1ccc(-c2nc3ccc4ccccc4c3nc2-c2ccc3cc4oc5ccccc5c4cc3c2)cc1]

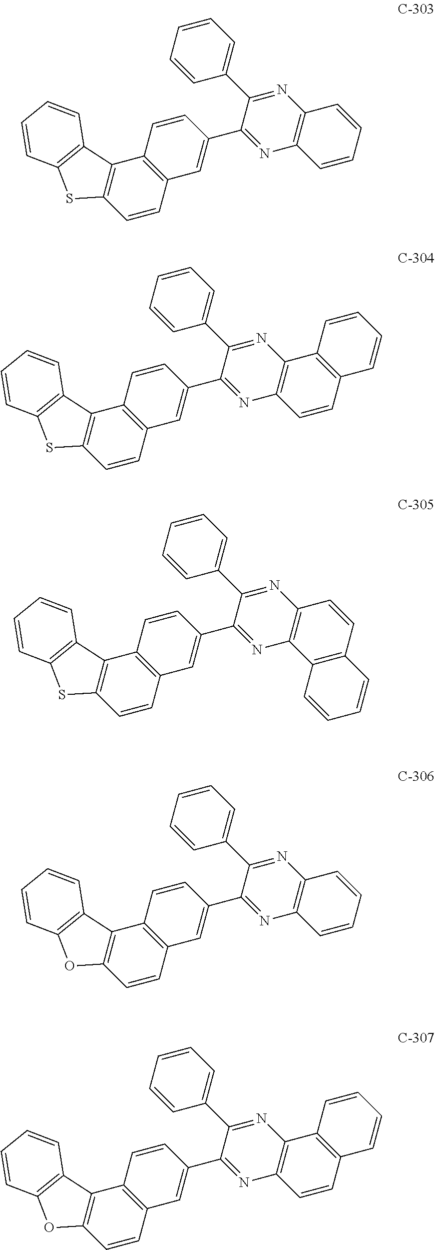 [SMILES: c1ccc(-c2nc3ccccc3nc2-c2ccc3c(ccc4sc5ccccc5c43)c2)cc1 ; c1ccc(-c2nc3c(ccc4ccccc43)nc2-c2ccc3c(ccc4sc5ccccc5c43)c2)cc1 ; c1ccc(-c2nc3ccc4ccccc4c3nc2-c2ccc3c(ccc4sc5ccccc5c43)c2)cc1 ; c1ccc(-c2nc3ccccc3nc2-c2ccc3c(ccc4oc5ccccc5c43)c2)cc1 ; c1ccc(-c2nc3c(ccc4ccccc43)nc2-c2ccc3c(ccc4oc5ccccc5c43)c2)cc1]

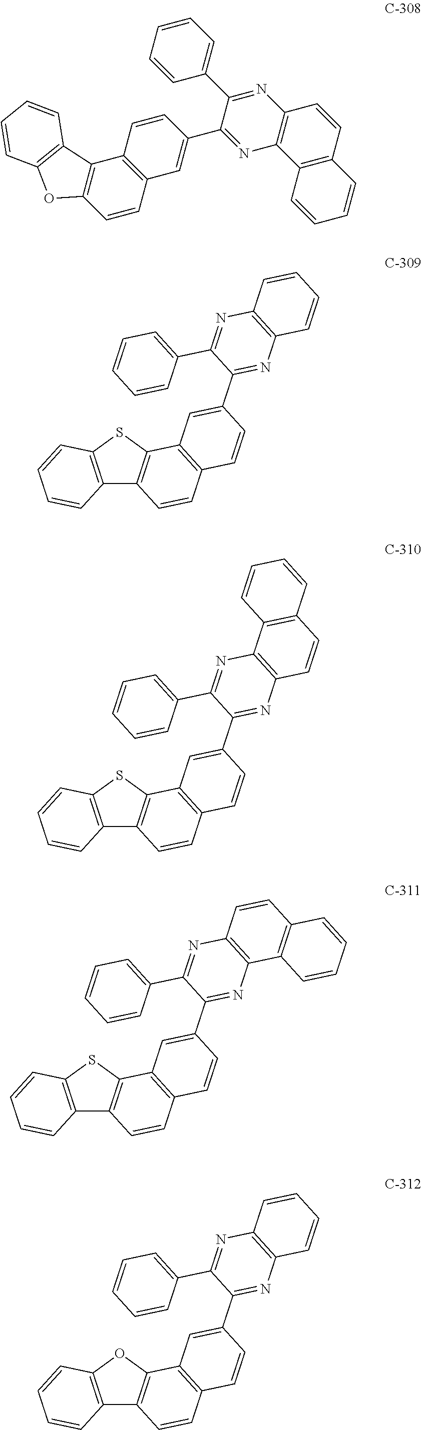 [SMILES: c1ccc(-c2nc3ccc4ccccc4c3nc2-c2ccc3c(ccc4oc5ccccc5c43)c2)cc1 ; c1ccc(-c2nc3ccccc3nc2-c2ccc3ccc4c5ccccc5sc4c3c2)cc1 ; c1ccc(-c2nc3c(ccc4ccccc43)nc2-c2ccc3ccc4c5ccccc5sc4c3c2)cc1 ; c1ccc(-c2nc3ccc4ccccc4c3nc2-c2ccc3ccc4c5ccccc5sc4c3c2)cc1 ; c1ccc(-c2nc3ccccc3nc2-c2ccc3ccc4c5ccccc5oc4c3c2)cc1]

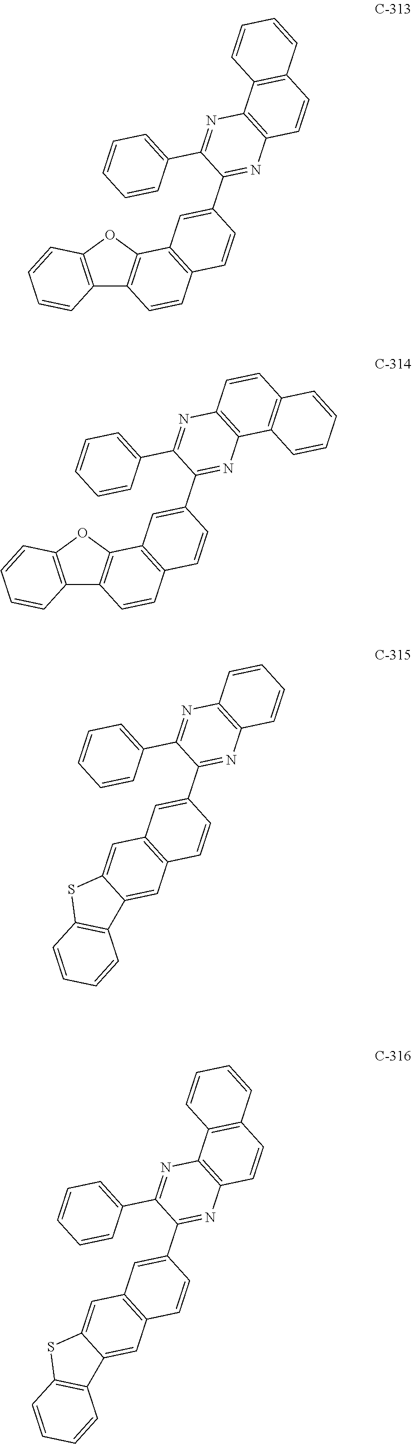 [SMILES: c1ccc(-c2nc3c(ccc4ccccc43)nc2-c2ccc3ccc4c5ccccc5oc4c3c2)cc1 ; c1ccc(-c2nc3ccc4ccccc4c3nc2-c2ccc3ccc4c5ccccc5oc4c3c2)cc1 ; c1ccc(-c2nc3ccccc3nc2-c2ccc3cc4c(cc3c2)sc2ccccc24)cc1 ; c1ccc(-c2nc3c(ccc4ccccc43)nc2-c2ccc3cc4c(cc3c2)sc2ccccc24)cc1]

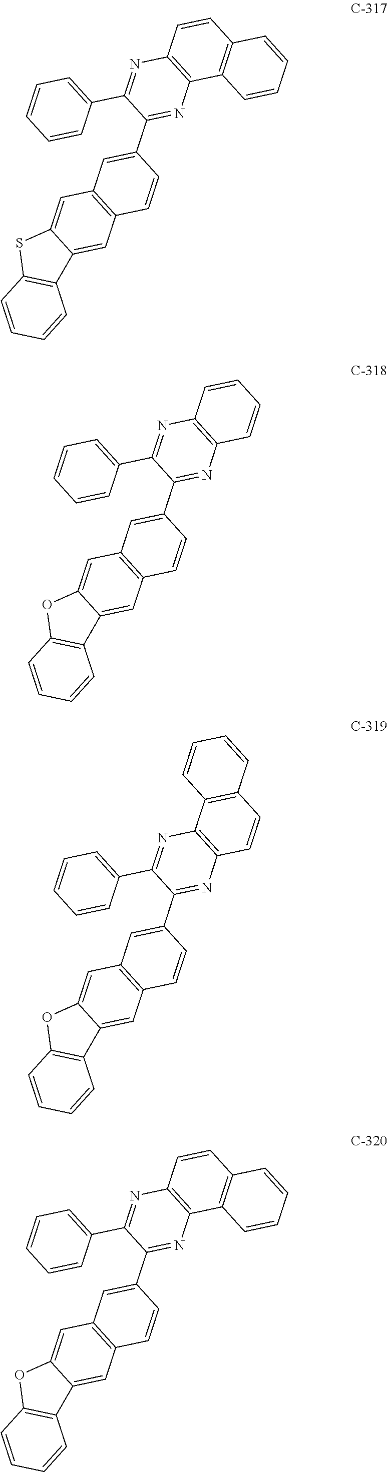 [SMILES: c1ccc(-c2nc3ccc4ccccc4c3nc2-c2ccc3cc4c(cc3c2)sc2ccccc24)cc1 ; c1ccc(-c2nc3ccccc3nc2-c2ccc3cc4c(cc3c2)oc2ccccc24)cc1 ; c1ccc(-c2nc3c(ccc4ccccc43)nc2-c2ccc3cc4c(cc3c2)oc2ccccc24)cc1 ; c1ccc(-c2nc3ccc4ccccc4c3nc2-c2ccc3cc4c(cc3c2)oc2ccccc24)cc1]

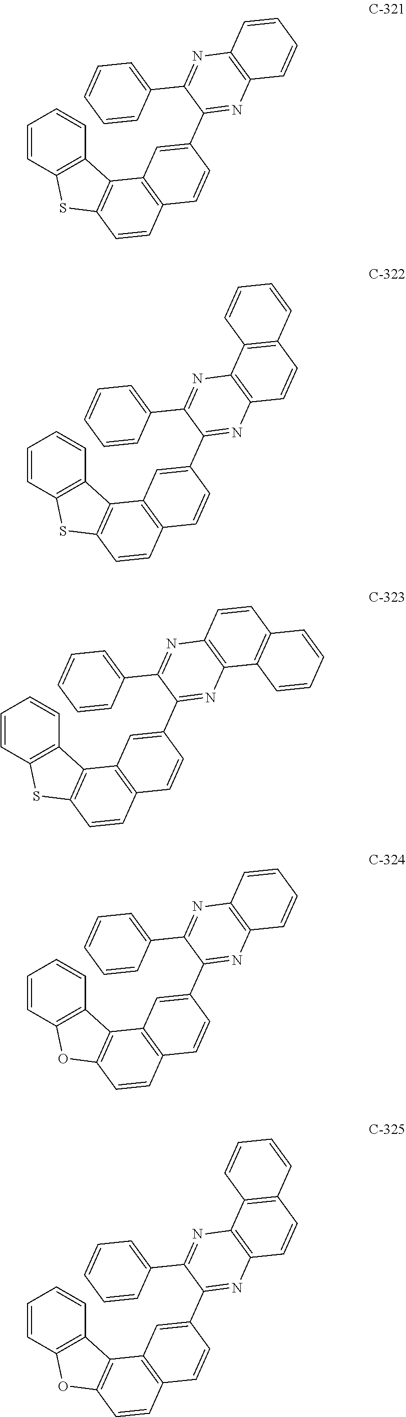 [SMILES: c1ccc(-c2nc3ccccc3nc2-c2ccc3ccc4sc5ccccc5c4c3c2)cc1 ; c1ccc(-c2nc3c(ccc4ccccc43)nc2-c2ccc3ccc4sc5ccccc5c4c3c2)cc1 ; c1ccc(-c2nc3ccc4ccccc4c3nc2-c2ccc3ccc4sc5ccccc5c4c3c2)cc1 ; c1ccc(-c2nc3ccccc3nc2-c2ccc3ccc4oc5ccccc5c4c3c2)cc1 ; c1ccc(-c2nc3c(ccc4ccccc43)nc2-c2ccc3ccc4oc5ccccc5c4c3c2)cc1]

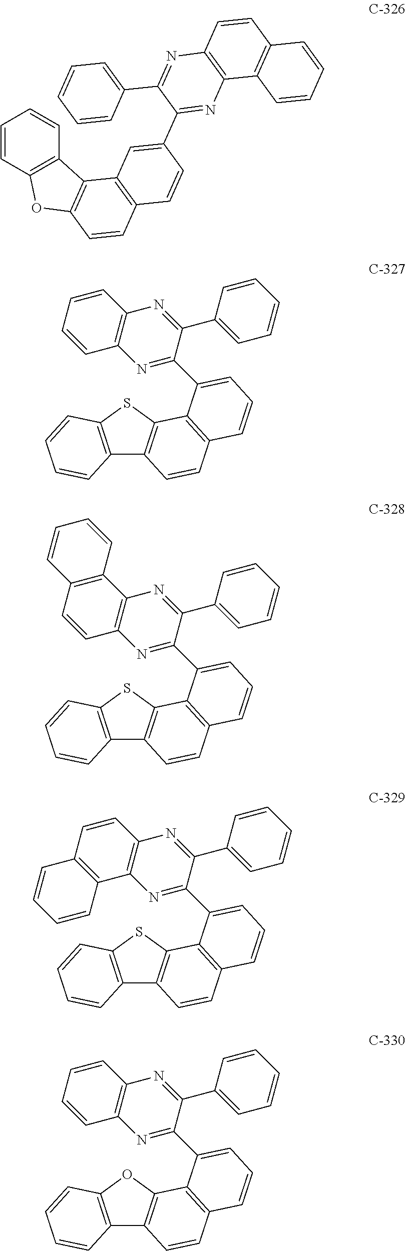

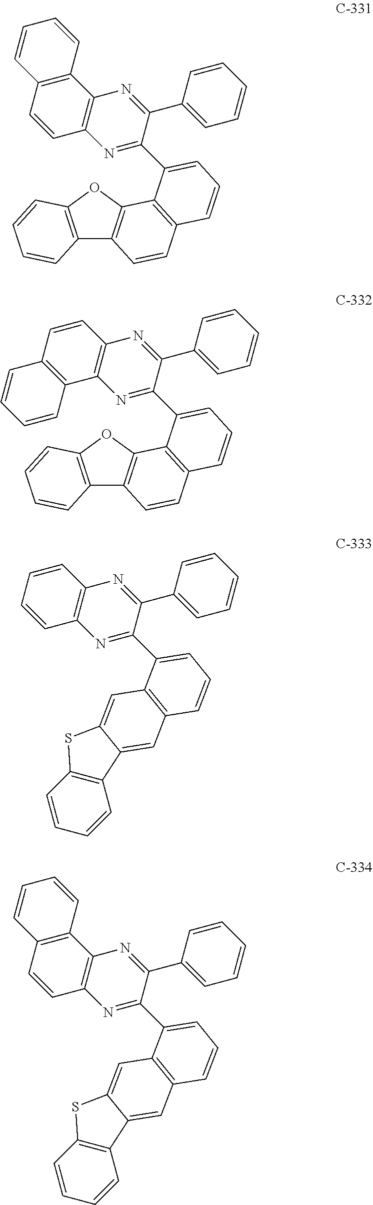 [SMILES: c1ccc(-c2nc3c(ccc4ccccc43)nc2-c2cccc3ccc4c5ccccc5oc4c23)cc1 ; c1ccc(-c2nc3ccc4ccccc4c3nc2-c2cccc3ccc4c5ccccc5oc4c23)cc1 ; c1ccc(-c2nc3ccccc3nc2-c2cccc3cc4c(cc23)sc2ccccc24)cc1 ; c1ccc(-c2nc3c(ccc4ccccc43)nc2-c2cccc3cc4c(cc23)sc2ccccc24)cc1]

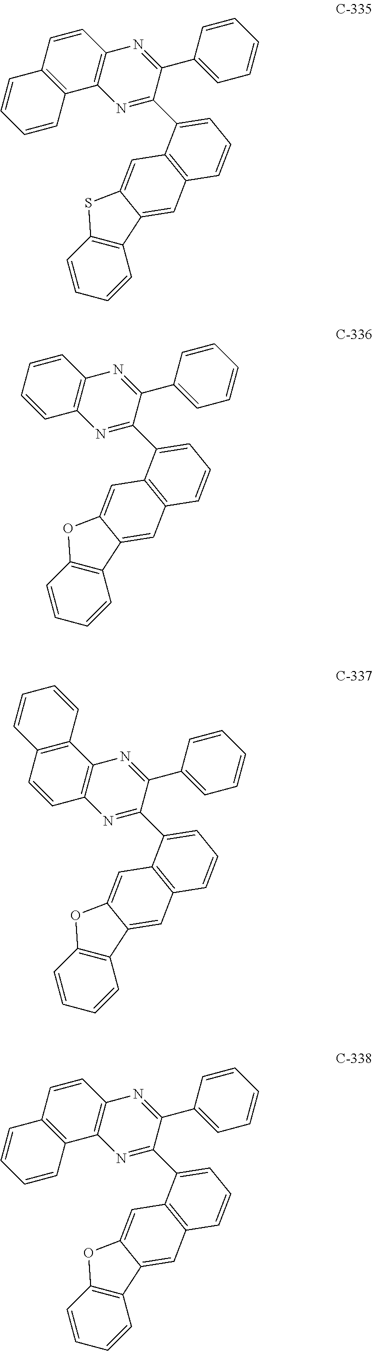 [SMILES: c1ccc(-c2nc3ccc4ccccc4c3nc2-c2cccc3cc4c(cc23)sc2ccccc24)cc1 ; c1ccc(-c2nc3ccccc3nc2-c2cccc3cc4c(cc23)oc2ccccc24)cc1 ; c1ccc(-c2nc3c(ccc4ccccc43)nc2-c2cccc3cc4c(cc23)oc2ccccc24)cc1 ; c1ccc(-c2nc3ccc4ccccc4c3nc2-c2cccc3cc4c(cc23)oc2ccccc24)cc1]

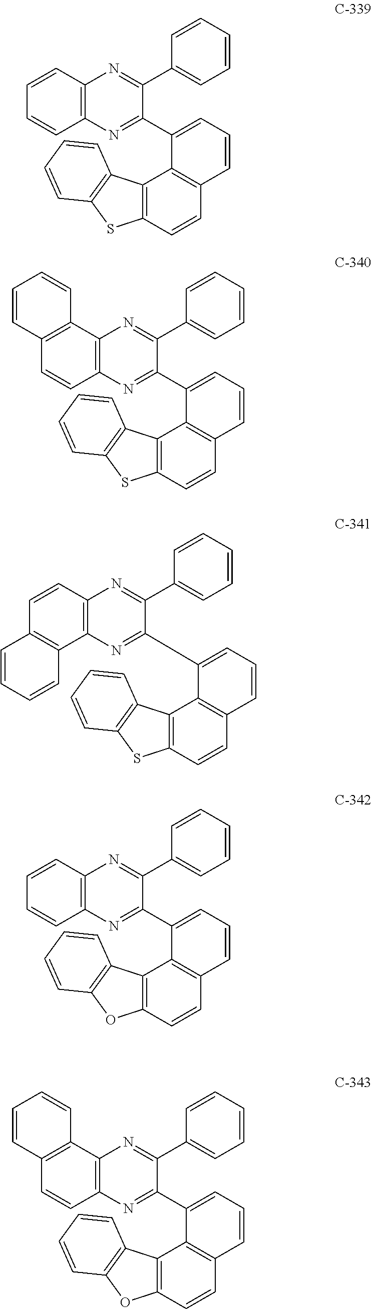

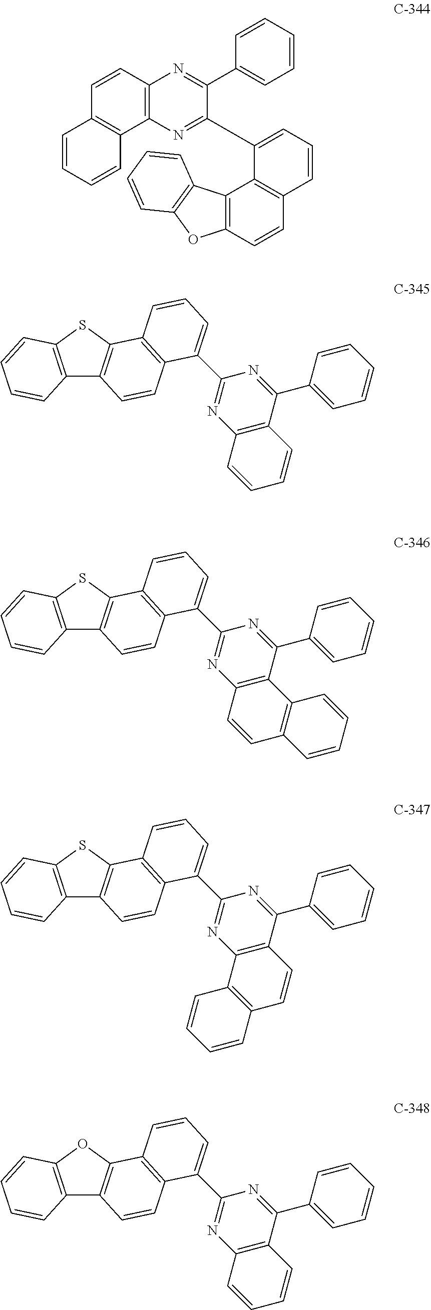 [SMILES: c1ccc(-c2nc3ccc4ccccc4c3nc2-c2cccc3ccc4oc5ccccc5c4c23)cc1 ; c1ccc(-c2nc(-c3cccc4c3ccc3c5ccccc5sc43)nc3ccccc23)cc1 ; c1ccc(-c2nc(-c3cccc4c3ccc3c5ccccc5sc43)nc3ccc4ccccc4c23)cc1 ; c1ccc(-c2nc(-c3cccc4c3ccc3c5ccccc5sc43)nc3c2ccc2ccccc23)cc1 ; c1ccc(-c2nc(-c3cccc4c3ccc3c5ccccc5oc43)nc3ccccc23)cc1]

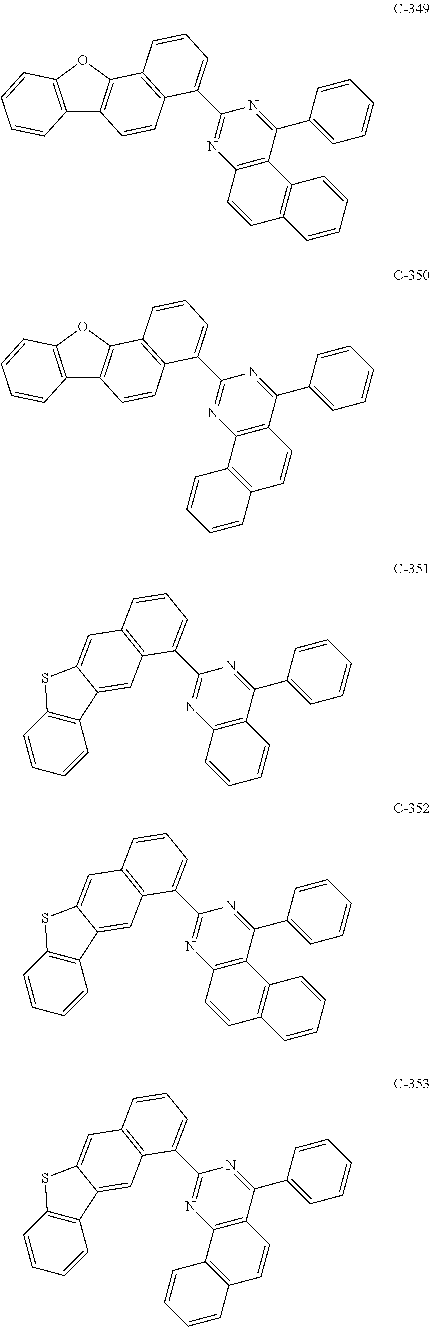 [SMILES: c1ccc(-c2nc(-c3cccc4c3ccc3c5ccccc5oc43)nc3ccc4ccccc4c23)cc1 ; c1ccc(-c2nc(-c3cccc4c3ccc3c5ccccc5oc43)nc3c2ccc2ccccc23)cc1 ; c1ccc(-c2nc(-c3cccc4cc5sc6ccccc6c5cc34)nc3ccccc23)cc1 ; c1ccc(-c2nc(-c3cccc4cc5sc6ccccc6c5cc34)nc3ccc4ccccc4c23)cc1 ; c1ccc(-c2nc(-c3cccc4cc5sc6ccccc6c5cc34)nc3c2ccc2ccccc23)cc1]

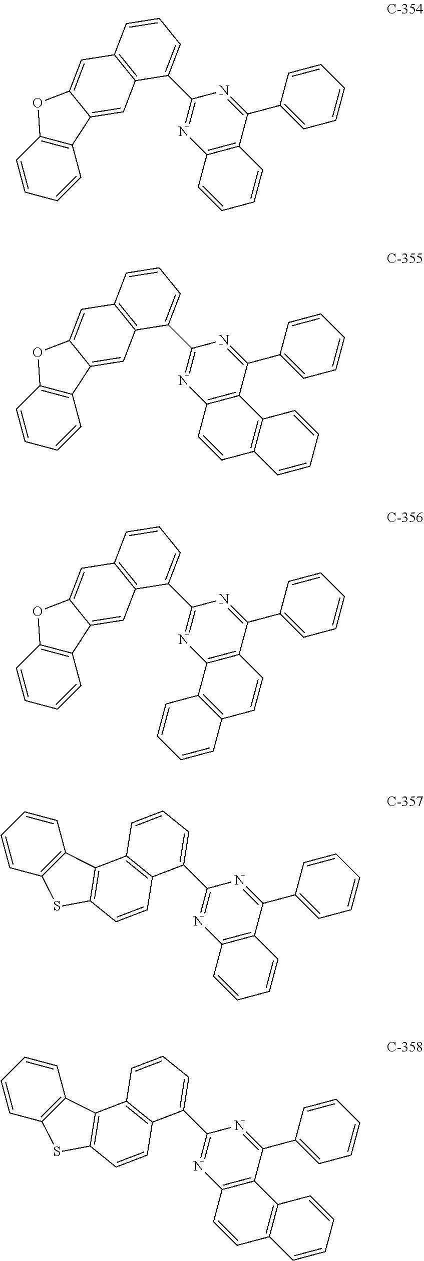 [SMILES: c1ccc(-c2nc(-c3cccc4cc5oc6ccccc6c5cc34)nc3ccccc23)cc1 ; c1ccc(-c2nc(-c3cccc4cc5oc6ccccc6c5cc34)nc3ccc4ccccc4c23)cc1 ; c1ccc(-c2nc(-c3cccc4cc5oc6ccccc6c5cc34)nc3c2ccc2ccccc23)cc1 ; c1ccc(-c2nc(-c3cccc4c3ccc3sc5ccccc5c34)nc3ccccc23)cc1 ; c1ccc(-c2nc(-c3cccc4c3ccc3sc5ccccc5c34)nc3ccc4ccccc4c23)cc1]

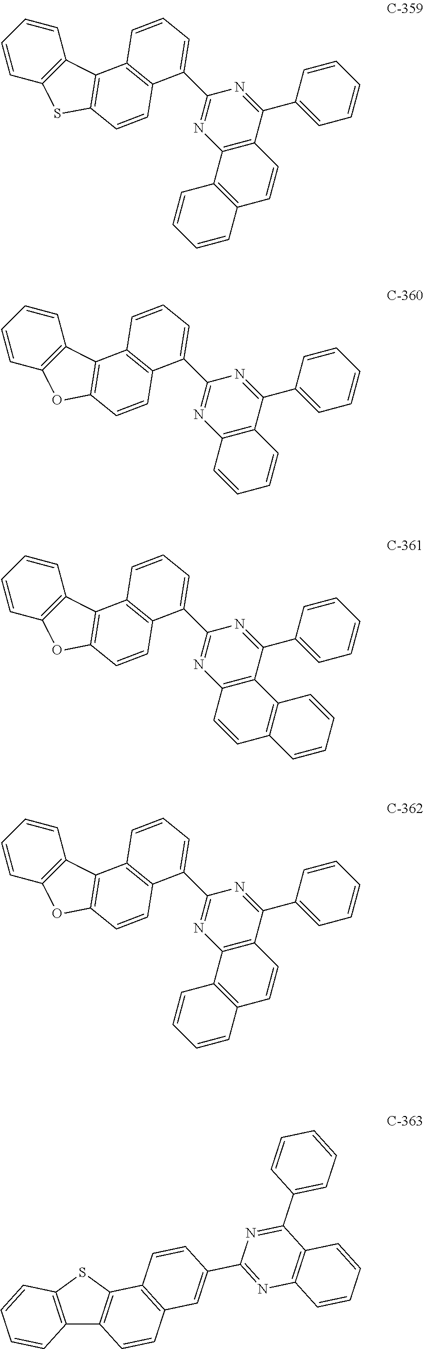 [SMILES: c1ccc(-c2nc(-c3cccc4c3ccc3sc5ccccc5c34)nc3c2ccc2ccccc23)cc1 ; c1ccc(-c2nc(-c3cccc4c3ccc3oc5ccccc5c34)nc3ccccc23)cc1 ; c1ccc(-c2nc(-c3cccc4c3ccc3oc5ccccc5c34)nc3ccc4ccccc4c23)cc1 ; c1ccc(-c2nc(-c3cccc4c3ccc3oc5ccccc5c34)nc3c2ccc2ccccc23)cc1 ; c1ccc(-c2nc(-c3ccc4c(ccc5c6ccccc6sc45)c3)nc3ccccc23)cc1]

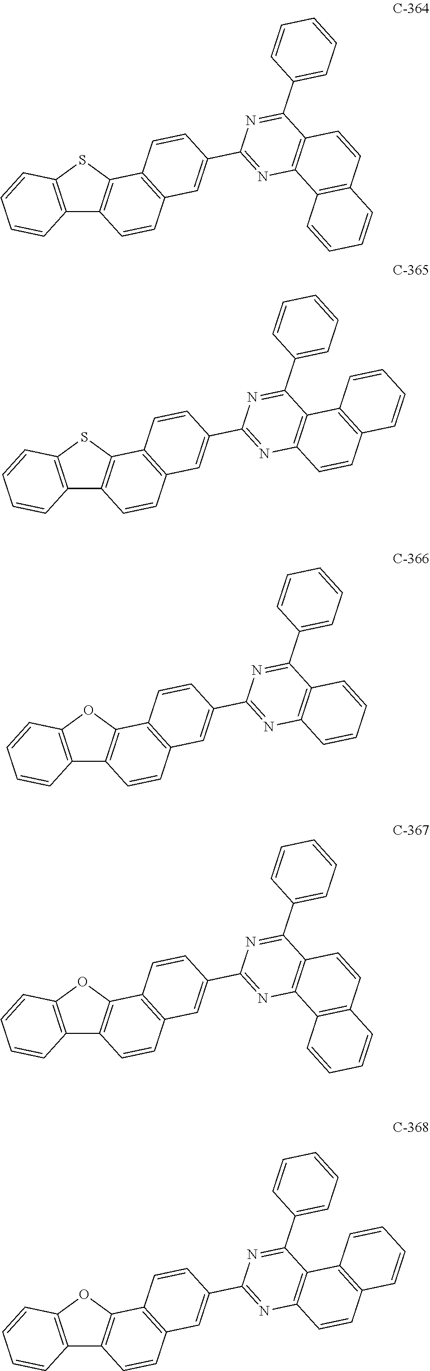

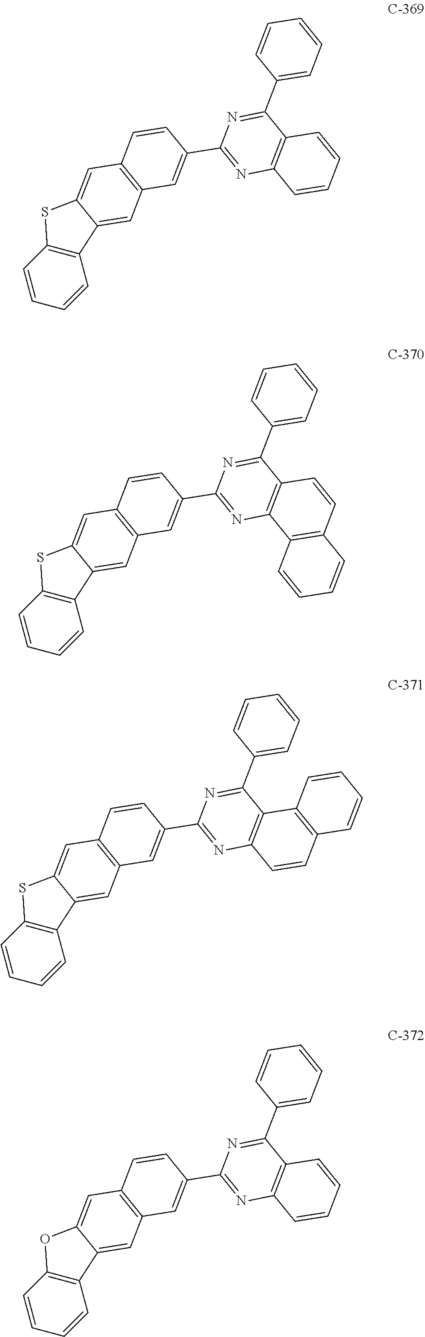 [SMILES: c1ccc(-c2nc(-c3ccc4cc5sc6ccccc6c5cc4c3)nc3ccccc23)cc1 ; c1ccc(-c2nc(-c3ccc4cc5sc6ccccc6c5cc4c3)nc3c2ccc2ccccc23)cc1 ; c1ccc(-c2nc(-c3ccc4cc5sc6ccccc6c5cc4c3)nc3ccc4ccccc4c23)cc1 ; c1ccc(-c2nc(-c3ccc4cc5oc6ccccc6c5cc4c3)nc3ccccc23)cc1]

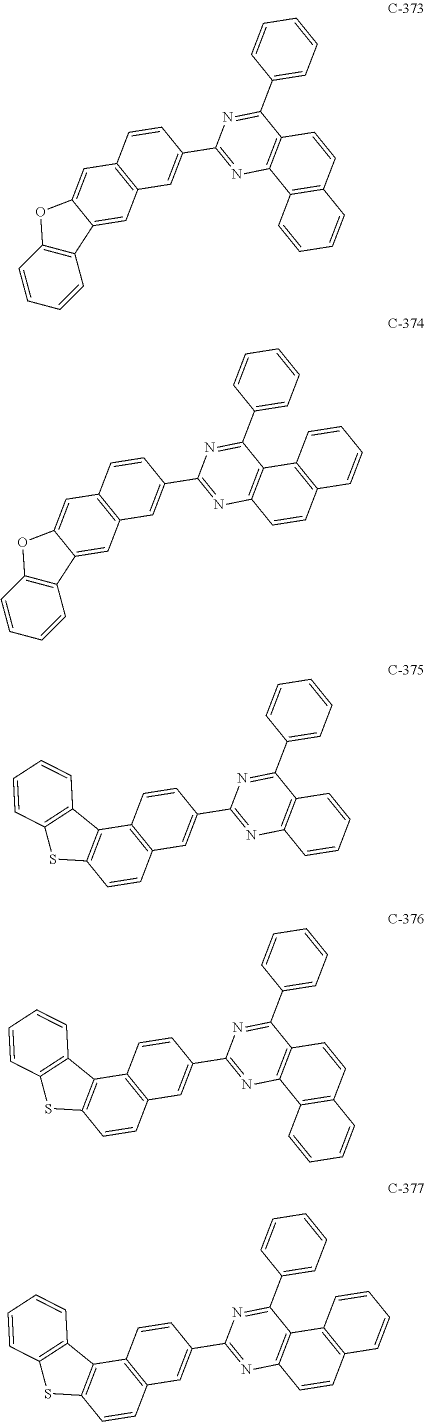 [SMILES: c1ccc(-c2nc(-c3ccc4cc5oc6ccccc6c5cc4c3)nc3c2ccc2ccccc23)cc1 ; c1ccc(-c2nc(-c3ccc4cc5oc6ccccc6c5cc4c3)nc3ccc4ccccc4c23)cc1 ; c1ccc(-c2nc(-c3ccc4c(ccc5sc6ccccc6c54)c3)nc3ccccc23)cc1 ; c1ccc(-c2nc(-c3ccc4c(ccc5sc6ccccc6c54)c3)nc3c2ccc2ccccc23)cc1 ; c1ccc(-c2nc(-c3ccc4c(ccc5sc6ccccc6c54)c3)nc3ccc4ccccc4c23)cc1]

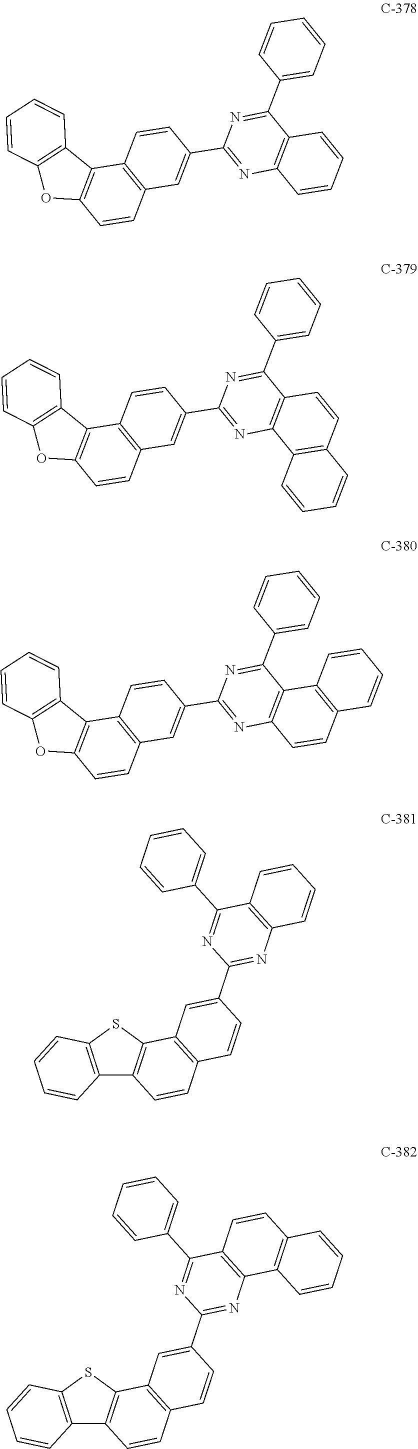 [SMILES: c1ccc(-c2nc(-c3ccc4c(ccc5oc6ccccc6c54)c3)nc3ccccc23)cc1 ; c1ccc(-c2nc(-c3ccc4c(ccc5oc6ccccc6c54)c3)nc3c2ccc2ccccc23)cc1 ; c1ccc(-c2nc(-c3ccc4c(ccc5oc6ccccc6c54)c3)nc3ccc4ccccc4c23)cc1 ; c1ccc(-c2nc(-c3ccc4ccc5c6ccccc6sc5c4c3)nc3ccccc23)cc1 ; c1ccc(-c2nc(-c3ccc4ccc5c6ccccc6sc5c4c3)nc3c2ccc2ccccc23)cc1]

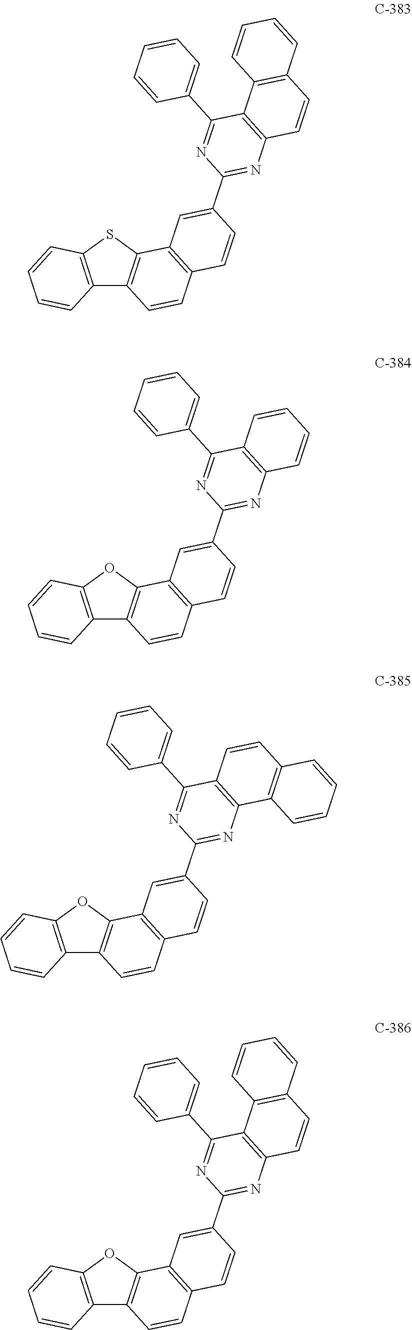 [SMILES: c1ccc(-c2nc(-c3ccc4ccc5c6ccccc6sc5c4c3)nc3ccc4ccccc4c23)cc1 ; c1ccc(-c2nc(-c3ccc4ccc5c6ccccc6oc5c4c3)nc3ccccc23)cc1 ; c1ccc(-c2nc(-c3ccc4ccc5c6ccccc6oc5c4c3)nc3c2ccc2ccccc23)cc1 ; c1ccc(-c2nc(-c3ccc4ccc5c6ccccc6oc5c4c3)nc3ccc4ccccc4c23)cc1]

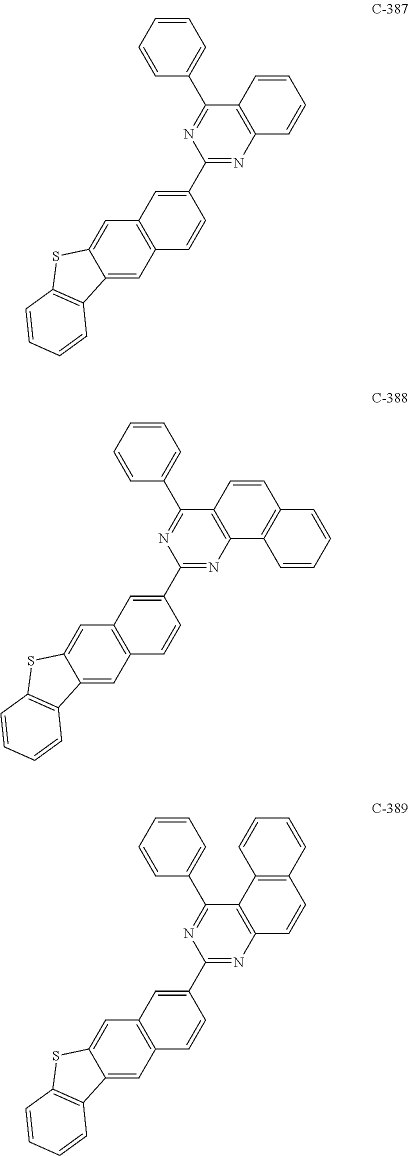 [SMILES: c1ccc(-c2nc(-c3ccc4cc5c(cc4c3)sc3ccccc35)nc3ccccc23)cc1 ; c1ccc(-c2nc(-c3ccc4cc5c(cc4c3)sc3ccccc35)nc3c2ccc2ccccc23)cc1 ; c1ccc(-c2nc(-c3ccc4cc5c(cc4c3)sc3ccccc35)nc3ccc4ccccc4c23)cc1]

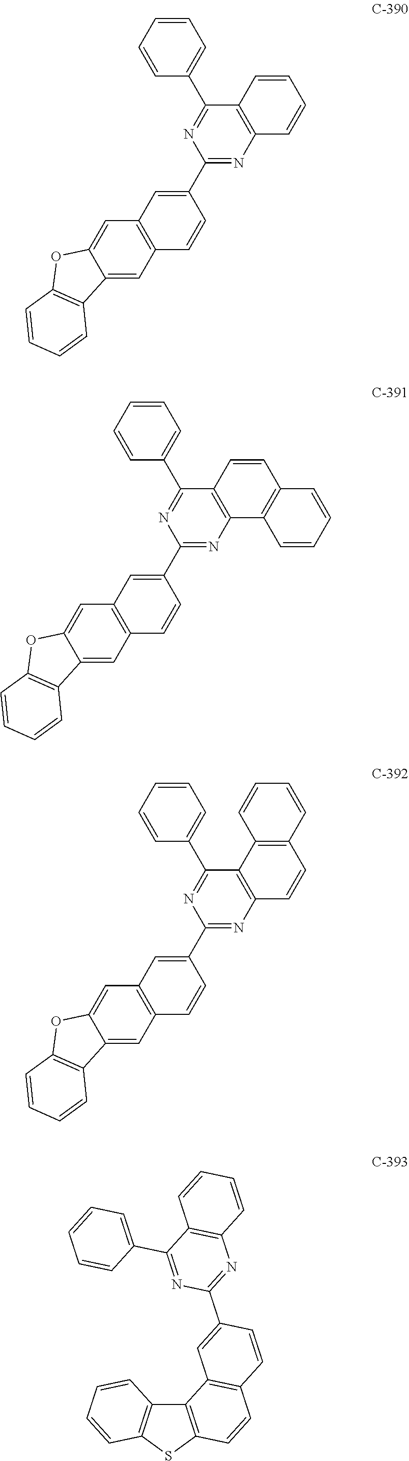 [SMILES: c1ccc(-c2nc(-c3ccc4cc5c(cc4c3)oc3ccccc35)nc3ccccc23)cc1 ; c1ccc(-c2nc(-c3ccc4cc5c(cc4c3)oc3ccccc35)nc3c2ccc2ccccc23)cc1 ; c1ccc(-c2nc(-c3ccc4cc5c(cc4c3)oc3ccccc35)nc3ccc4ccccc4c23)cc1 ; c1ccc(-c2nc(-c3ccc4ccc5sc6ccccc6c5c4c3)nc3ccccc23)cc1]

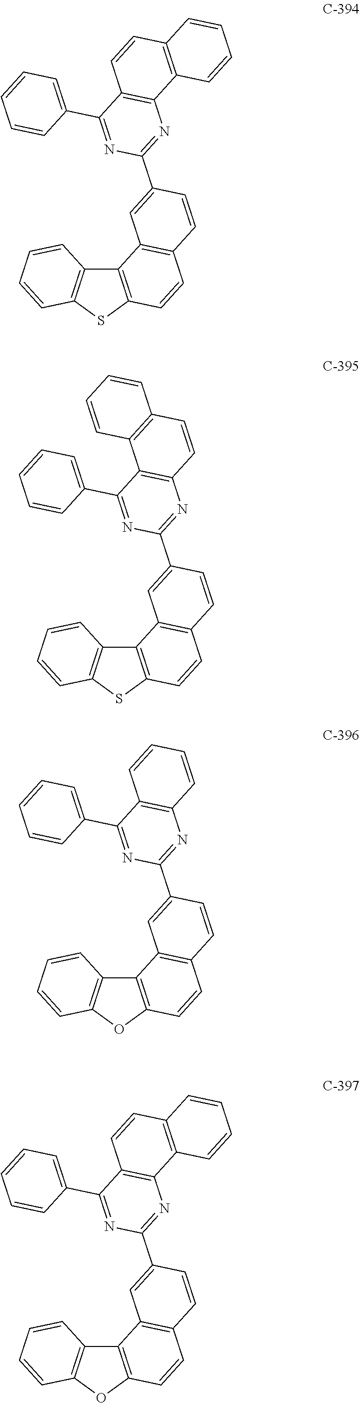 [SMILES: c1ccc(-c2nc(-c3ccc4ccc5sc6ccccc6c5c4c3)nc3c2ccc2ccccc23)cc1 ; c1ccc(-c2nc(-c3ccc4ccc5sc6ccccc6c5c4c3)nc3ccc4ccccc4c23)cc1 ; c1ccc(-c2nc(-c3ccc4ccc5oc6ccccc6c5c4c3)nc3ccccc23)cc1 ; c1ccc(-c2nc(-c3ccc4ccc5oc6ccccc6c5c4c3)nc3c2ccc2ccccc23)cc1]

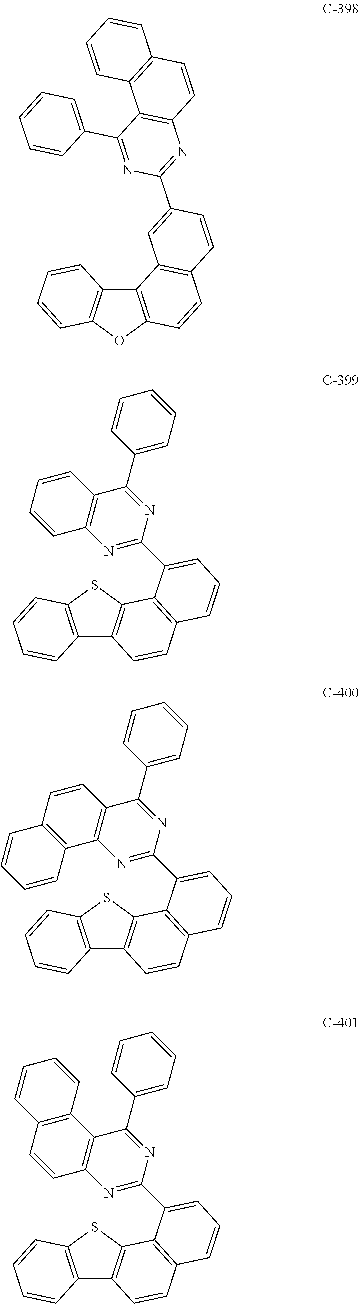 [SMILES: c1ccc(-c2nc(-c3ccc4ccc5oc6ccccc6c5c4c3)nc3ccc4ccccc4c23)cc1 ; c1ccc(-c2nc(-c3cccc4ccc5c6ccccc6sc5c34)nc3ccccc23)cc1 ; c1ccc(-c2nc(-c3cccc4ccc5c6ccccc6sc5c34)nc3c2ccc2ccccc23)cc1 ; c1ccc(-c2nc(-c3cccc4ccc5c6ccccc6sc5c34)nc3ccc4ccccc4c23)cc1]

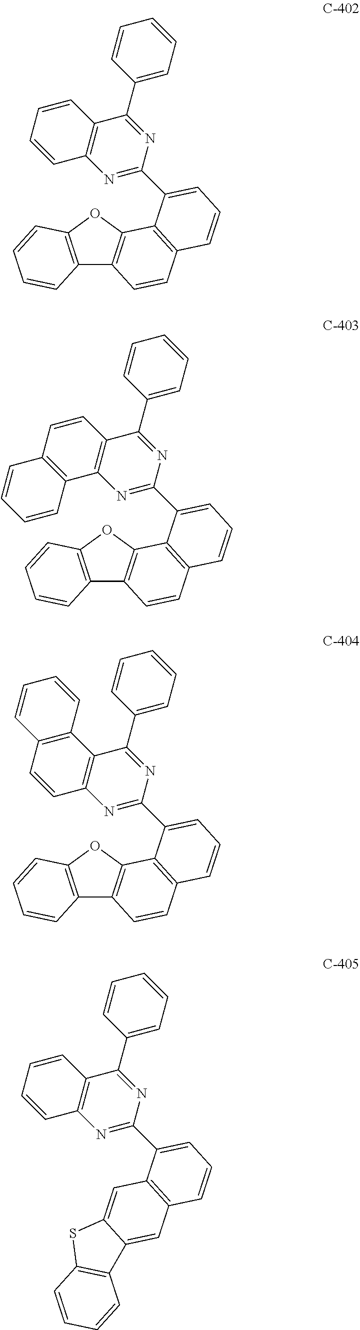

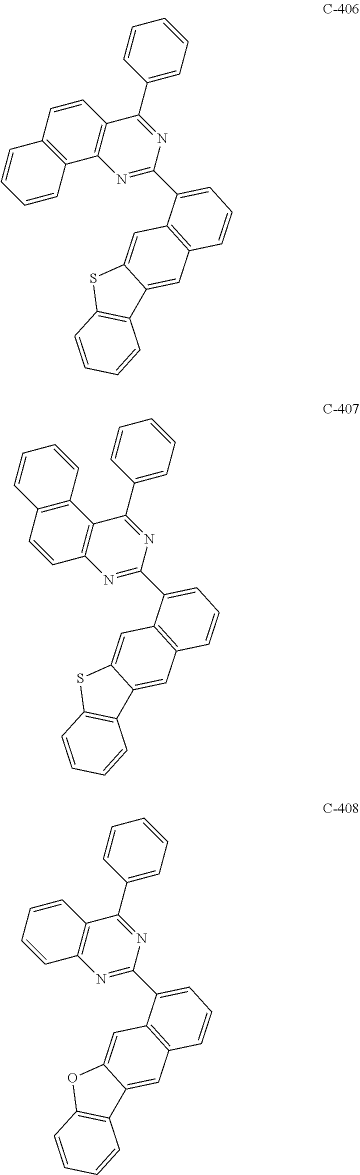 [SMILES: c1ccc(-c2nc(-c3cccc4cc5c(cc34)sc3ccccc35)nc3c2ccc2ccccc23)cc1 ; c1ccc(-c2nc(-c3cccc4cc5c(cc34)sc3ccccc35)nc3ccc4ccccc4c23)cc1 ; c1ccc(-c2nc(-c3cccc4cc5c(cc34)oc3ccccc35)nc3ccccc23)cc1]

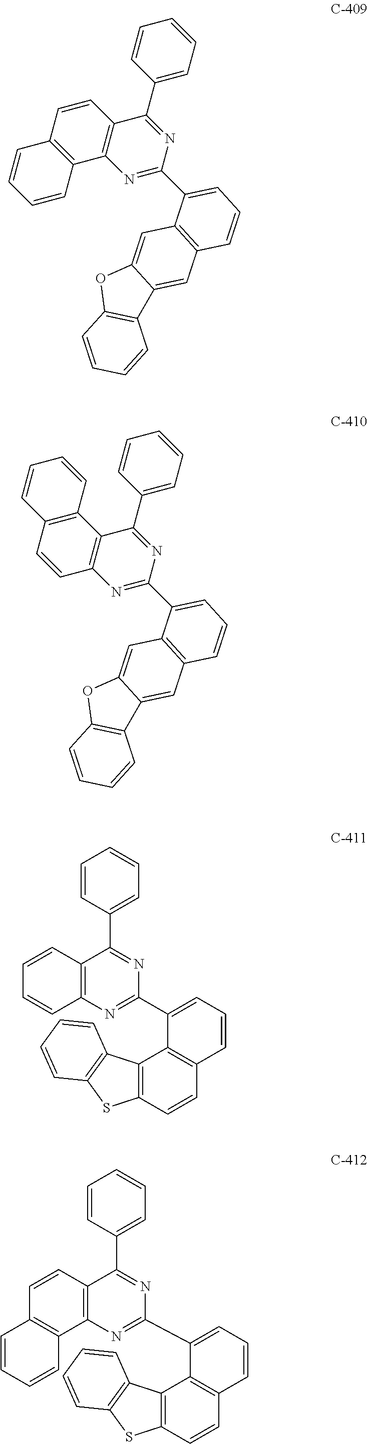 [SMILES: c1ccc(-c2nc(-c3cccc4cc5c(cc34)oc3ccccc35)nc3c2ccc2ccccc23)cc1 ; c1ccc(-c2nc(-c3cccc4cc5c(cc34)oc3ccccc35)nc3ccc4ccccc4c23)cc1 ; c1ccc(-c2nc(-c3cccc4ccc5sc6ccccc6c5c34)nc3ccccc23)cc1 ; c1ccc(-c2nc(-c3cccc4ccc5sc6ccccc6c5c34)nc3c2ccc2ccccc23)cc1]

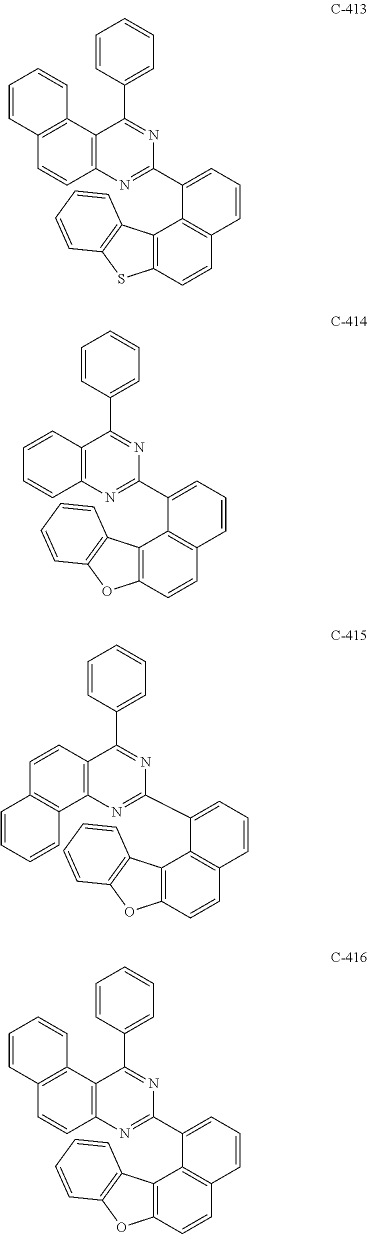 [SMILES: c1ccc(-c2nc(-c3cccc4ccc5sc6ccccc6c5c34)nc3ccc4ccccc4c23)cc1 ; c1ccc(-c2nc(-c3cccc4ccc5oc6ccccc6c5c34)nc3ccccc23)cc1 ; c1ccc(-c2nc(-c3cccc4ccc5oc6ccccc6c5c34)nc3c2ccc2ccccc23)cc1 ; c1ccc(-c2nc(-c3cccc4ccc5oc6ccccc6c5c34)nc3ccc4ccccc4c23)cc1]

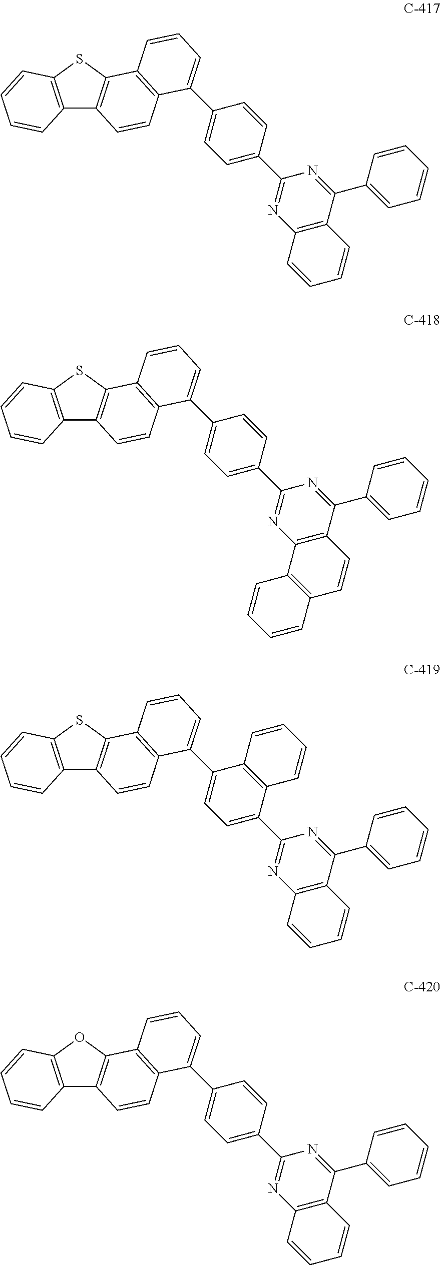 [SMILES: c1ccc(-c2nc(-c3ccc(-c4cccc5c4ccc4c6ccccc6sc54)cc3)nc3ccccc23)cc1 ; c1ccc(-c2nc(-c3ccc(-c4cccc5c4ccc4c6ccccc6sc54)cc3)nc3c2ccc2ccccc23)cc1 ; c1ccc(-c2nc(-c3ccc(-c4cccc5c4ccc4c6ccccc6sc54)c4ccccc34)nc3ccccc23)cc1 ; c1ccc(-c2nc(-c3ccc(-c4cccc5c4ccc4c6ccccc6oc54)cc3)nc3ccccc23)cc1]

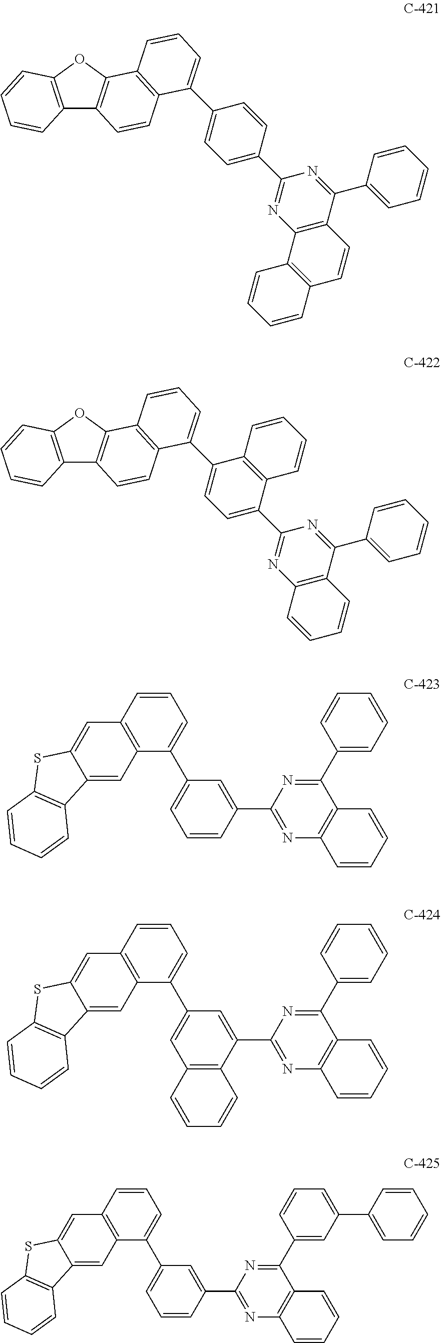 [SMILES: c1ccc(-c2nc(-c3ccc(-c4cccc5c4ccc4c6ccccc6oc54)cc3)nc3c2ccc2ccccc23)cc1 ; c1ccc(-c2nc(-c3ccc(-c4cccc5c4ccc4c6ccccc6oc54)c4ccccc34)nc3ccccc23)cc1 ; c1ccc(-c2nc(-c3cccc(-c4cccc5cc6sc7ccccc7c6cc45)c3)nc3ccccc23)cc1 ; c1ccc(-c2nc(-c3cc(-c4cccc5cc6sc7ccccc7c6cc45)cc4ccccc34)nc3ccccc23)cc1 ; c1ccc(-c2cccc(-c3nc(-c4cccc(-c5cccc6cc7sc8ccccc8c7cc56)c4)nc4ccccc34)c2)cc1]

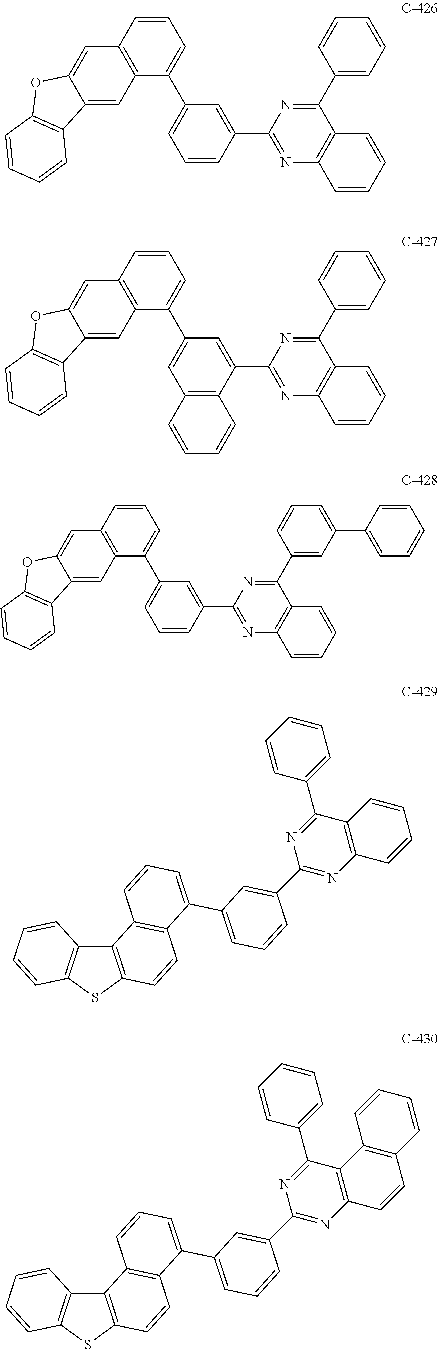 [SMILES: c1ccc(-c2nc(-c3cccc(-c4cccc5cc6oc7ccccc7c6cc45)c3)nc3ccccc23)cc1 ; c1ccc(-c2nc(-c3cc(-c4cccc5cc6oc7ccccc7c6cc45)cc4ccccc34)nc3ccccc23)cc1 ; c1ccc(-c2cccc(-c3nc(-c4cccc(-c5cccc6cc7oc8ccccc8c7cc56)c4)nc4ccccc34)c2)cc1 ; c1ccc(-c2nc(-c3cccc(-c4cccc5c4ccc4sc6ccccc6c45)c3)nc3ccccc23)cc1 ; c1ccc(-c2nc(-c3cccc(-c4cccc5c4ccc4sc6ccccc6c45)c3)nc3ccc4ccccc4c23)cc1]

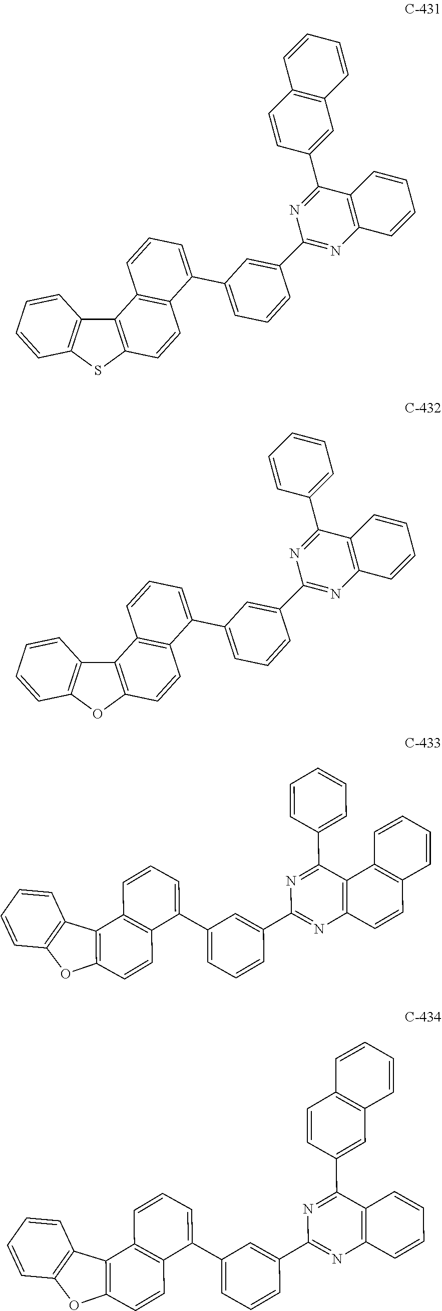 [SMILES: c1cc(-c2nc(-c3ccc4ccccc4c3)c3ccccc3n2)cc(-c2cccc3c2ccc2sc4ccccc4c23)c1 ; c1ccc(-c2nc(-c3cccc(-c4cccc5c4ccc4oc6ccccc6c45)c3)nc3ccccc23)cc1 ; c1ccc(-c2nc(-c3cccc(-c4cccc5c4ccc4oc6ccccc6c45)c3)nc3ccc4ccccc4c23)cc1 ; c1cc(-c2nc(-c3ccc4ccccc4c3)c3ccccc3n2)cc(-c2cccc3c2ccc2oc4ccccc4c23)c1]

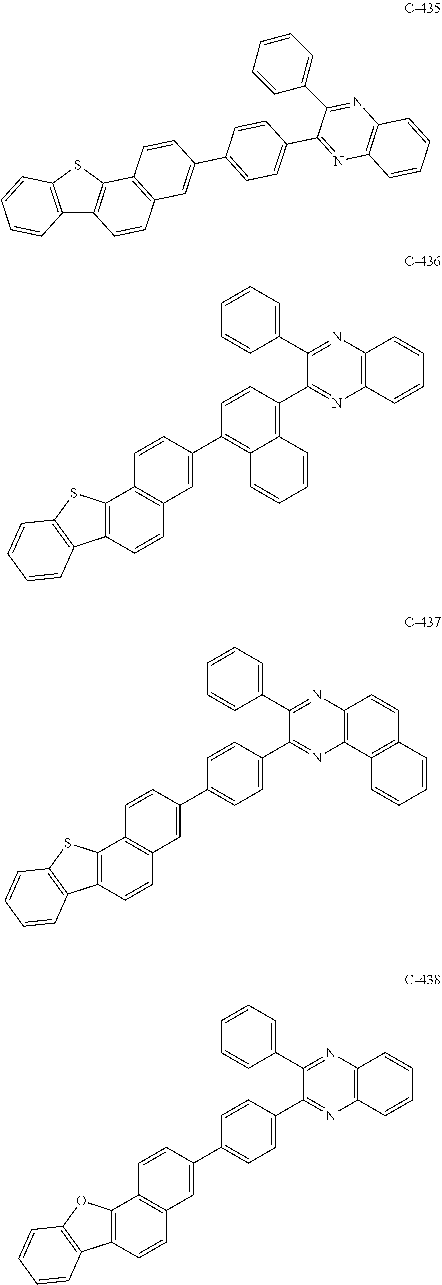 [SMILES: c1ccc(-c2nc3ccccc3nc2-c2ccc(-c3ccc4c(ccc5c6ccccc6sc45)c3)cc2)cc1 ; c1ccc(-c2nc3ccccc3nc2-c2ccc(-c3ccc4c(ccc5c6ccccc6sc45)c3)c3ccccc23)cc1 ; c1ccc(-c2nc3ccc4ccccc4c3nc2-c2ccc(-c3ccc4c(ccc5c6ccccc6sc45)c3)cc2)cc1 ; c1ccc(-c2nc3ccccc3nc2-c2ccc(-c3ccc4c(ccc5c6ccccc6oc45)c3)cc2)cc1]

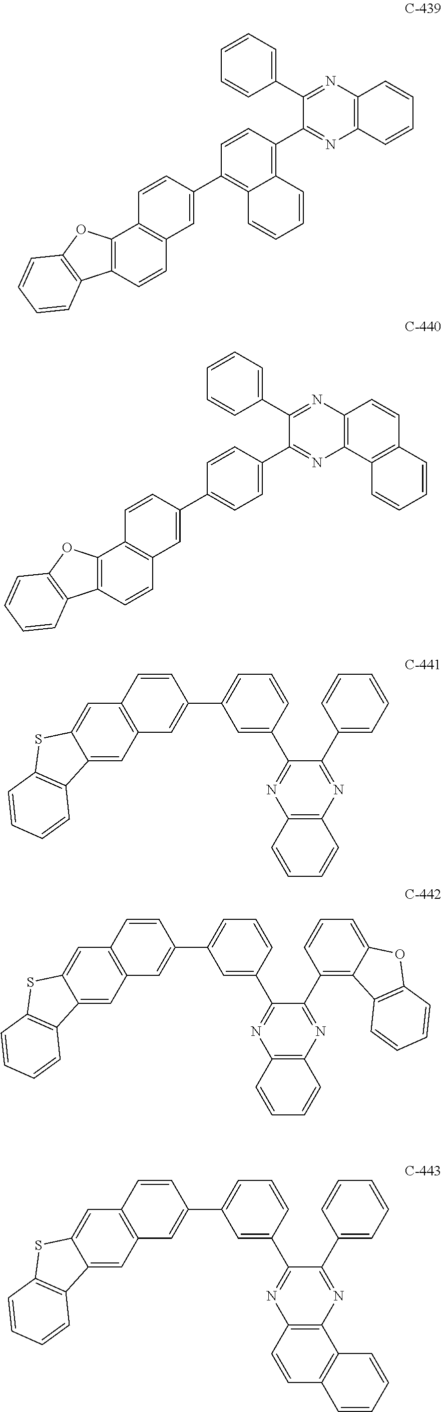

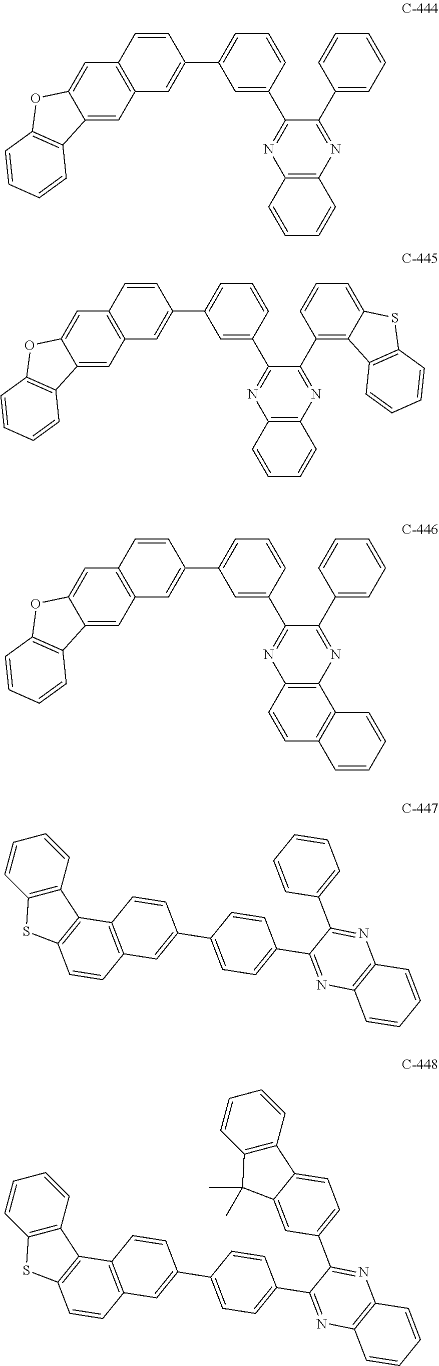 [SMILES: c1ccc(-c2nc3ccccc3nc2-c2cccc(-c3ccc4cc5oc6ccccc6c5cc4c3)c2)cc1 ; c1cc(-c2ccc3cc4oc5ccccc5c4cc3c2)cc(-c2nc3ccccc3nc2-c2cccc3sc4ccccc4c23)c1 ; c1ccc(-c2nc3c(ccc4ccccc43)nc2-c2cccc(-c3ccc4cc5oc6ccccc6c5cc4c3)c2)cc1 ; c1ccc(-c2nc3ccccc3nc2-c2ccc(-c3ccc4c(ccc5sc6ccccc6c54)c3)cc2)cc1 ; CC1(C)c2ccccc2-c2ccc(-c3nc4ccccc4nc3-c3ccc(-c4ccc5c(ccc6sc7ccccc7c65)c4)cc3)cc21]

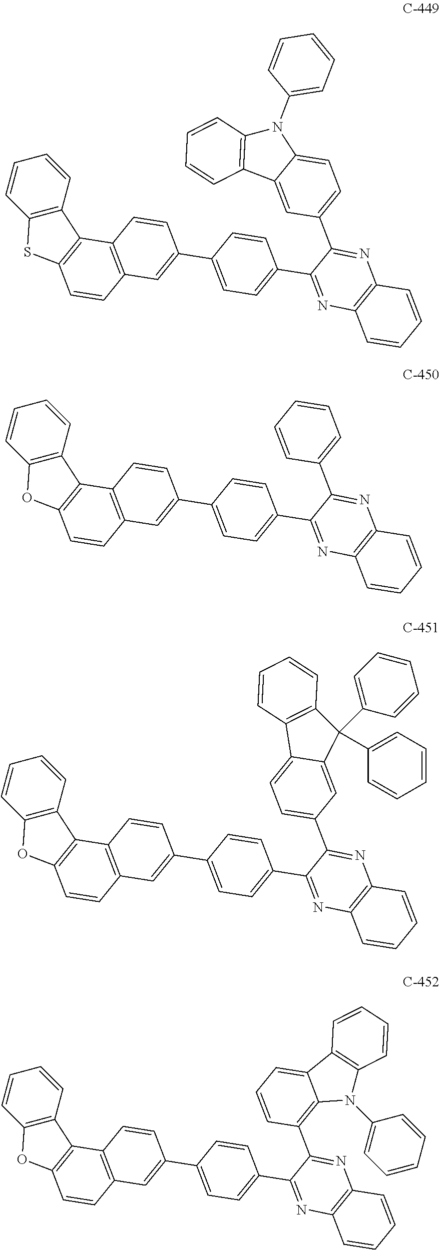 [SMILES: c1ccc(-n2c3ccccc3c3cc(-c4nc5ccccc5nc4-c4ccc(-c5ccc6c(ccc7sc8ccccc8c76)c5)cc4)ccc32)cc1 ; c1ccc(-c2nc3ccccc3nc2-c2ccc(-c3ccc4c(ccc5oc6ccccc6c54)c3)cc2)cc1 ; c1ccc(C2(c3ccccc3)c3ccccc3-c3ccc(-c4nc5ccccc5nc4-c4ccc(-c5ccc6c(ccc7oc8ccccc8c76)c5)cc4)cc32)cc1 ; c1ccc(-n2c3ccccc3c3cccc(-c4nc5ccccc5nc4-c4ccc(-c5ccc6c(ccc7oc8ccccc8c76)c5)cc4)c32)cc1]

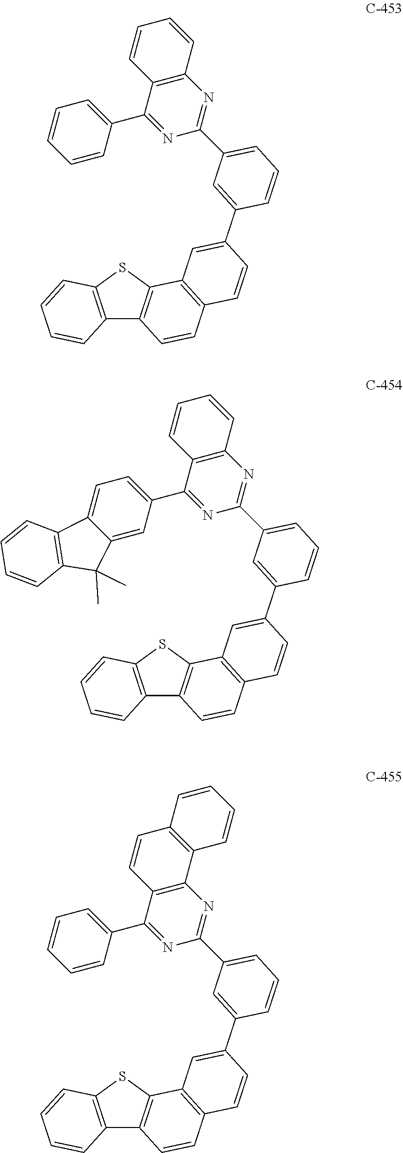 [SMILES: c1ccc(-c2nc(-c3cccc(-c4ccc5ccc6c7ccccc7sc6c5c4)c3)nc3ccccc23)cc1 ; CC1(C)c2ccccc2-c2ccc(-c3nc(-c4cccc(-c5ccc6ccc7c8ccccc8sc7c6c5)c4)nc4ccccc34)cc21 ; c1ccc(-c2nc(-c3cccc(-c4ccc5ccc6c7ccccc7sc6c5c4)c3)nc3c2ccc2ccccc23)cc1]

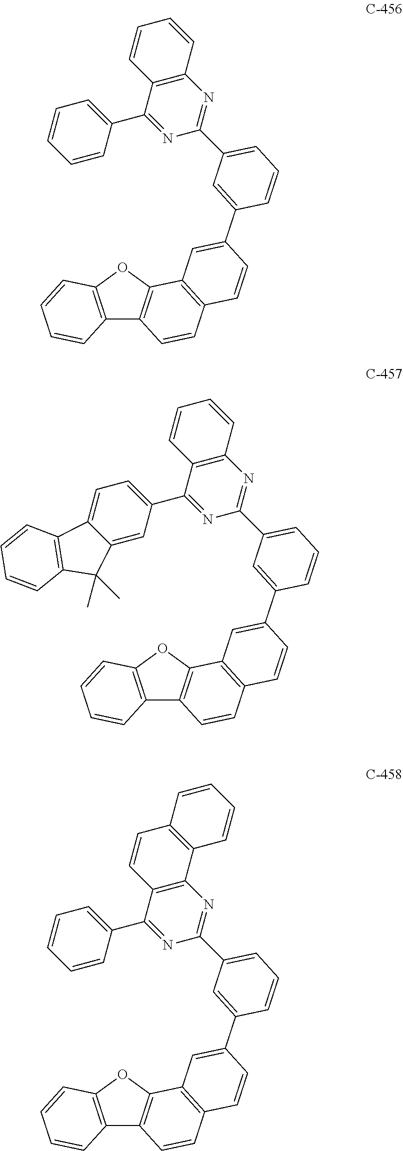 [SMILES: c1ccc(-c2nc(-c3cccc(-c4ccc5ccc6c7ccccc7oc6c5c4)c3)nc3ccccc23)cc1 ; CC1(C)c2ccccc2-c2ccc(-c3nc(-c4cccc(-c5ccc6ccc7c8ccccc8oc7c6c5)c4)nc4ccccc34)cc21 ; c1ccc(-c2nc(-c3cccc(-c4ccc5ccc6c7ccccc7oc6c5c4)c3)nc3c2ccc2ccccc23)cc1]

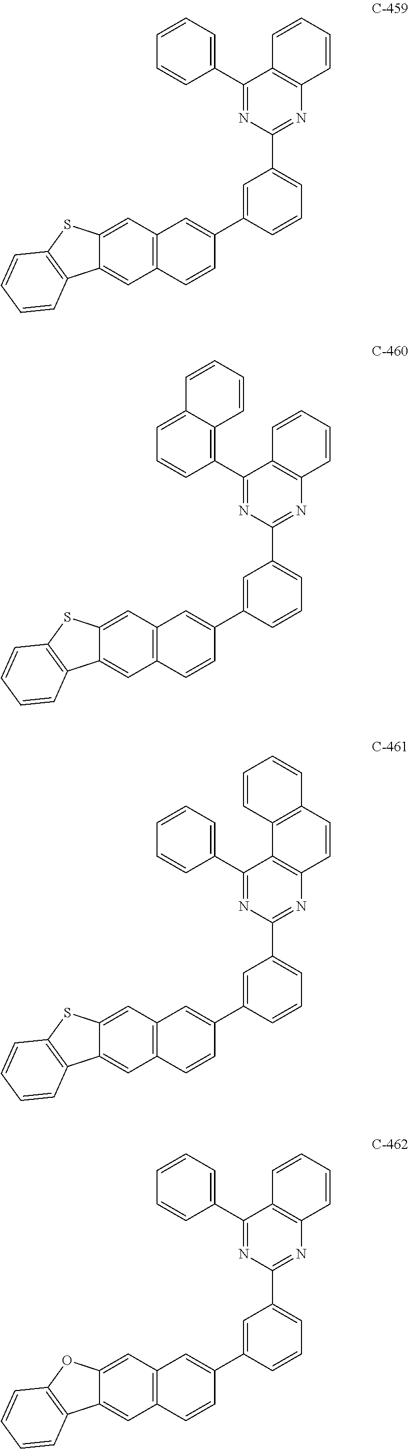 [SMILES: c1ccc(-c2nc(-c3cccc(-c4ccc5cc6c(cc5c4)sc4ccccc46)c3)nc3ccccc23)cc1 ; c1cc(-c2ccc3cc4c(cc3c2)sc2ccccc24)cc(-c2nc(-c3cccc4ccccc34)c3ccccc3n2)c1 ; c1ccc(-c2nc(-c3cccc(-c4ccc5cc6c(cc5c4)sc4ccccc46)c3)nc3ccc4ccccc4c23)cc1 ; c1ccc(-c2nc(-c3cccc(-c4ccc5cc6c(cc5c4)oc4ccccc46)c3)nc3ccccc23)cc1]

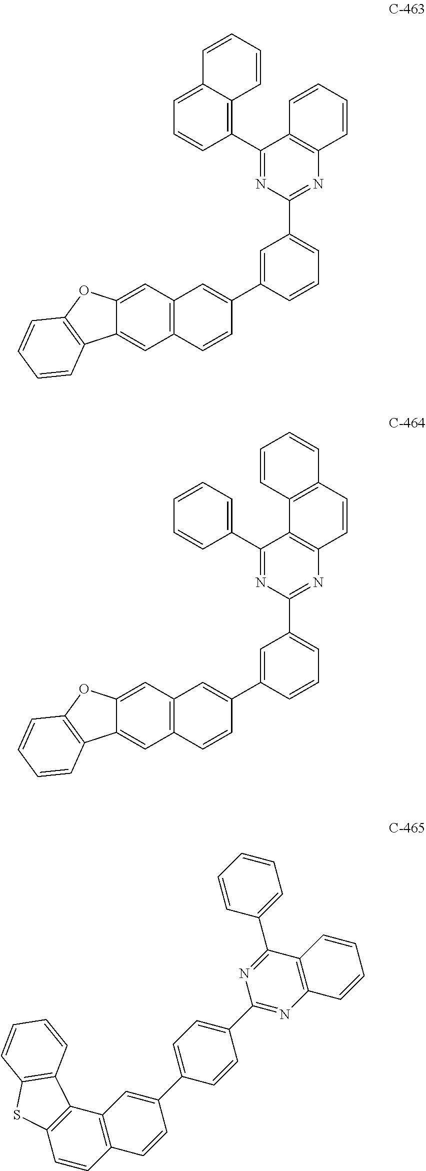 [SMILES: c1cc(-c2ccc3cc4c(cc3c2)oc2ccccc24)cc(-c2nc(-c3cccc4ccccc34)c3ccccc3n2)c1 ; c1ccc(-c2nc(-c3cccc(-c4ccc5cc6c(cc5c4)oc4ccccc46)c3)nc3ccc4ccccc4c23)cc1 ; c1ccc(-c2nc(-c3ccc(-c4ccc5ccc6sc7ccccc7c6c5c4)cc3)nc3ccccc23)cc1]

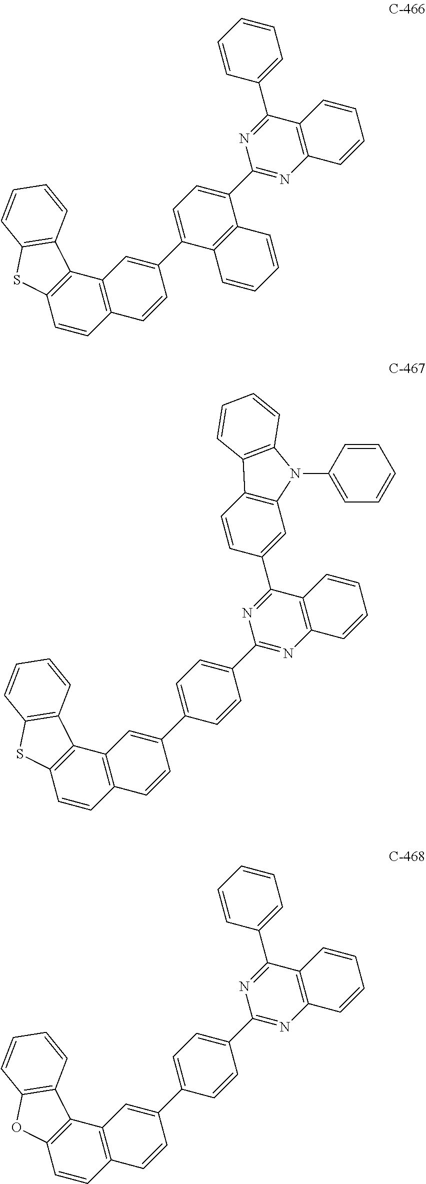 [SMILES: c1ccc(-c2nc(-c3ccc(-c4ccc5ccc6sc7ccccc7c6c5c4)c4ccccc34)nc3ccccc23)cc1 ; c1ccc(-n2c3ccccc3c3ccc(-c4nc(-c5ccc(-c6ccc7ccc8sc9ccccc9c8c7c6)cc5)nc5ccccc45)cc32)cc1 ; c1ccc(-c2nc(-c3ccc(-c4ccc5ccc6oc7ccccc7c6c5c4)cc3)nc3ccccc23)cc1]

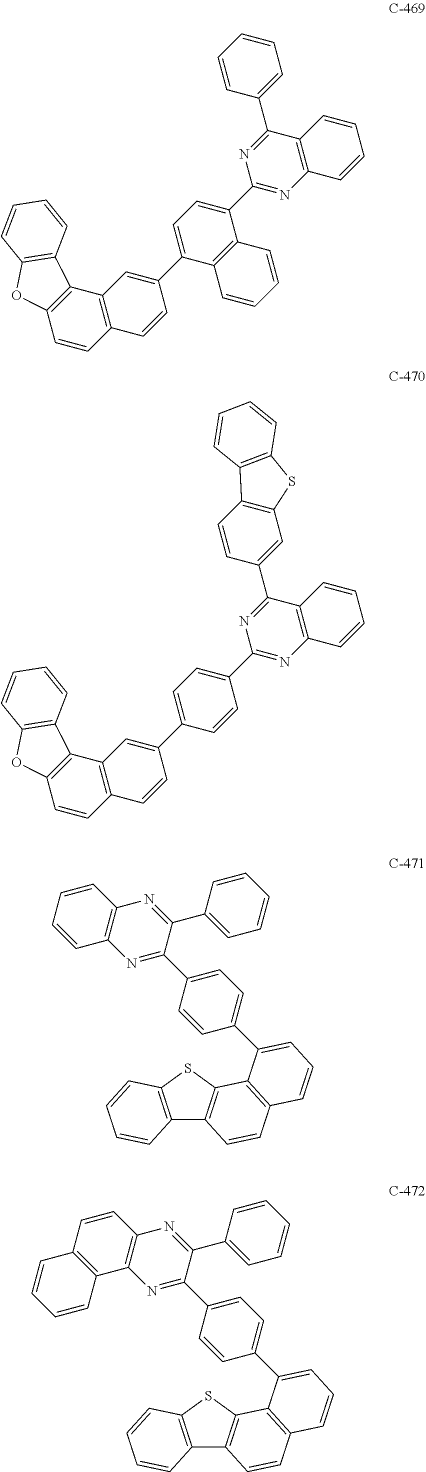 [SMILES: c1ccc(-c2nc(-c3ccc(-c4ccc5ccc6oc7ccccc7c6c5c4)c4ccccc34)nc3ccccc23)cc1 ; c1ccc2c(-c3ccc4c(c3)sc3ccccc34)nc(-c3ccc(-c4ccc5ccc6oc7ccccc7c6c5c4)cc3)nc2c1 ; c1ccc(-c2nc3ccccc3nc2-c2ccc(-c3cccc4ccc5c6ccccc6sc5c34)cc2)cc1 ; c1ccc(-c2nc3ccc4ccccc4c3nc2-c2ccc(-c3cccc4ccc5c6ccccc6sc5c34)cc2)cc1]

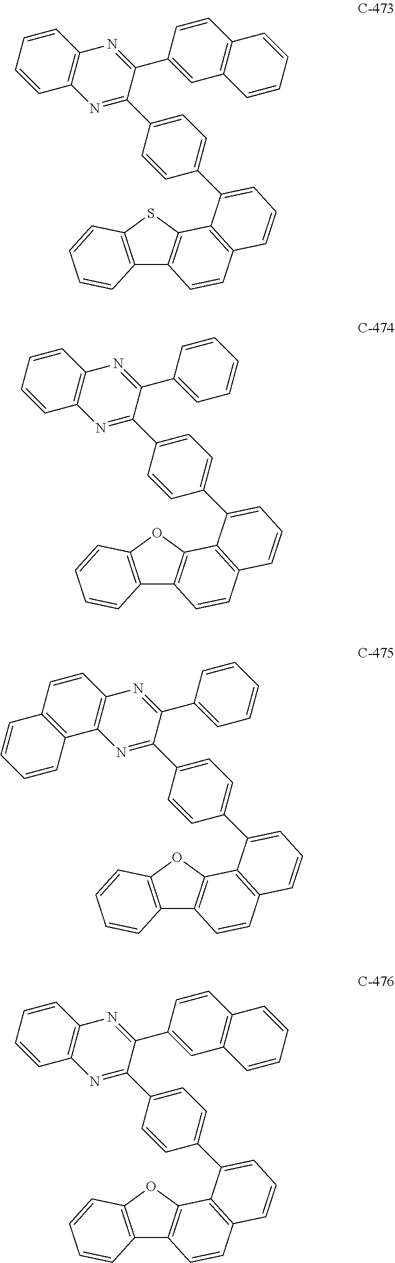 [SMILES: c1ccc2cc(-c3nc4ccccc4nc3-c3ccc(-c4cccc5ccc6c7ccccc7sc6c45)cc3)ccc2c1 ; c1ccc(-c2nc3ccccc3nc2-c2ccc(-c3cccc4ccc5c6ccccc6oc5c34)cc2)cc1 ; c1ccc(-c2nc3ccc4ccccc4c3nc2-c2ccc(-c3cccc4ccc5c6ccccc6oc5c34)cc2)cc1 ; c1ccc2cc(-c3nc4ccccc4nc3-c3ccc(-c4cccc5ccc6c7ccccc7oc6c45)cc3)ccc2c1]

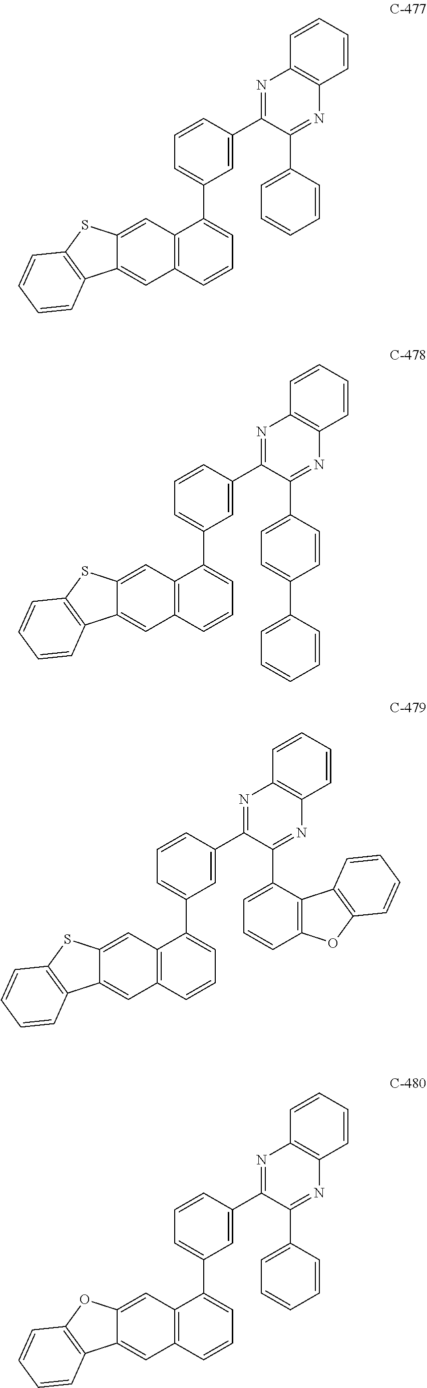 [SMILES: c1ccc(-c2nc3ccccc3nc2-c2cccc(-c3cccc4cc5c(cc34)sc3ccccc35)c2)cc1 ; c1ccc(-c2ccc(-c3nc4ccccc4nc3-c3cccc(-c4cccc5cc6c(cc45)sc4ccccc46)c3)cc2)cc1 ; c1cc(-c2nc3ccccc3nc2-c2cccc3oc4ccccc4c23)cc(-c2cccc3cc4c(cc23)sc2ccccc24)c1 ; c1ccc(-c2nc3ccccc3nc2-c2cccc(-c3cccc4cc5c(cc34)oc3ccccc35)c2)cc1]

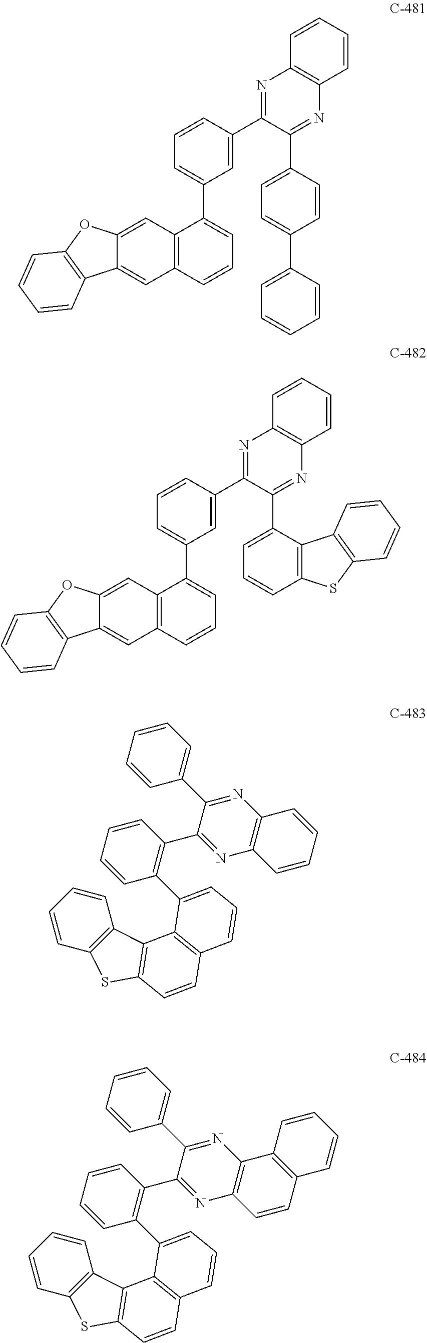

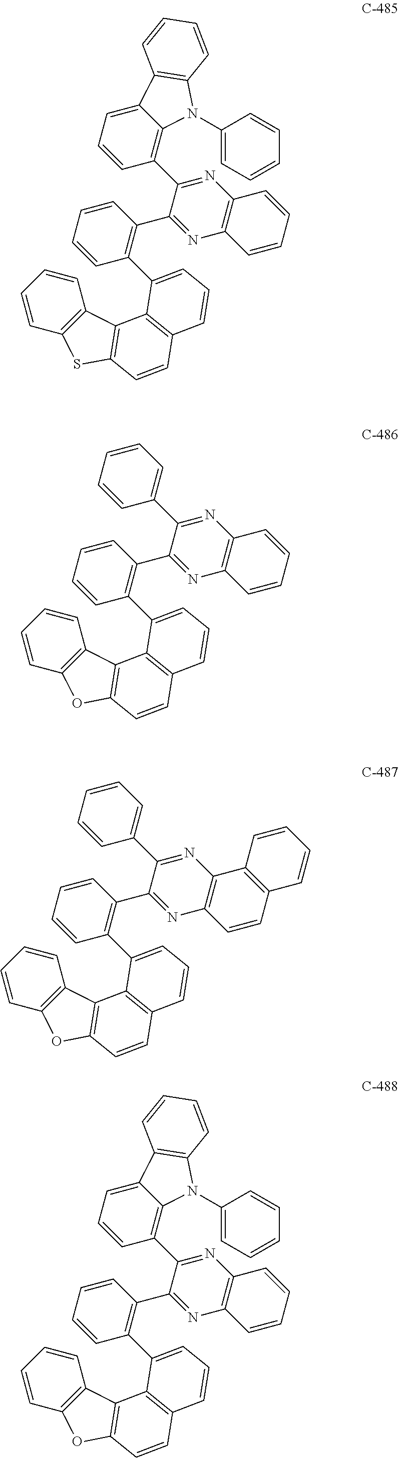 [SMILES: c1ccc(-n2c3ccccc3c3cccc(-c4nc5ccccc5nc4-c4ccccc4-c4cccc5ccc6sc7ccccc7c6c45)c32)cc1 ; c1ccc(-c2nc3ccccc3nc2-c2ccccc2-c2cccc3ccc4oc5ccccc5c4c23)cc1 ; c1ccc(-c2nc3c(ccc4ccccc43)nc2-c2ccccc2-c2cccc3ccc4oc5ccccc5c4c23)cc1 ; c1ccc(-n2c3ccccc3c3cccc(-c4nc5ccccc5nc4-c4ccccc4-c4cccc5ccc6oc7ccccc7c6c45)c32)cc1]

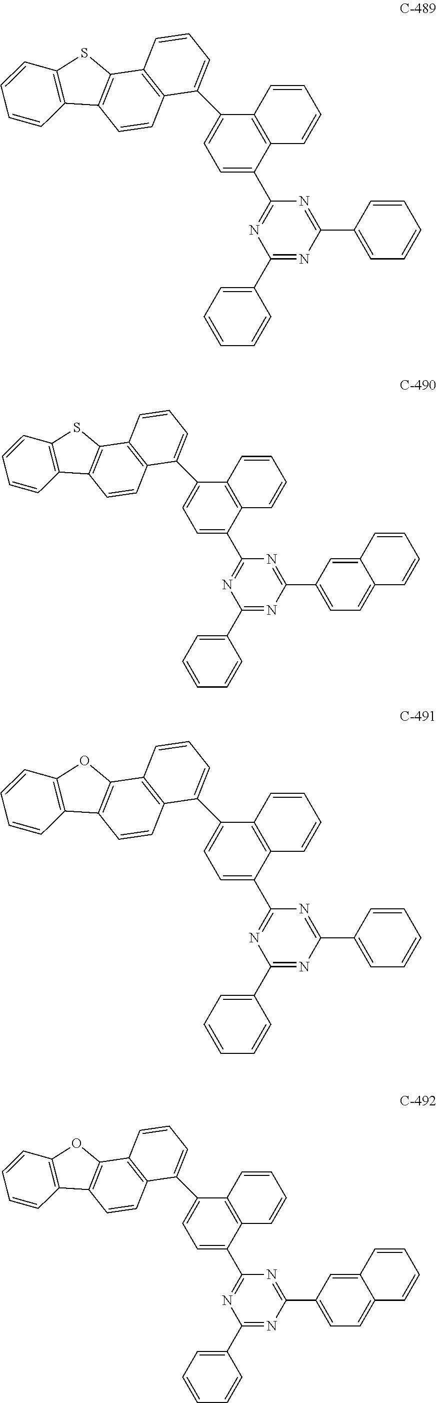 [SMILES: c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1]

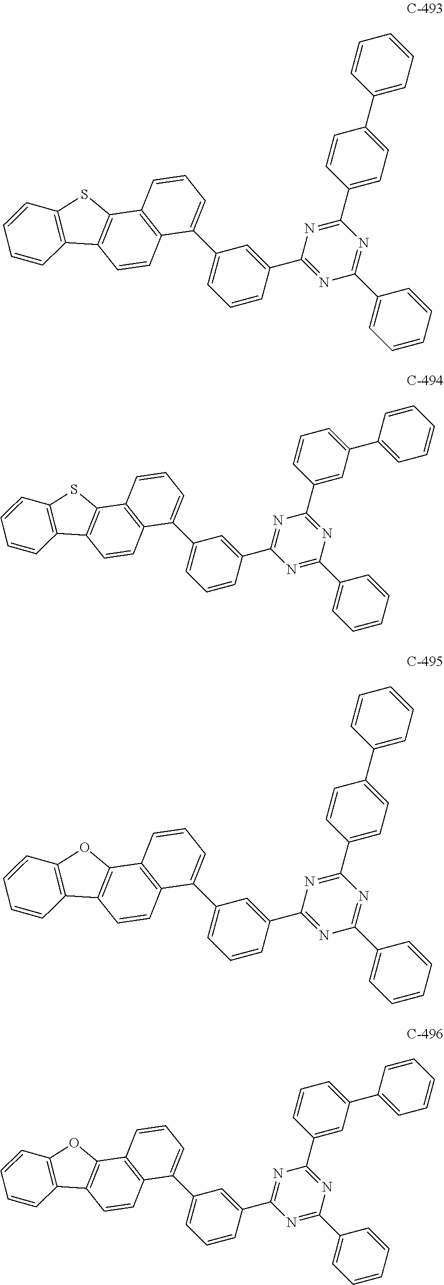 [SMILES: c1ccc(-c2ccc(-c3nc(-c4ccccc4)nc(-c4cccc(-c5cccc6c5ccc5c7ccccc7sc65)c4)n3)cc2)cc1 ; c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1]

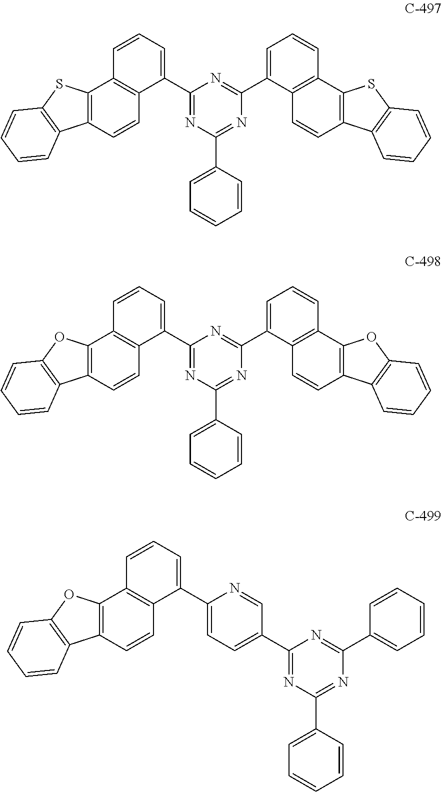 [SMILES: c1ccc(-c2nc(-c3cccc4c3ccc3c5ccccc5sc43)nc(-c3cccc4c3ccc3c5ccccc5sc43)n2)cc1 ; c1ccc(-c2nc(-c3cccc4c3ccc3c5ccccc5oc43)nc(-c3cccc4c3ccc3c5ccccc5oc43)n2)cc1 ; c1ccc(-c2nc(-c3ccccc3)nc(-c3ccc(-c4cccc5c4ccc4c6ccccc6oc54)nc3)n2)cc1]

The organic electroluminescent compound according to the present disclosure may be prepared by a synthetic method known to one skilled in the art, and for example may be prepared as shown in the following reaction schemes 1 to 4, but is not limited thereto.

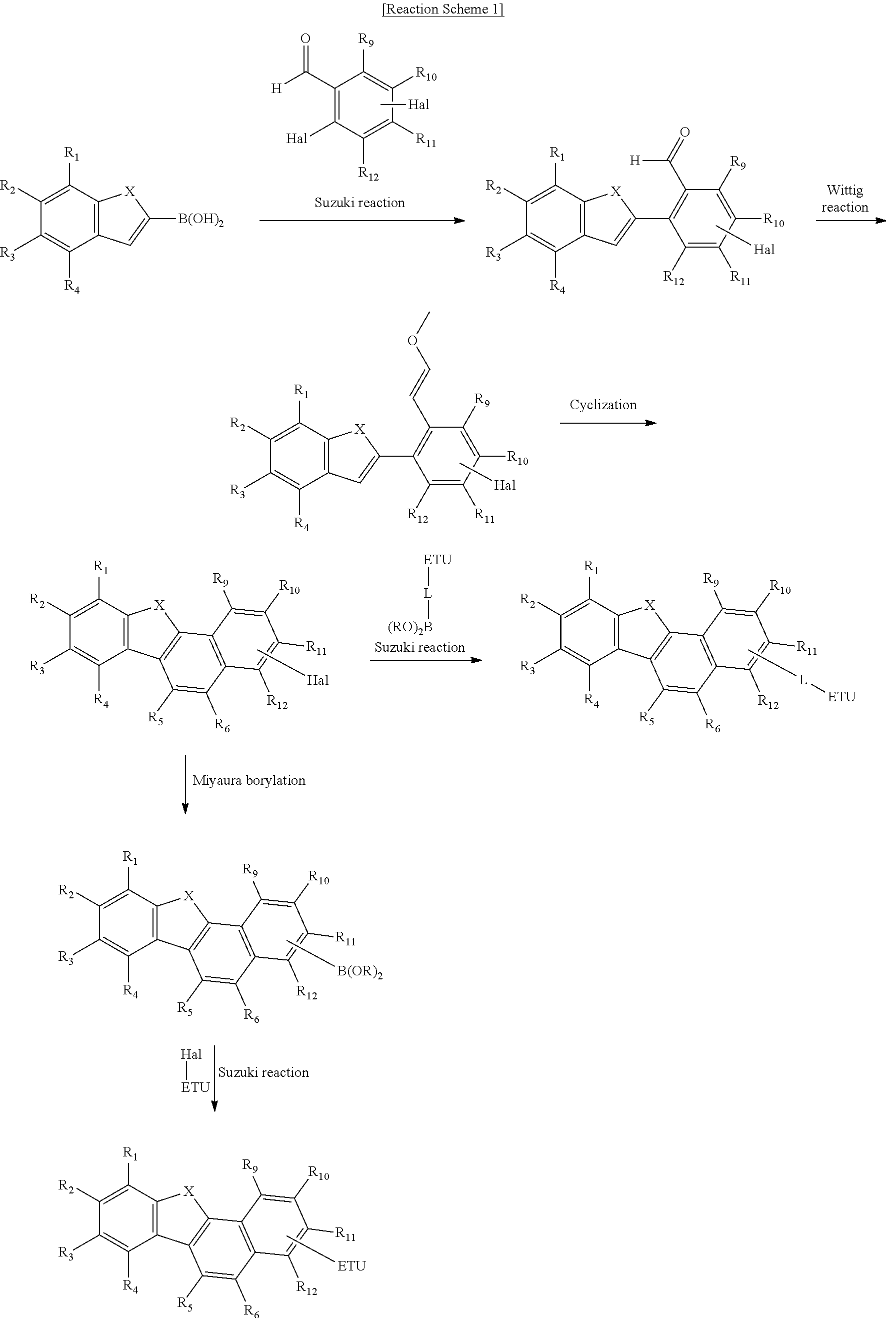

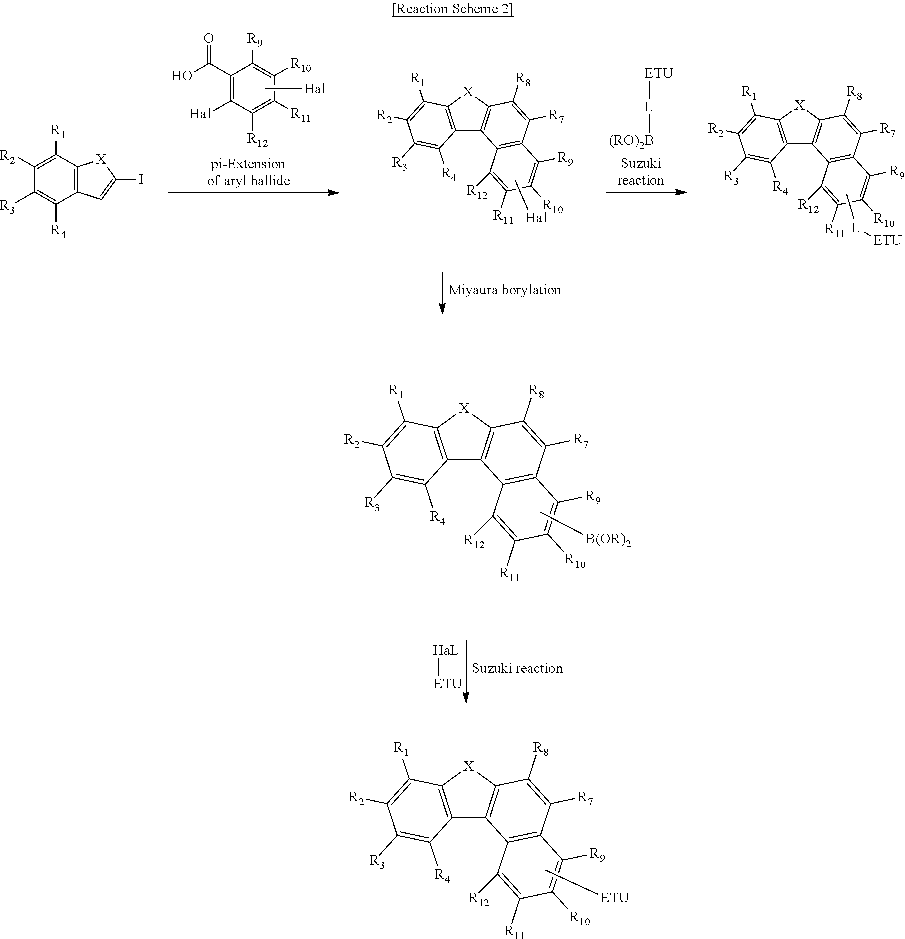

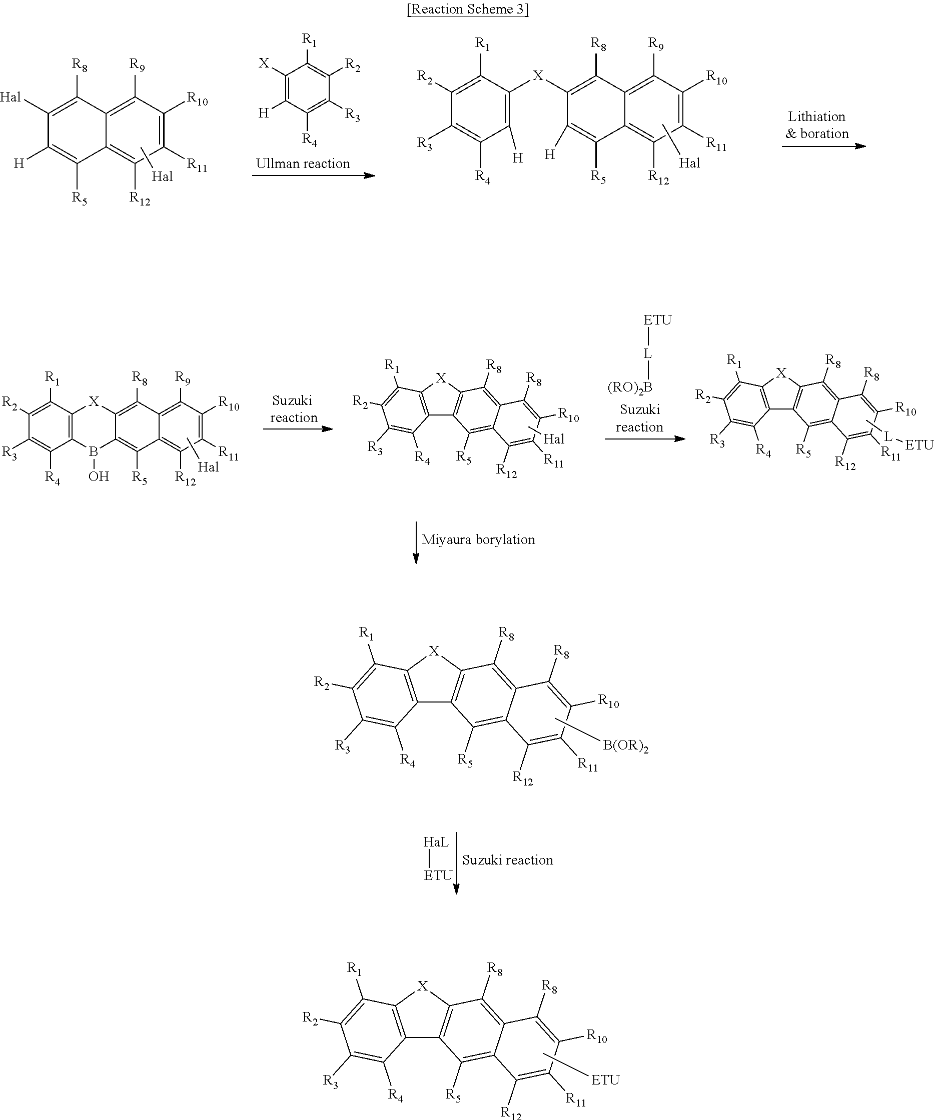

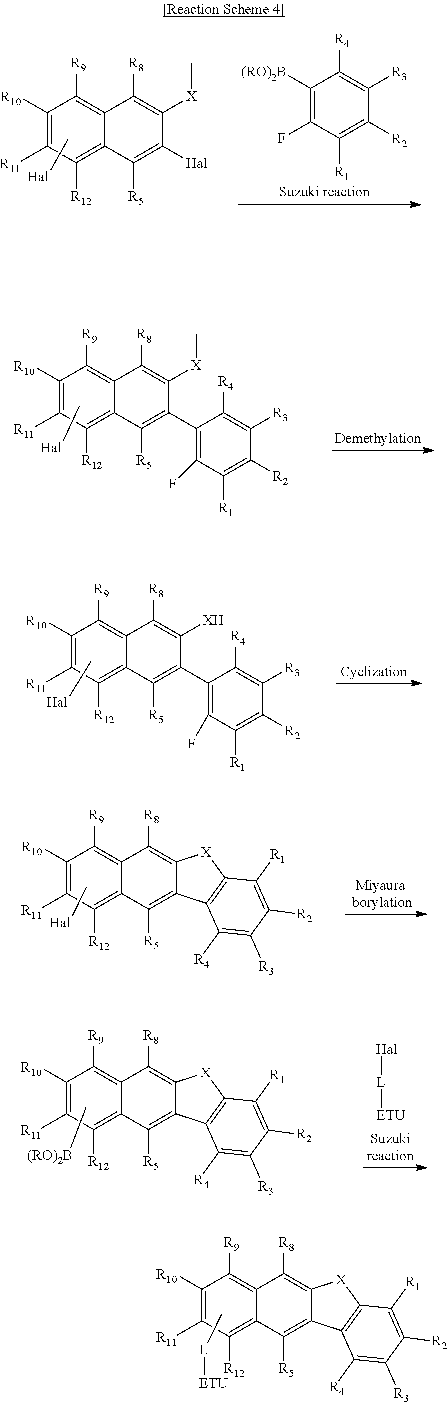

In reaction schemes 1 to 4, R₁ to R₁₂, X, L, and ETU are as defined in formula 1, and Hal represents a halogen.

Although illustrative synthesis examples of the compound represented by formula 1 are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 1 above but are not specified in the specific synthesis examples, are bonded.

The dopant that can be used in combination with the compound according to the present disclosure may be at least one phosphorescent or fluorescent dopant, preferably at least one phosphorescent dopant. The phosphorescent dopant material is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise the compound represented by the following formula 101, but is not limited thereto.

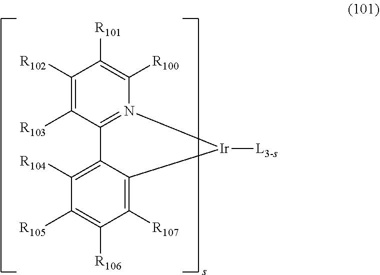

In formula 101, L is any one selected from the following structures 1 to 3:

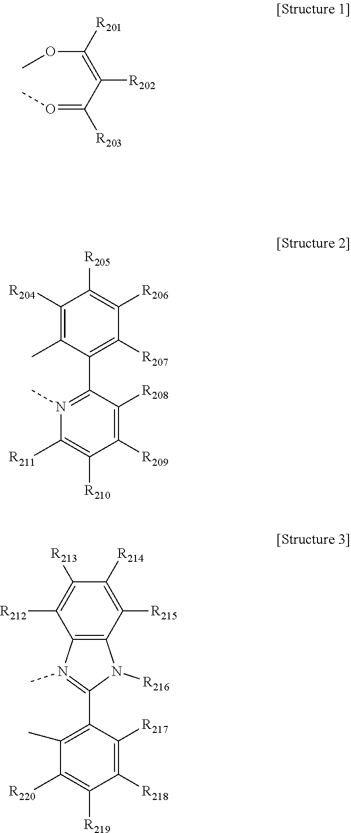

R₁₀₀ to R₁₀₃, each independently, represent hydrogen, deuterium, a halogen, a (C1-C30)alkyl unsubstituted or substituted with deuterium or a halogen, a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to adjacent one(s) of R₁₀₀ to R₁₀₃, to form a substituted or unsubstituted fused ring with a pyridine, e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline or a substituted or unsubstituted indenoquinoline;

R₁₀₄ to R₁₀₇, each independently, represent hydrogen, deuterium, a halogen, a (C1-C30)alkyl unsubstituted or substituted with deuterium or a halogen, a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to adjacent one(s) of R₁₀₄ to R₁₀₇ to form a substituted or unsubstituted fused ring with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₂₀₁ to R₂₂₀, each independently, represent hydrogen, deuterium, a halogen, a (C1-C30)alkyl unsubstituted or substituted with deuterium or a halogen, a substituted or unsubstituted (C3-C30)cycloalkyl, or a substituted or unsubstituted (C6-C30)aryl; or may be linked to adjacent one(s) of R₂₀₁ to R₂₂₀ to form a substituted or unsubstituted fused ring; and

s represents an integer of 1 to 3.

The specific examples of the dopant compound are as follows, but are not limited thereto.

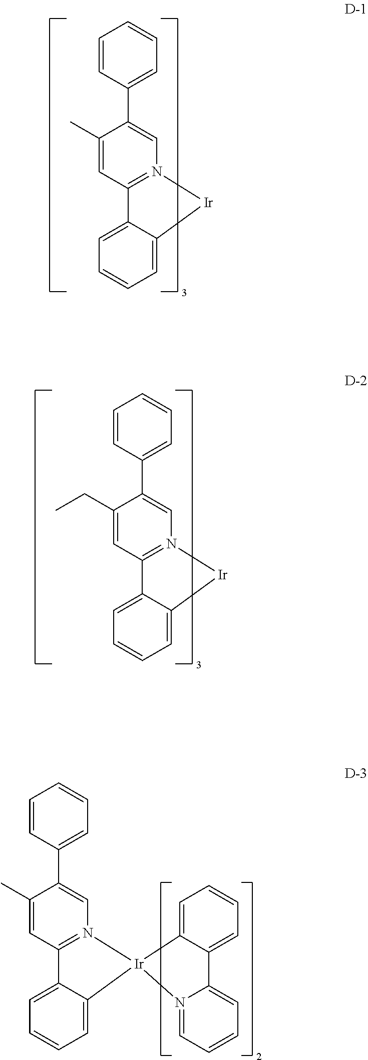

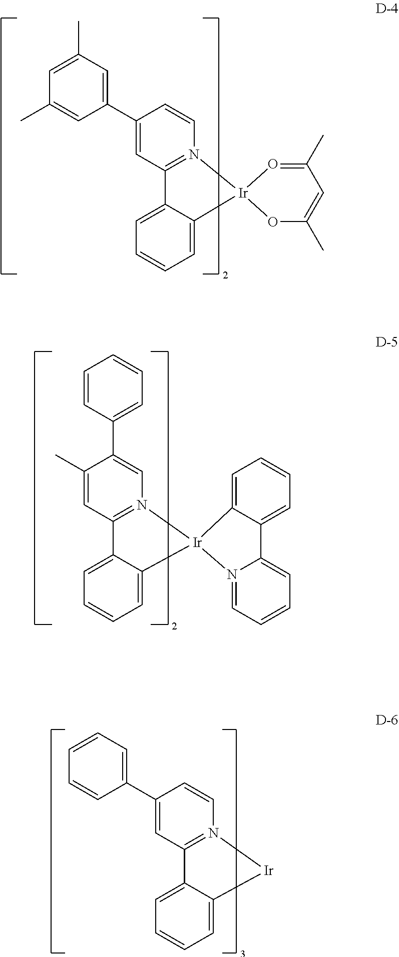

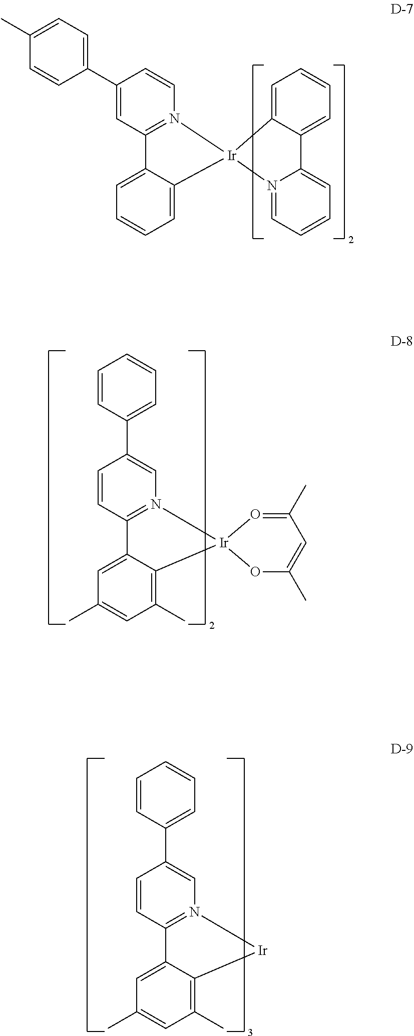

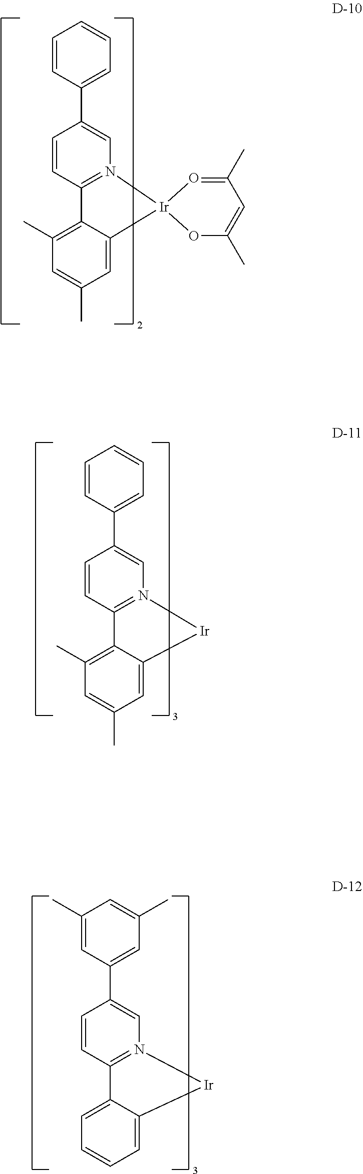

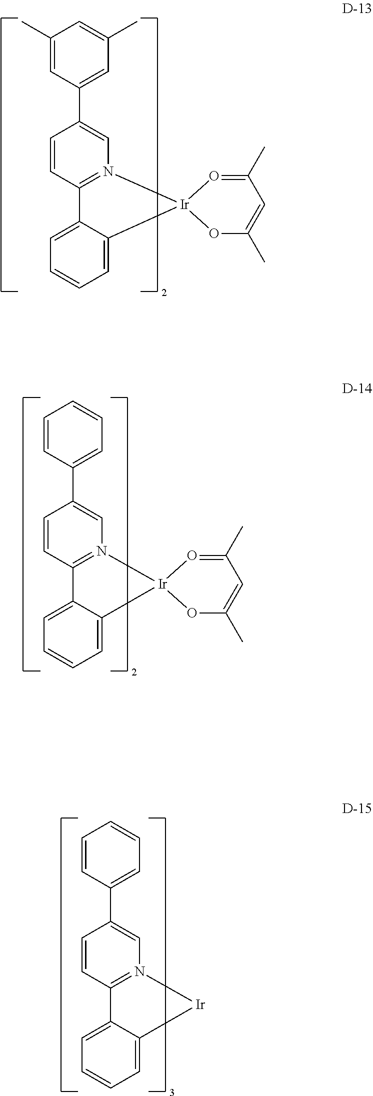

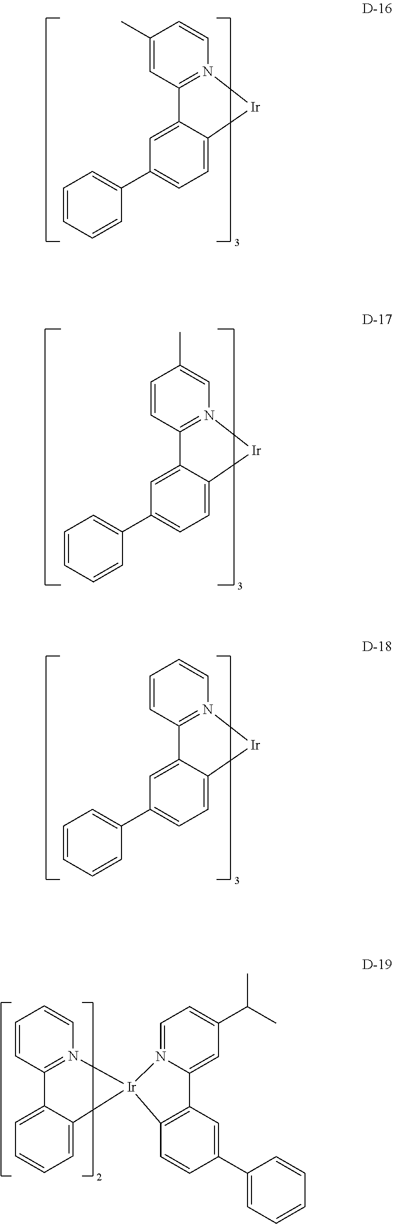

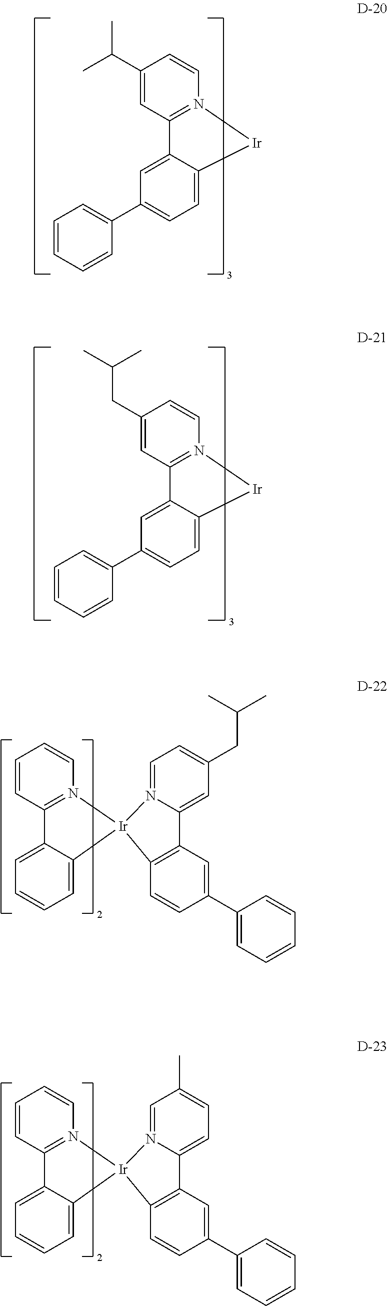

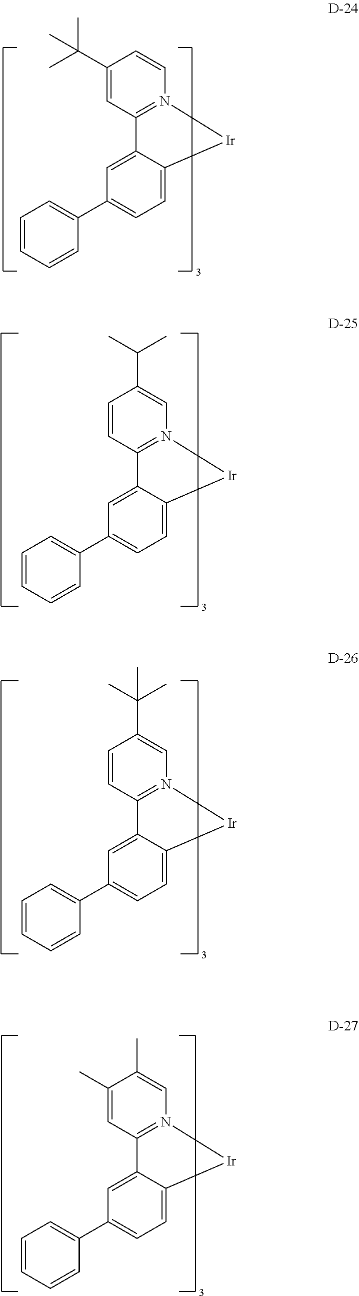

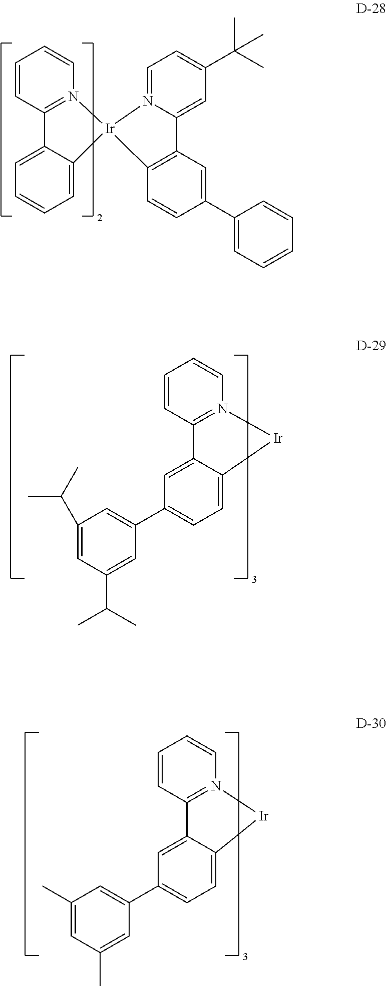

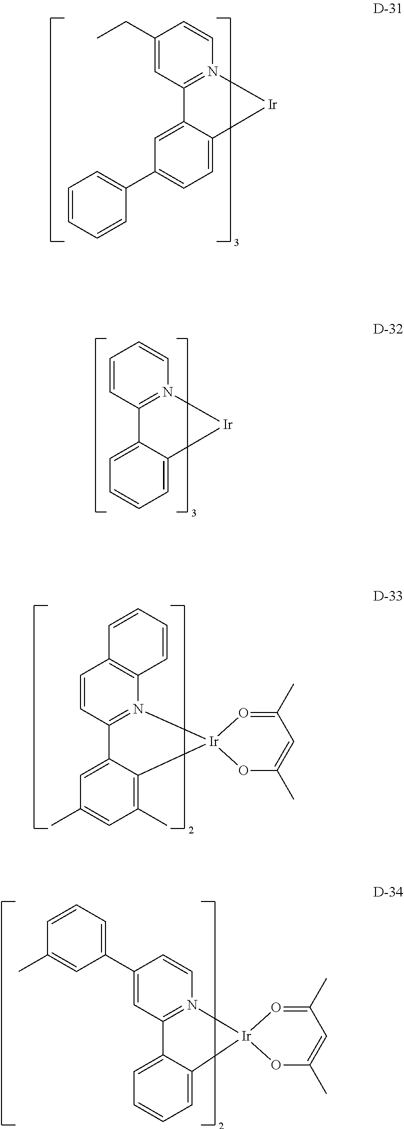

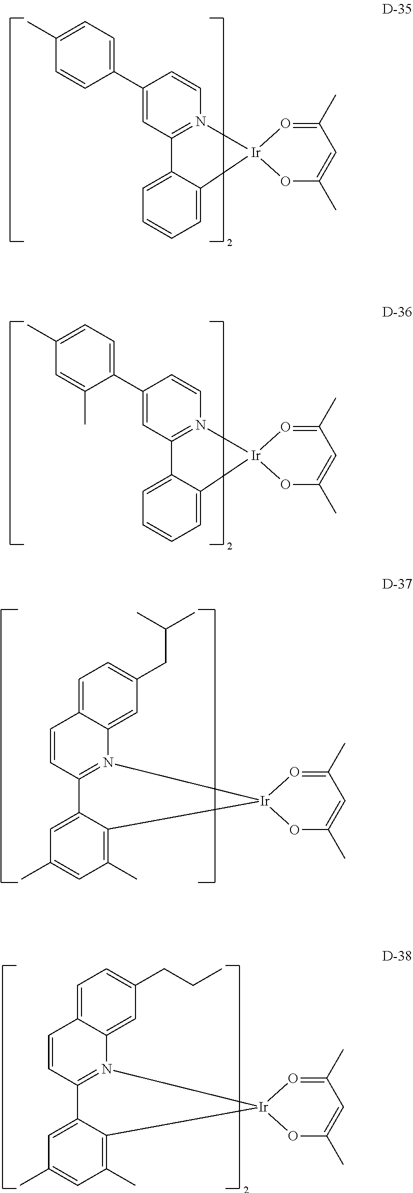

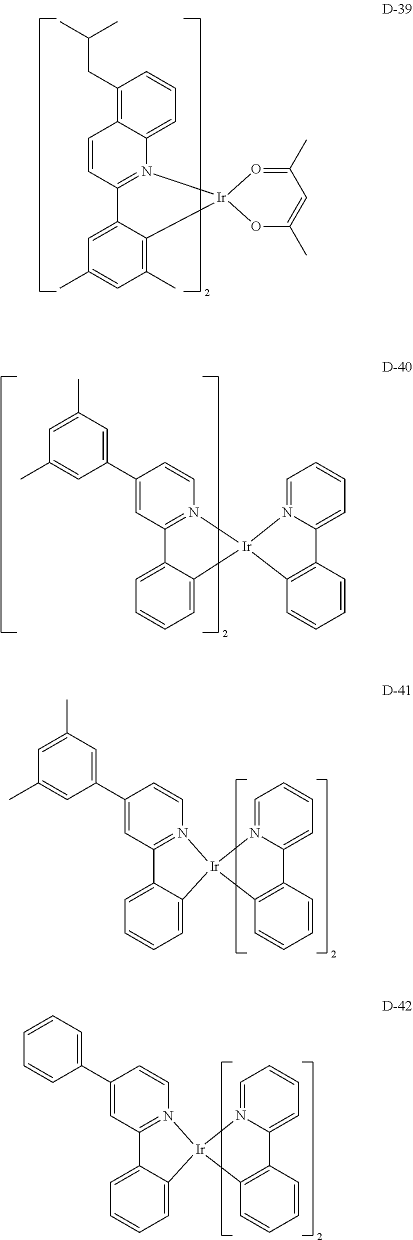

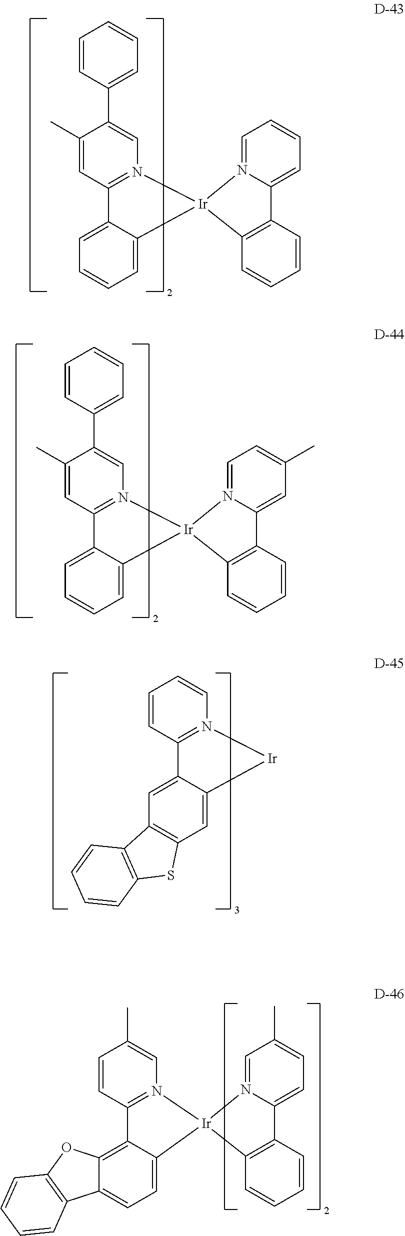

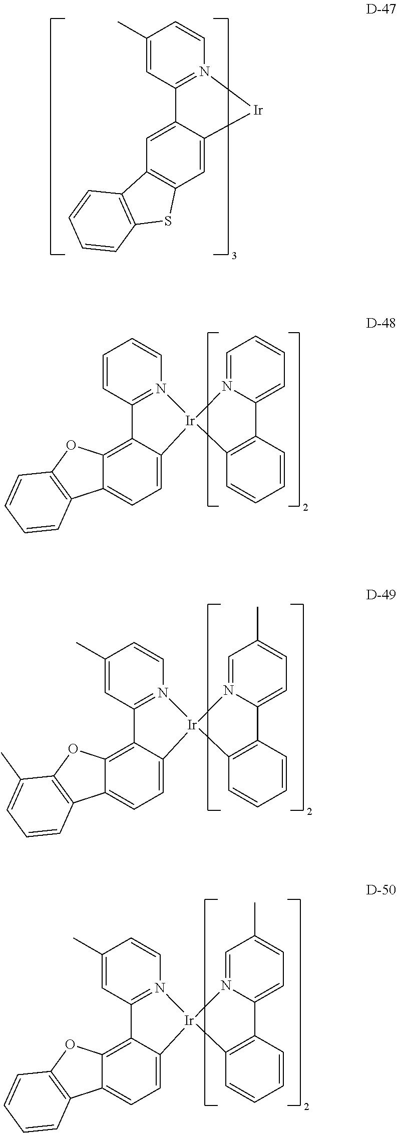

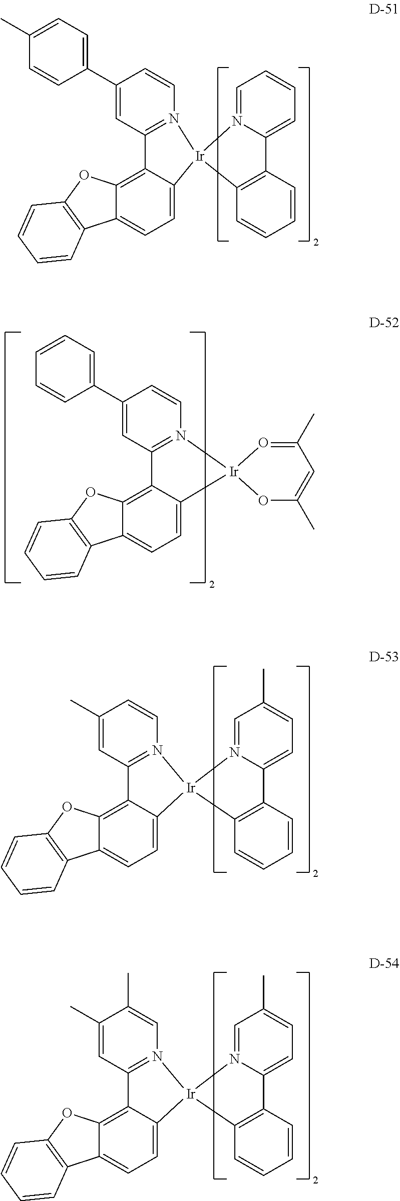

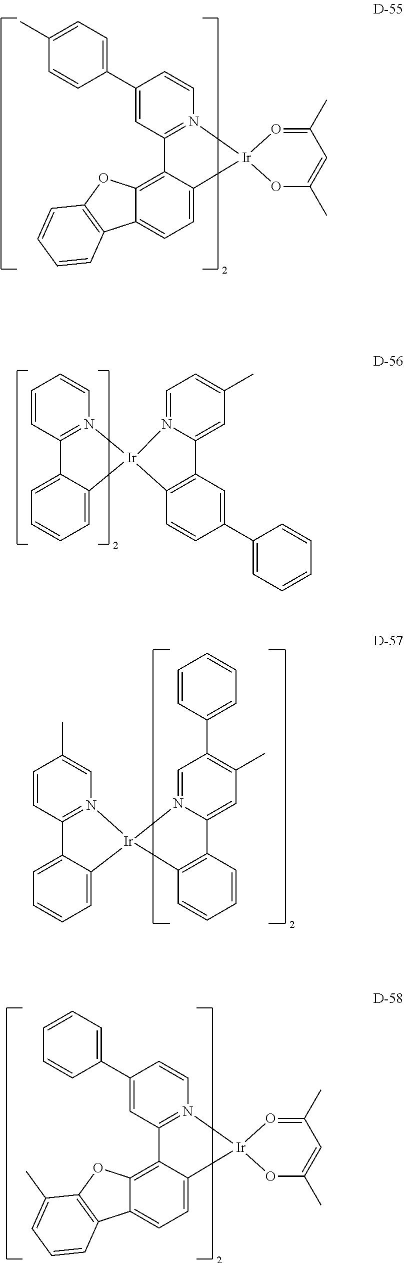

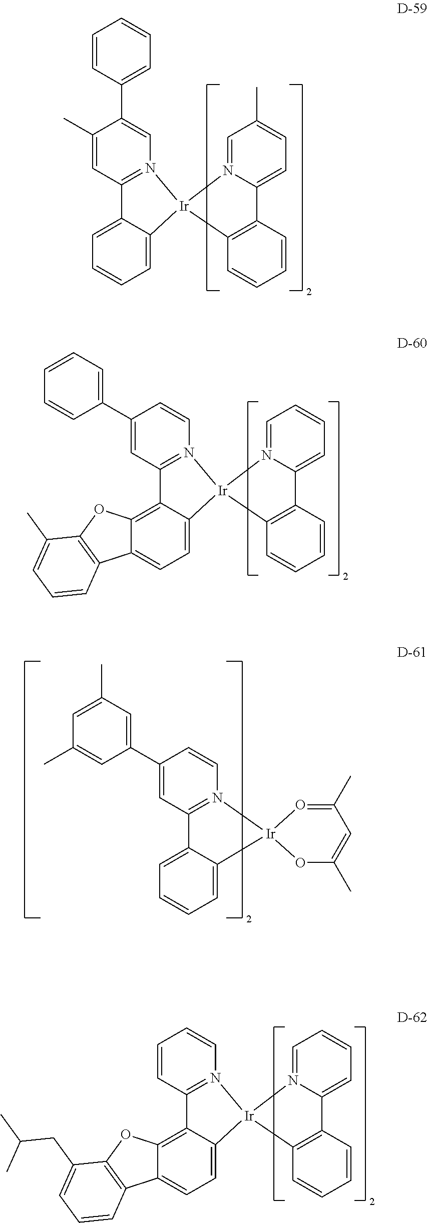

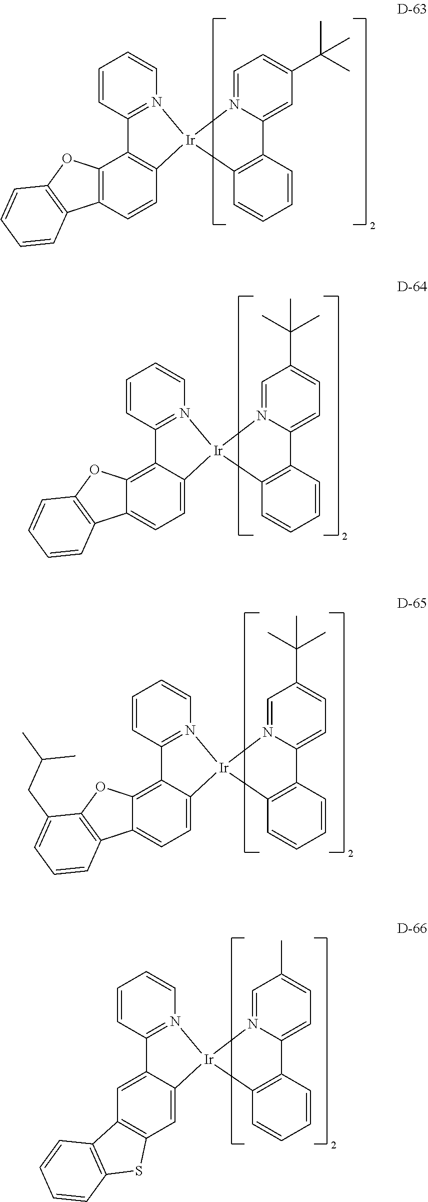

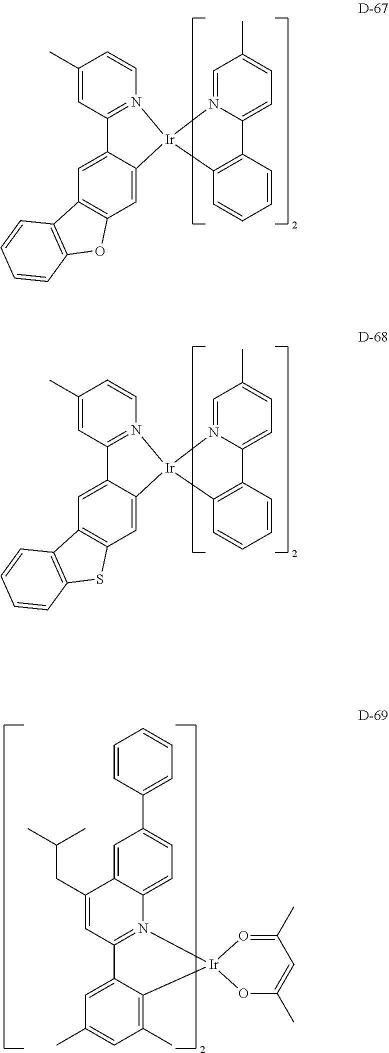

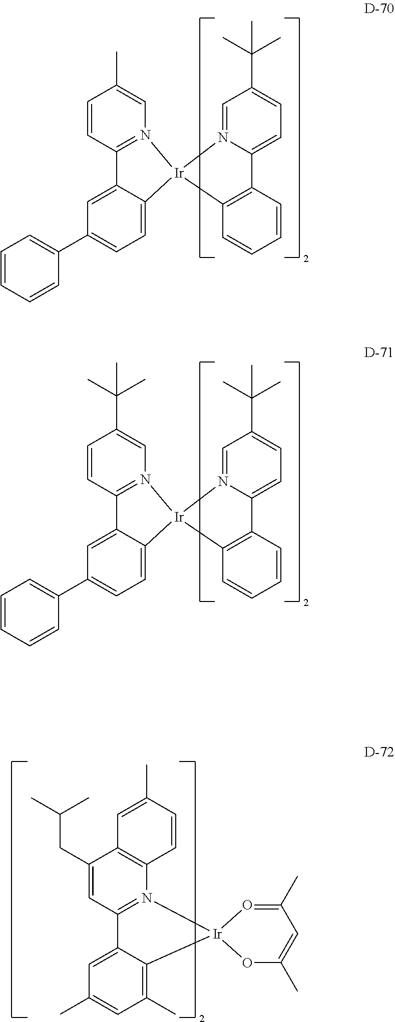

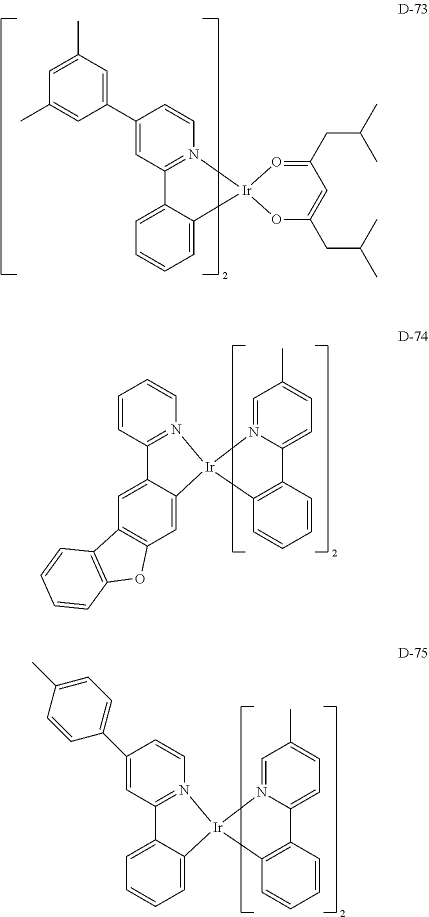

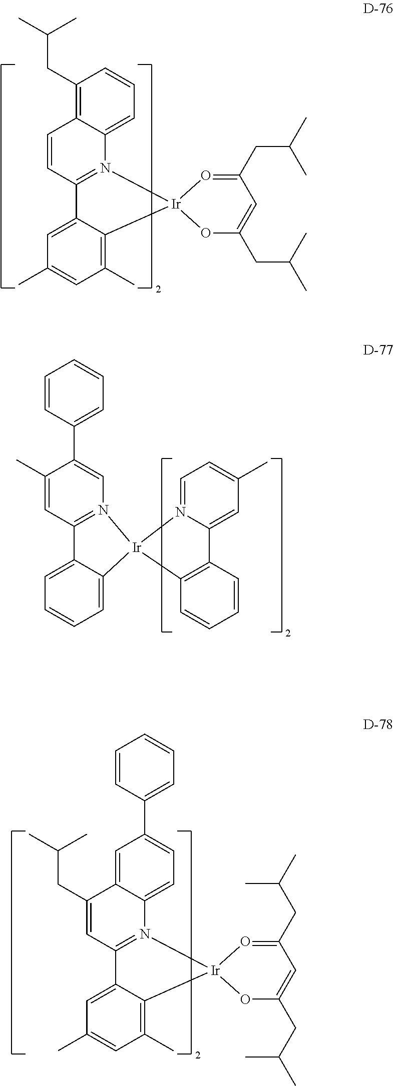

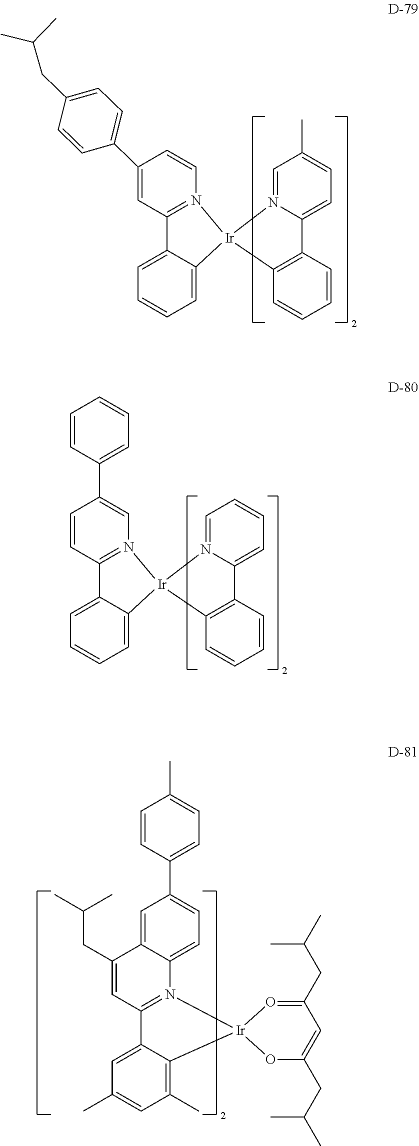

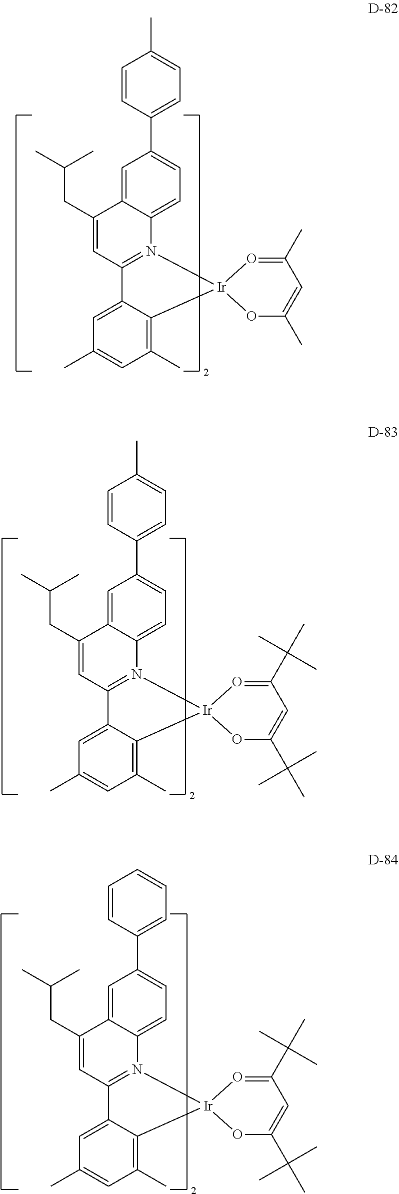

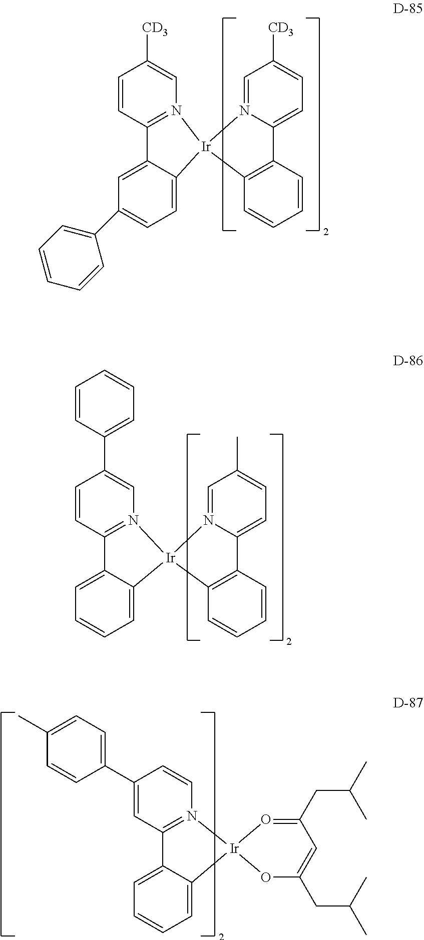

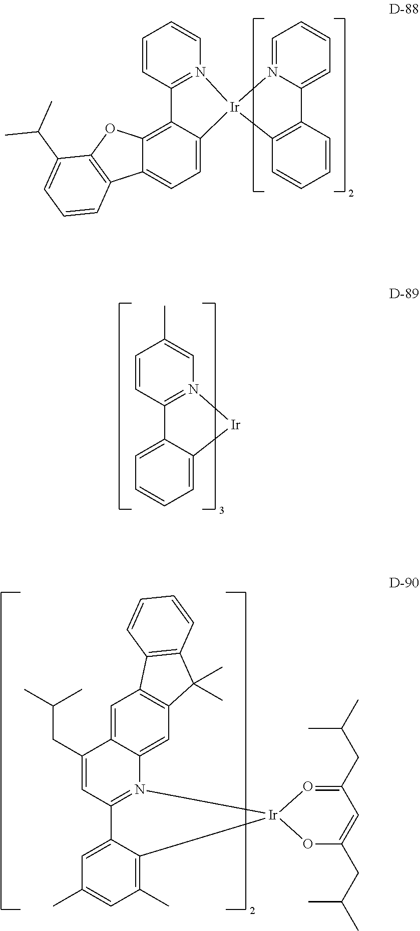

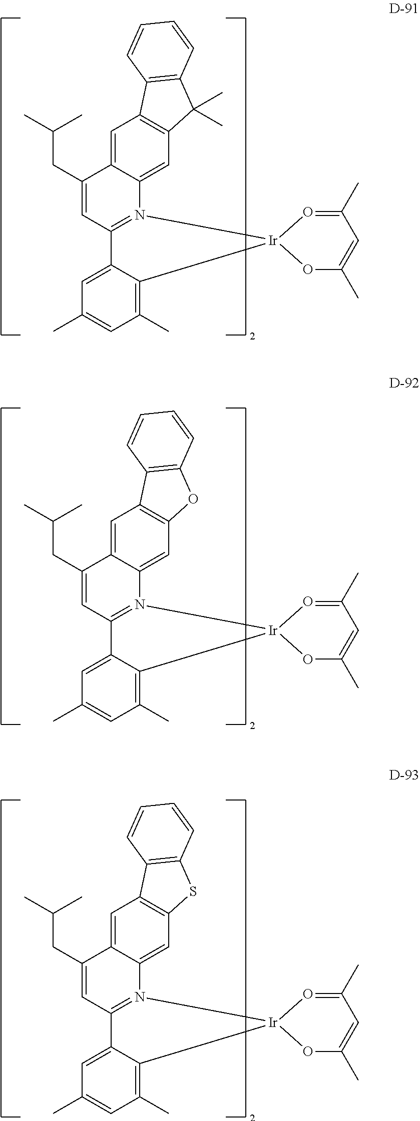

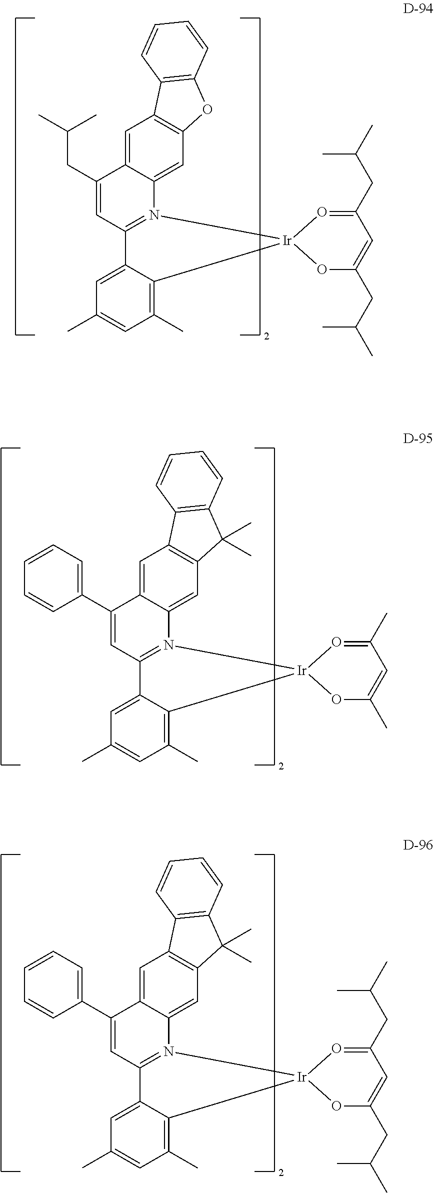

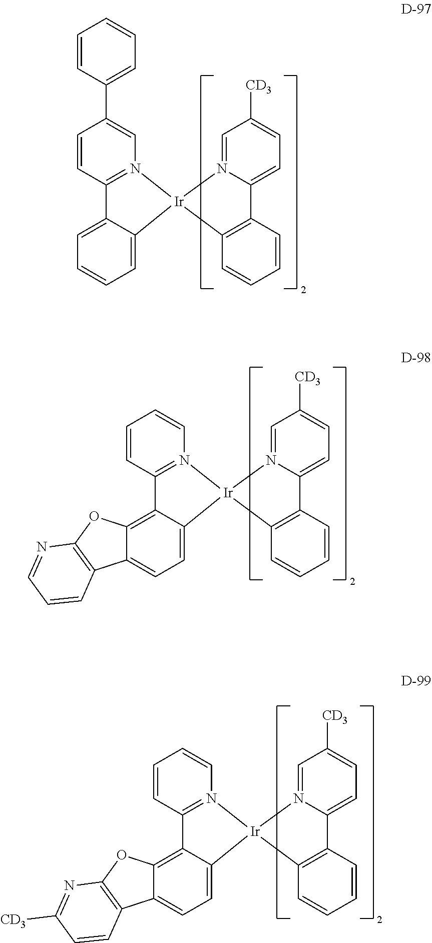

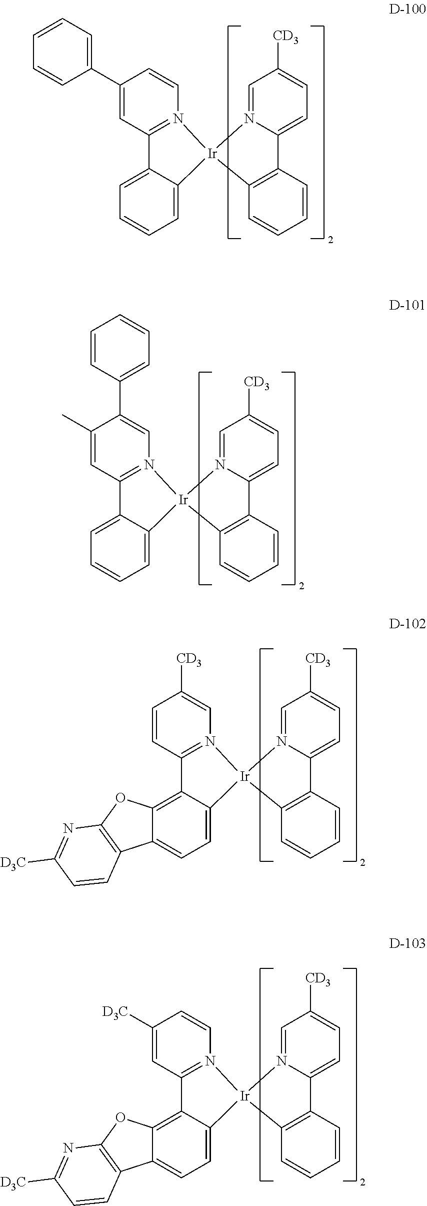

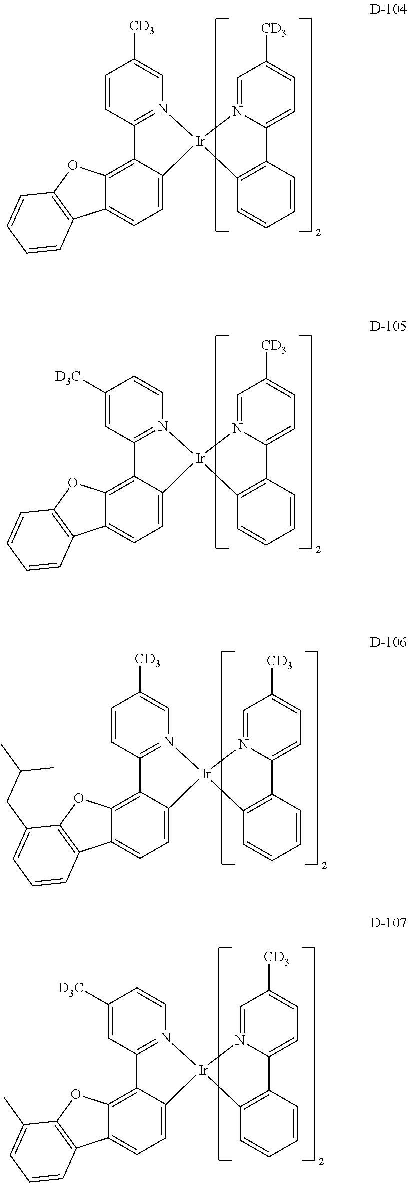

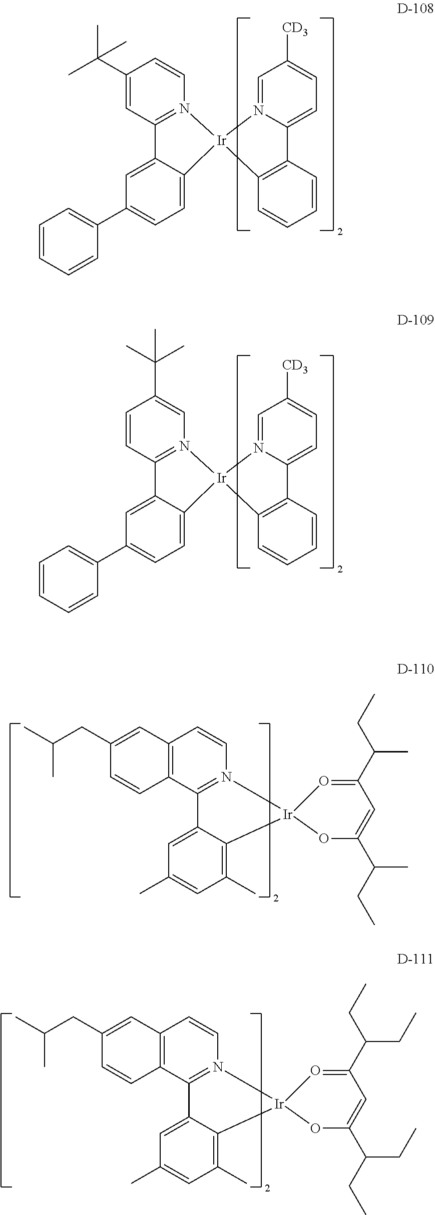

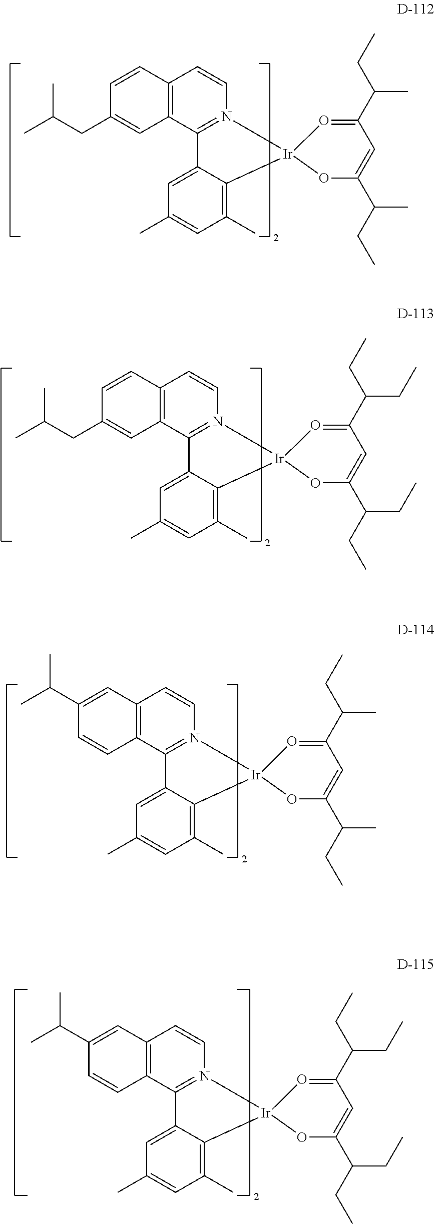

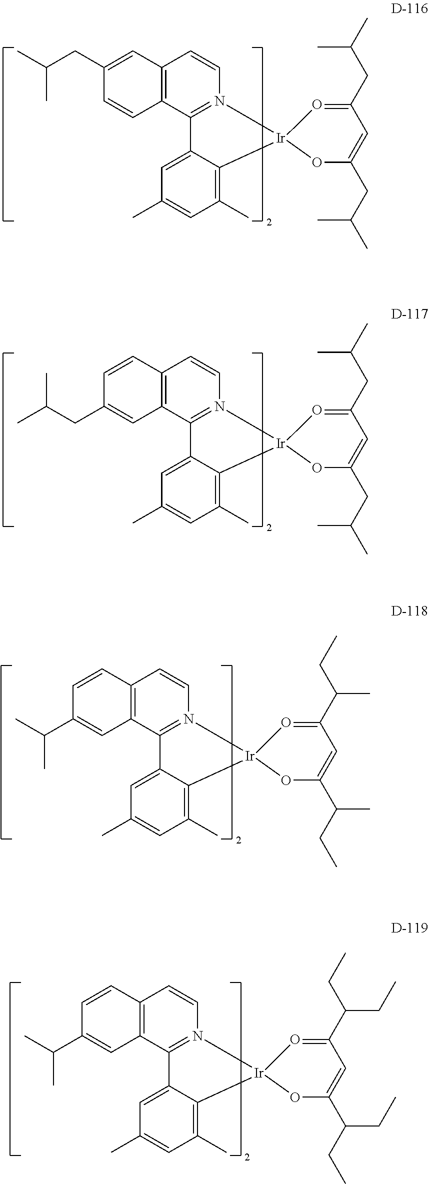

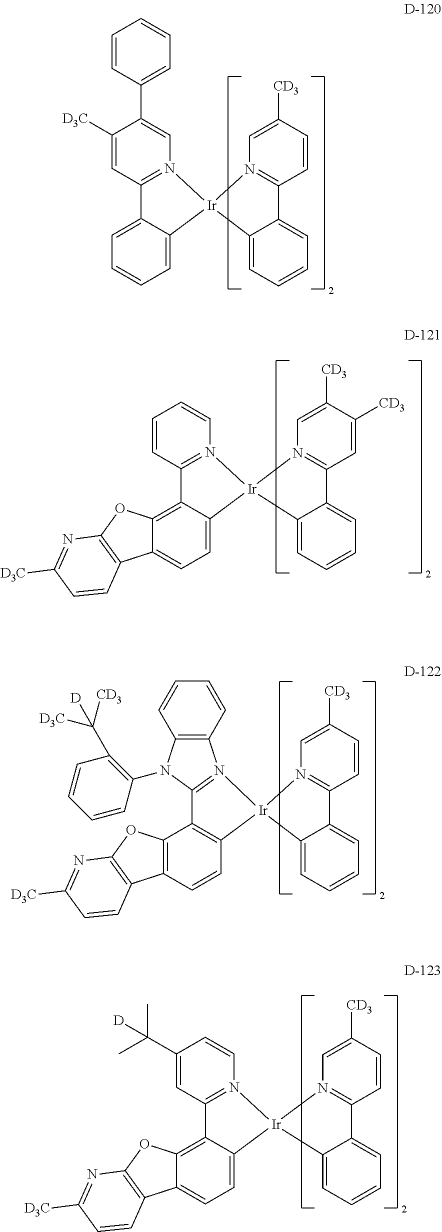

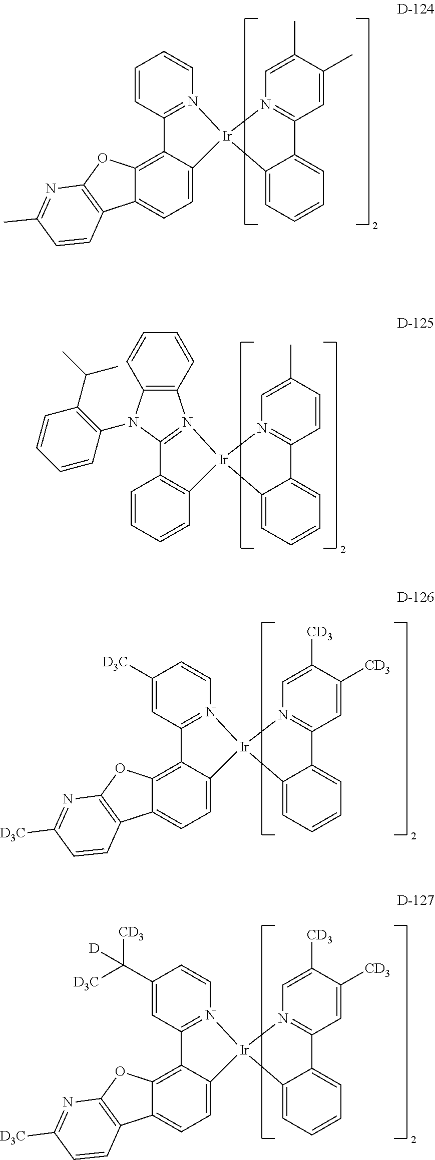

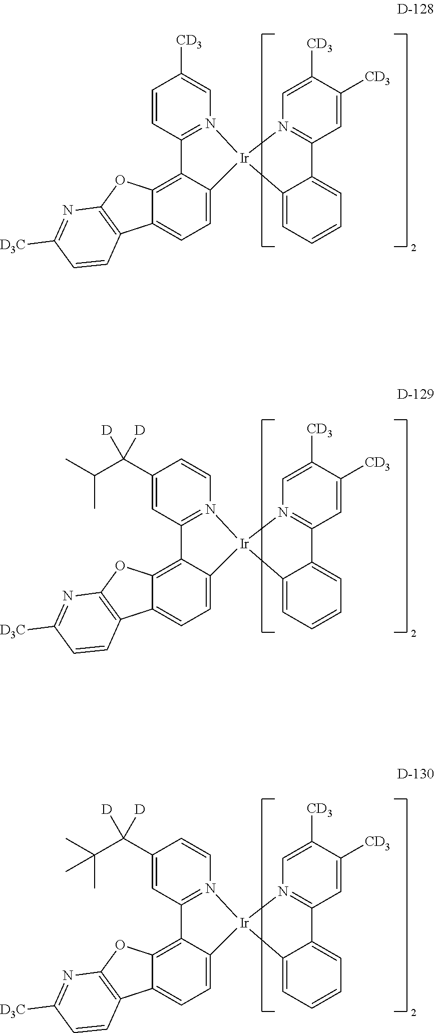

The compound represented by formula 1 of the present disclosure may be comprised in at least one layer constituting an organic electroluminescent device, and for example, at least one layer selected from a hole injection layer, a hole transport layer, a hole auxiliary layer, a light-emitting auxiliary layer, a light-emitting layer, an electron transport layer, an electron buffer layer, an electron injection layer, an interlayer, a hole blocking layer, and an electron blocking layer. Each of the layers may further consist of multi-layers.

In addition, the compound represented by formula 1 of the present disclosure is not limited thereto, but may be comprised in the light-emitting layer and/or an electron transport zone. The compound represented by formula 1 of the present disclosure may be comprised in the light-emitting layer as a host material, and simultaneously or optionally, in the electron transport zone as an electron buffer material(s) and/or an electron blocking material(s).

The electron transport zone of the present disclosure may consist of at least one layer selected from the group consisting of an electron buffer layer, a hole blocking layer, an electron transport layer and an electron injection layer, and each of the layers may consist of one or more layers. Preferably, the electron transport zone may comprise an electron buffer layer and/or a hole blocking layer. In addition, the electron transport zone may further comprise at least one layer of an electron transport layer(s) and an electron injection layer(s).

The organic electroluminescent materials of the present disclosure, for example, at least one of a hole injection material, a hole transport material, a hole auxiliary material, a light-emitting auxiliary material, an electron blocking material, a light-emitting material, an electron buffer material, a hole blocking material, an electron transport material, and an electron injection material, may comprise the compound represented by formula 1 The organic electroluminescent material may be at least one of a light-emitting material, an electron buffer material, and a hole blocking material. The organic electroluminescent material may consist of only the compound represented by formula 1, and may further comprise a conventional material(s) included in organic electroluminescent materials. When two or more materials are included in one layer, they may be mixed deposited or may be separately co-deposited to form a layer.

The organic electroluminescent device according to the present disclosure comprises a first electrode, a second electrode, and at least one organic layer between the first and second electrodes. One of the first and second electrodes may be an anode, and the other may be a cathode. The organic layer may comprise at least one light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The first electrode and the second electrode may each be formed with a transmissive conductive material, a transflective conductive material, or a reflective conductive material. The organic electroluminescent device may be a top emission type, a bottom emission type, or both-sides emission type according to the kinds of the material forming the first electrode and the second electrode. In addition, the hole injection layer may be further doped with a p-dopant, and the electron injection layer may be further doped with an n-dopant.

The organic electroluminescent device of the present disclosure may comprise the compound represented by formula 1, and may further comprise a conventional material(s) included in organic electroluminescent devices. The organic electroluminescent device comprising the organic electroluminescent compound represented by formula 1 of the present disclosure may exhibit high luminous efficiency and/or long lifetime properties.

In addition, an organic electroluminescent material according to one embodiment of the present disclosure may be used as light-emitting materials for a white organic light-emitting device. The white organic light-emitting device has been suggested to have various structures such as a parallel arrangement (side-by-side) method, a stacking method, or a color conversion material (CCM) method, etc., according to the arrangement of R (red), G (green), YG (yellowish green), or B (blue) light-emitting units. The organic electroluminescent compound according to the present disclosure may also be applied to the white organic light-emitting device.

The organic electroluminescent material according to one embodiment of the present disclosure may also be applied to the organic electroluminescent device comprising QD (quantum dot).

In addition, the present disclosure may provide a display system by using the compound represented by formula I. In addition, it is possible to produce a display system or a lighting system by using the compound of the present disclosure. Specifically, it is possible to produce a display system, e.g., a display system for smartphones, tablets, notebooks, PCs, TVs, or cars, or a lighting system, e.g., an outdoor or indoor lighting system, by using the organic electroluminescent compound of the present disclosure.

Hereinafter, the preparation method of the compound of the present disclosure, and the properties thereof will be explained in detail with reference to the representative compounds of the present disclosure. However, the present disclosure is not limited to the following examples.

### EXAMPLE 1

### Preparation of Compound C-160

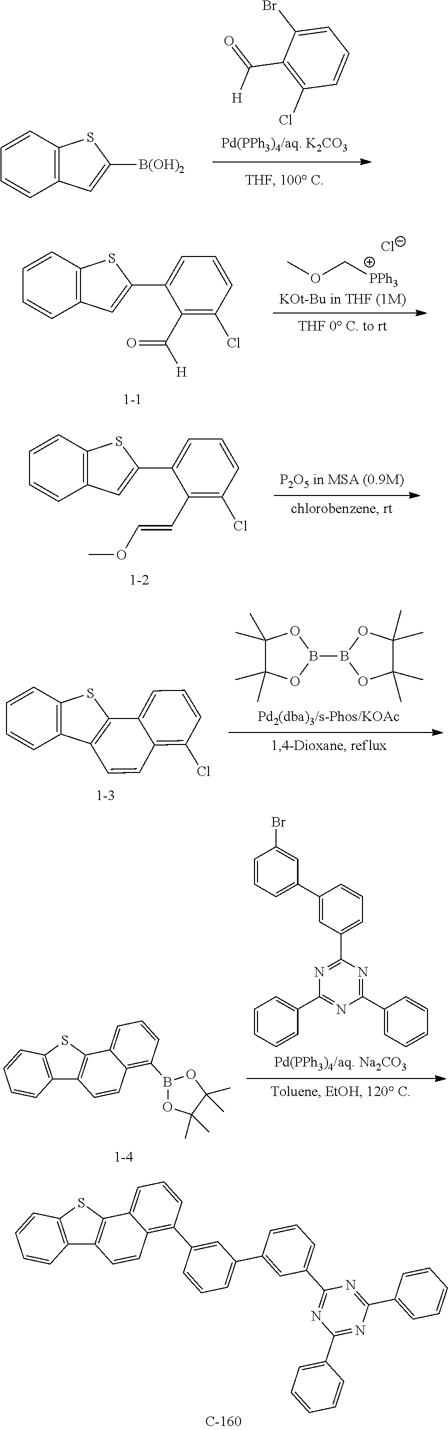

Synthesis of Compound 1-1

In a reaction vessel, 37 g of benzo[b]thiophen-2-yl boronic acid (205.05 mmol), g of 2-bromo-6-chlorobenzaldehyde (136.7 mmol), 4.7 g of tetrakis(triphenylphosphine)palladium (4.1 mmol), 47.2 g of potassium carbonate (341.75 mmol), 400 mL of tetrahydrofuran, and 100 mL of distilled water were added, and the mixture was stirred at 100° C. for 4 hours. After completion of the reaction, the reaction mixture was washed with distilled water, and an organic layer was extracted with ethyl acetate. The extracted organic layer was dried with magnesium sulfate and the solvent was removed by a rotary evaporator. The residue was separated by column chromatography to obtain 35 g of compound 1-1 (yield: 94%).

Synthesis of Compound 1-2

In a reaction vessel, 35 g of compound 1-1 (128.32 mmol), and 66 g of (methoxymethyl)triphenylphosphonium chloride (192.48 mmol) were added to 350 mL of tetrahydrofuran, and 193 mL of 1M potassium-tert-butoxide was added dropwise to the mixture at 0° C. After completion of the dropwise addition, the reaction temperature was gradually raised to room temperature and the mixture was further stirred for 2 hours. After completion of the reaction, an organic layer was extracted with ethyl acetate. The extracted organic layer was dried with magnesium sulfate and the solvent was removed by a rotary evaporator. The residue was separated by column chromatography to obtain 31 g of compound 1-2 (yield: 80%).

Synthesis of Compound 1-3

In a reaction vessel, 31 g of compound 1-2 (103.06 mmol) was dissolved in chlorobenzene, and 3.1 mL of Eaton's reagent was slowly added dropwise. After completion of the dropwise addition, the mixture was further stirred at room temperature for 2 hours. After completion of the reaction, the reaction mixture was washed with distilled water, and an organic layer was extracted with ethyl acetate. The extracted organic layer was dried with magnesium sulfate and the solvent was removed by a rotary evaporator. The residue was separated by column chromatography to obtain 24.4 g of compound 1-3 (yield: 88%).

Synthesis of Compound 1-4

In a reaction vessel, 9.0 g of compound 1-3 (29,77 mmol), 9.1 g of bis(pinacolato)diboron (35.72 mmol), 1.1 g of tris(dibenzylideneacetone)dipalladium (1.19 mmol), 1.0 g of 2-dicyclohexylphosphino-2′,6′-dimethoxybiphenyl (s-phos) (2.38 mmol), 8.8 g of potassium acetate (89.31 mmol) and 150 of 1,4-dioxane were added, and the mixture was stirred under reflux at 130° C. for 6 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was separated by column chromatography to obtain 9.0 g of compound 1-4 (yield: 84%).

Synthesis of Compound C-160

In a reaction vessel, 4.5 g of compound 1-4 (12.49 mmol), 6.6 g of 2-(3′-bromo-[1,1′-biphenyl]-3-yl)-4,6-diphenyl-1,3,5-triazine (14.20 mmol), 0.4 g of tetrakis(triphenylphosphine)palladium (0.34 mmol), 3.0 g of sodium carbonate (28.38 mmol), 55 of toluene, 14 mL of ethanol and 14 mL of distilled water were added, and the mixture was stirred at 120° C. for 4 hours. After completion of the reaction, the precipitated solid was washed with distilled water and methanol. The residue was separated by column chromatography to obtain 3.9 g of compound C-160 (yield: 51%). The physical properties of the synthesized compound C-160 are as follows.

| | MW | M.P. |
|---|---|---|
| C-160 | 617.7 | 268° C. |

### EXAMPLE 2

### Preparation of Compound C-5

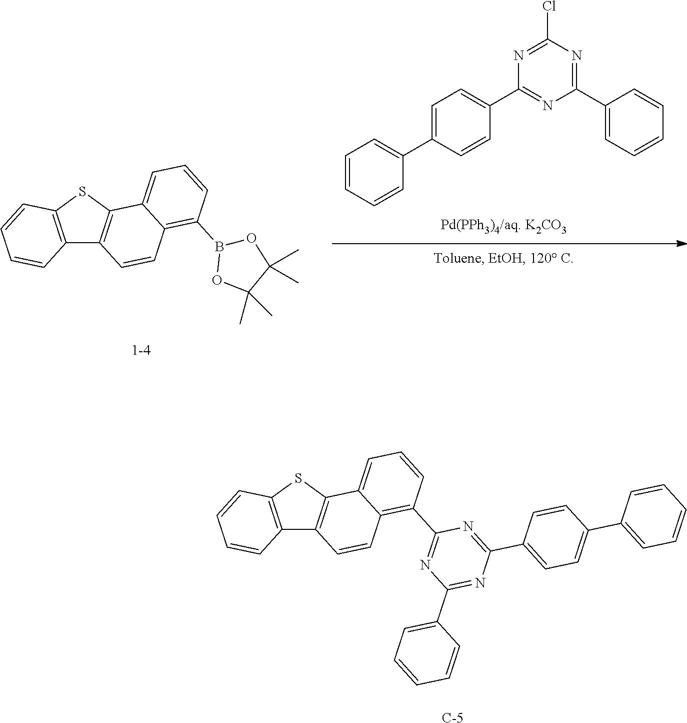

4.0 g of compound 1-4 (11.1 mmol), 4.6 g of 2-([1,1′-biphenyl]-4-yl)-4-chloro-6-phenyl-1,3,5-triazine (13.3 mmol), 0.6 g of Pd(PPh₃)₄ (0.56 mmol), and 3.1 g of K₂CO₃ (22.2 mmol) were added to 5.0 mL of EtOH, 40 mL of toluene, and 11 mL of distilled water, and the mixture was stirred under reflux for 6 hours. After completion of the reaction, the reaction mixture was cooled to room temperature and stirred at room temperature. MeOH was added thereto, and the resultant solid was filtered under reduced pressure. The residue was separated by column chromatography with MC/Hex to obtain 4.9 g of compound C-5 (yield: 81%).

| | MW | M.P. |
|---|---|---|
| C-5 | 541.7 | 280° C. |

### EXAMPLE 3

### Preparation of Compound C-146

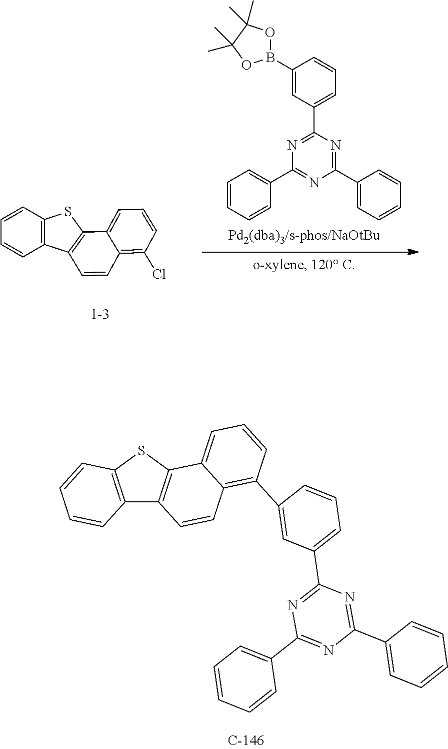 [SMILES: Clc1cccc2c1ccc1c3ccccc3sc21 ; CC1(C)OB(c2cccc(-c3nc(-c4ccccc4)nc(-c4ccccc4)n3)c2)OC1(C)C ; c1ccc(-c2nc(-c3ccccc3)nc(-c3cccc(-c4cccc5c4ccc4c6ccccc6sc54)c3)n2)cc1]

4.0 g of compound 1-3 (14.9 mmol), 7.1 g of 2,4-diphenyl-6-(3-(4,4,5,5-tetramethyl-1,3,2-dioxaborolan-2-yl)phenyl)-1,3,5-triazine (16.4 mmol), 0.7 g of Pd₂(dba)₃ (0.8 mmol), 0.6 g of s-phos (1.5 mmol), and 3.5 g of NaOtBu (37.3 mmol) were added to 80 mL of o-xylene, and the mixture was stirred under reflux for 6 hours. After completion of the reaction, the reaction mixture was cooled to room temperature and stirred at ordinary temperature. MeOH was added thereto, and the resultant solid was filtered under reduced pressure. The residue was separated by column chromatography with MC/Hex to obtain 3.6 g of compound C-146 (yield: 45%).

| | MW | M.P. |
|---|---|---|
| C-146 | 541.7 | 261° C. |

### EXAMPLE 4

### Preparation of Compound C-499

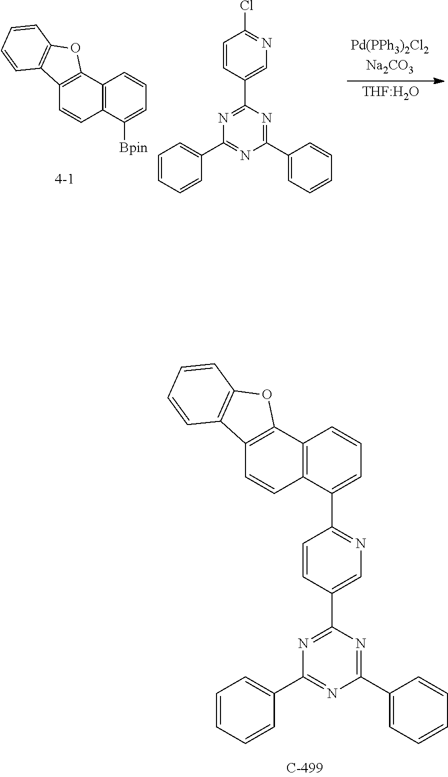

In a flask, 5.40 g of compound 4-1 (15.7 mmol), 5.41 g of 2-(6-chloropyridin-3-yl)-4,6-diphenyl-1,3,5-triazine (15.7 mmol), 551 mg of bis(triphenylphosphine)palladium(II) dichloride (0.78 mmol), and 2.5 g of sodium carbonate (23.5 mmol) were dissolved in 80 mL of THF:distilled water (10:1 mixed solution), and the mixture was stirred under reflux for 6 hours. After completion of the reaction, an organic layer was extracted with ethyl acetate. The residue was separated by column chromatography to obtain 3.0 g of compound C-499 (yield: 36%).

| | MW | M.P. |
|---|---|---|
| C-499 | 526.6 | 305° C. |

### EXAMPLE 5

### Preparation of Compound C-230

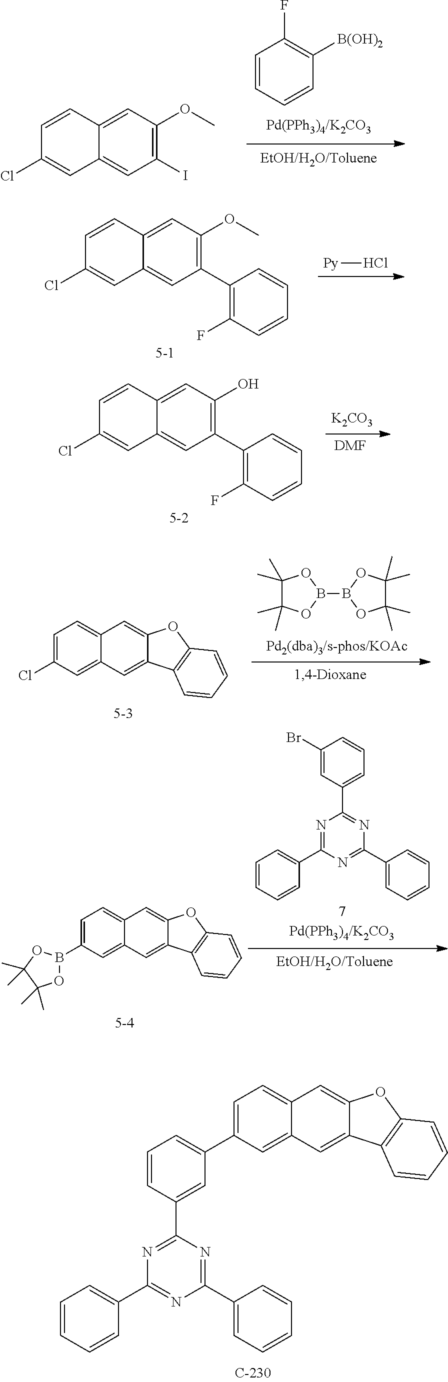

Synthesis of Compound 5-1

In a flask, 30 g of 6-chloro-3-iodo-2-methoxynaphthalene (94.19 mmol), 13.1 g of (2-fluorophenyl)boronic add (94.19 mmol), 5.4 g of tetrakis(triphenylphosphine)palladium (4.709 mmol), and 39 g of potassium carbonate (282.5 mmol) were dissolved in 580 mL of toluene, 145 mL of ethanol, and 145 mL of water, and the mixture was stirred under reflux for 4 hours. After completion of the reaction, the reaction mixture was cooled to room temperature, and an organic layer was extracted with ethyl acetate. The residue was separated by column chromatography to obtain 18.5 g of compound 5-1 (yield: 68%).

Synthesis of Compound 5-2

In a flask, 18.5 g of compound 5-1 (64.52 mmol) and 112 g of pyridine hydrochloride (967.9 mmol) were added, and the mixture was stirred under reflux at 230° C. for 3 hours. After completion of the reaction, the reaction mixture was cooled to room temperature, and an organic layer was extracted with dimethylchloride. After distillation under reduced pressure, hexane was added dropwise and filtered to obtain 14.8 g of compound 5-2 (yield: 84%).

Synthesis of Compound 5-3

In a flask, 14.8 g of compound 5-2 (54.27 mmol), 3.75 g of potassium carbonate (27.13 mmol), and 360 mL of dimethylformamide were added, and the mixture was stirred under reflux for 1 hour. After completion of the reaction, the reaction mixture was cooled to room temperature, and water was added dropwise and filtered to obtain 13 g of compound 54 (yield: 94%).

Synthesis of Compound 5-4

In a flask, 10 g of compound 54 (39.57 mmol), 12 g of bis(pinacolato)diboron (47.48 mmol), 1.4 g of tris(dibenzylideneacetone)dipalladium(0) (1.582 mmol), 1.3 g of 2-dicyclohexylphosphino-2′,6′-dimethobiphenyl (3.165 mmol), 11.6 g of potassium acetate (118.7 mmol), and 200 mL of 1,4-dioxane were added, and the mixture was stirred under reflux for 3 hours. After completion of the reaction, an organic layer was extracted with ethyl acetate. The residue was separated by column chromatography to obtain 7.8 g of compound 5-4 (yield: 54%).

Synthesis of Compound C-230

In a flask, 4.5 g of compound 5-4 (13.07 mmol), 5 g of 2-(3-bromophenyl)-4,6-diphenyl-1,3,5-triazine (13.07 mmol), 0.75 g of tetrakis(triphenylphosphine)palladium (0.653 mmol), 5.4 g of potassium carbonate (39.22 mmol), 80 mL of toluene, 20 mL of ethanol, and 20 mL of water were added, and the mixture was stirred under reflux for 2 hours. After completion of the reaction, the reaction mixture was cooled to room temperature, and methanol was added dropwise and filtered. The residue was dissolved in dimethyl chloride and separated by column chromatography to obtain 3.7 g of compound C-230 (yield: 53%).

| | MW | M.P. |
|---|---|---|
| C-230 | 525.6 | 272° C. |

Meanwhile, the present inventors have found the following facts by comparing the following B-type compounds, which are according to the present disclosure, with the following A-type compounds, which are not according to the present disclosure.

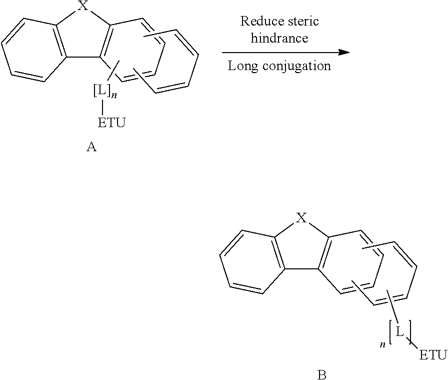

A device comprising a B-type compound as a red host material can have improved lifetime properties compared to a device comprising an A-type compound as a red host material. Without intending to be limited by theory, B-type compounds have longer conjugation and lower steric-hindrance energy than A-type compounds, where compounds with long conjugation can stabilize electrons. It is thought that this is because a compound having low steric-hindrance energy is difficult to decompose at high temperature.

Hereinafter, the properties of the organic electroluminescent device (OLED) comprising the compound according to the present disclosure will be explained in detail. However, the following examples merely illustrate the properties of an OLED according to the present disclosure in detail, but the present disclosure is not limited to the following examples.

### DEVICE EXAMPLES 1 AND 2

### Producing an OLED Using the Compound According to the Present Disclosure

An OLED was produced comprising the compound according to the present disclosure, as follows: A transparent electrode indium tin oxide (ITO) thin film (10 Ω/sq) on a glass substrate for an OLED (GEOMATEC CO., LTD., Japan) was subjected to an ultrasonic washing with acetone, ethanol and distilled water, sequentially, and then was stored in isopropanol. The ITO substrate was mounted on a substrate holder of a vacuum vapor deposition apparatus. Compound HI-1 was introduced into a cell of the vacuum vapor deposition apparatus, and the pressure in the chamber of the apparatus was then controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into another cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The compound shown as a host in Table 1 below was introduced into one cell of the vacuum vapor depositing apparatus as a host, and compound D-71 was introduced into another cell as a dopant. The two materials were evaporated at different rates and were deposited in a doping amount of 3 wt % based on the total amount of the host and dopant to form a light-emitting layer having a thickness of 40 nm on the second hole transport layer. Compound ET-1 and compound EI-1 were then introduced into two other cells, evaporated at the rate of 1:1, and deposited to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited by another vacuum vapor deposition apparatus on the electron injection layer. Thus, an OLED device was produced.

### DEVICE EXAMPLE 3

### Producing an OLED Using the Compound According to the Present Disclosure

An OLED device was produced in the same manner as in Device Example 1, except that the first hole injection layer was deposited to a thickness of 60 nm, the first hole transport layer was deposited to a thickness of 20 nm, compound HT-3 instead of compound HT-2 was used to form the second hole transport layer having a thickness of 5 nm, and the light-emitting layer to the electron transport layer were formed as follows: Compound BH was introduced into one cell of the vacuum vapor depositing apparatus as a host, and compound BD was introduced into another cell as a dopant. The two materials were evaporated at different rates and were deposited in a doping amount of 2 wt % based on the total amount of the host and dopant to form a light-emitting layer having a thickness of 20 nm on the second hole transport layer. Next, compound C-160 was deposited to form an electron buffer layer (or a hole blocking layer) having a thickness of 5 nm on the light-emitting layer. Compound ET-1 and compound EI-1 were then introduced into two other cells, evaporated at the rate of 1:1, and deposited to form an electron transport layer having a thickness of 30 nm on the electron buffer layer (or the hole blocking layer).

### COMPARATIVE EXAMPLE 1

### Producing an OLED Using the Compound Not According to the Present Disclosure

An OLED device was produced in the same manner as in Device Example 1, except that compound A was used as the host of the light-emitting layer.

### COMPARATIVE EXAMPLE 2

### Producing an OLED Using the Compound Not According to the Present Disclosure

An OLED device was produced in the same manner as in Device Example 1, except that compound B was used as the host of the light-emitting layer.

### COMPARATIVE EXAMPLE 3

### Producing an OLED Using the Compound Not According to the Present Disclosure

An OLED device was produced in the same manner as in Device Example 3, except that no electron buffer layer (or hole blocking layer) was deposited, and compound ET-1 and compound EI-1 were evaporated at a rate of 1:1 and deposited to form a electron transport layer having a thickness of 35 nm on the light-emitting layer.

The driving voltage and the CIE color coordinates at a luminance of 1,000 nit, and the time taken for luminance to decrease from 100% to 95% at a luminance of 5,000 nit (lifetime; T95) of the OLEDs produced in Device Examples 1 and 2 and Comparative Examples 1 and 2 are provided in Table 1 below.

TABLE 1

| | | Driving | CIE Color | | |
|---|---|---|---|---|---|
| | | Voltage | Coordinates | | Lifetime |
| | Host | [V] | x | y | (T95) [hr] |
| Comparative | A | 9.2 | 0.663 | 0.334 | 0.24 |
| Example 1 | | | | | |
| Comparative | B | 3.5 | 0.665 | 0.334 | 1.6 |
| Example 2 | | | | | |
| Device Example | C-160 | 3.7 | 0.666 | 0.333 | 6.8 |
| 1 | | | | | |
| Device Example | C-5 | 3.0 | 0.666 | 0.334 | 11.2 |
| 2 | | | | | |

From Table 1, it can be confirmed that the OLED comprising the compound according to the present disclosure as a host has lifetime properties longer than the OLED comprising the compound not according to the present disclosure as a host.

The driving voltage, luminous efficiency, and the CIE color coordinates at a luminance of 1,000 nit of the OLEDs produced in Device Example 3 and Comparative Example 3 are provided in Table 2 below.

TABLE 2

| | Driving | Luminous | CIE Color | |
|---|---|---|---|---|
| | Voltage | Efficiency | Coordinates | |
| | [V] | [cd/A] | x | y |
| Comparative Example 3 | 3.6 | 6.8 | 0.139 | 0.102 |
| Device Example 3 | 3.5 | 7.5 | 0.139 | 0.101 |

From Table 2, it can be confirmed that the OLED comprising the compound according to the present disclosure in the electron buffer layer (or hole blocking layer) has luminous efficiency properties higher than the OLED not according to the present disclosure.

The compounds used in the Device Examples and the Comparative Examples are shown in Table 3 below.

TABLE 3

| | |
|---|---|
| Hole Injec- tion Layer/ Hole Trans- port Layer | 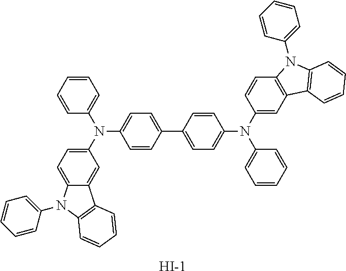 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 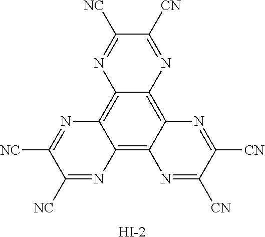 |
| | 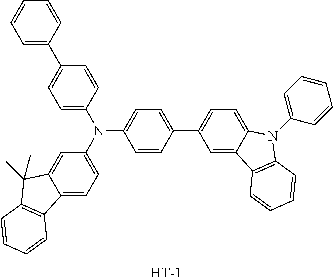 |
| | 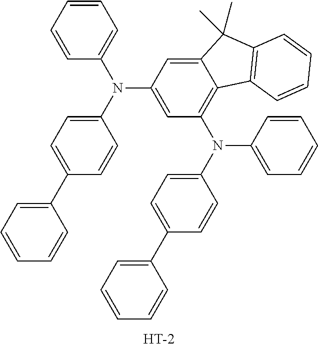 |
| | 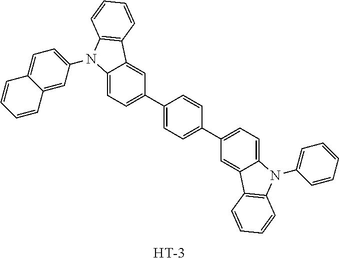 |
| Light- Emit- ting Layer | 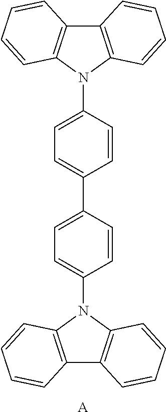 |
| | 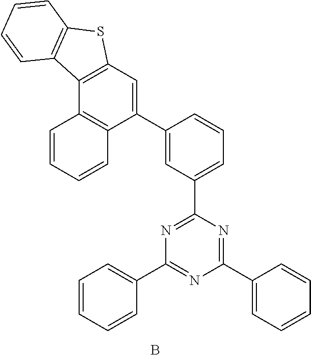 |
| | 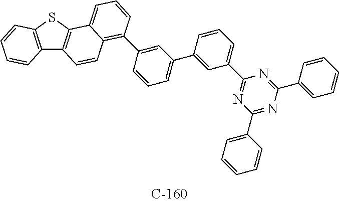 [SMILES: c1ccc(-c2nc(-c3ccccc3)nc(-c3cccc(-c4cccc(-c5cccc6c5ccc5c7ccccc7sc65)c4)c3)n2)cc1] |
| | 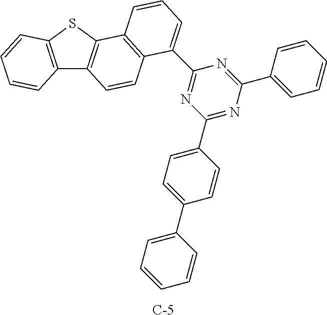 |
| | 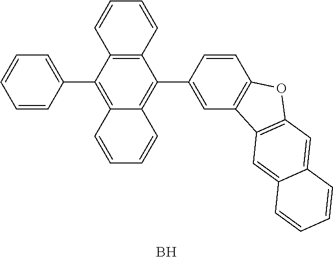 |
| | 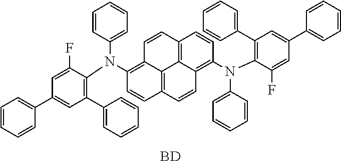 [SMILES: Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43 ; "BD"] |
| | 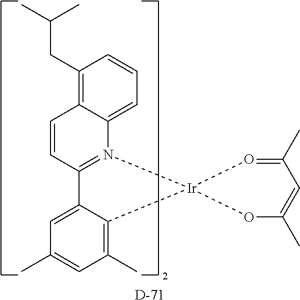 |
| Hole Block- ing Layer/ Elec- tron Buffer Layer | 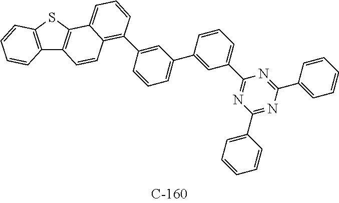 [SMILES: c1ccc(-c2nc(-c3ccccc3)nc(-c3cccc(-c4cccc(-c5cccc6c5ccc5c7ccccc7sc65)c4)c3)n2)cc1] |
| Elec- tron Trans- port Layer/ Elec- tron Injec- tion Layer | 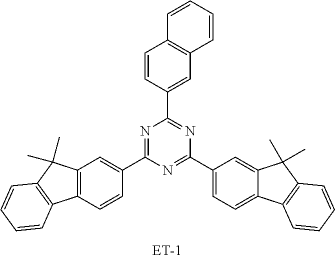 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 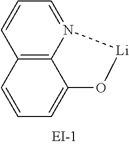 |

1. An organic electroluminescent compound represented by the following formula 1: 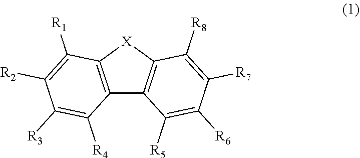
wherein
X represents O or S;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino; or may be linked to an adjacent substituent(s) to form a ring(s); and
at least one group of group R₅ and R₆, group R₆ and R₇, and group R₇ and R₈ are fused to the following formula 2 to form a ring(s): 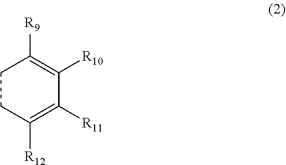
wherein
R₅ to R₈, which do not form a ring, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino;
R₉ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino, or *-L-ETU; with the proviso that at least one of R₉ to R₁₂ represents *-L-ETU;
L represents a single bond, a substituted or unsubstituted (C6-C30)arylene, or a substituted or unsubstituted (3- to 30-membered)heteroarylene; and
ETU represents a substituted or unsubstituted triazinyl, a substituted or unsubstituted quinazolinyl, a substituted or unsubstituted quinoxalinyl, a substituted or unsubstituted benzoquinoxalinyl, a substituted or unsubstituted dibenzoquinoxalinyl, a substituted or unsubstituted benzoquinazolinyl, a substituted or unsubstituted dibenzoquinazolinyl, a substituted or unsubstituted benzofuropyrazinyl, a substituted or unsubstituted benzothiopyrazinyl, a substituted or unsubstituted benzofuropyrimidinyl, or a substituted or unsubstituted benzothiopyrimidinyl.

2. The organic electroluminescent compound according to claim 1, wherein the substituents of the substituted alkyl, the substituted aryl(ene), the substituted heteroaryl(ene), the substituted silyl, the substituted amino, the substituted triazinyl, the substituted quinazolinyl, the substituted quinoxalinyl, the substituted benzoquinoxalinyl, the substituted dibenzoquinoxalinyl, the substituted benzoquinazolinyl, the substituted dibenzoquinazolinyl, the substituted benzofuropyrazinyl, the substituted benzothiopyrazinyl, the substituted benzofuropyrimidinyl and the substituted benzothiopyrimidinyl, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl, a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl;
a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl, a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The organic electroluminescent compound according to claim 1, wherein the formula 1 is represented by any one of the following formulas 1-1 to 1-3: 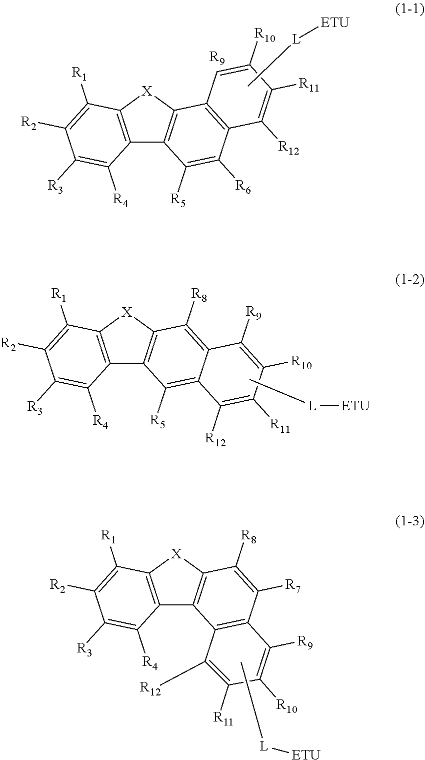
wherein
R₅ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted silyl, or a substituted or unsubstituted amino; and
R₁ to R₄, L, ETU and X are as defined in claim 1.

4. The organic electroluminescent compound according to claim 1, wherein ETU is represented by any one of the following: 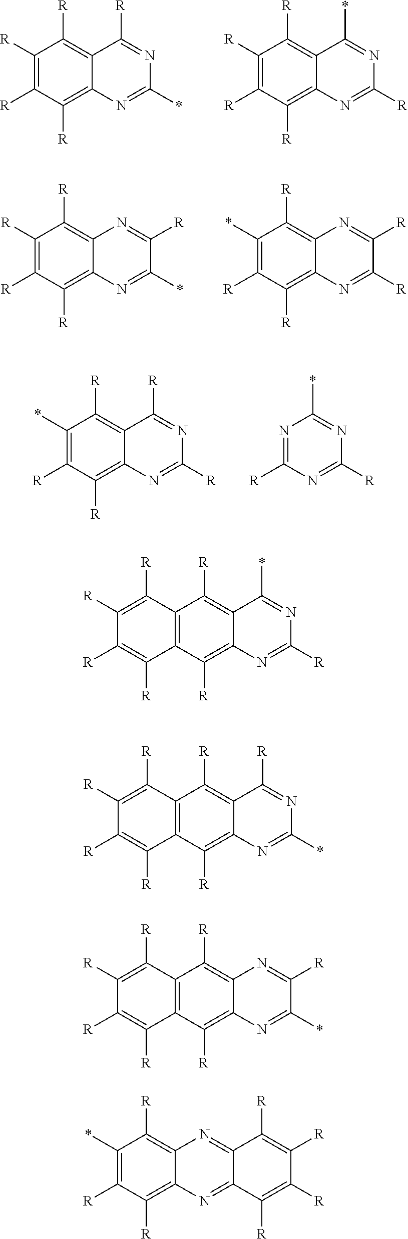 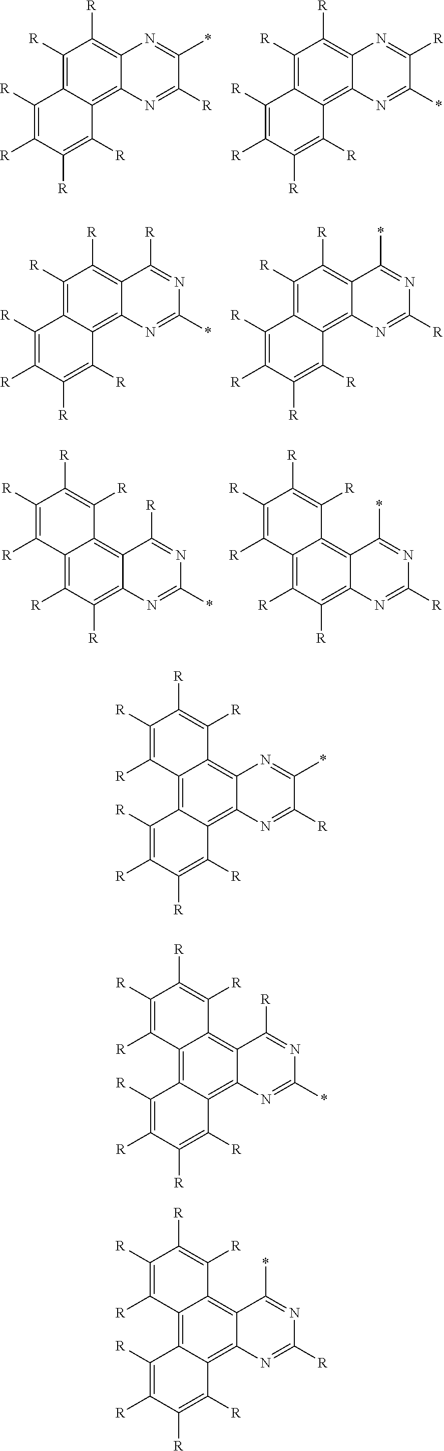 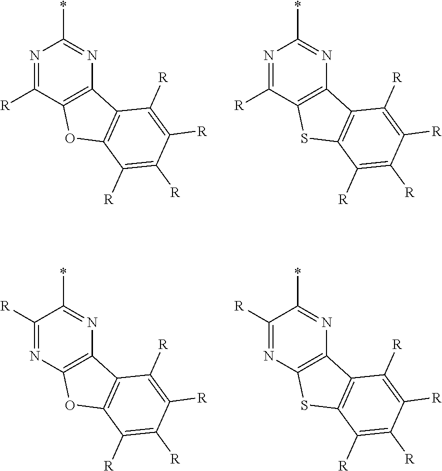
wherein
R, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted silyl, or a substituted or unsubstituted amino.

5. The organic electroluminescent compound according to claim 1, wherein the compound represented by formula 1 is any one selected from the group consisting of the following compounds:

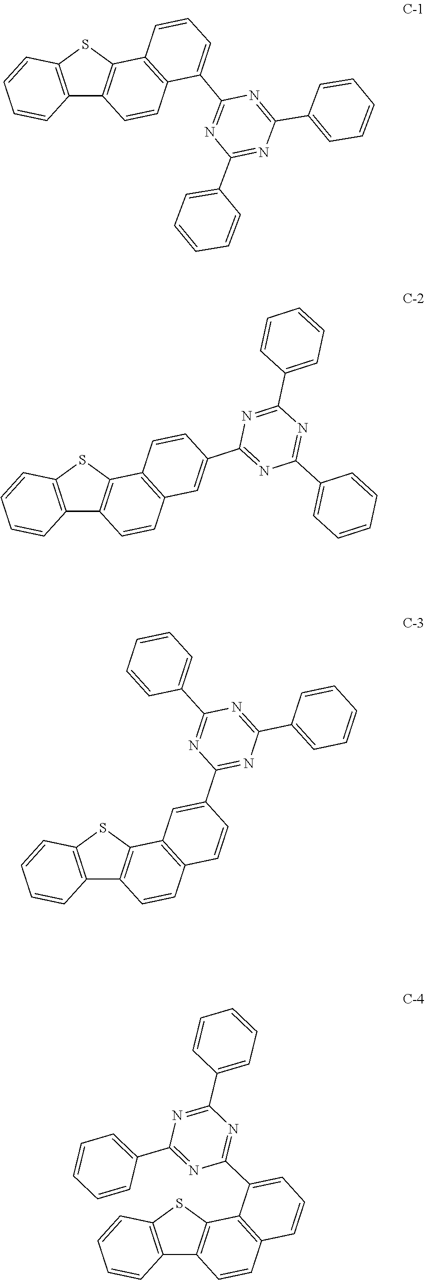 [SMILES: c1ccc(-c2nc(-c3ccccc3)nc(-c3cccc4c3ccc3c5ccccc5sc43)n2)cc1 ; c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1]

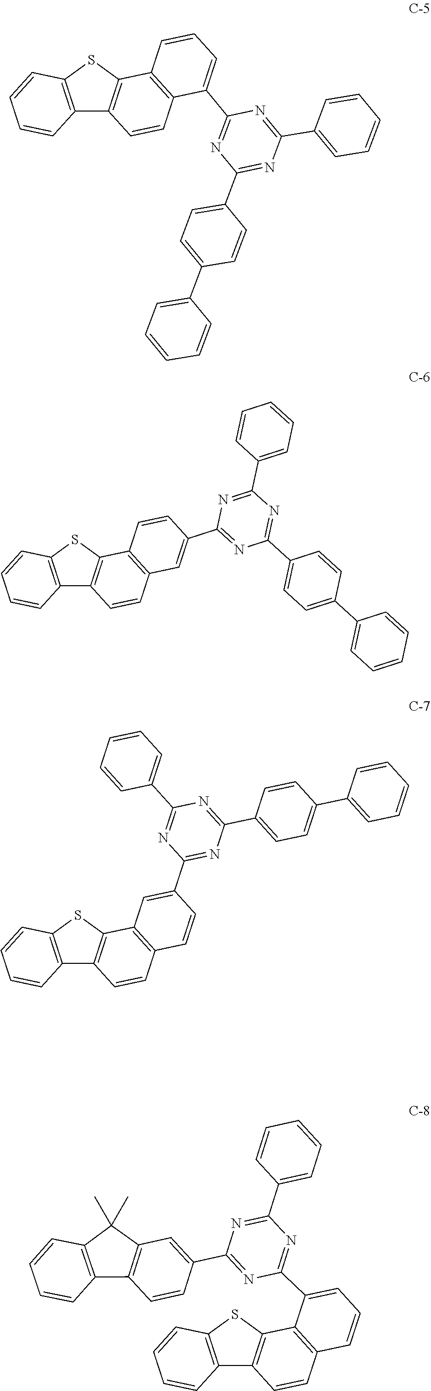 [SMILES: c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1 ; c1ccc(-c2ccc(-c3nc(-c4ccccc4)nc(-c4ccc5ccc6c7ccccc7sc6c5c4)n3)cc2)cc1 ; CC1(C)c2ccccc2-c2ccc(-c3nc(-c4ccccc4)nc(-c4cccc5ccc6c7ccccc7sc6c45)n3)cc21]

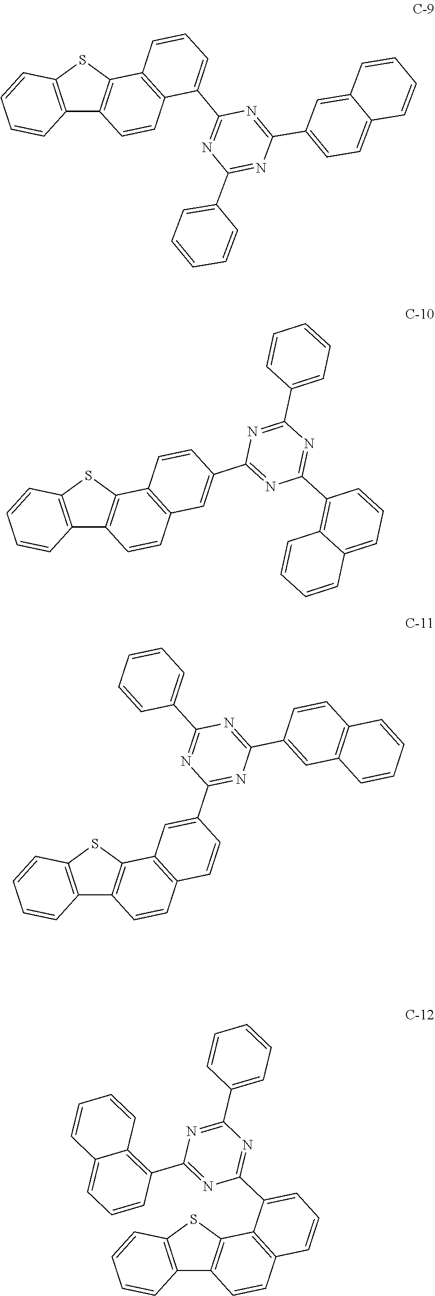 [SMILES: c1ccc(-c2nc(-c3ccc4ccccc4c3)nc(-c3cccc4c3ccc3c5ccccc5sc43)n2)cc1 ; c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1 ; c1ccc(-c2nc(-c3cccc4ccccc34)nc(-c3cccc4ccc5c6ccccc6sc5c34)n2)cc1]

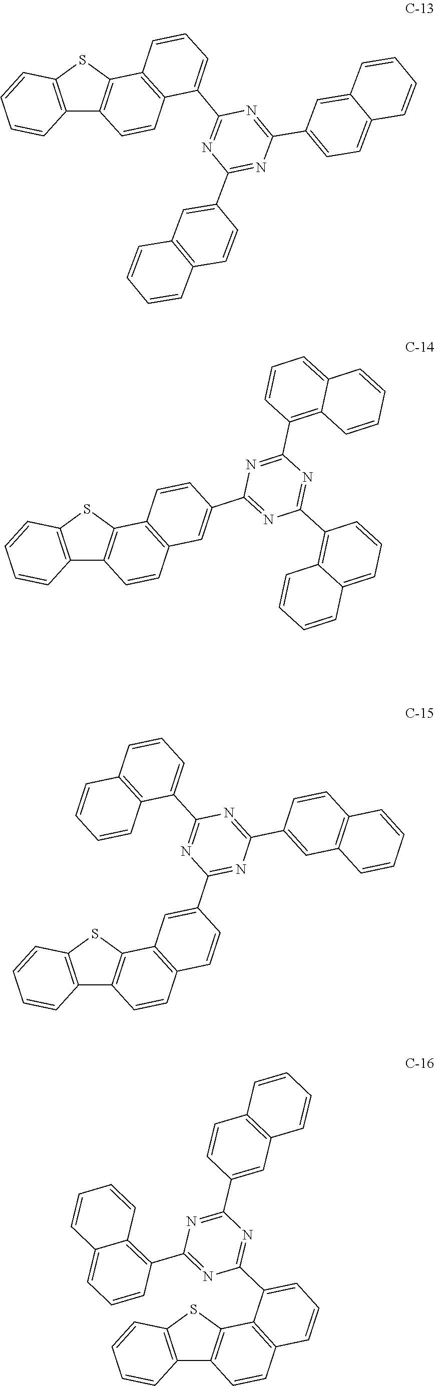 [SMILES: c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1]

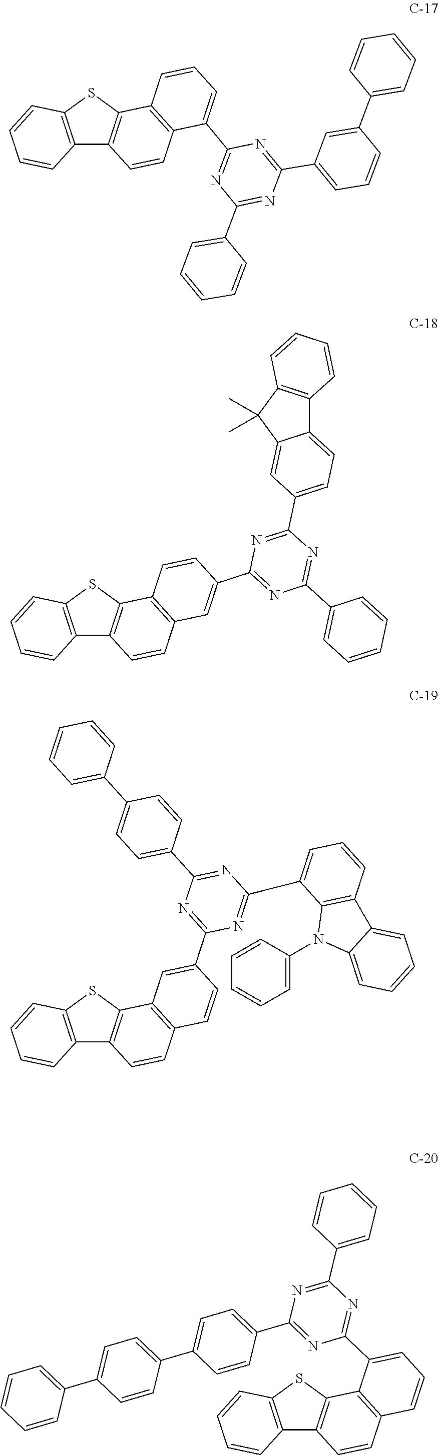 [SMILES: c1ccc(-c2cccc(-c3nc(-c4ccccc4)nc(-c4cccc5c4ccc4c6ccccc6sc54)n3)c2)cc1 ; CC1(C)c2ccccc2-c2ccc(-c3nc(-c4ccccc4)nc(-c4ccc5c(ccc6c7ccccc7sc56)c4)n3)cc21 ; c1ccc(-c2ccc(-c3nc(-c4ccc5ccc6c7ccccc7sc6c5c4)nc(-c4cccc5c6ccccc6n(-c6ccccc6)c45)n3)cc2)cc1 ; c1ccc(-c2ccc(-c3ccc(-c4nc(-c5ccccc5)nc(-c5cccc6ccc7c8ccccc8sc7c56)n4)cc3)cc2)cc1]

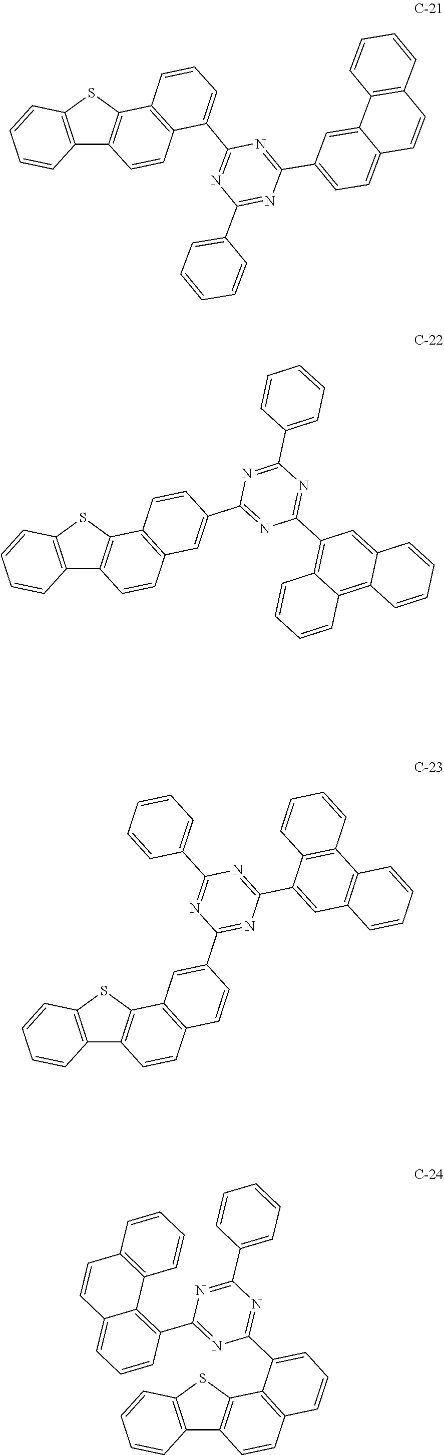 [SMILES: c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1 ; c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1]

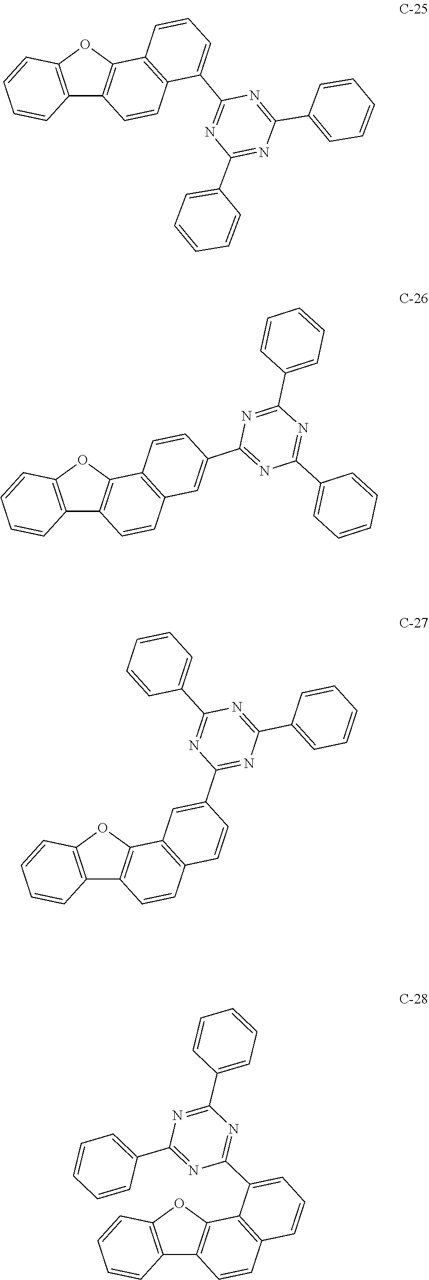 [SMILES: c1ccc(-c2nc(-c3ccccc3)nc(-c3cccc4c3ccc3c5ccccc5oc43)n2)cc1 ; c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1 ; c1ccc(-c2nc(-c3ccccc3)nc(-c3cccc4ccc5c6ccccc6oc5c34)n2)cc1]

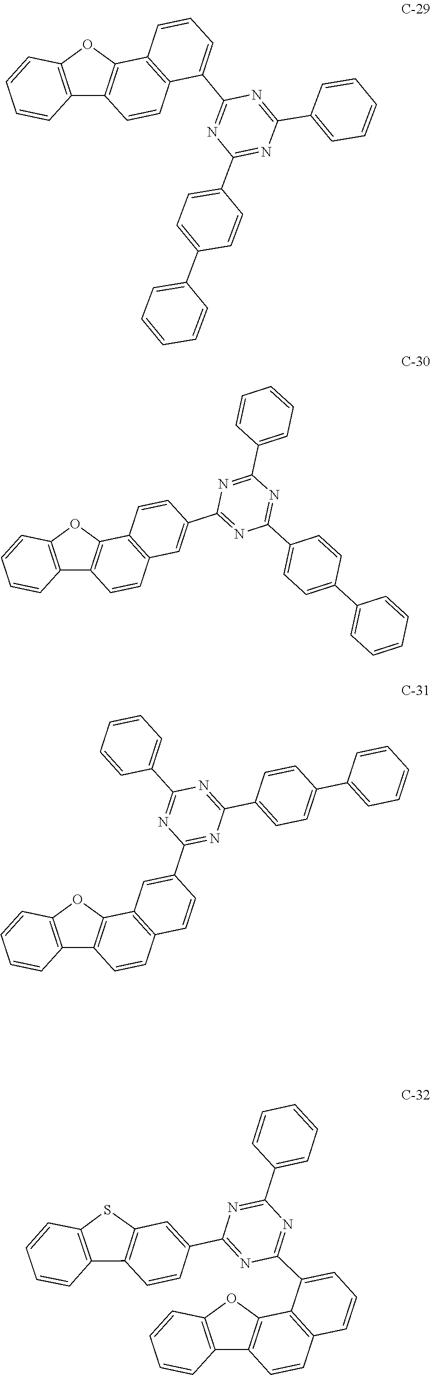 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1]

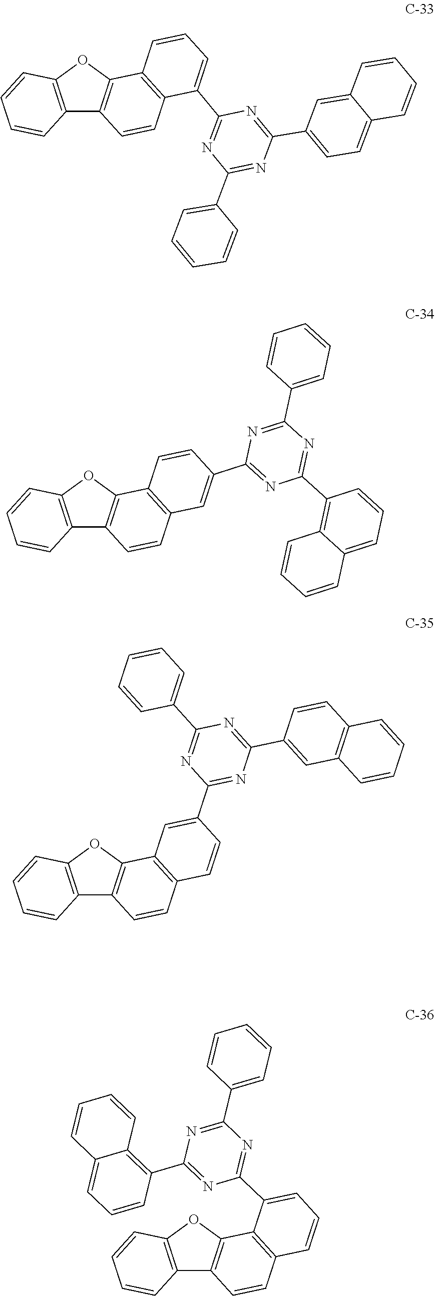 [SMILES: c1ccc(-c2nc(-c3ccc4ccccc4c3)nc(-c3cccc4c3ccc3c5ccccc5oc43)n2)cc1 ; c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1]

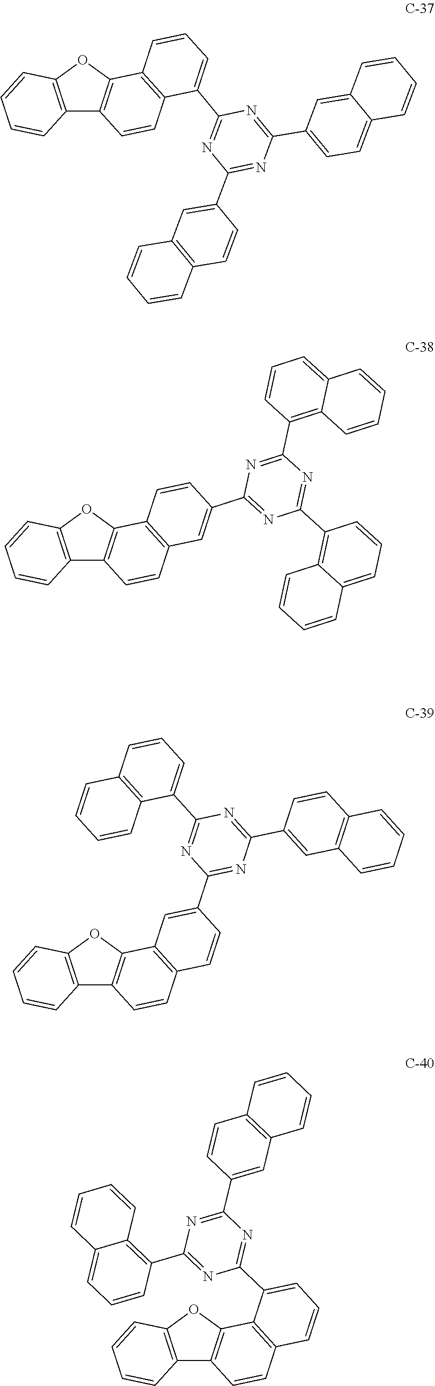 [SMILES: c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1 ; c1ccc2cc(-c3nc(-c4ccc5ccc6c7ccccc7oc6c5c4)nc(-c4cccc5ccccc45)n3)ccc2c1 ; c1ccc2cc(-c3nc(-c4cccc5ccccc45)nc(-c4cccc5ccc6c7ccccc7oc6c45)n3)ccc2c1]

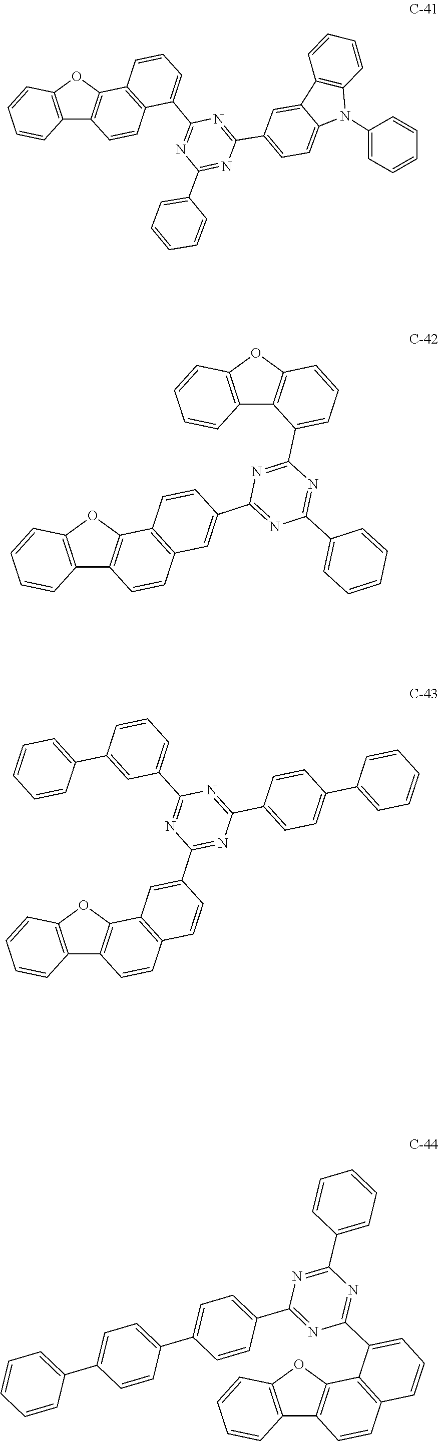 [SMILES: c1ccc(-c2nc(-c3ccc4c(c3)c3ccccc3n4-c3ccccc3)nc(-c3cccc4c3ccc3c5ccccc5oc43)n2)cc1 ; c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

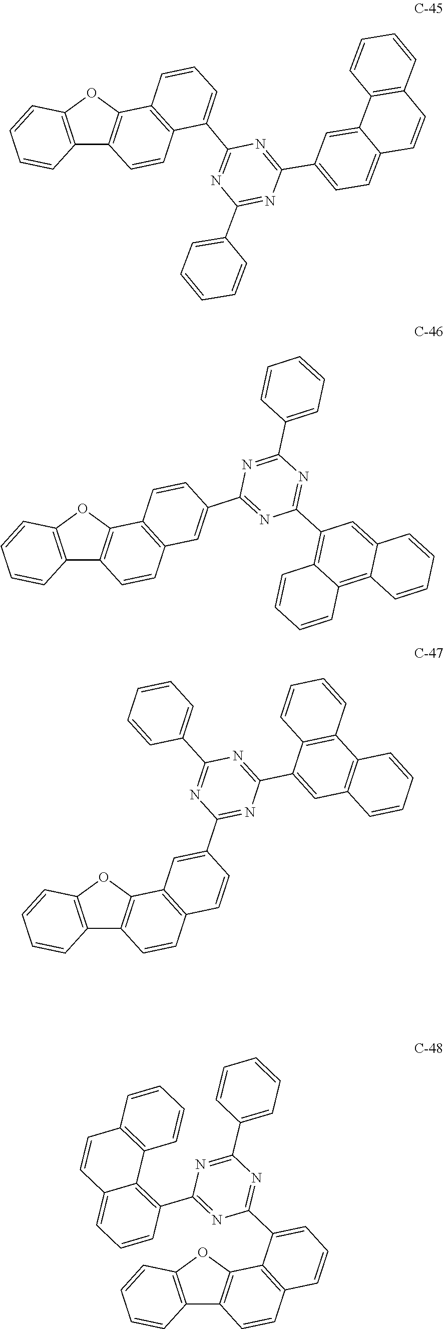 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3ccc4ccc5c6ccccc6oc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1]

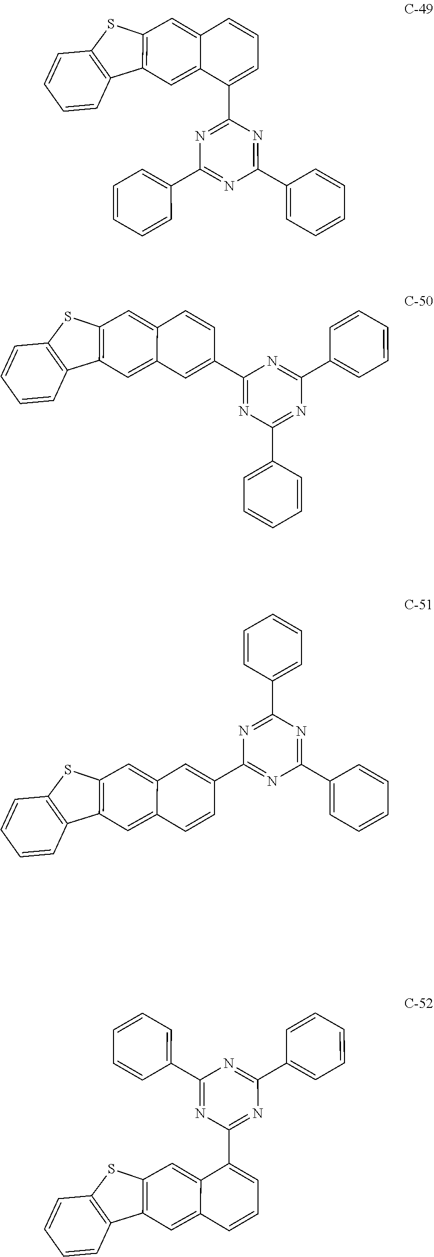 [SMILES: c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1 ; c1ccc(-c2nc(-c3ccccc3)nc(-c3ccc4cc5c(cc4c3)sc3ccccc35)n2)cc1 ; c1ccc(-c2nc(-c3ccccc3)nc(-c3cccc4cc5c(cc34)sc3ccccc35)n2)cc1]

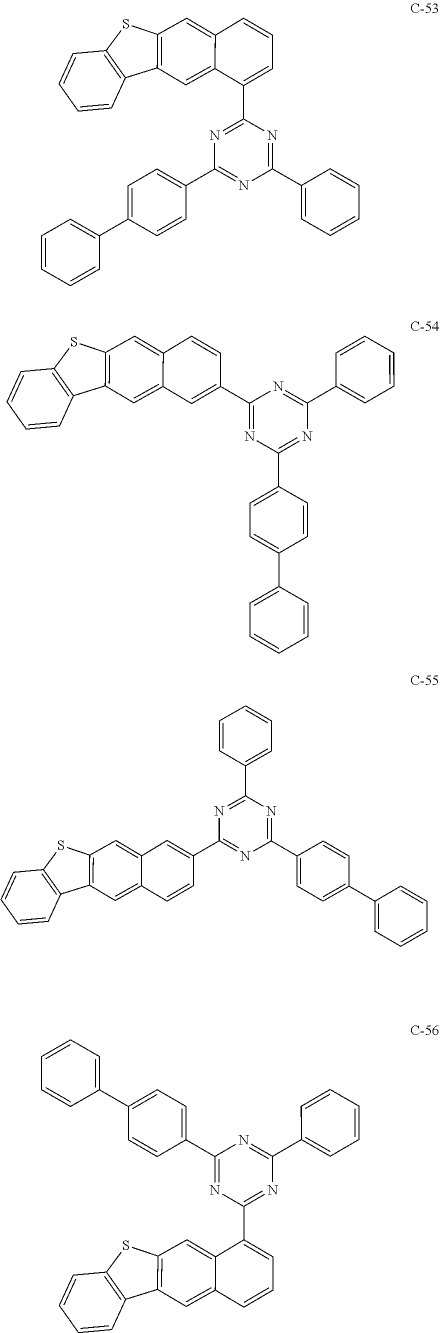 [SMILES: c1ccc(-c2ccc(-c3nc(-c4ccccc4)nc(-c4cccc5cc6sc7ccccc7c6cc45)n3)cc2)cc1 ; c1ccc(-c2ccc(-c3nc(-c4ccccc4)nc(-c4ccc5cc6sc7ccccc7c6cc5c4)n3)cc2)cc1 ; c1ccc(-c2ccc(-c3nc(-c4ccccc4)nc(-c4ccc5cc6c(cc5c4)sc4ccccc46)n3)cc2)cc1 ; c1ccc(-c2ccc(-c3nc(-c4ccccc4)nc(-c4cccc5cc6c(cc45)sc4ccccc46)n3)cc2)cc1]

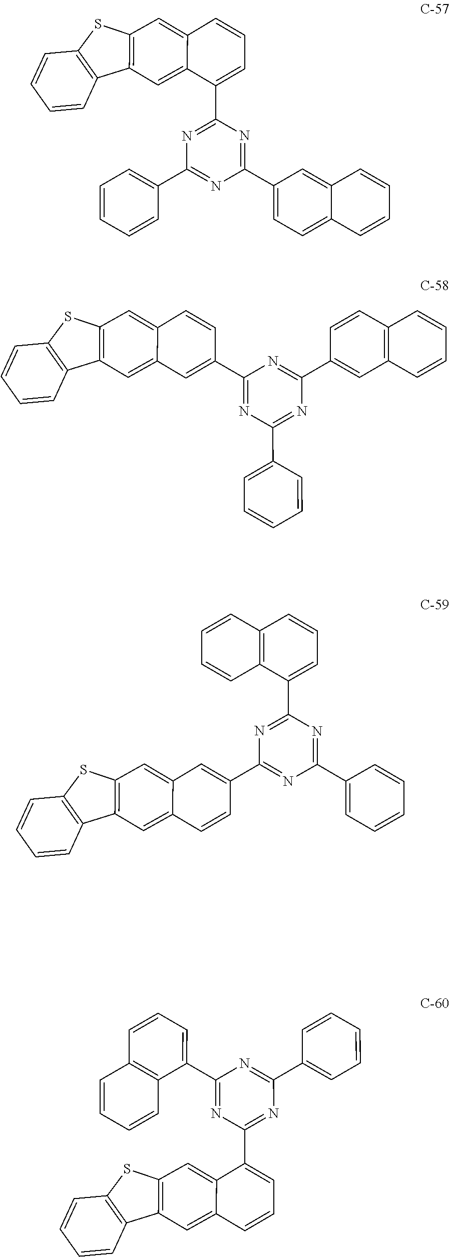 [SMILES: c1ccc(-c2nc(-c3ccc4ccccc4c3)nc(-c3cccc4cc5sc6ccccc6c5cc34)n2)cc1 ; c1ccc(-c2nc(-c3ccc4ccccc4c3)nc(-c3ccc4cc5sc6ccccc6c5cc4c3)n2)cc1 ; c1ccc(-c2nc(-c3ccc4cc5c(cc4c3)sc3ccccc35)nc(-c3cccc4ccccc34)n2)cc1 ; c1ccc(-c2nc(-c3cccc4ccccc34)nc(-c3cccc4cc5c(cc34)sc3ccccc35)n2)cc1]

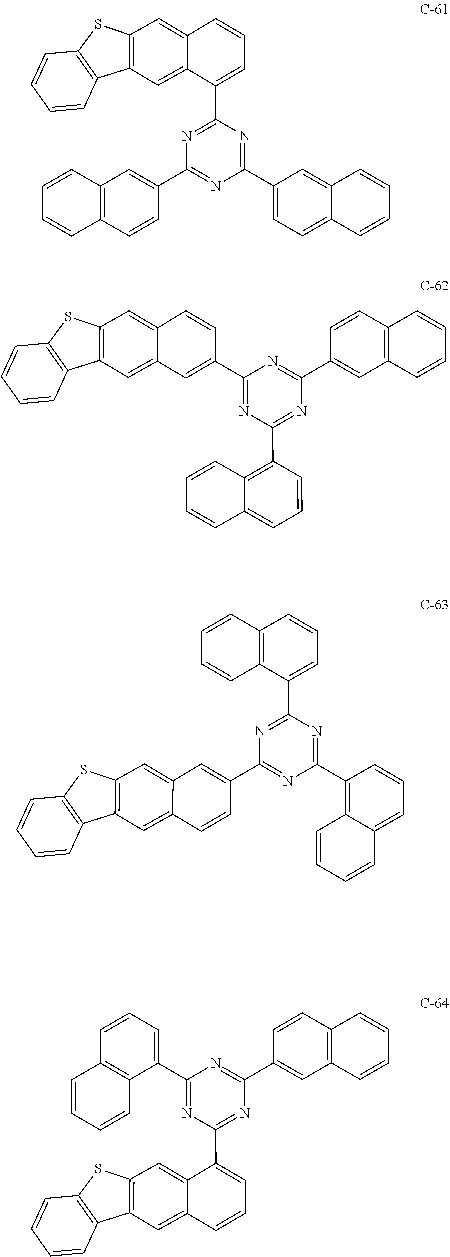 [SMILES: c1ccc2cc(-c3nc(-c4ccc5ccccc5c4)nc(-c4cccc5cc6sc7ccccc7c6cc45)n3)ccc2c1 ; c1ccc2cc(-c3nc(-c4ccc5cc6sc7ccccc7c6cc5c4)nc(-c4cccc5ccccc45)n3)ccc2c1 ; c1ccc2c(-c3nc(-c4ccc5cc6c(cc5c4)sc4ccccc46)nc(-c4cccc5ccccc45)n3)cccc2c1 ; c1ccc2cc(-c3nc(-c4cccc5ccccc45)nc(-c4cccc5cc6c(cc45)sc4ccccc46)n3)ccc2c1]

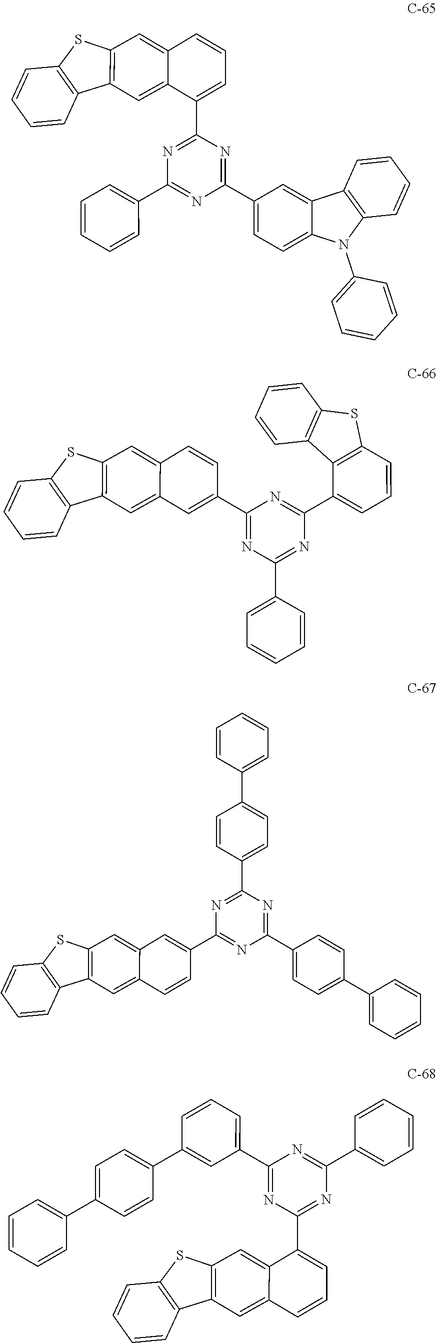 [SMILES: c1ccc(-c2nc(-c3ccc4c(c3)c3ccccc3n4-c3ccccc3)nc(-c3cccc4cc5sc6ccccc6c5cc34)n2)cc1 ; c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1 ; c1ccc(-c2ccc(-c3cccc(-c4nc(-c5ccccc5)nc(-c5cccc6cc7c(cc56)sc5ccccc57)n4)c3)cc2)cc1]

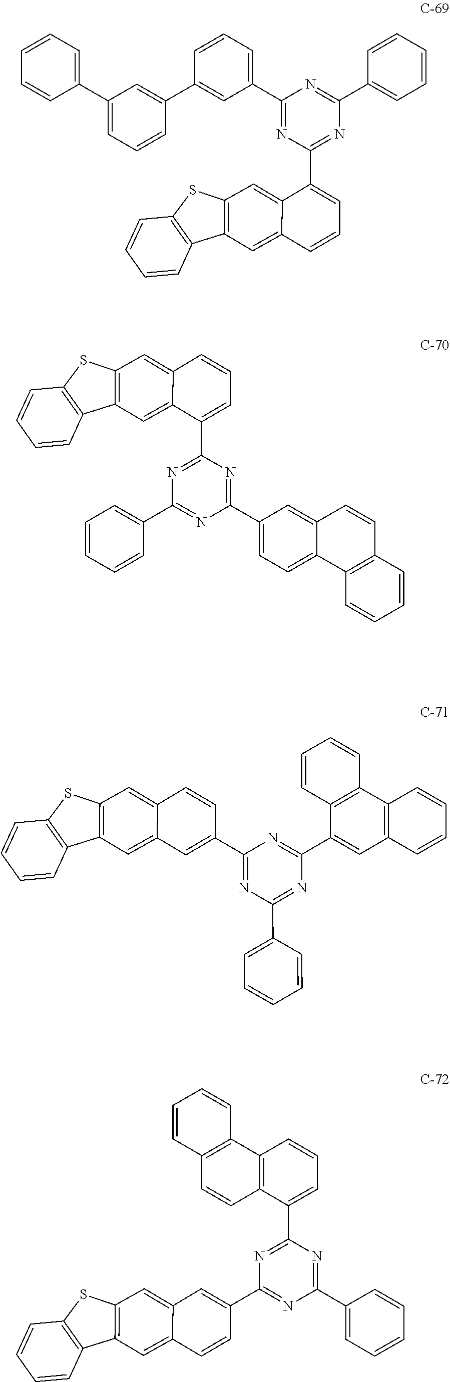 [SMILES: c1ccc(-c2cccc(-c3cccc(-c4nc(-c5ccccc5)nc(-c5cccc6cc7c(cc56)sc5ccccc57)n4)c3)c2)cc1 ; c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1]

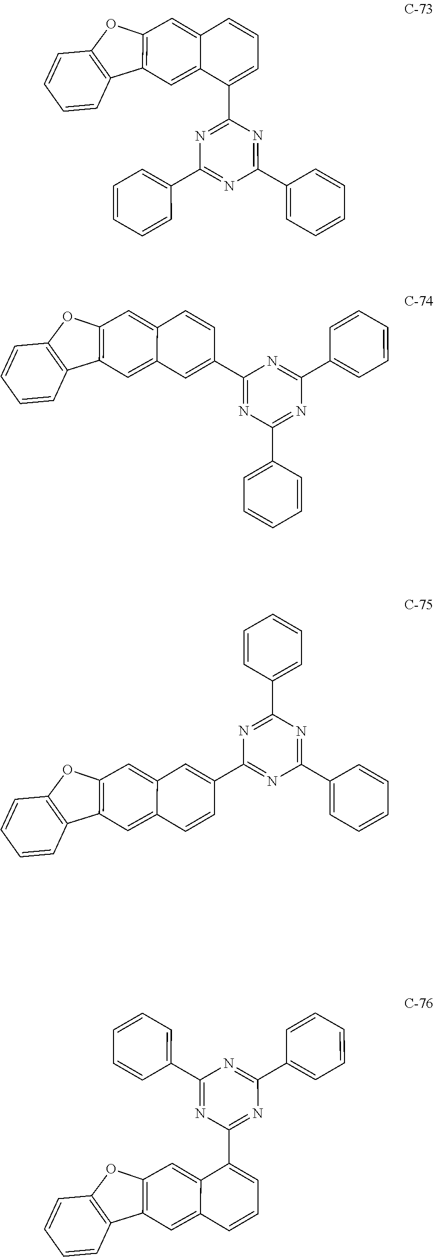 [SMILES: c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1 ; c1ccc(-c2nc(-c3ccccc3)nc(-c3ccc4cc5c(cc4c3)oc3ccccc35)n2)cc1 ; c1ccc(-c2nc(-c3ccccc3)nc(-c3cccc4cc5c(cc34)oc3ccccc35)n2)cc1]

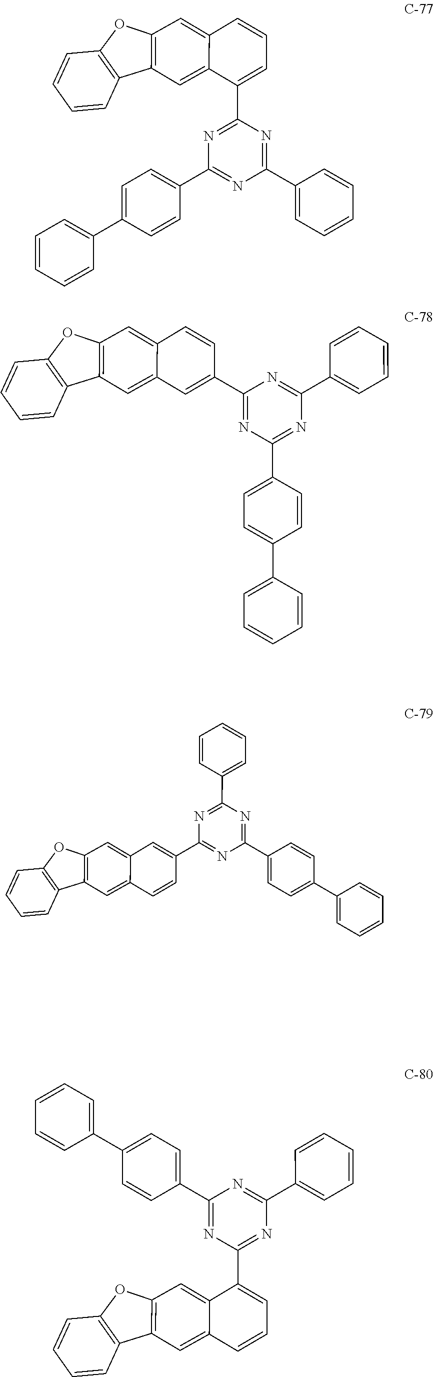

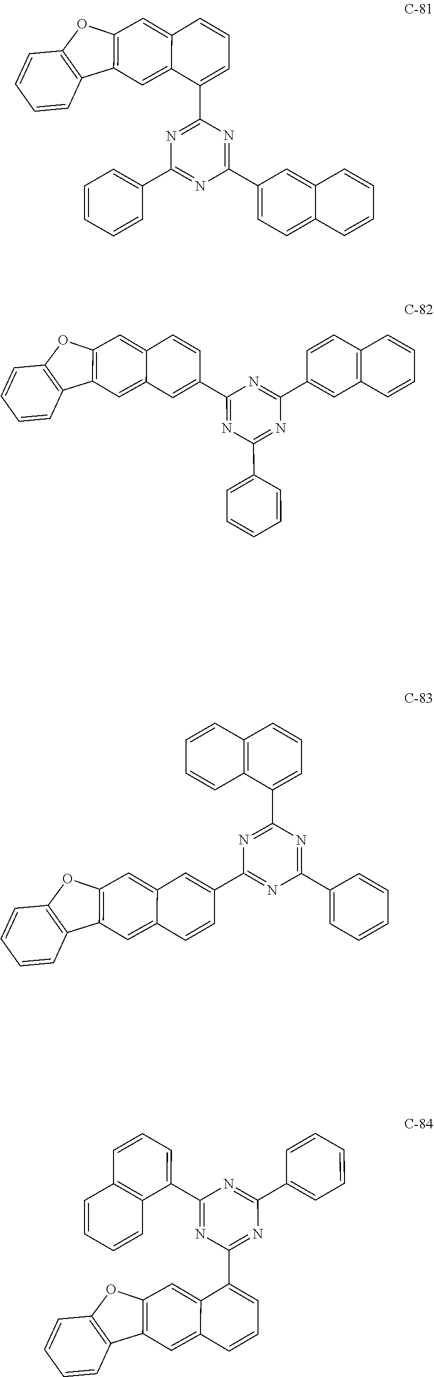 [SMILES: c1ccc(-c2nc(-c3ccc4ccccc4c3)nc(-c3cccc4cc5oc6ccccc6c5cc34)n2)cc1 ; c1ccc(-c2nc(-c3ccc4ccccc4c3)nc(-c3ccc4cc5oc6ccccc6c5cc4c3)n2)cc1 ; c1ccc(-c2nc(-c3ccc4cc5c(cc4c3)oc3ccccc35)nc(-c3cccc4ccccc34)n2)cc1 ; c1ccc(-c2nc(-c3cccc4ccccc34)nc(-c3cccc4cc5c(cc34)oc3ccccc35)n2)cc1]

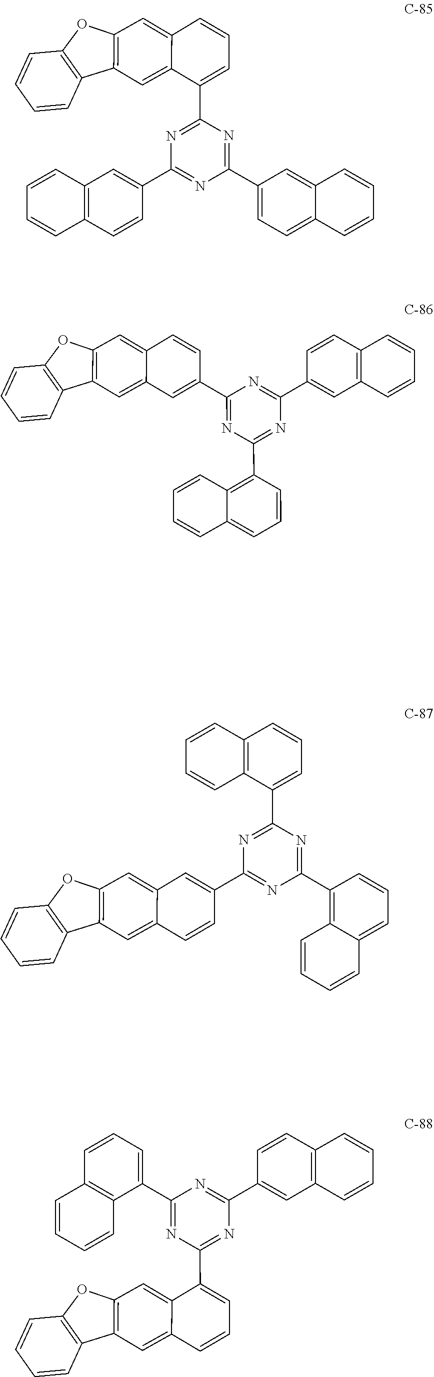 [SMILES: c1ccc2cc(-c3nc(-c4ccc5ccccc5c4)nc(-c4cccc5cc6oc7ccccc7c6cc45)n3)ccc2c1 ; c1ccc2cc(-c3nc(-c4ccc5cc6oc7ccccc7c6cc5c4)nc(-c4cccc5ccccc45)n3)ccc2c1 ; c1ccc2c(-c3nc(-c4ccc5cc6c(cc5c4)oc4ccccc46)nc(-c4cccc5ccccc45)n3)cccc2c1 ; c1ccc2cc(-c3nc(-c4cccc5ccccc45)nc(-c4cccc5cc6c(cc45)oc4ccccc46)n3)ccc2c1]

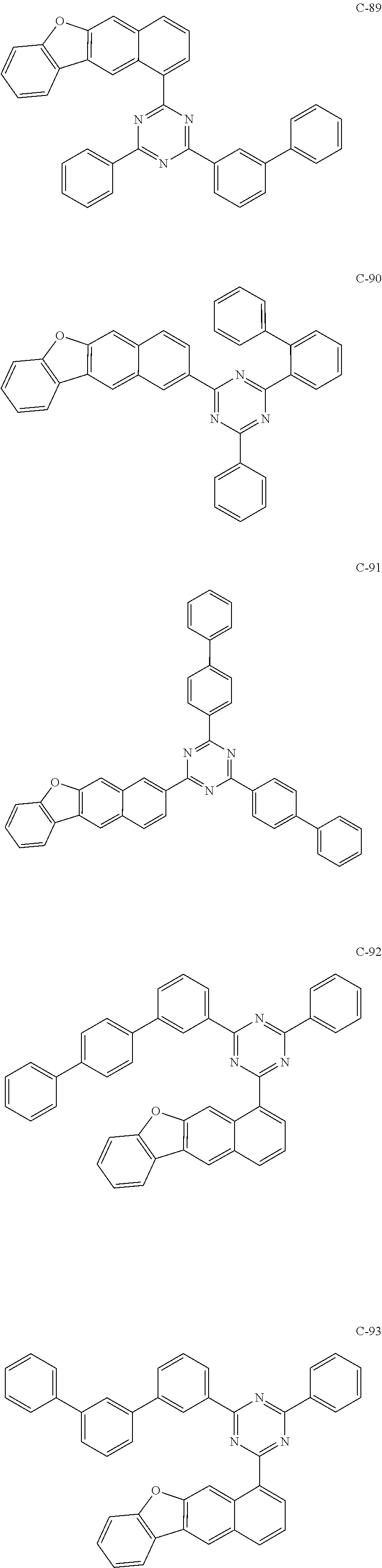 [SMILES: c1ccc(-c2cccc(-c3nc(-c4ccccc4)nc(-c4cccc5cc6oc7ccccc7c6cc45)n3)c2)cc1 ; c1ccc(-c2nc(-c3ccc4cc5oc6ccccc6c5cc4c3)nc(-c3ccccc3-c3ccccc3)n2)cc1 ; c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1]

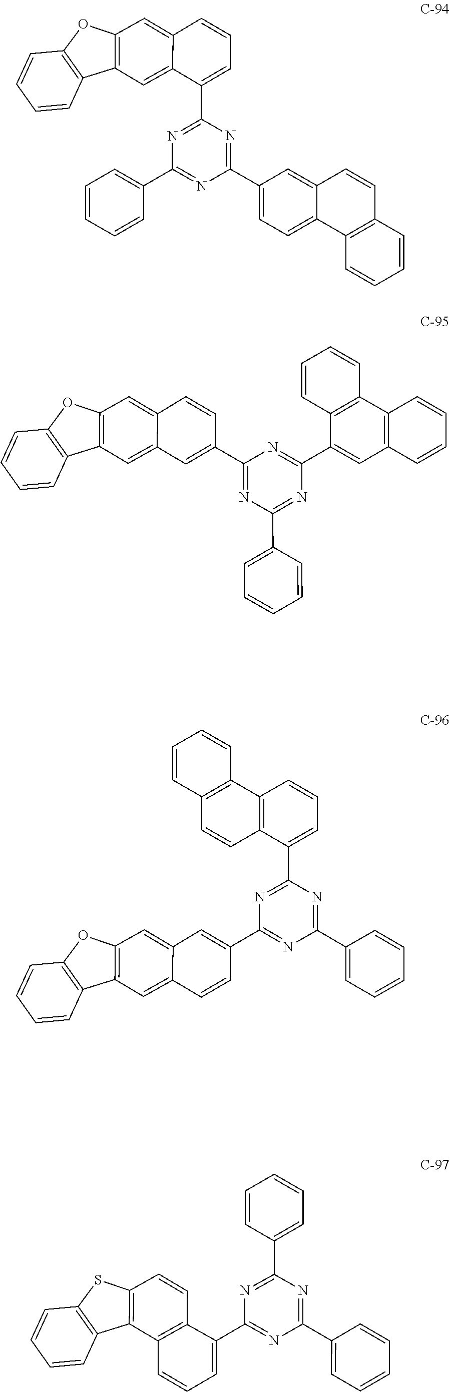 [SMILES: c1ccc(-c2nc(-c3ccc4c(ccc5ccccc54)c3)nc(-c3cccc4cc5oc6ccccc6c5cc34)n2)cc1 ; c1ccc(-c2nc(-c3ccc4cc5oc6ccccc6c5cc4c3)nc(-c3cc4ccccc4c4ccccc34)n2)cc1 ; c1ccc(-c2nc(-c3ccc4cc5c(cc4c3)oc3ccccc35)nc(-c3cccc4c3ccc3ccccc34)n2)cc1 ; c1ccc(-c2nc(-c3ccccc3)nc(-c3cccc4c3ccc3sc5ccccc5c34)n2)cc1]

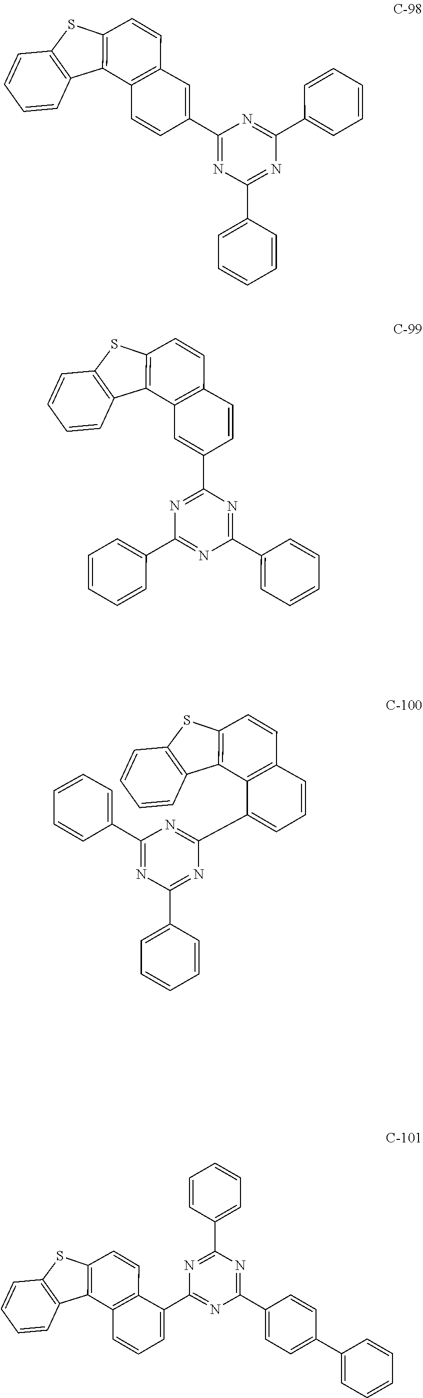 [SMILES: c1ccc(-c2nc(-c3ccccc3)nc(-c3ccc4c(ccc5sc6ccccc6c54)c3)n2)cc1 ; c1ccc(-c2nc(-c3ccccc3)nc(-c3ccc4ccc5sc6ccccc6c5c4c3)n2)cc1 ; c1ccc(-c2nc(-c3ccccc3)nc(-c3cccc4ccc5sc6ccccc6c5c34)n2)cc1 ; c1ccc(-c2ccc(-c3nc(-c4ccccc4)nc(-c4cccc5c4ccc4sc6ccccc6c45)n3)cc2)cc1]

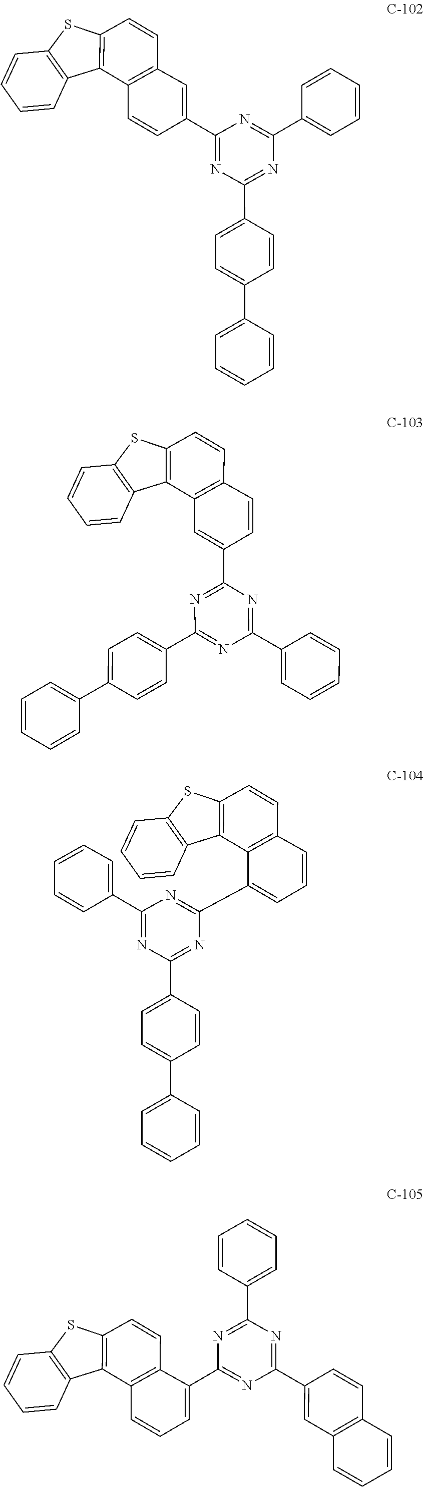 [SMILES: c1ccc(-c2ccc(-c3nc(-c4ccccc4)nc(-c4ccc5c(ccc6sc7ccccc7c65)c4)n3)cc2)cc1 ; c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1]

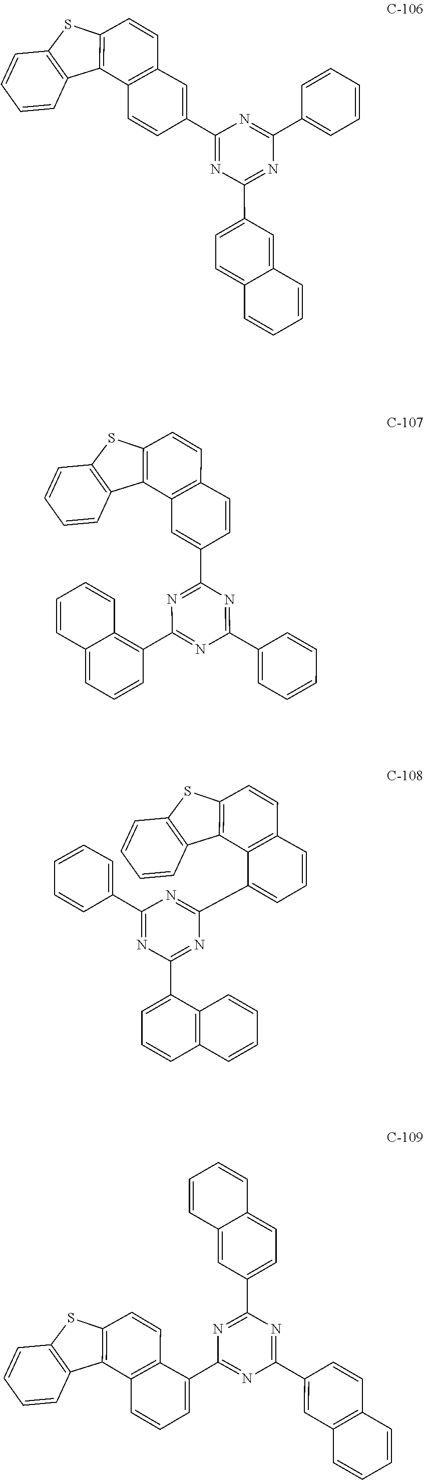 [SMILES: c1ccc(-c2nc(-c3ccc4ccccc4c3)nc(-c3ccc4c(ccc5sc6ccccc6c54)c3)n2)cc1 ; c1ccc(-c2nc(-c3ccc4ccc5sc6ccccc6c5c4c3)nc(-c3cccc4ccccc34)n2)cc1 ; c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1]

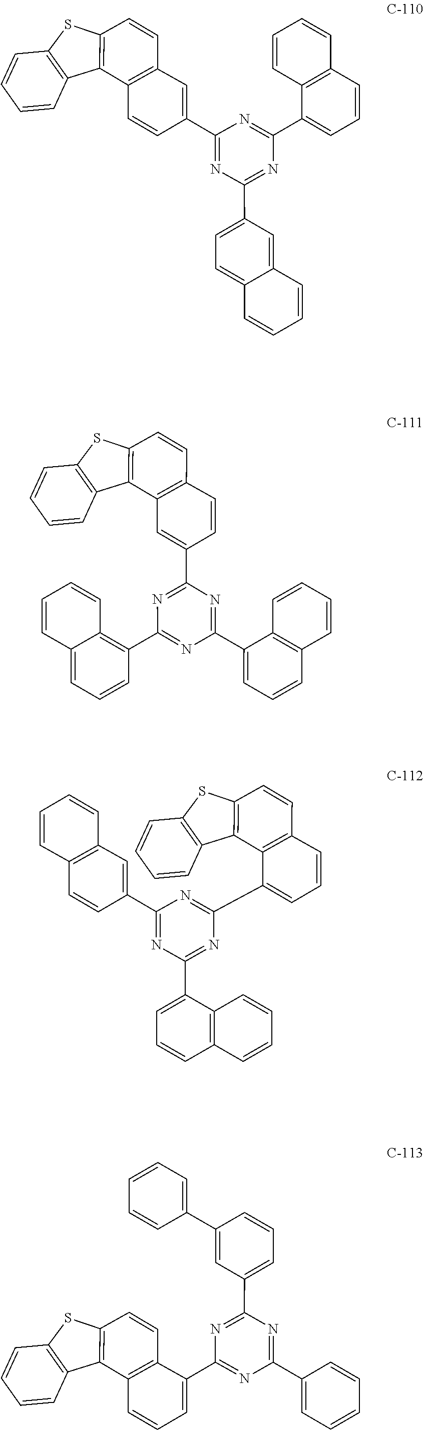 [SMILES: c1ccc2cc(-c3nc(-c4ccc5c(ccc6sc7ccccc7c65)c4)nc(-c4cccc5ccccc45)n3)ccc2c1 ; c1ccc2c(-c3nc(-c4ccc5ccc6sc7ccccc7c6c5c4)nc(-c4cccc5ccccc45)n3)cccc2c1 ; c1ccc2cc(-c3nc(-c4cccc5ccccc45)nc(-c4cccc5ccc6sc7ccccc7c6c45)n3)ccc2c1 ; c1ccc(-c2cccc(-c3nc(-c4ccccc4)nc(-c4cccc5c4ccc4sc6ccccc6c45)n3)c2)cc1]

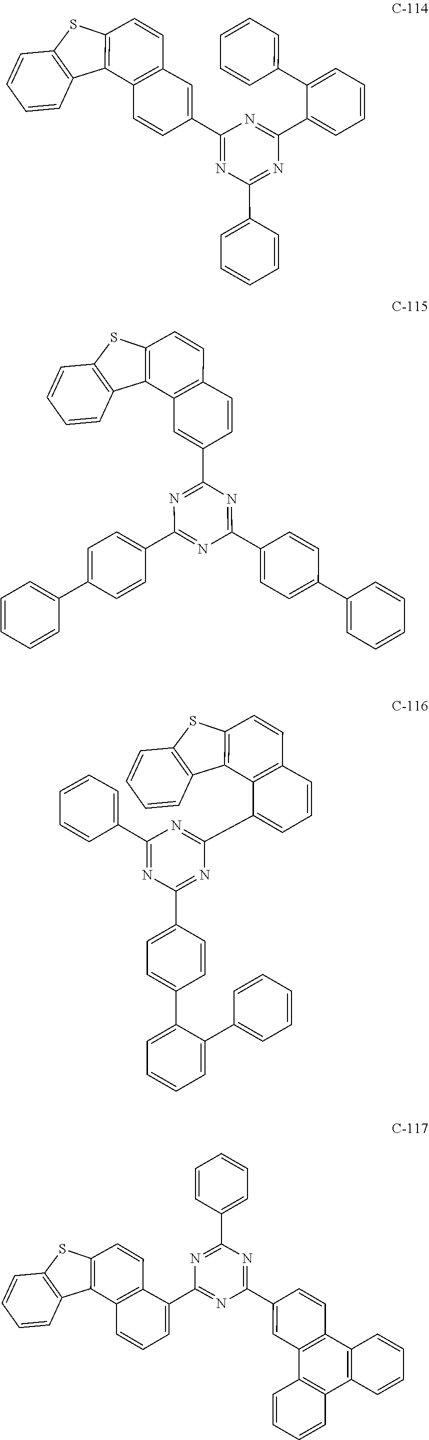 [SMILES: c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1]

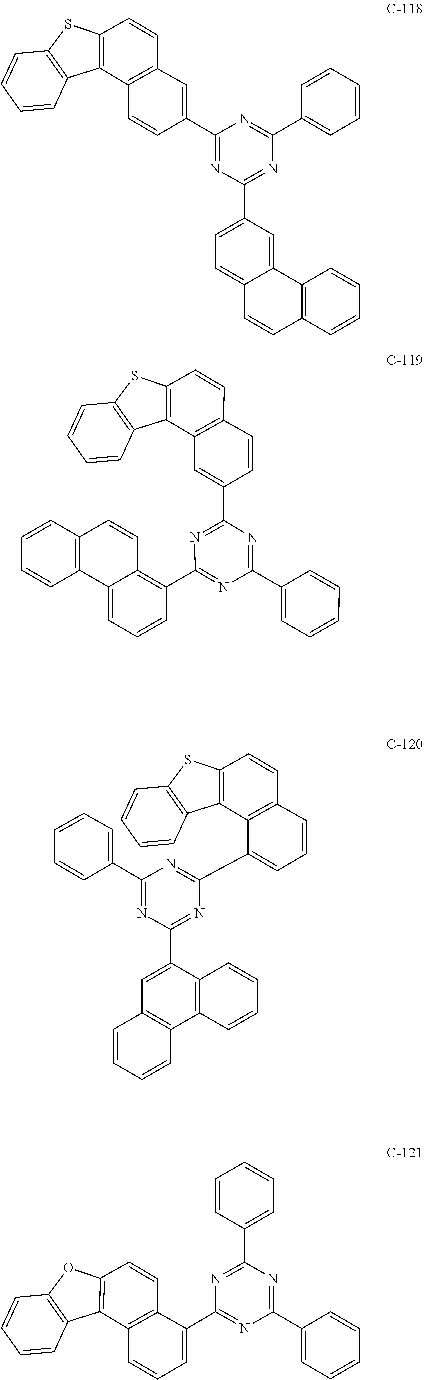 [SMILES: c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1 ; c1ccc(-c2nc(-c3ccccc3)nc(-c3cccc4c3ccc3oc5ccccc5c34)n2)cc1]

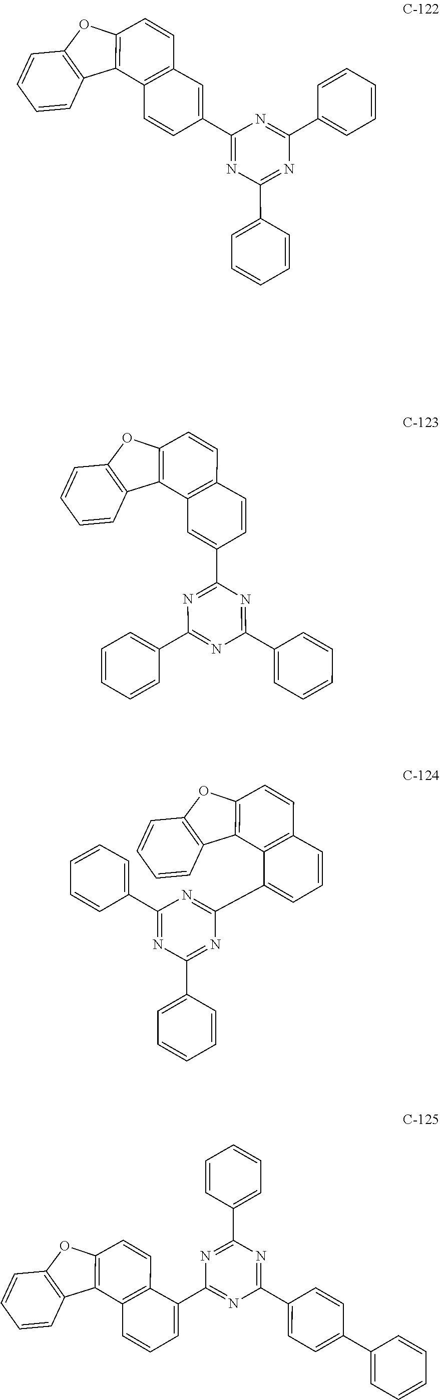 [SMILES: c1ccc(-c2nc(-c3ccccc3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1 ; c1ccc(-c2nc(-c3ccccc3)nc(-c3cccc4ccc5oc6ccccc6c5c34)n2)cc1 ; c1ccc(-c2ccc(-c3nc(-c4ccccc4)nc(-c4cccc5c4ccc4oc6ccccc6c45)n3)cc2)cc1]

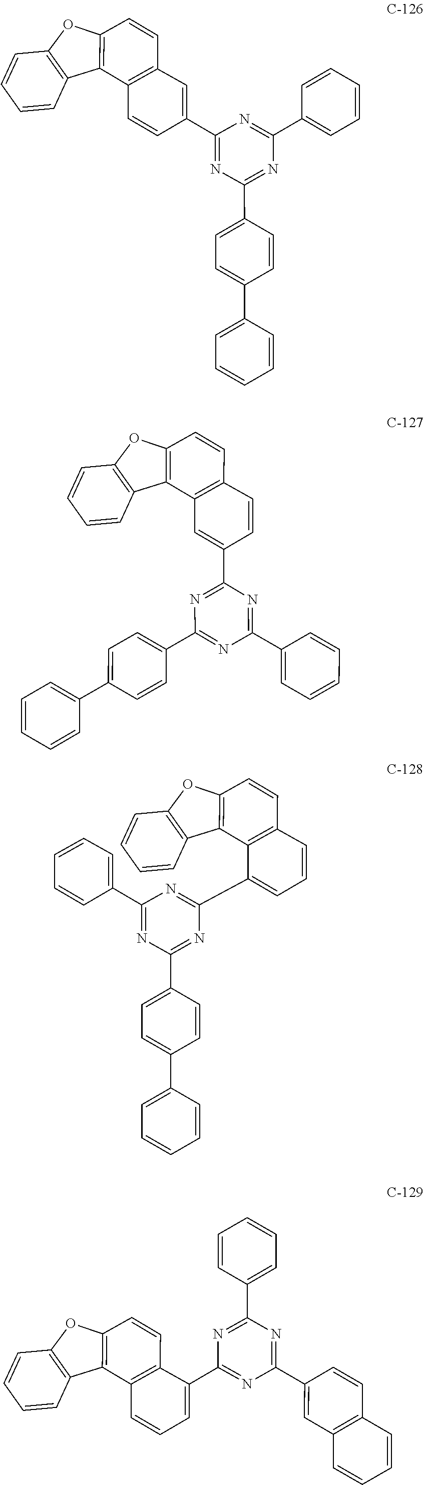 [SMILES: c1ccc(-c2ccc(-c3nc(-c4ccccc4)nc(-c4ccc5c(ccc6oc7ccccc7c65)c4)n3)cc2)cc1 ; c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1 ; c1ccc(-c2nc(-c3ccc4ccccc4c3)nc(-c3cccc4c3ccc3oc5ccccc5c34)n2)cc1]

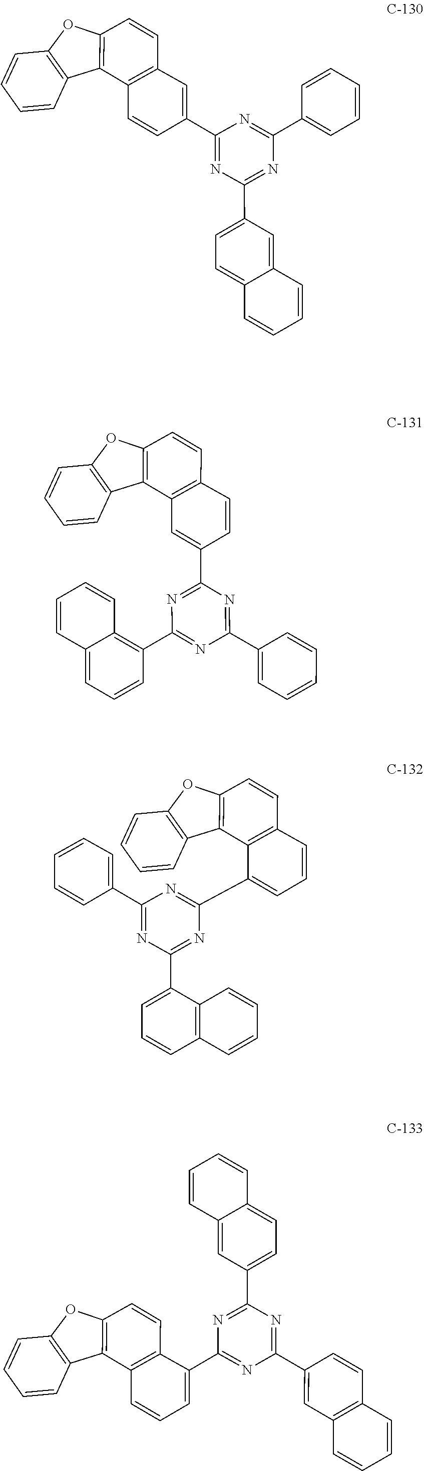 [SMILES: c1ccc(-c2nc(-c3ccc4ccccc4c3)nc(-c3ccc4c(ccc5oc6ccccc6c54)c3)n2)cc1 ; c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1 ; c1ccc2cc(-c3nc(-c4ccc5ccccc5c4)nc(-c4cccc5c4ccc4oc6ccccc6c45)n3)ccc2c1]

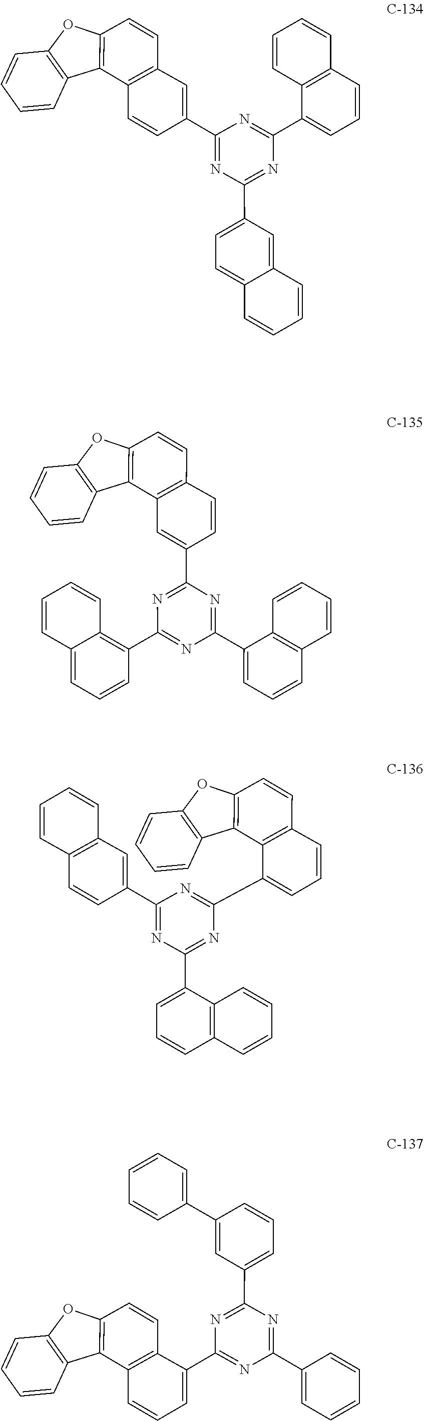 [SMILES: c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1 ; c1ccc2cc(-c3nc(-c4cccc5ccccc45)nc(-c4cccc5ccc6oc7ccccc7c6c45)n3)ccc2c1 ; c1ccc(-c2cccc(-c3nc(-c4ccccc4)nc(-c4cccc5c4ccc4oc6ccccc6c45)n3)c2)cc1]

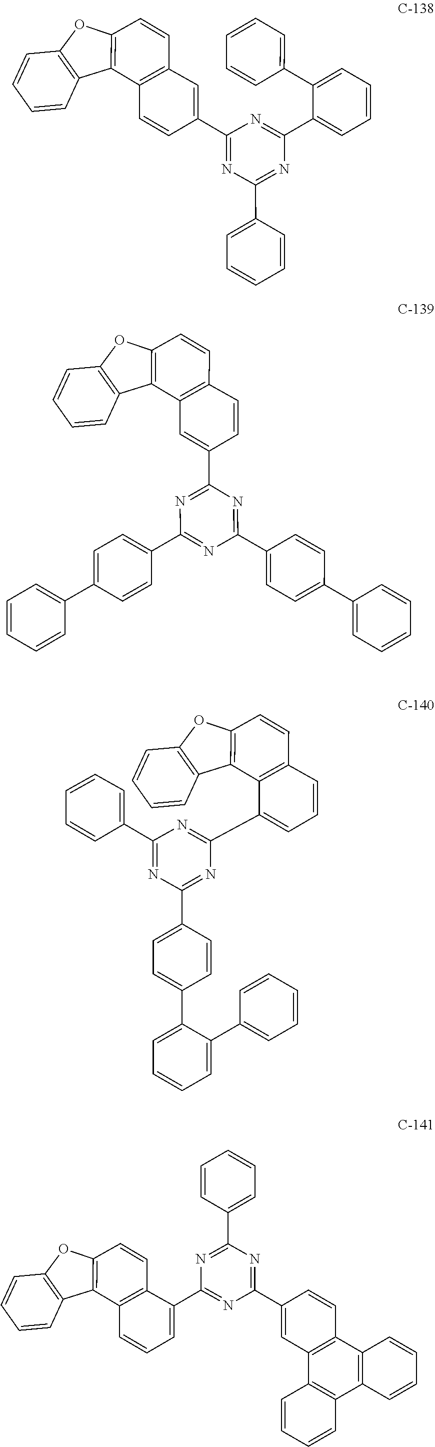 [SMILES: c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1 ; c1ccc(-c2nc(-c3ccc4c5ccccc5c5ccccc5c4c3)nc(-c3cccc4c3ccc3oc5ccccc5c34)n2)cc1]

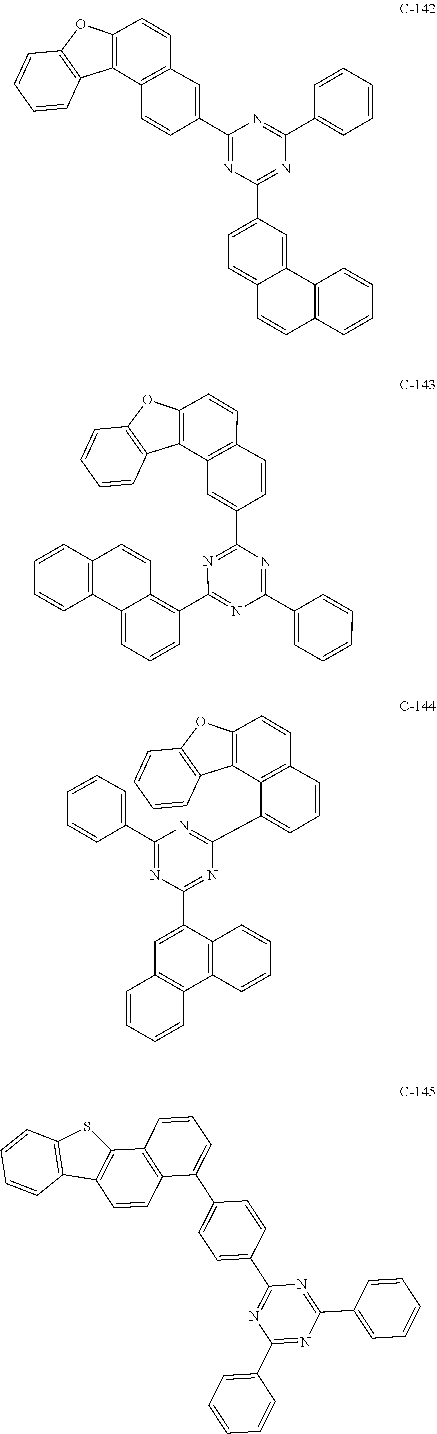 [SMILES: c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1 ; c1ccc(-c2nc(-c3ccccc3)nc(-c3ccc(-c4cccc5c4ccc4c6ccccc6sc54)cc3)n2)cc1]

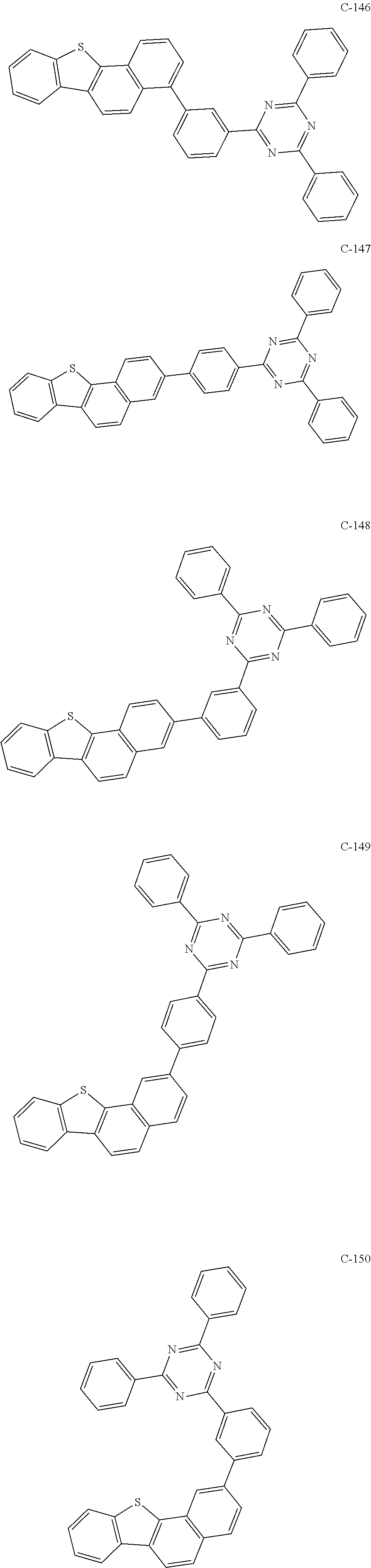 [SMILES: c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1 ; c1ccc(-c2nc(-c3ccccc3)nc(-c3cccc(-c4ccc5ccc6c7ccccc7sc6c5c4)c3)n2)cc1]

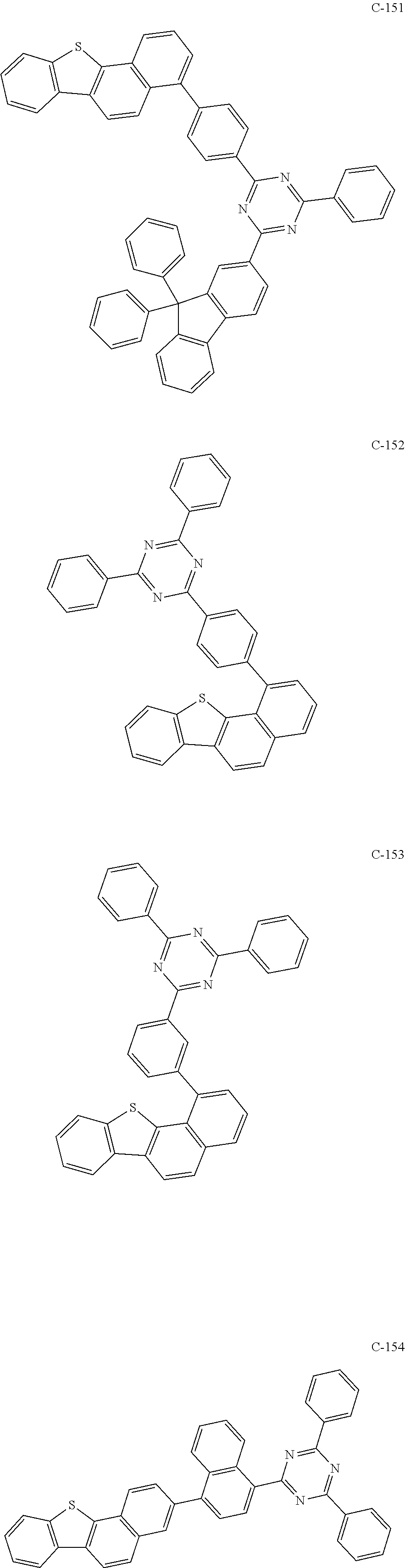 [SMILES: c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1 ; c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc(-c4cccc5ccc6c7ccccc7sc6c45)c3)n2)cc1 ; c1ccc(-c2nc(-c3ccccc3)nc(-c3ccc(-c4ccc5c(ccc6c7ccccc7sc56)c4)c4ccccc34)n2)cc1]

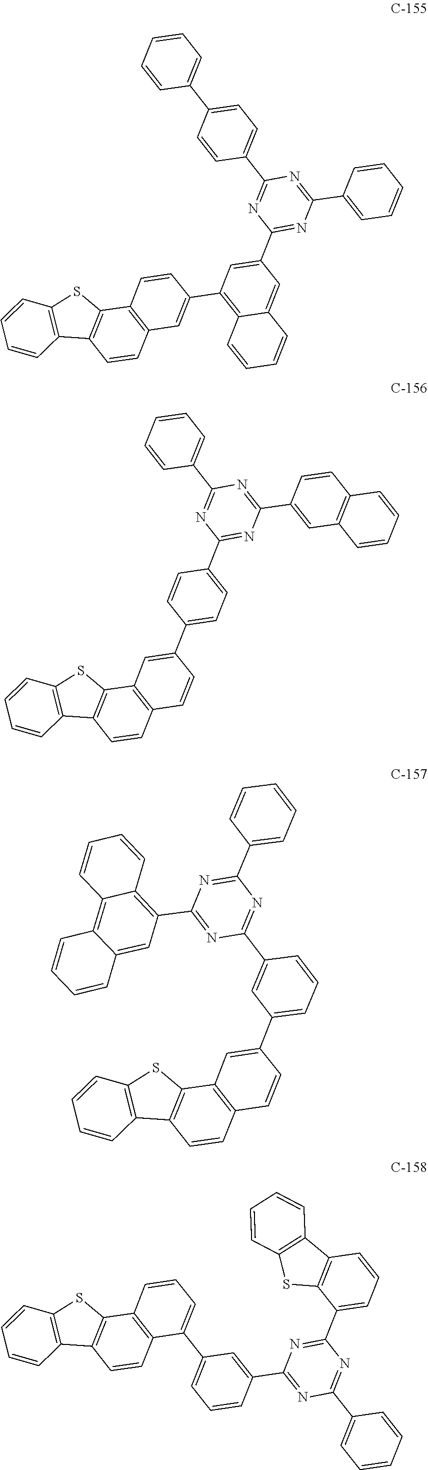 [SMILES: c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1 ; c1ccc(-c2nc(-c3cccc(-c4cccc5c4ccc4c6ccccc6sc54)c3)nc(-c3cccc4c3sc3ccccc34)n2)cc1]

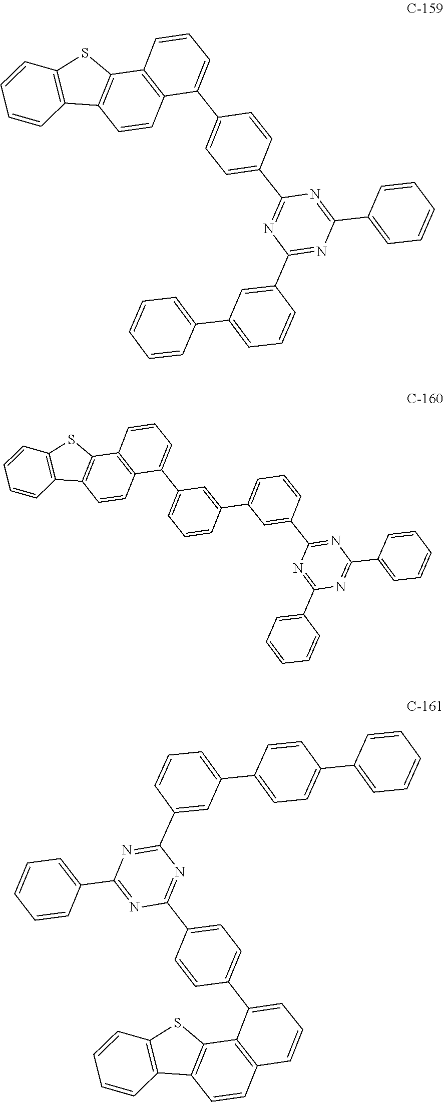 [SMILES: c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1]

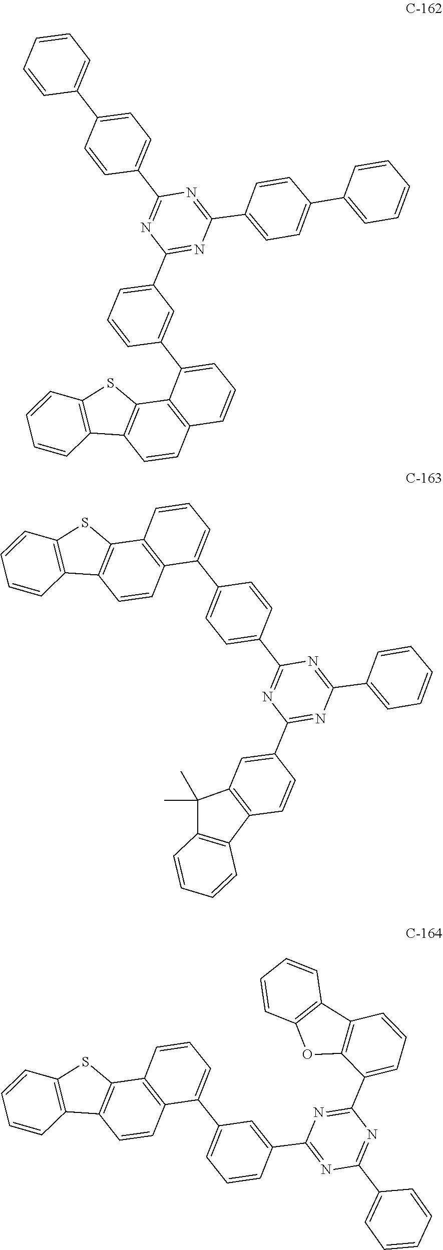 [SMILES: c1ccc(-c2ccc(-c3nc(-c4ccc(-c5ccccc5)cc4)nc(-c4cccc(-c5cccc6ccc7c8ccccc8sc7c56)c4)n3)cc2)cc1 ; CC1(C)c2ccccc2-c2ccc(-c3nc(-c4ccccc4)nc(-c4ccc(-c5cccc6c5ccc5c7ccccc7sc65)cc4)n3)cc21 ; c1ccc(-c2nc(-c3cccc(-c4cccc5c4ccc4c6ccccc6sc54)c3)nc(-c3cccc4c3oc3ccccc34)n2)cc1]

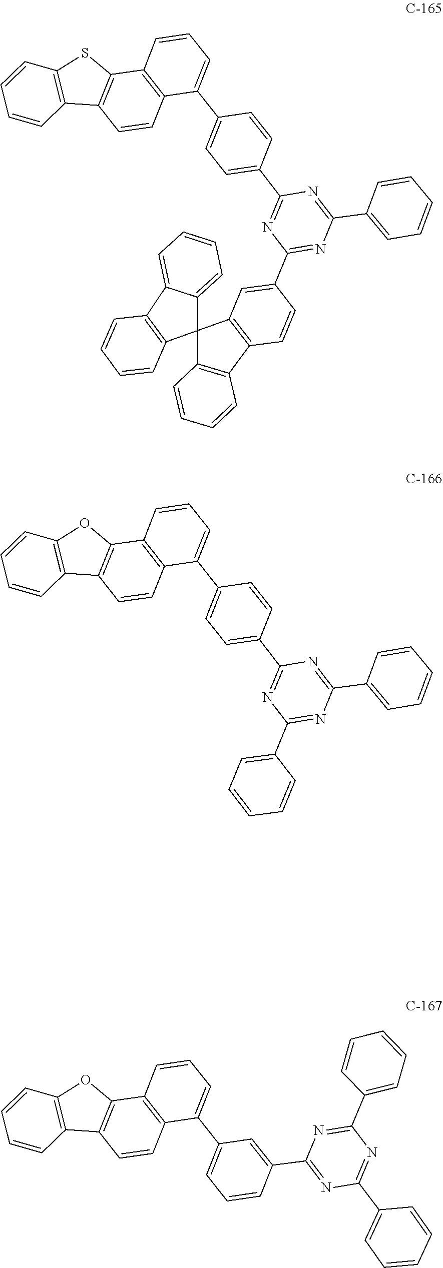 [SMILES: c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1]

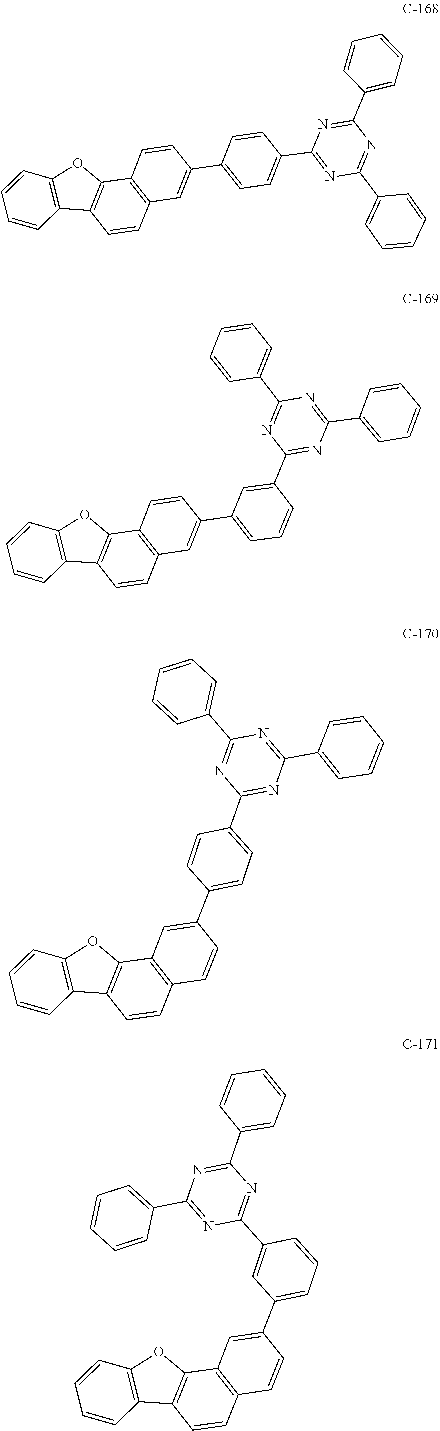 [SMILES: c1ccc(-c2nc(-c3ccccc3)nc(-c3ccc(-c4ccc5c(ccc6c7ccccc7oc56)c4)cc3)n2)cc1 ; c1ccc(-c2nc(-c3ccccc3)nc(-c3cccc(-c4ccc5c(ccc6c7ccccc7oc56)c4)c3)n2)cc1 ; c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1]

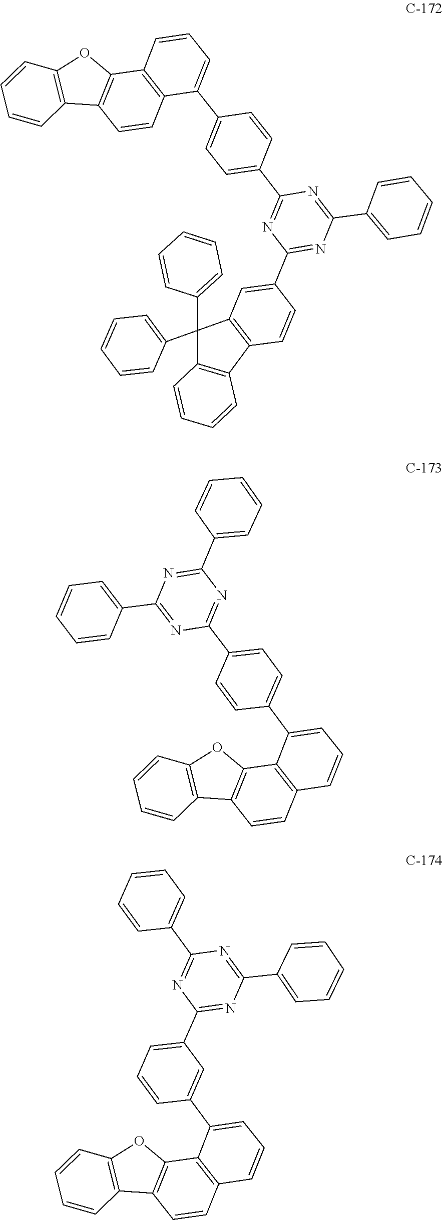 [SMILES: c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1 ; c1ccc(-c2nc(-c3ccccc3)nc(-c3ccc(-c4cccc5ccc6c7ccccc7oc6c45)cc3)n2)cc1 ; c1ccc(-c2nc(-c3ccccc3)nc(-c3cccc(-c4cccc5ccc6c7ccccc7oc6c45)c3)n2)cc1]

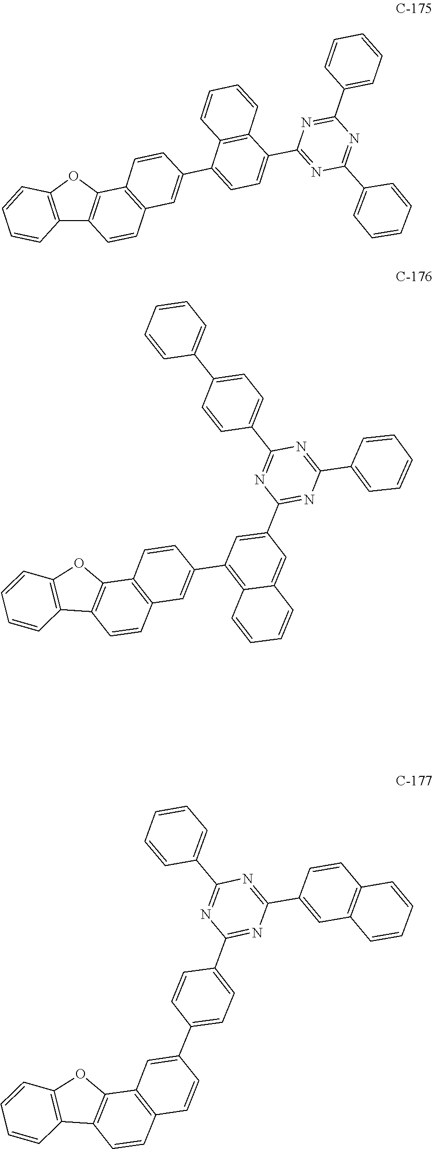 [SMILES: c1ccc(-c2nc(-c3ccccc3)nc(-c3ccc(-c4ccc5c(ccc6c7ccccc7oc56)c4)c4ccccc34)n2)cc1 ; c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1]

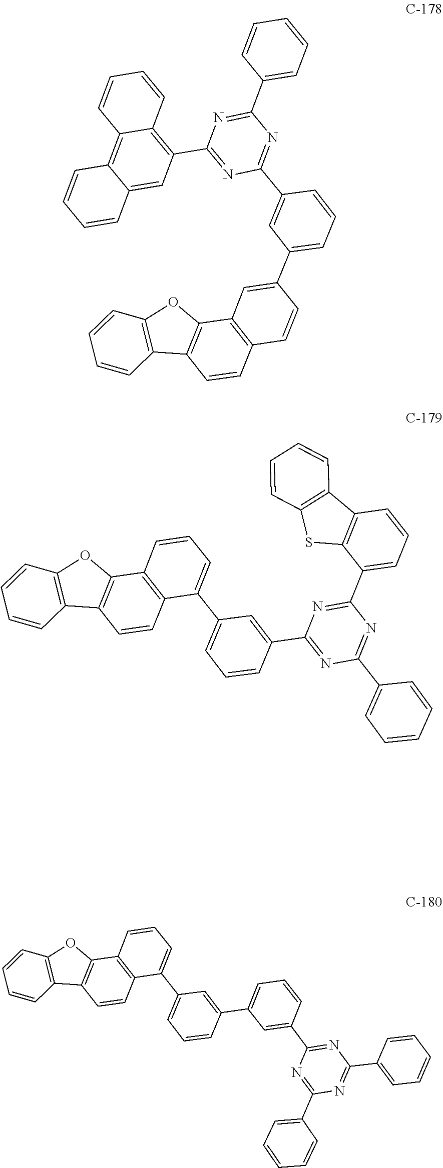 [SMILES: c1ccc(-c2nc(-c3cccc(-c4ccc5ccc6c7ccccc7oc6c5c4)c3)nc(-c3cc4ccccc4c4ccccc34)n2)cc1 ; c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3cccc(-c4cccc(-c5cccc6c5ccc5c7ccccc7oc65)c4)c3)n2)cc1]

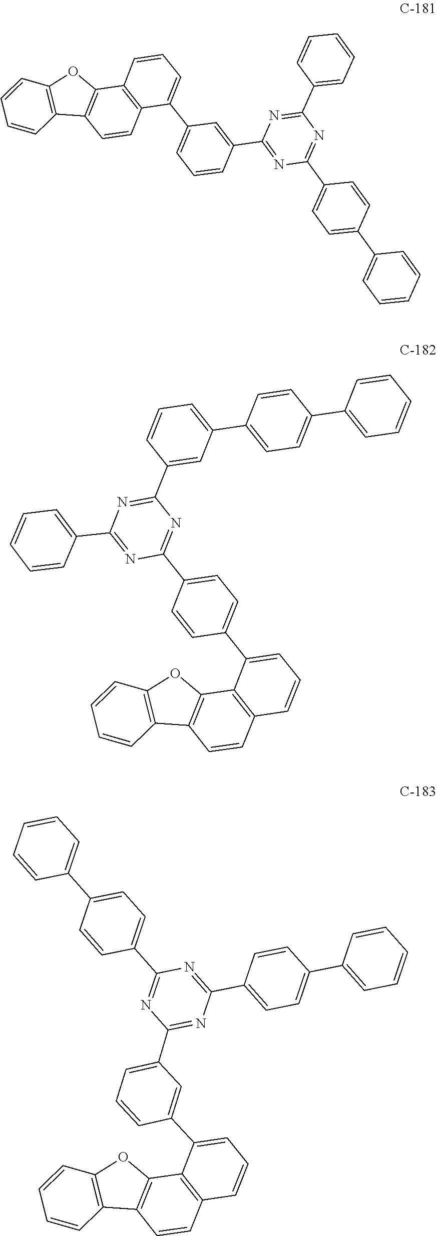 [SMILES: c1ccc(-c2ccc(-c3nc(-c4ccccc4)nc(-c4cccc(-c5cccc6c5ccc5c7ccccc7oc65)c4)n3)cc2)cc1 ; c1ccc(-c2ccc(-c3cccc(-c4nc(-c5ccccc5)nc(-c5ccc(-c6cccc7ccc8c9ccccc9oc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8oc7c56)c4)n3)cc2)cc1]

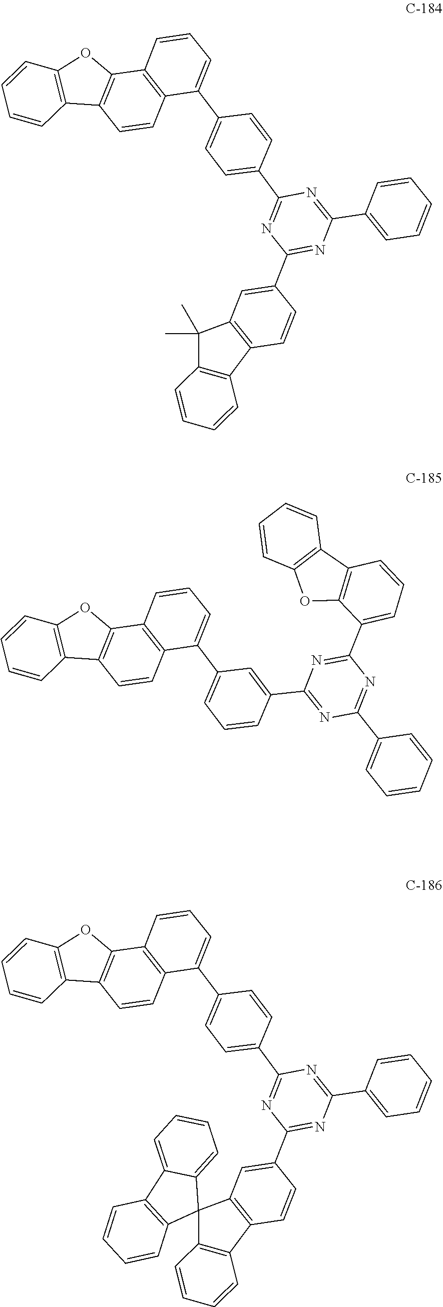 [SMILES: CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1]

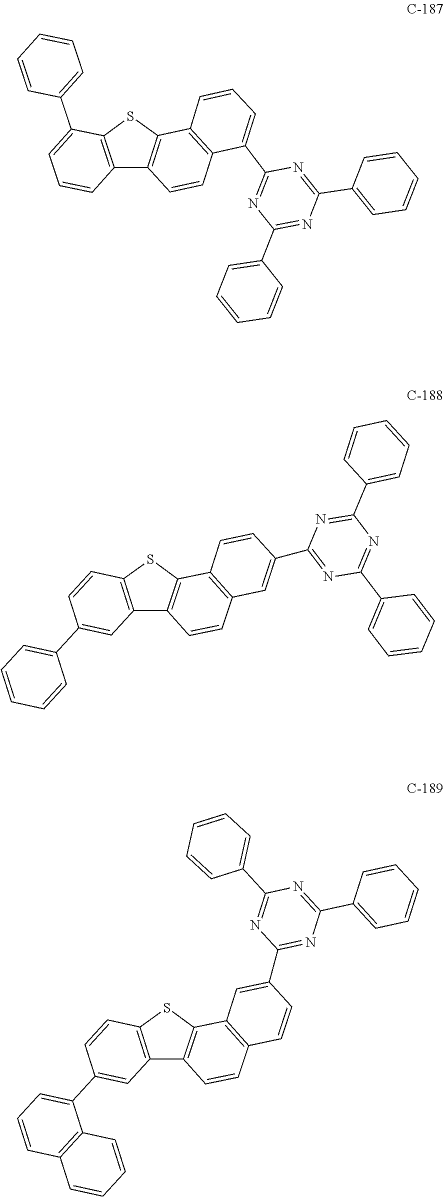 [SMILES: c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1]

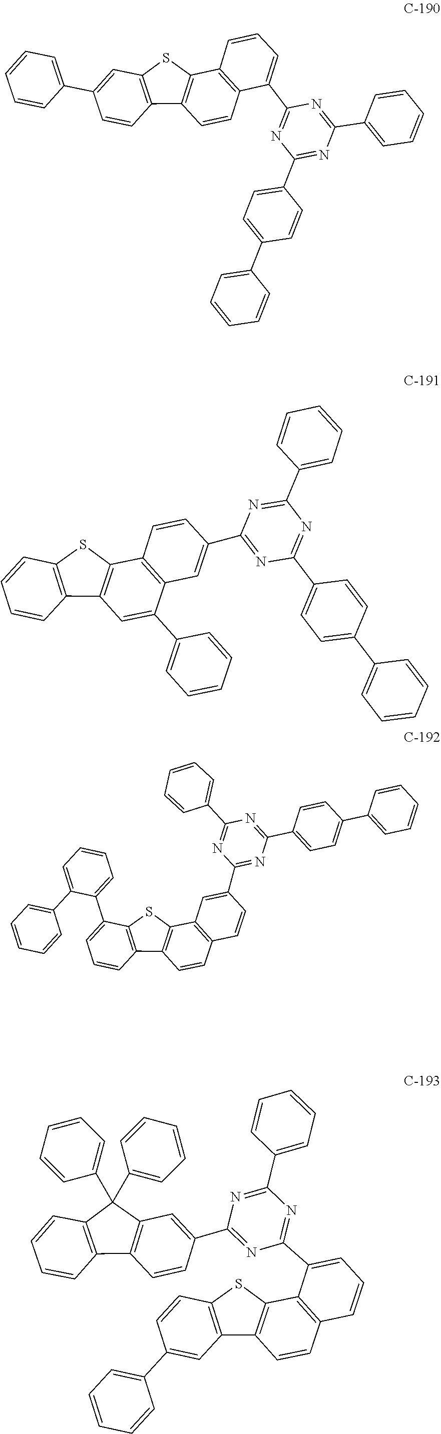 [SMILES: c1ccc(-c2ccc(-c3nc(-c4ccccc4)nc(-c4cccc5c4ccc4c6ccc(-c7ccccc7)cc6sc54)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1]

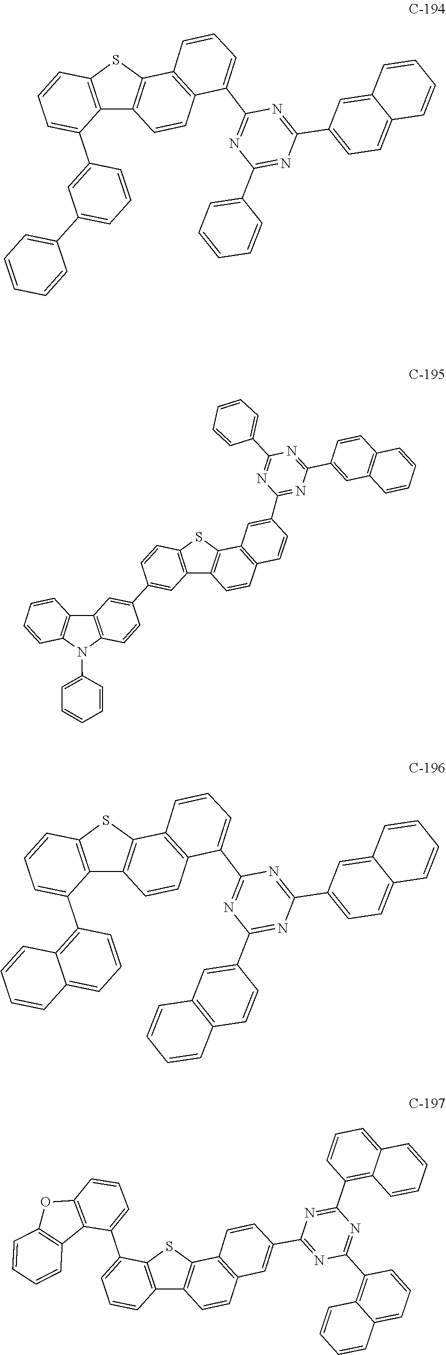 [SMILES: c1ccc(-c2cccc(-c3cccc4sc5c6cccc(-c7nc(-c8ccccc8)nc(-c8ccc9ccccc9c8)n7)c6ccc5c34)c2)cc1 ; c1ccc(-c2nc(-c3ccc4ccccc4c3)nc(-c3ccc4ccc5c6cc(-c7ccc8c(c7)c7ccccc7n8-c7ccccc7)ccc6sc5c4c3)n2)cc1 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1]

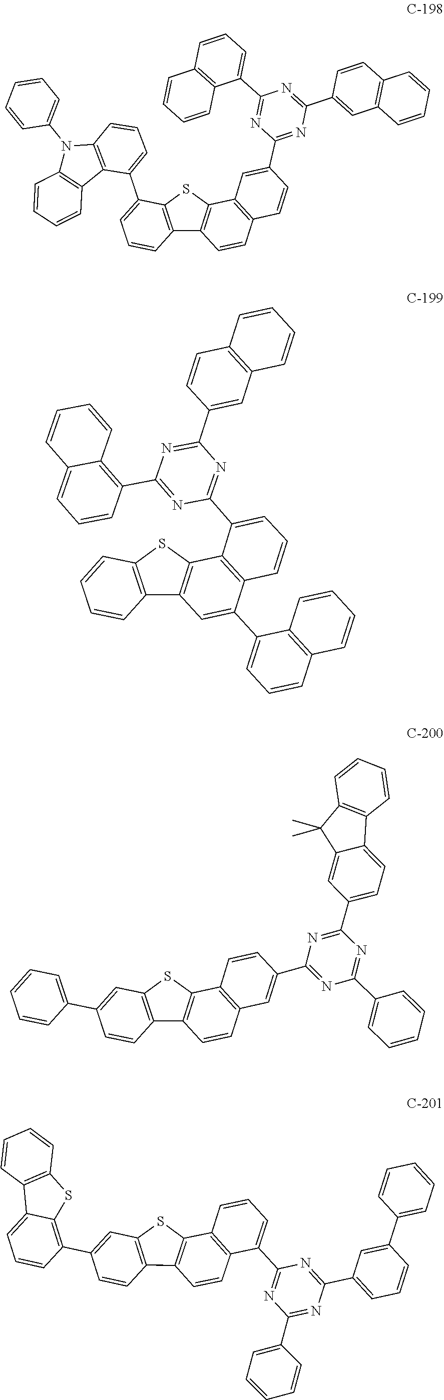 [SMILES: c1ccc(-n2c3ccccc3c3c(-c4cccc5c4sc4c6cc(-c7nc(-c8ccc9ccccc9c8)nc(-c8cccc9ccccc89)n7)ccc6ccc54)cccc32)cc1 ; c1ccc2cc(-c3nc(-c4cccc5ccccc45)nc(-c4cccc5c(-c6cccc7ccccc67)cc6c7ccccc7sc6c45)n3)ccc2c1 ; CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1]

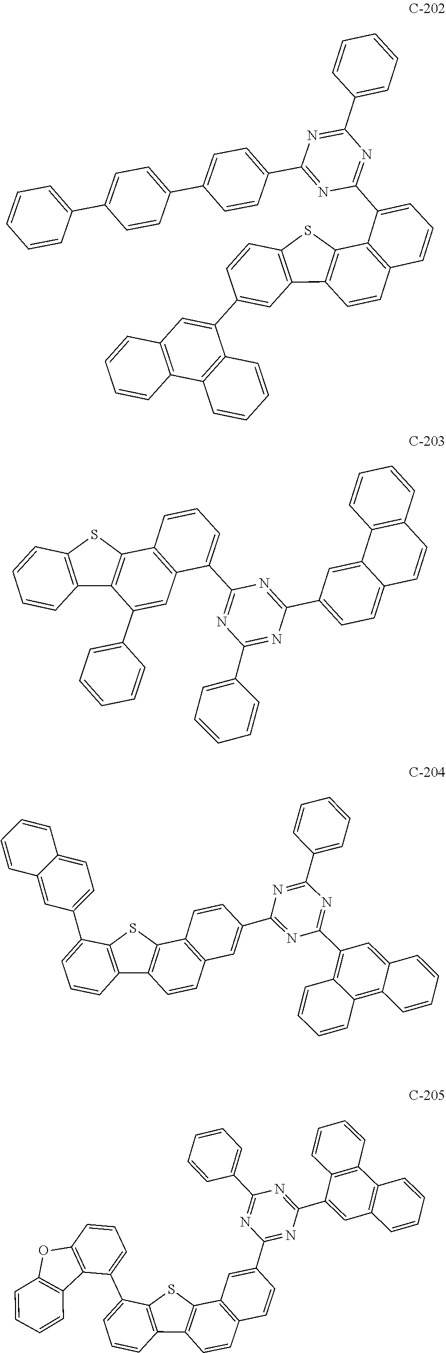 [SMILES: c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1]

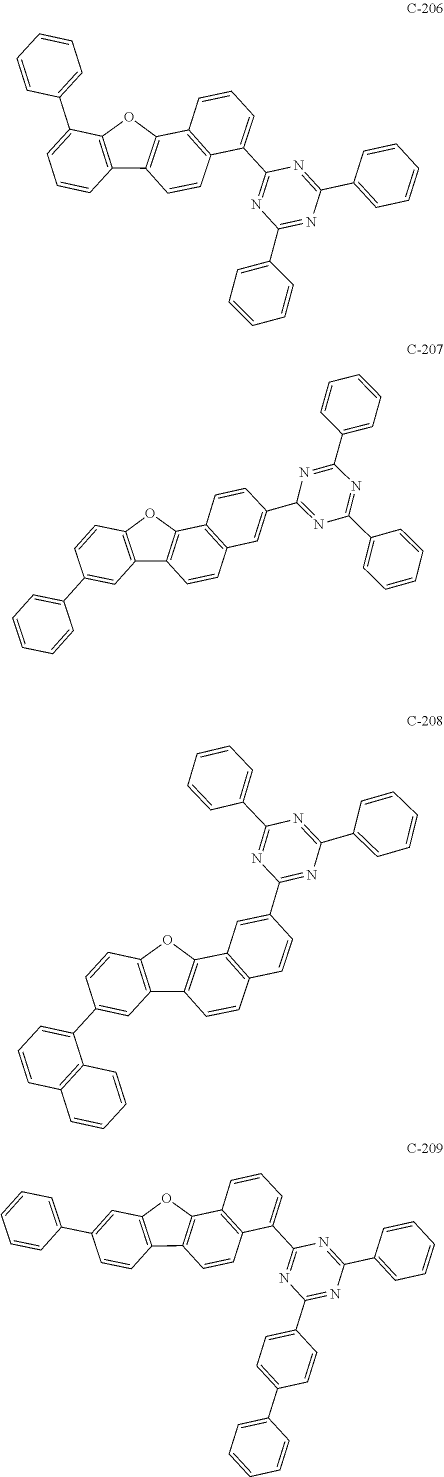 [SMILES: c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1 ; c1ccc(-c2ccc(-c3nc(-c4ccccc4)nc(-c4cccc5c4ccc4c6ccc(-c7ccccc7)cc6oc54)n3)cc2)cc1]

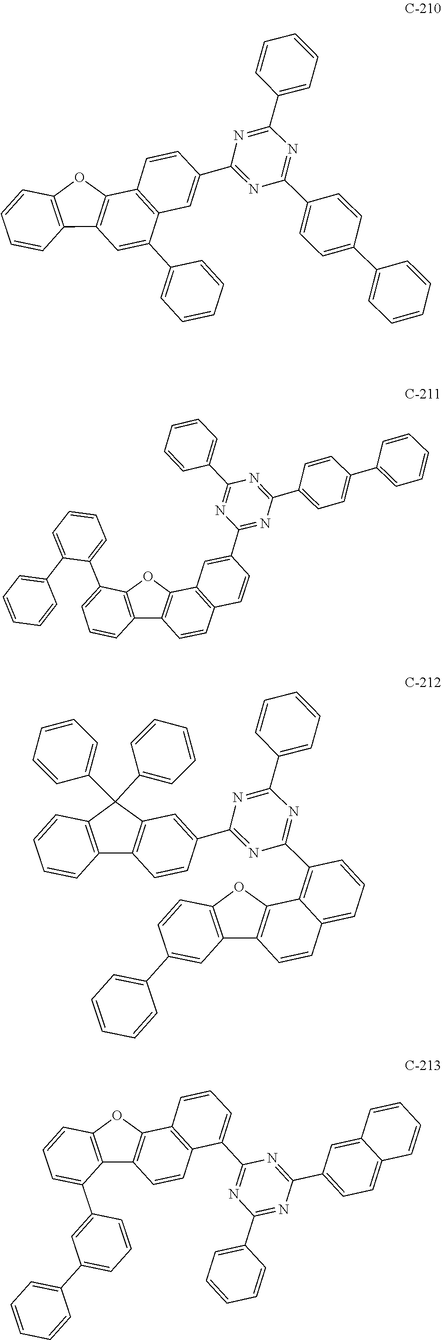 [SMILES: c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1 ; c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4oc5c6cccc(-c7nc(-c8ccccc8)nc(-c8ccc9ccccc9c8)n7)c6ccc5c34)c2)cc1]

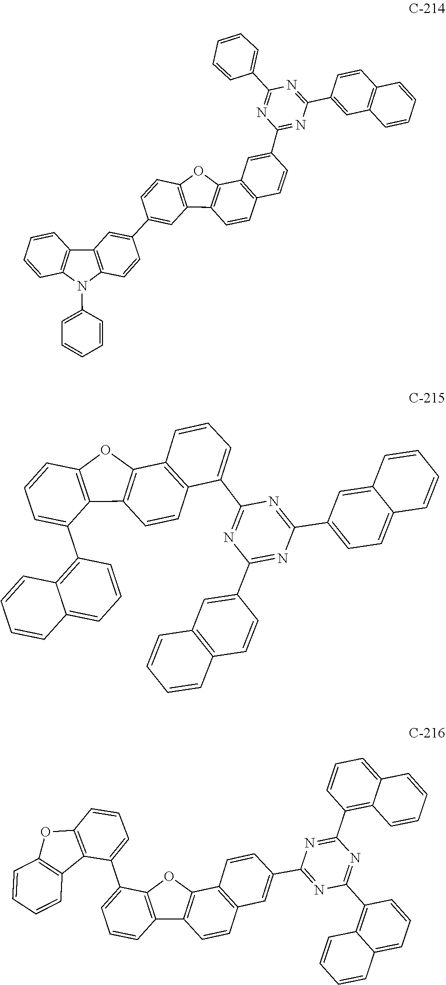 [SMILES: c1ccc(-c2nc(-c3ccc4ccccc4c3)nc(-c3ccc4ccc5c6cc(-c7ccc8c(c7)c7ccccc7n8-c7ccccc7)ccc6oc5c4c3)n2)cc1 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1]

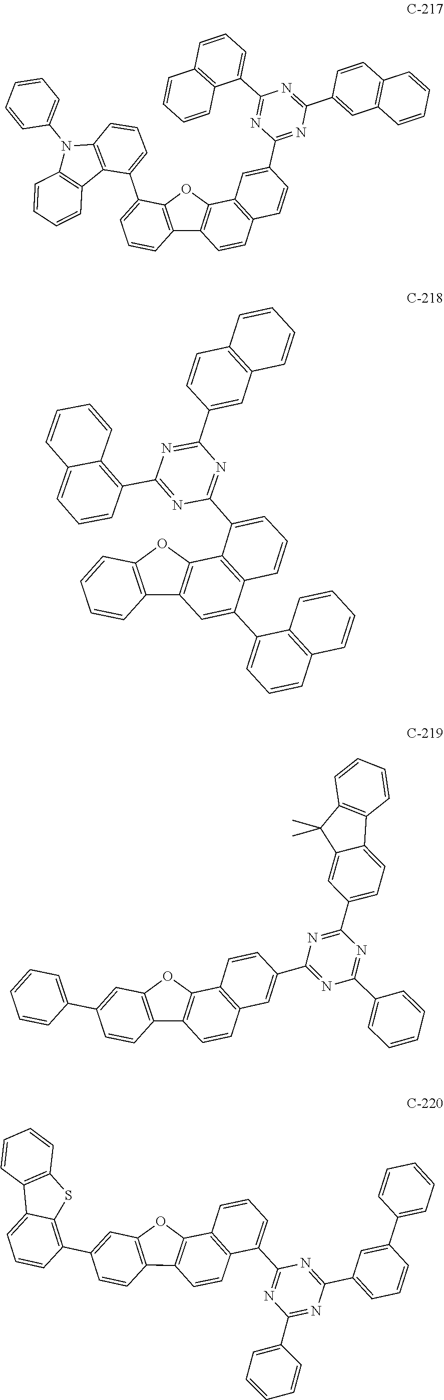 [SMILES: c1ccc(-n2c3ccccc3c3c(-c4cccc5c4oc4c6cc(-c7nc(-c8ccc9ccccc9c8)nc(-c8cccc9ccccc89)n7)ccc6ccc54)cccc32)cc1 ; c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1]

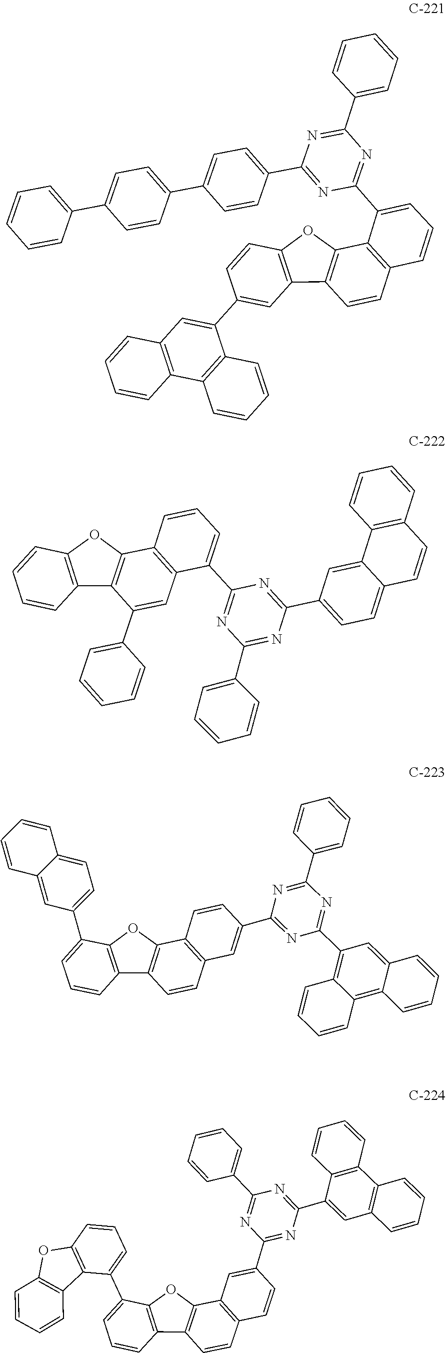 [SMILES: c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1]

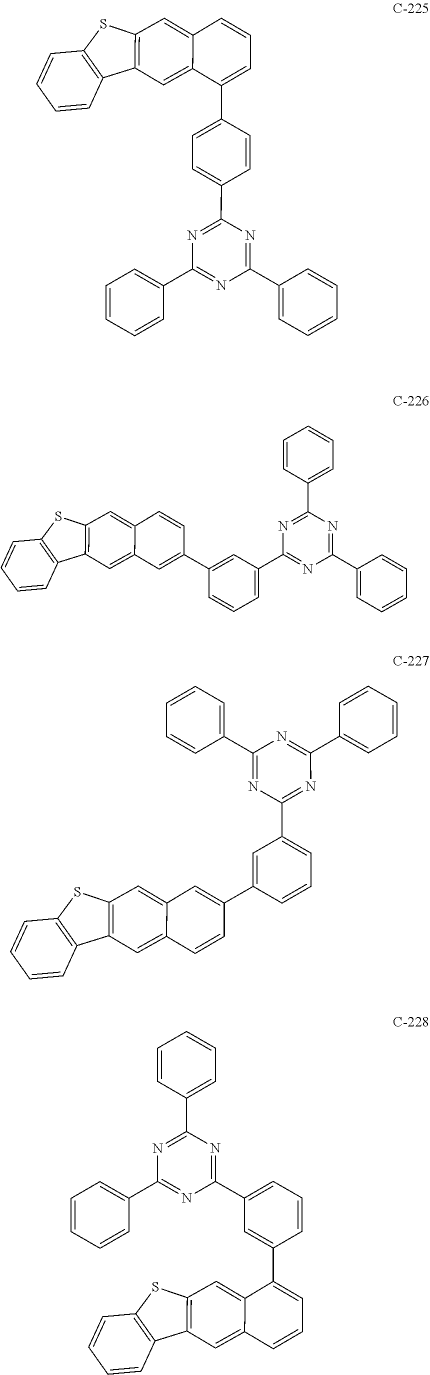 [SMILES: c1ccc(-c2nc(-c3ccccc3)nc(-c3ccc(-c4cccc5cc6sc7ccccc7c6cc45)cc3)n2)cc1 ; c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1 ; c1ccc(-c2nc(-c3ccccc3)nc(-c3cccc(-c4cccc5cc6c(cc45)sc4ccccc46)c3)n2)cc1]

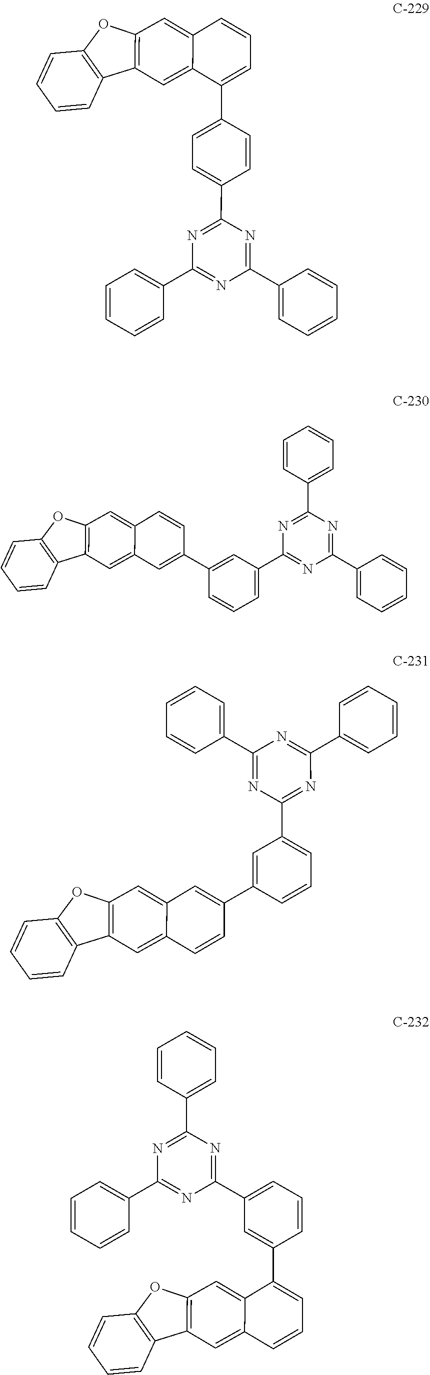 [SMILES: c1ccc(-c2nc(-c3ccccc3)nc(-c3ccc(-c4cccc5cc6oc7ccccc7c6cc45)cc3)n2)cc1 ; c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1]

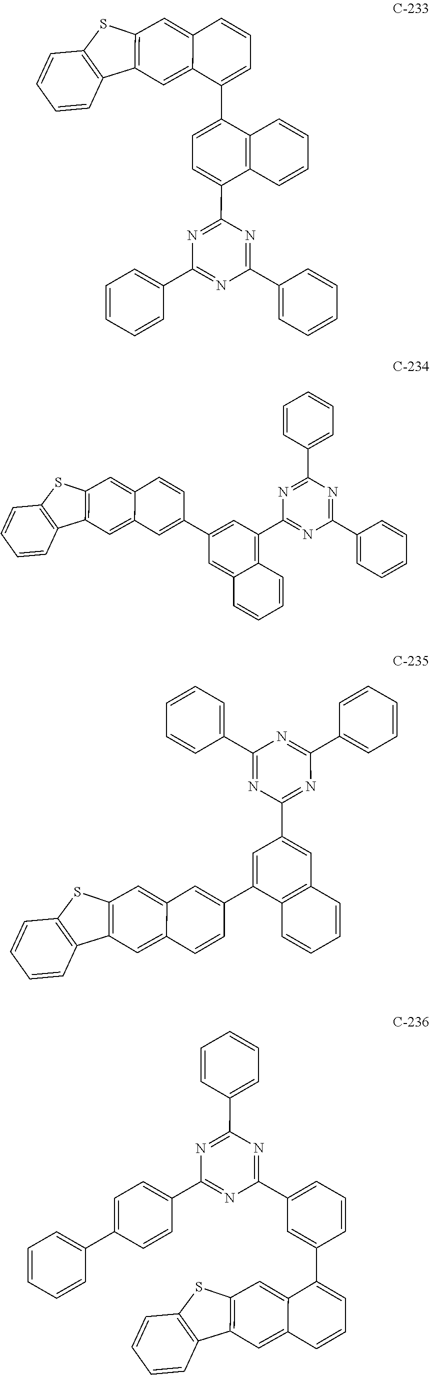 [SMILES: c1ccc(-c2nc(-c3ccccc3)nc(-c3ccc(-c4cccc5cc6sc7ccccc7c6cc45)c4ccccc34)n2)cc1 ; c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1]

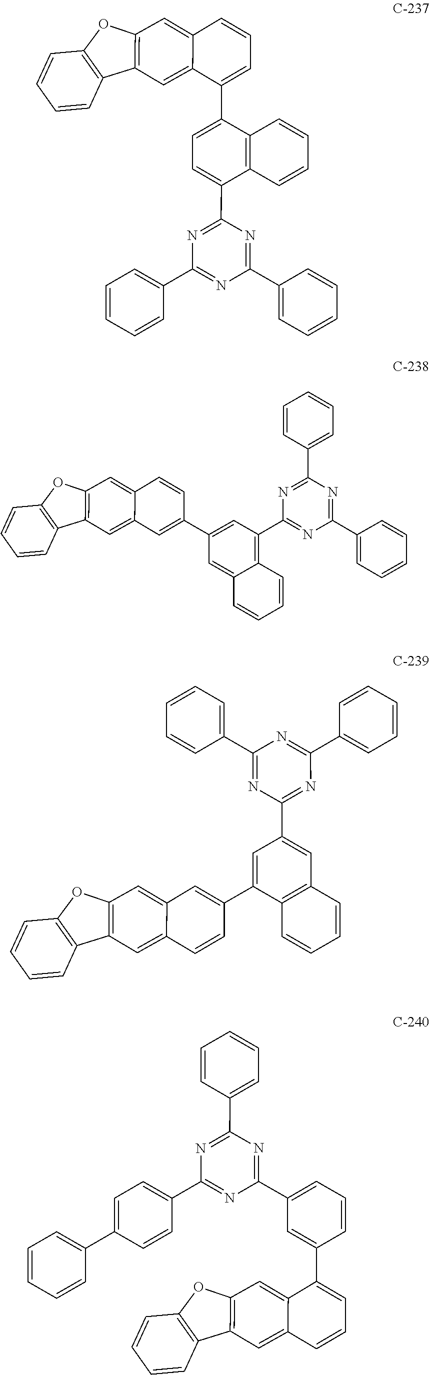 [SMILES: c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1]

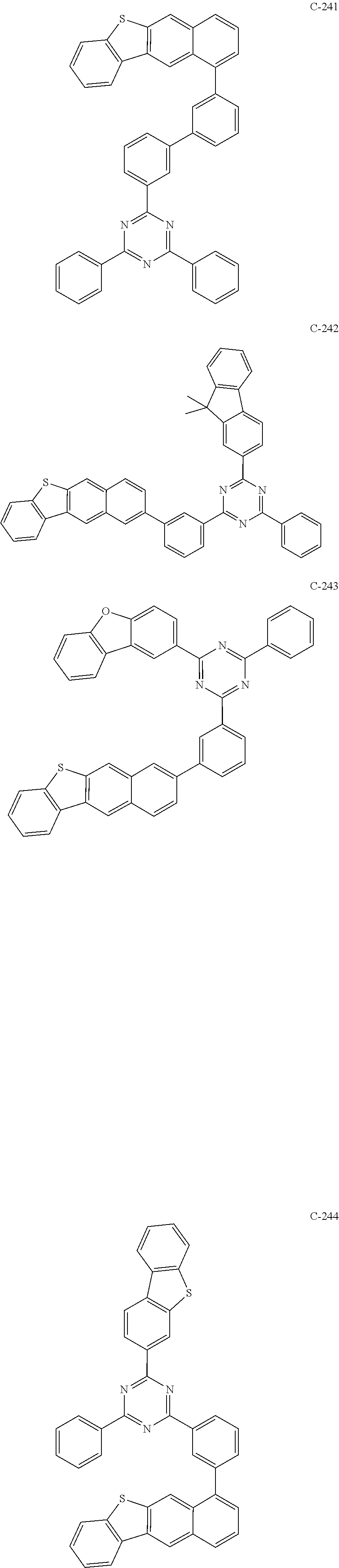 [SMILES: c1ccc(-c2nc(-c3ccccc3)nc(-c3cccc(-c4cccc(-c5cccc6cc7sc8ccccc8c7cc56)c4)c3)n2)cc1 ; CC1(C)c2ccccc2-c2ccc(-c3nc(-c4ccccc4)nc(-c4cccc(-c5ccc6cc7sc8ccccc8c7cc6c5)c4)n3)cc21 ; c1ccc(-c2nc(-c3cccc(-c4ccc5cc6c(cc5c4)sc4ccccc46)c3)nc(-c3ccc4oc5ccccc5c4c3)n2)cc1 ; c1ccc(-c2nc(-c3cccc(-c4cccc5cc6c(cc45)sc4ccccc46)c3)nc(-c3ccc4c(c3)sc3ccccc34)n2)cc1]

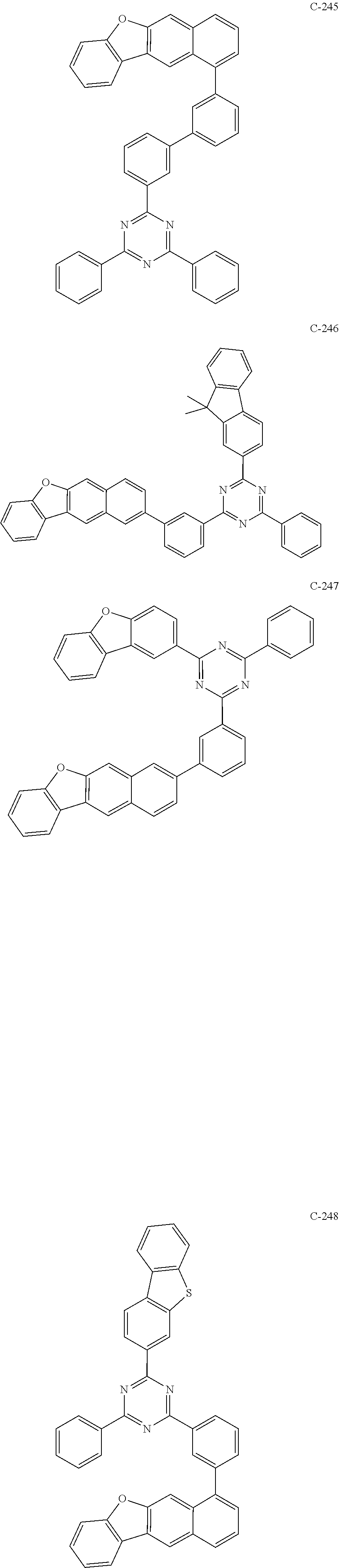 [SMILES: c1ccc(-c2nc(-c3ccccc3)nc(-c3cccc(-c4cccc(-c5cccc6cc7oc8ccccc8c7cc56)c4)c3)n2)cc1 ; CC1(C)c2ccccc2-c2ccc(-c3nc(-c4ccccc4)nc(-c4cccc(-c5ccc6cc7oc8ccccc8c7cc6c5)c4)n3)cc21 ; c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1]

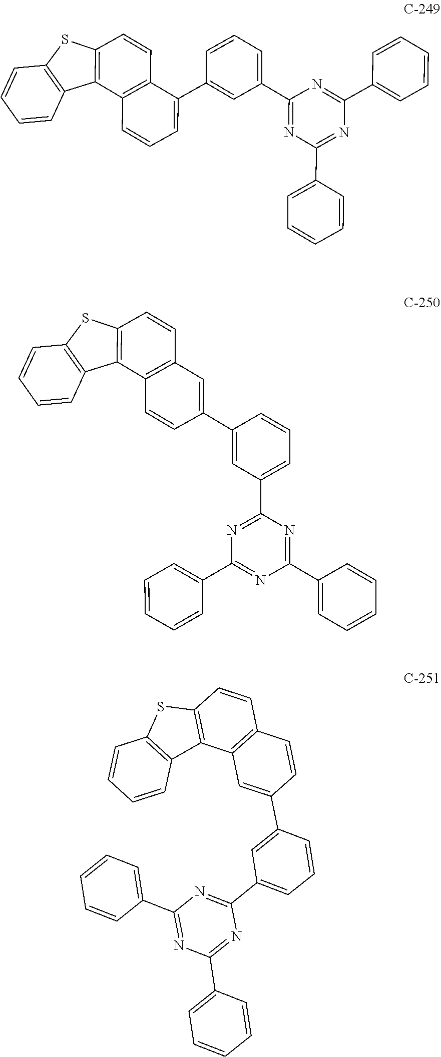 [SMILES: c1ccc(-c2nc(-c3ccccc3)nc(-c3cccc(-c4cccc5c4ccc4sc6ccccc6c45)c3)n2)cc1 ; c1ccc(-c2nc(-c3ccccc3)nc(-c3cccc(-c4ccc5c(ccc6sc7ccccc7c65)c4)c3)n2)cc1 ; c1ccc(-c2nc(-c3ccccc3)nc(-c3cccc(-c4ccc5ccc6sc7ccccc7c6c5c4)c3)n2)cc1]

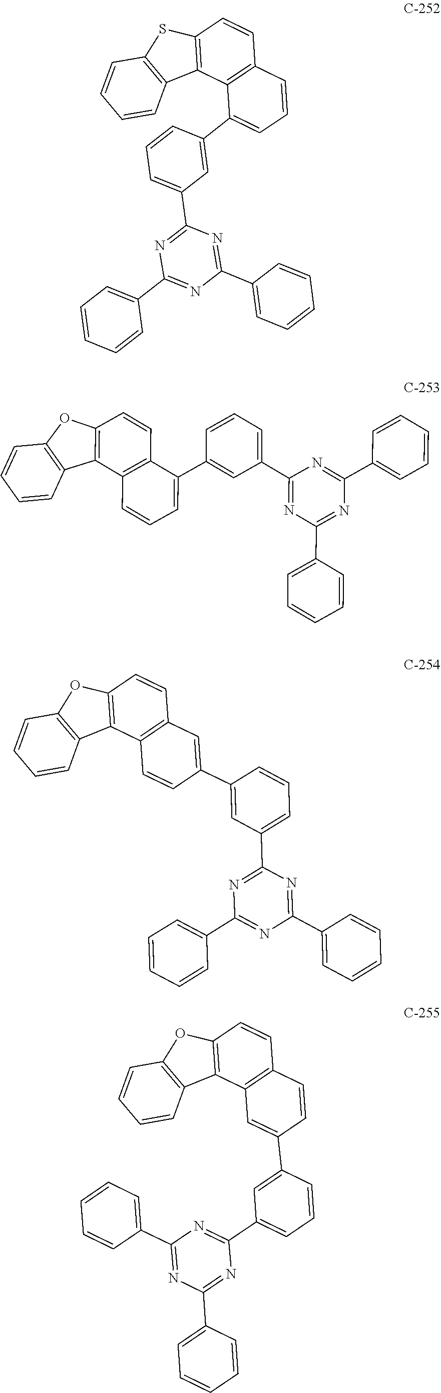 [SMILES: c1ccc(-c2nc(-c3ccccc3)nc(-c3cccc(-c4cccc5ccc6sc7ccccc7c6c45)c3)n2)cc1 ; c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1 ; c1ccc(-c2nc(-c3ccccc3)nc(-c3cccc(-c4ccc5ccc6oc7ccccc7c6c5c4)c3)n2)cc1]

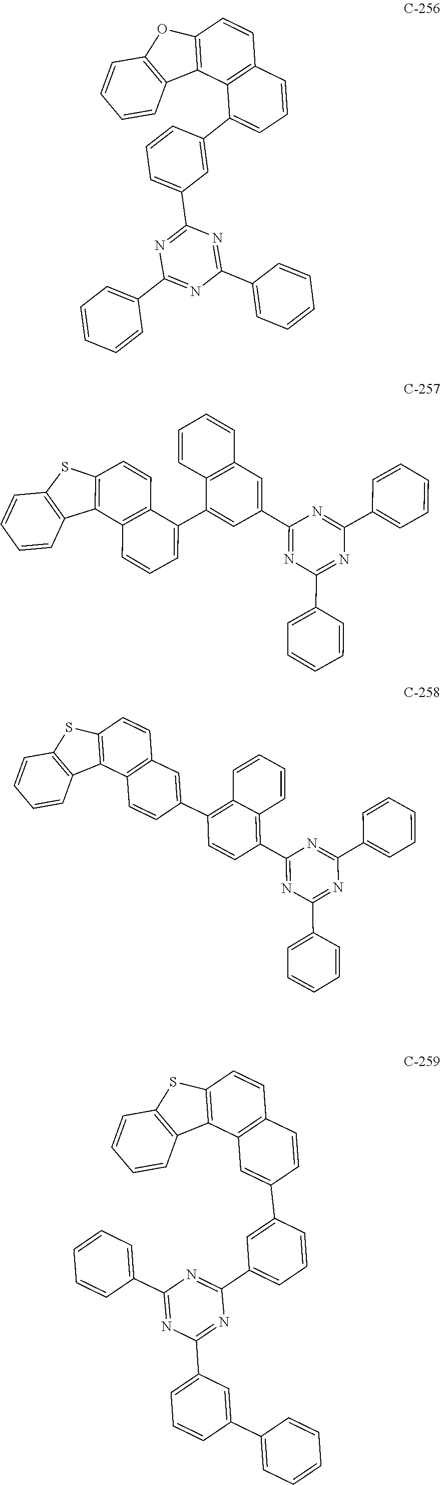 [SMILES: c1ccc(-c2nc(-c3ccccc3)nc(-c3cccc(-c4cccc5ccc6oc7ccccc7c6c45)c3)n2)cc1 ; c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1]

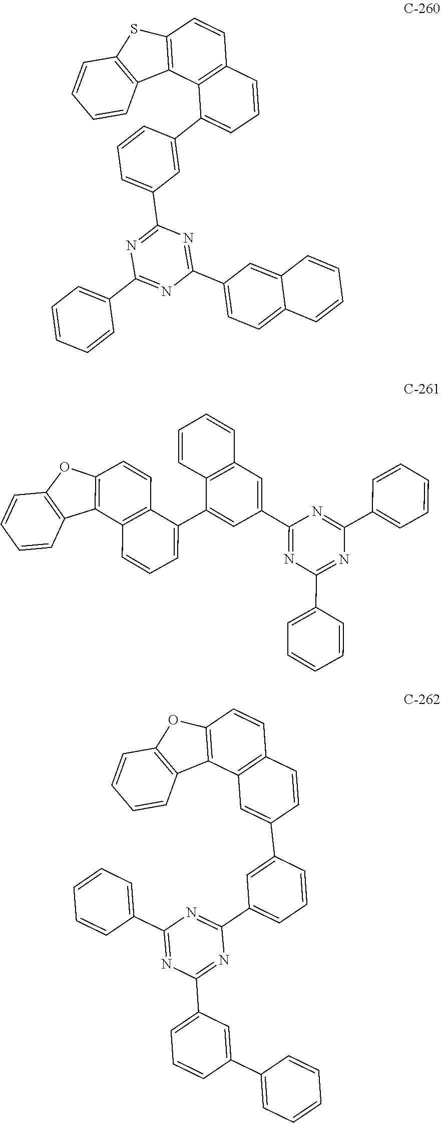 [SMILES: c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1 ; c1ccc(-c2cccc(-c3nc(-c4ccccc4)nc(-c4cccc(-c5ccc6ccc7oc8ccccc8c7c6c5)c4)n3)c2)cc1]

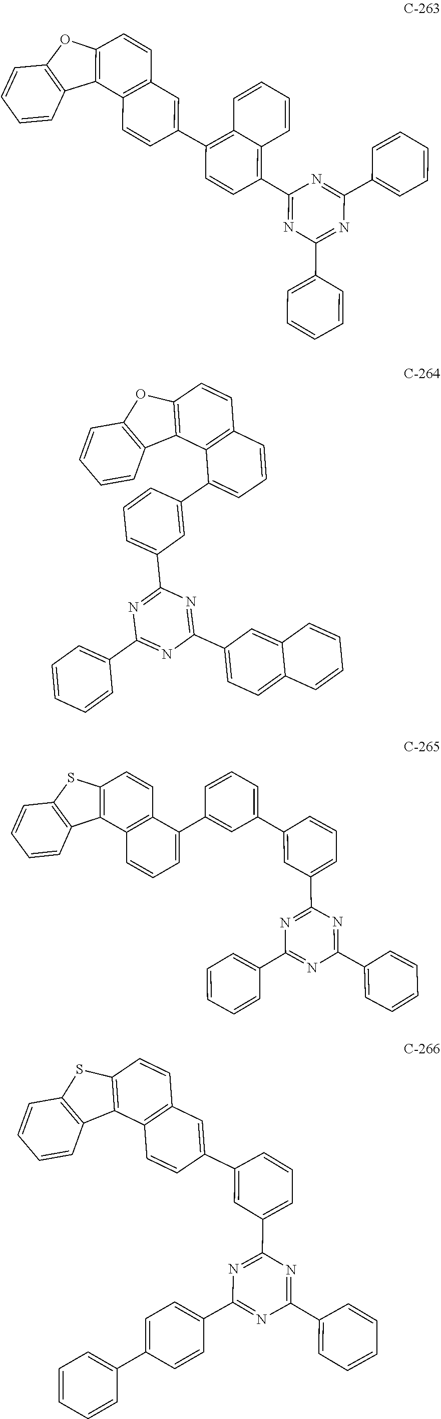 [SMILES: c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1 ; c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1]

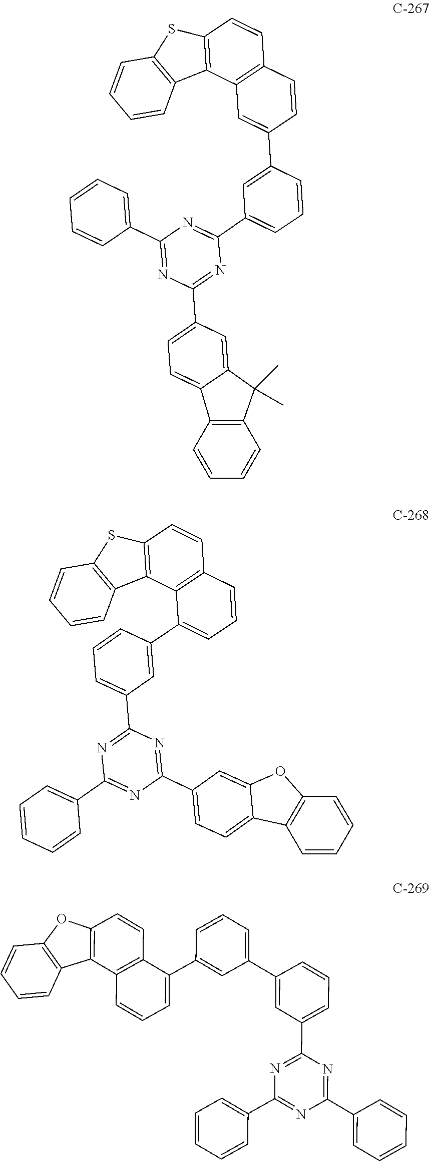 [SMILES: CC1(C)c2ccccc2-c2ccc(-c3nc(-c4ccccc4)nc(-c4cccc(-c5ccc6ccc7sc8ccccc8c7c6c5)c4)n3)cc21 ; c1ccc(-c2nc(-c3cccc(-c4cccc5ccc6sc7ccccc7c6c45)c3)nc(-c3ccc4c(c3)oc3ccccc34)n2)cc1 ; c1ccc(-c2nc(-c3ccccc3)nc(-c3cccc(-c4cccc(-c5cccc6c5ccc5oc7ccccc7c56)c4)c3)n2)cc1]

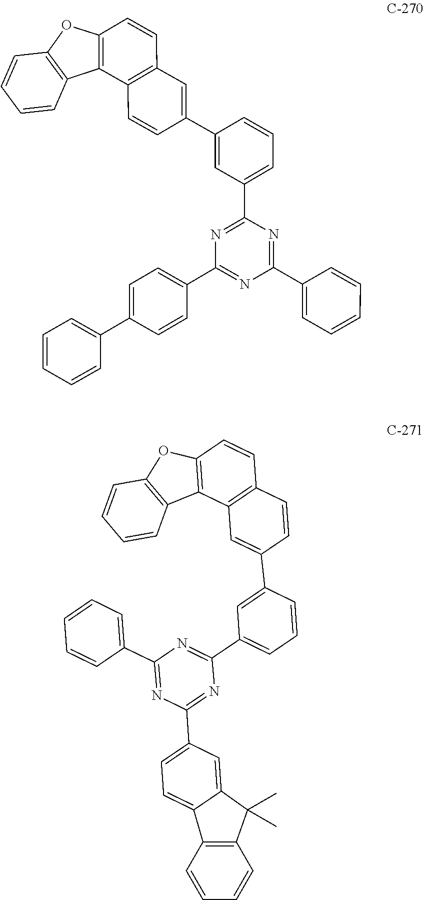 [SMILES: c1ccc(-c2ccc(-c3nc(-c4ccccc4)nc(-c4cccc(-c5ccc6c(ccc7oc8ccccc8c76)c5)c4)n3)cc2)cc1 ; CC1(C)c2ccccc2-c2ccc(-c3nc(-c4ccccc4)nc(-c4cccc(-c5ccc6ccc7oc8ccccc8c7c6c5)c4)n3)cc21]

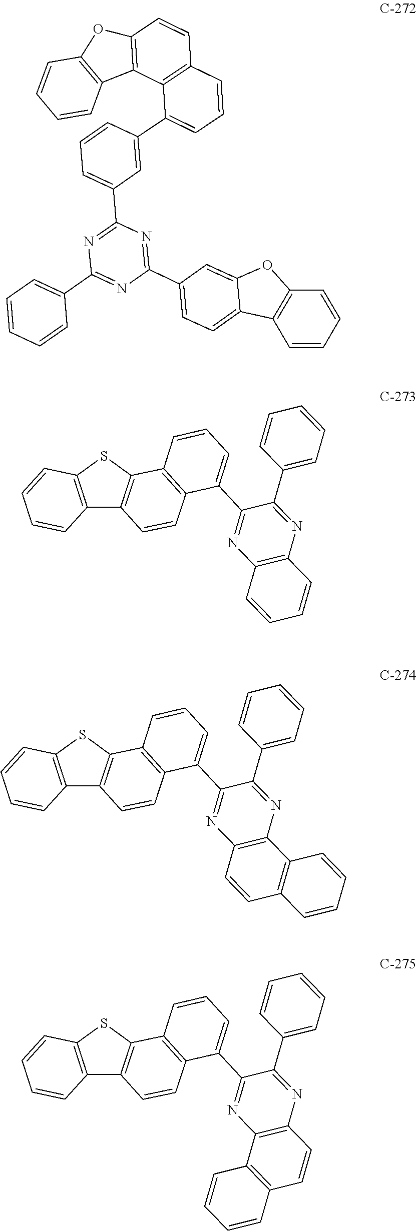 [SMILES: c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc3ccccc3nc2-c2cccc3c2ccc2c4ccccc4sc32)cc1 ; c1ccc(-c2nc3c(ccc4ccccc43)nc2-c2cccc3c2ccc2c4ccccc4sc32)cc1 ; c1ccc(-c2nc3ccc4ccccc4c3nc2-c2cccc3c2ccc2c4ccccc4sc32)cc1]

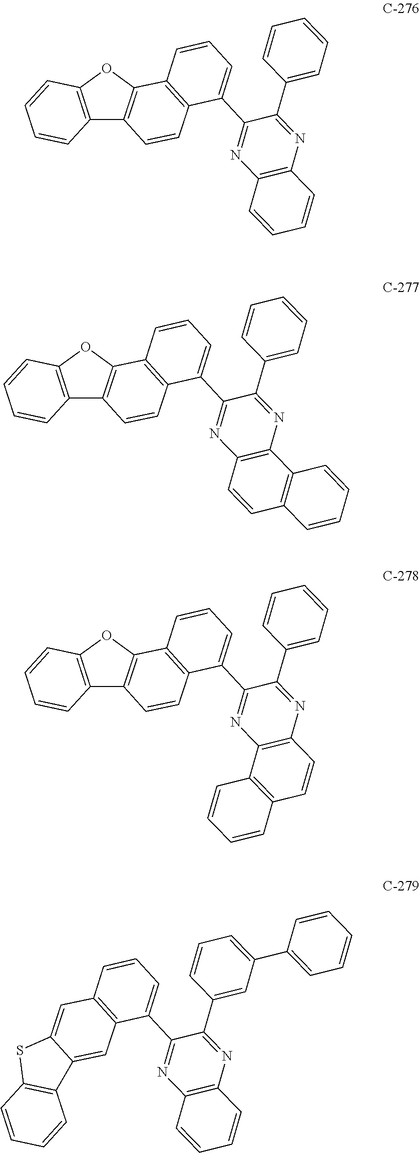 [SMILES: c1ccc(-c2nc3ccccc3nc2-c2cccc3c2ccc2c4ccccc4oc32)cc1 ; c1ccc(-c2nc3c(ccc4ccccc43)nc2-c2cccc3c2ccc2c4ccccc4oc32)cc1 ; c1ccc(-c2nc3ccc4ccccc4c3nc2-c2cccc3c2ccc2c4ccccc4oc32)cc1 ; c1ccc(-c2cccc(-c3nc4ccccc4nc3-c3cccc4cc5sc6ccccc6c5cc34)c2)cc1]

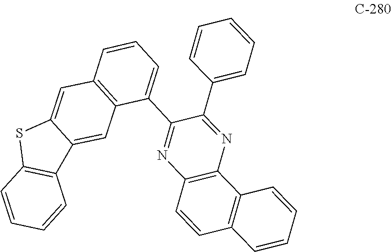 [SMILES: c1ccc(-c2nc3c(ccc4ccccc43)nc2-c2cccc3cc4sc5ccccc5c4cc23)cc1]

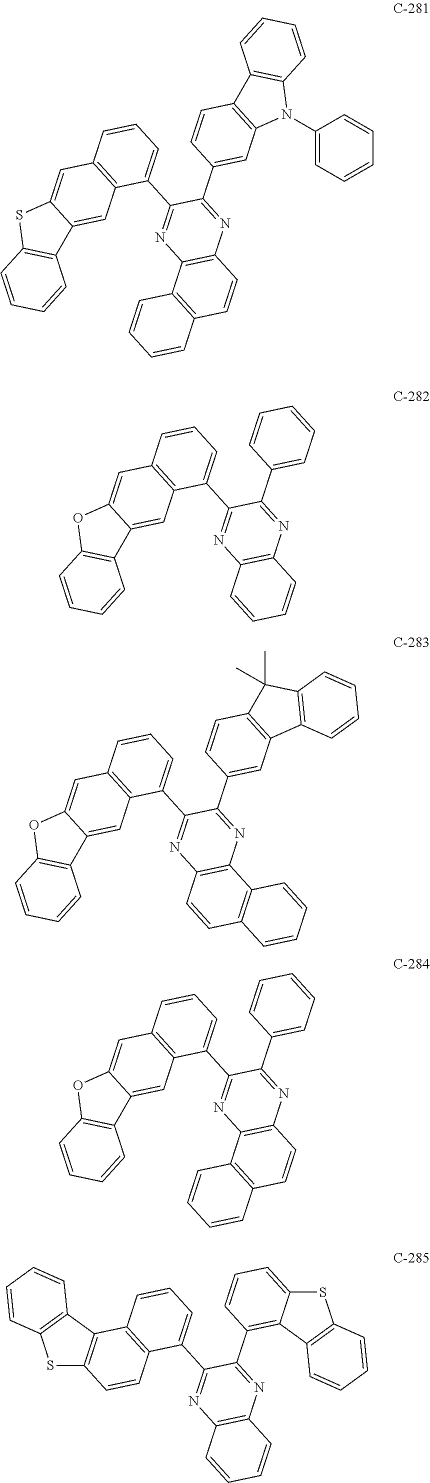

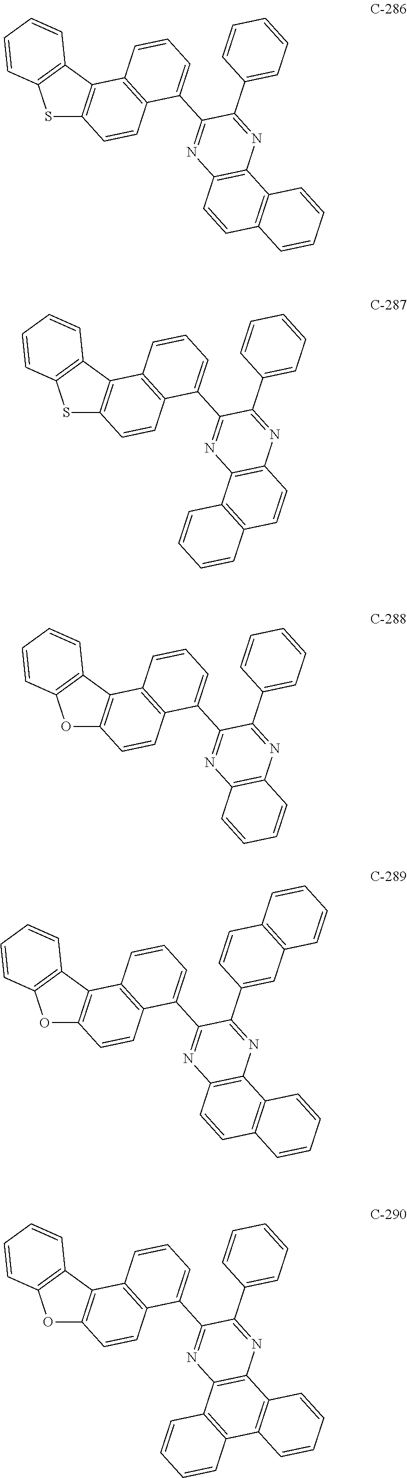 [SMILES: c1ccc(-c2nc3c(ccc4ccccc43)nc2-c2cccc3c2ccc2sc4ccccc4c23)cc1 ; c1ccc(-c2nc3ccc4ccccc4c3nc2-c2cccc3c2ccc2sc4ccccc4c23)cc1 ; c1ccc(-c2nc3ccccc3nc2-c2cccc3c2ccc2oc4ccccc4c23)cc1 ; c1ccc2cc(-c3nc4c(ccc5ccccc54)nc3-c3cccc4c3ccc3oc5ccccc5c34)ccc2c1 ; c1ccc(-c2nc3c4ccccc4c4ccccc4c3nc2-c2cccc3c2ccc2oc4ccccc4c23)cc1]

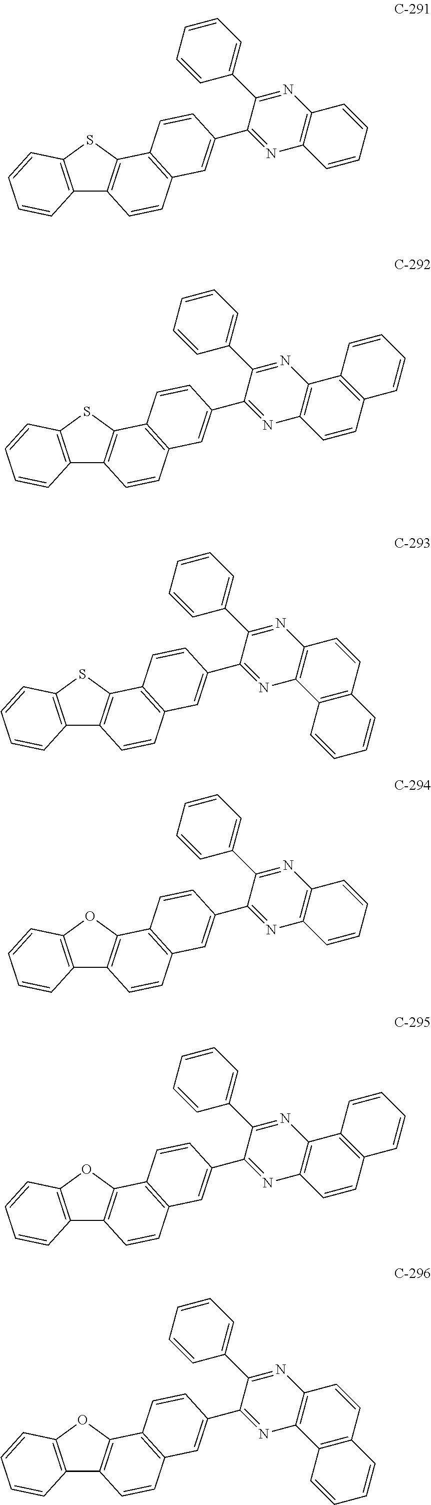 [SMILES: c1ccc(-c2nc3ccccc3nc2-c2ccc3c(ccc4c5ccccc5sc34)c2)cc1 ; c1ccc(-c2nc3c(ccc4ccccc43)nc2-c2ccc3c(ccc4c5ccccc5sc34)c2)cc1 ; c1ccc(-c2nc3ccc4ccccc4c3nc2-c2ccc3c(ccc4c5ccccc5sc34)c2)cc1 ; c1ccc(-c2nc3ccccc3nc2-c2ccc3c(ccc4c5ccccc5oc34)c2)cc1 ; c1ccc(-c2nc3c(ccc4ccccc43)nc2-c2ccc3c(ccc4c5ccccc5oc34)c2)cc1 ; c1ccc(-c2nc3ccc4ccccc4c3nc2-c2ccc3c(ccc4c5ccccc5oc34)c2)cc1]

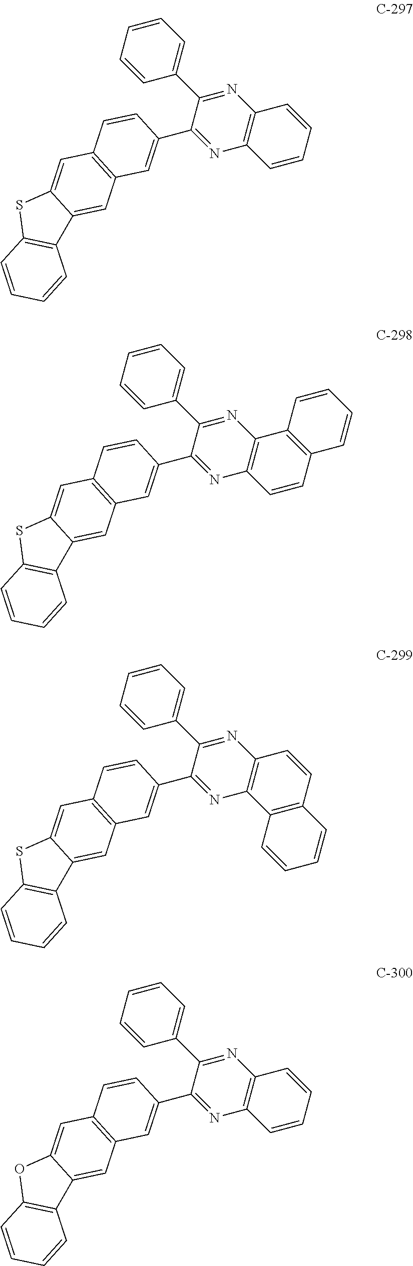 [SMILES: c1ccc(-c2nc3ccccc3nc2-c2ccc3cc4sc5ccccc5c4cc3c2)cc1 ; c1ccc(-c2nc3c(ccc4ccccc43)nc2-c2ccc3cc4sc5ccccc5c4cc3c2)cc1 ; c1ccc(-c2nc3ccc4ccccc4c3nc2-c2ccc3cc4sc5ccccc5c4cc3c2)cc1 ; c1ccc(-c2nc3ccccc3nc2-c2ccc3cc4oc5ccccc5c4cc3c2)cc1]

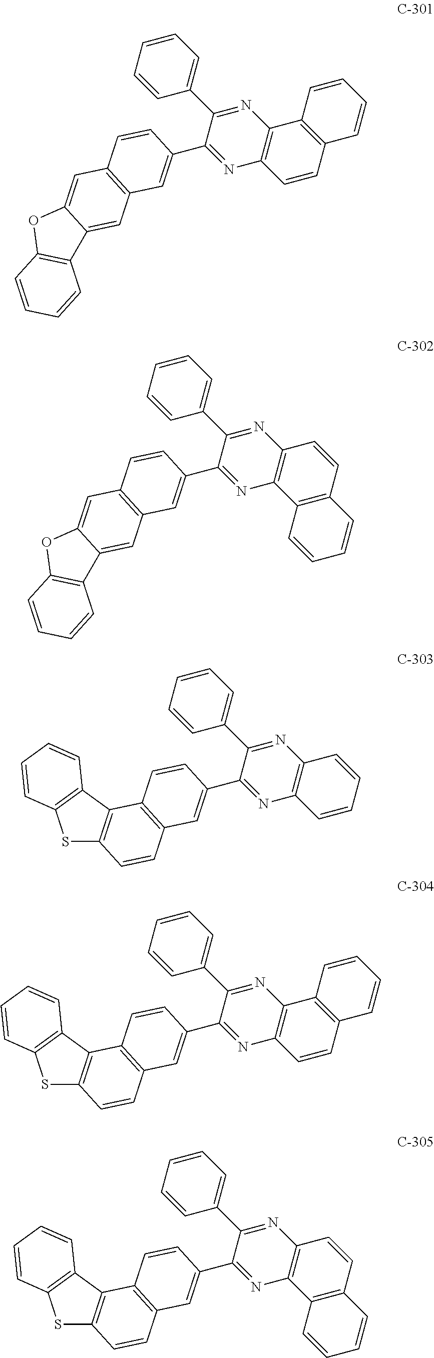 [SMILES: c1ccc(-c2nc3c(ccc4ccccc43)nc2-c2ccc3cc4oc5ccccc5c4cc3c2)cc1 ; c1ccc(-c2nc3ccc4ccccc4c3nc2-c2ccc3cc4oc5ccccc5c4cc3c2)cc1 ; c1ccc(-c2nc3ccccc3nc2-c2ccc3c(ccc4sc5ccccc5c43)c2)cc1 ; c1ccc(-c2nc3c(ccc4ccccc43)nc2-c2ccc3c(ccc4sc5ccccc5c43)c2)cc1 ; c1ccc(-c2nc3ccc4ccccc4c3nc2-c2ccc3c(ccc4sc5ccccc5c43)c2)cc1]

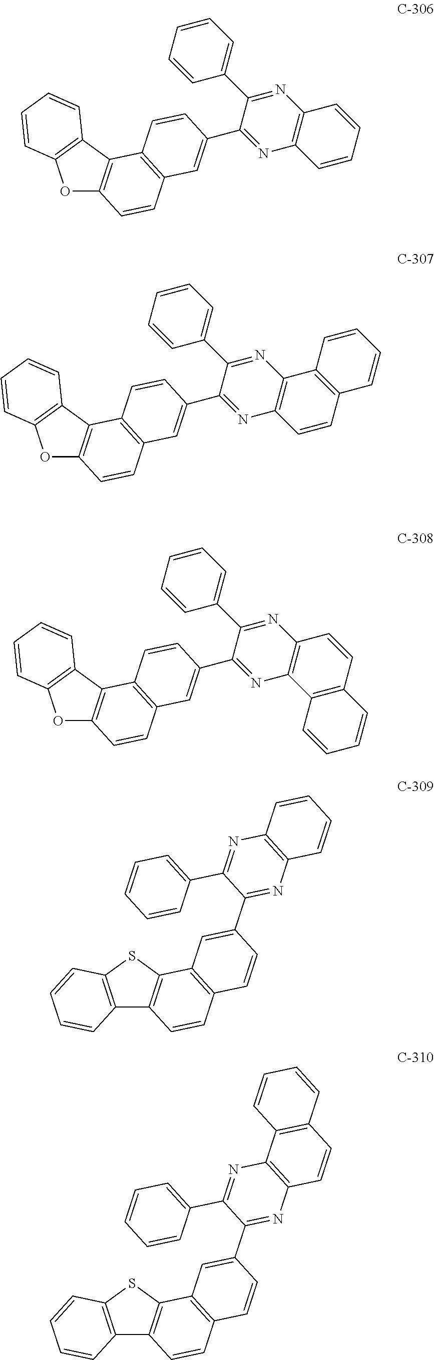

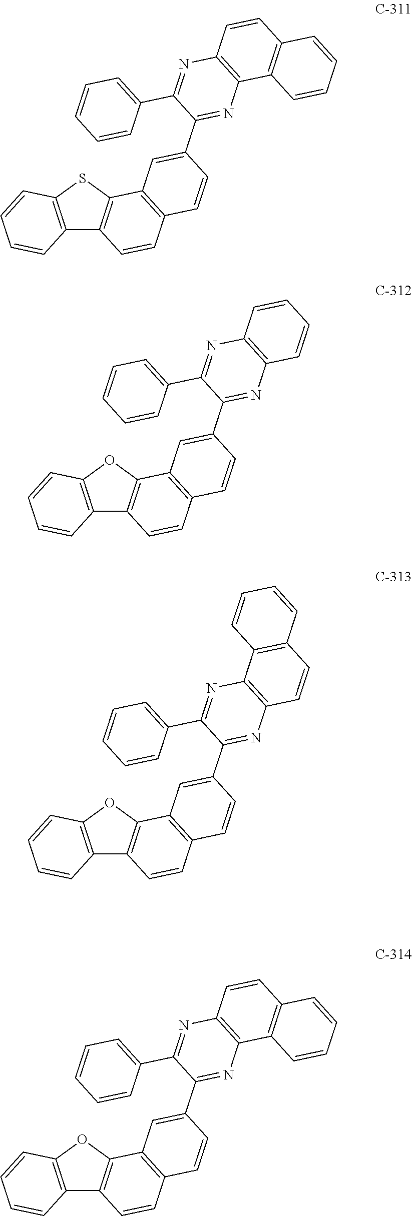 [SMILES: c1ccc(-c2nc3ccc4ccccc4c3nc2-c2ccc3ccc4c5ccccc5sc4c3c2)cc1 ; c1ccc(-c2nc3ccccc3nc2-c2ccc3ccc4c5ccccc5oc4c3c2)cc1 ; c1ccc(-c2nc3c(ccc4ccccc43)nc2-c2ccc3ccc4c5ccccc5oc4c3c2)cc1 ; c1ccc(-c2nc3ccc4ccccc4c3nc2-c2ccc3ccc4c5ccccc5oc4c3c2)cc1]

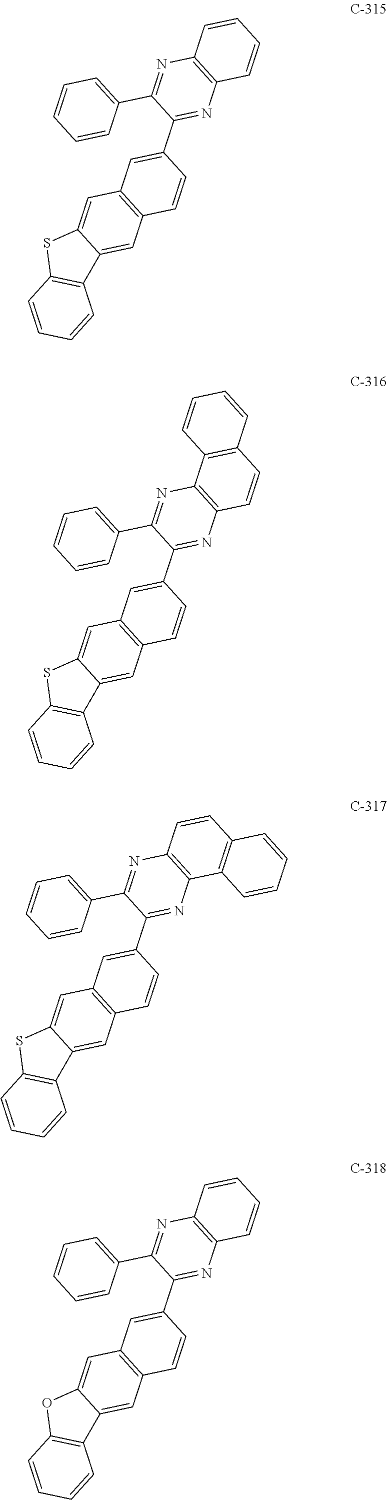 [SMILES: c1ccc(-c2nc3ccccc3nc2-c2ccc3cc4c(cc3c2)sc2ccccc24)cc1 ; c1ccc(-c2nc3c(ccc4ccccc43)nc2-c2ccc3cc4c(cc3c2)sc2ccccc24)cc1 ; c1ccc(-c2nc3ccc4ccccc4c3nc2-c2ccc3cc4c(cc3c2)sc2ccccc24)cc1 ; c1ccc(-c2nc3ccccc3nc2-c2ccc3cc4c(cc3c2)oc2ccccc24)cc1]

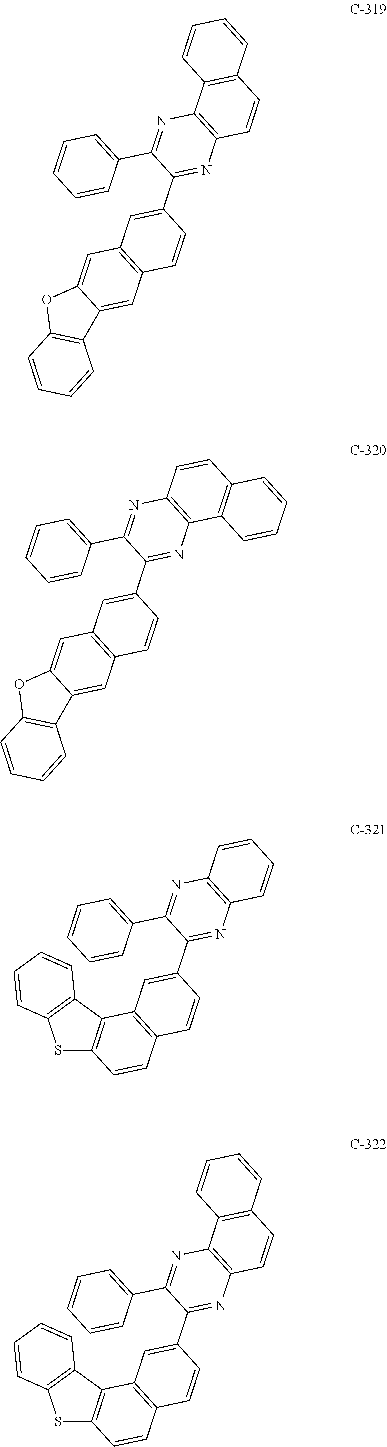 [SMILES: c1ccc(-c2nc3c(ccc4ccccc43)nc2-c2ccc3cc4c(cc3c2)oc2ccccc24)cc1 ; c1ccc(-c2nc3ccc4ccccc4c3nc2-c2ccc3cc4c(cc3c2)oc2ccccc24)cc1 ; c1ccc(-c2nc3ccccc3nc2-c2ccc3ccc4sc5ccccc5c4c3c2)cc1 ; c1ccc(-c2nc3c(ccc4ccccc43)nc2-c2ccc3ccc4sc5ccccc5c4c3c2)cc1]

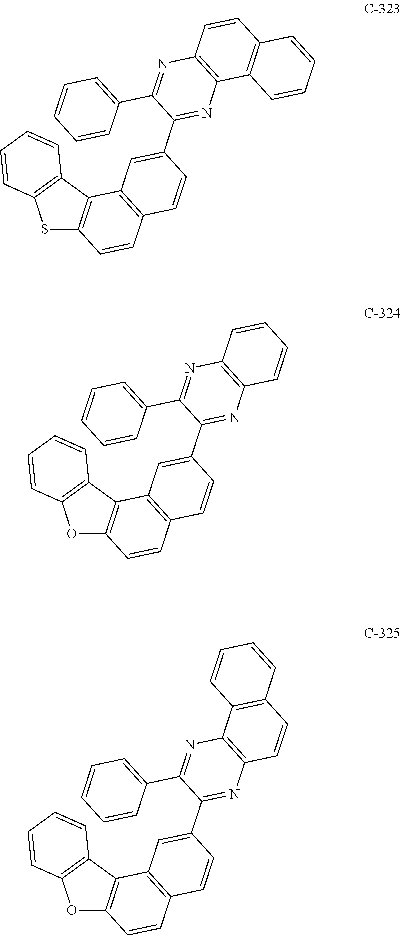 [SMILES: c1ccc(-c2nc3ccc4ccccc4c3nc2-c2ccc3ccc4sc5ccccc5c4c3c2)cc1 ; c1ccc(-c2nc3ccccc3nc2-c2ccc3ccc4oc5ccccc5c4c3c2)cc1 ; c1ccc(-c2nc3c(ccc4ccccc43)nc2-c2ccc3ccc4oc5ccccc5c4c3c2)cc1]

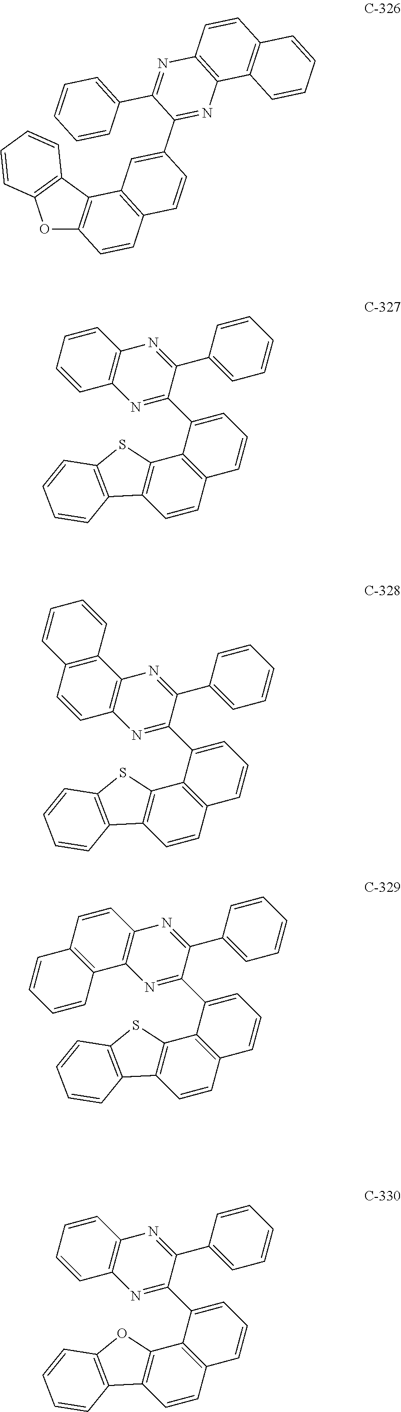 [SMILES: c1ccc(-c2nc3ccc4ccccc4c3nc2-c2ccc3ccc4oc5ccccc5c4c3c2)cc1 ; c1ccc(-c2nc3ccccc3nc2-c2cccc3ccc4c5ccccc5sc4c23)cc1 ; c1ccc(-c2nc3c(ccc4ccccc43)nc2-c2cccc3ccc4c5ccccc5sc4c23)cc1 ; c1ccc(-c2nc3ccc4ccccc4c3nc2-c2cccc3ccc4c5ccccc5sc4c23)cc1 ; c1ccc(-c2nc3ccccc3nc2-c2cccc3ccc4c5ccccc5oc4c23)cc1]

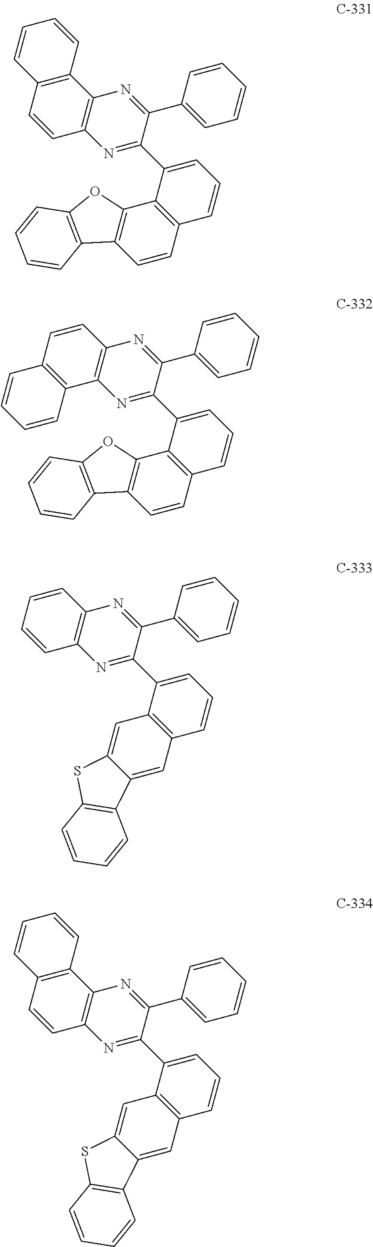 [SMILES: c1ccc(-c2nc3c(ccc4ccccc43)nc2-c2cccc3ccc4c5ccccc5oc4c23)cc1 ; c1ccc(-c2nc3ccc4ccccc4c3nc2-c2cccc3ccc4c5ccccc5oc4c23)cc1 ; c1ccc(-c2nc3ccccc3nc2-c2cccc3cc4c(cc23)sc2ccccc24)cc1 ; c1ccc(-c2nc3c(ccc4ccccc43)nc2-c2cccc3cc4c(cc23)sc2ccccc24)cc1]

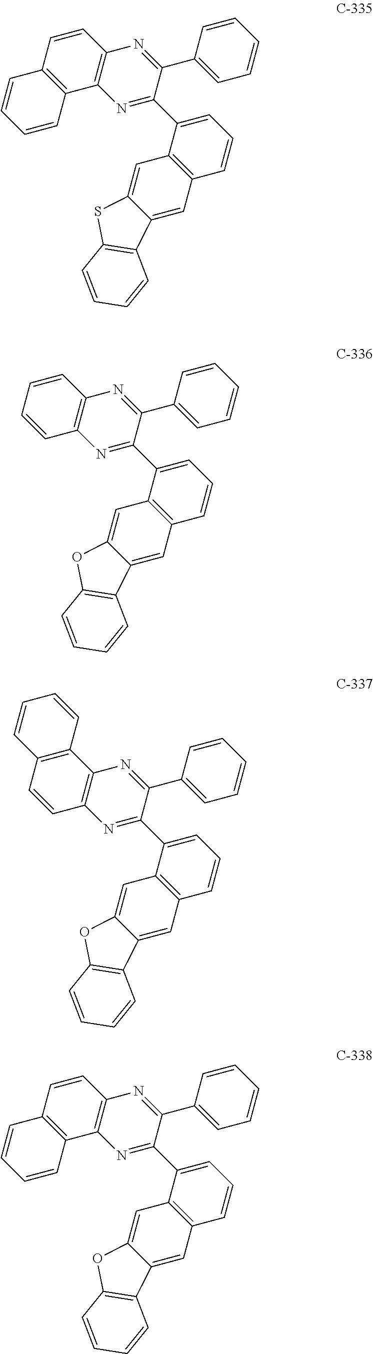 [SMILES: c1ccc(-c2nc3ccc4ccccc4c3nc2-c2cccc3cc4c(cc23)sc2ccccc24)cc1 ; c1ccc(-c2nc3ccccc3nc2-c2cccc3cc4c(cc23)oc2ccccc24)cc1 ; c1ccc(-c2nc3c(ccc4ccccc43)nc2-c2cccc3cc4c(cc23)oc2ccccc24)cc1 ; c1ccc(-c2nc3ccc4ccccc4c3nc2-c2cccc3cc4c(cc23)oc2ccccc24)cc1]

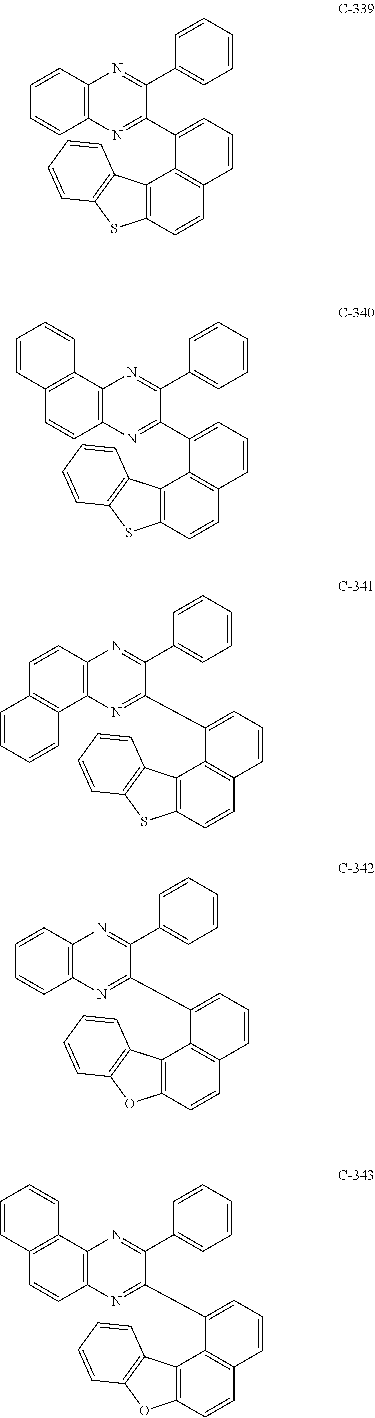 [SMILES: c1ccc(-c2nc3ccccc3nc2-c2cccc3ccc4sc5ccccc5c4c23)cc1 ; c1ccc(-c2nc3c(ccc4ccccc43)nc2-c2cccc3ccc4sc5ccccc5c4c23)cc1 ; c1ccc(-c2nc3ccc4ccccc4c3nc2-c2cccc3ccc4sc5ccccc5c4c23)cc1 ; c1ccc(-c2nc3ccccc3nc2-c2cccc3ccc4oc5ccccc5c4c23)cc1 ; c1ccc(-c2nc3c(ccc4ccccc43)nc2-c2cccc3ccc4oc5ccccc5c4c23)cc1]

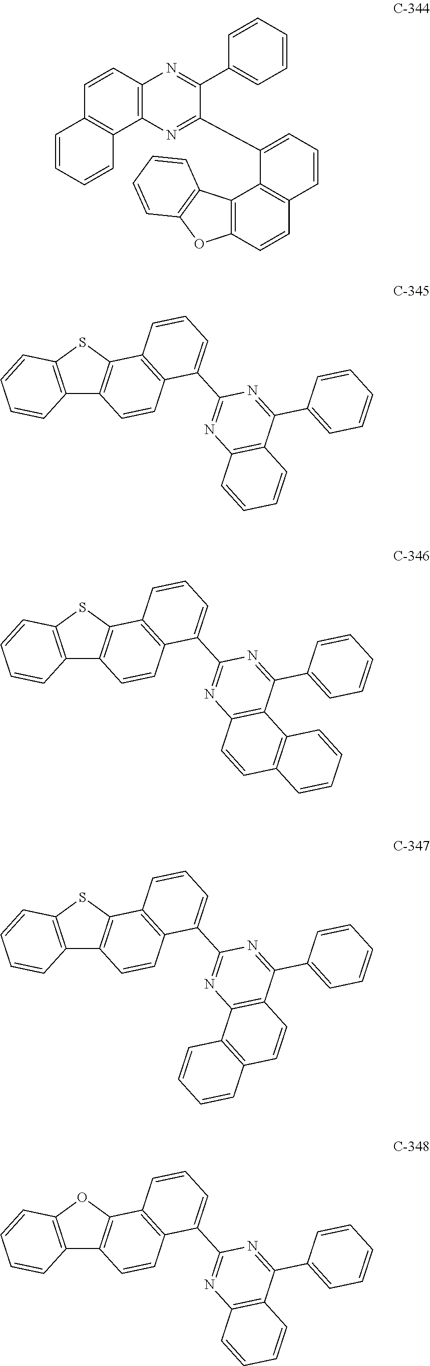 [SMILES: c1ccc(-c2nc3ccc4ccccc4c3nc2-c2cccc3ccc4oc5ccccc5c4c23)cc1 ; c1ccc(-c2nc(-c3cccc4c3ccc3c5ccccc5sc43)nc3ccccc23)cc1 ; c1ccc(-c2nc(-c3cccc4c3ccc3c5ccccc5sc43)nc3ccc4ccccc4c23)cc1 ; c1ccc(-c2nc(-c3cccc4c3ccc3c5ccccc5sc43)nc3c2ccc2ccccc23)cc1 ; c1ccc(-c2nc(-c3cccc4c3ccc3c5ccccc5oc43)nc3ccccc23)cc1]

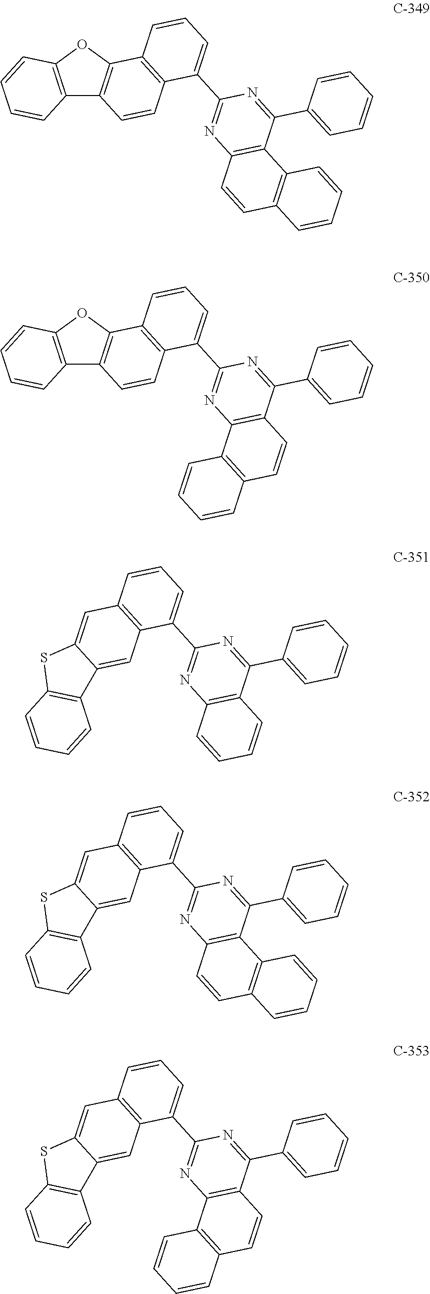 [SMILES: c1ccc(-c2nc(-c3cccc4c3ccc3c5ccccc5oc43)nc3ccc4ccccc4c23)cc1 ; c1ccc(-c2nc(-c3cccc4c3ccc3c5ccccc5oc43)nc3c2ccc2ccccc23)cc1 ; c1ccc(-c2nc(-c3cccc4cc5sc6ccccc6c5cc34)nc3ccccc23)cc1 ; c1ccc(-c2nc(-c3cccc4cc5sc6ccccc6c5cc34)nc3ccc4ccccc4c23)cc1 ; c1ccc(-c2nc(-c3cccc4cc5sc6ccccc6c5cc34)nc3c2ccc2ccccc23)cc1]

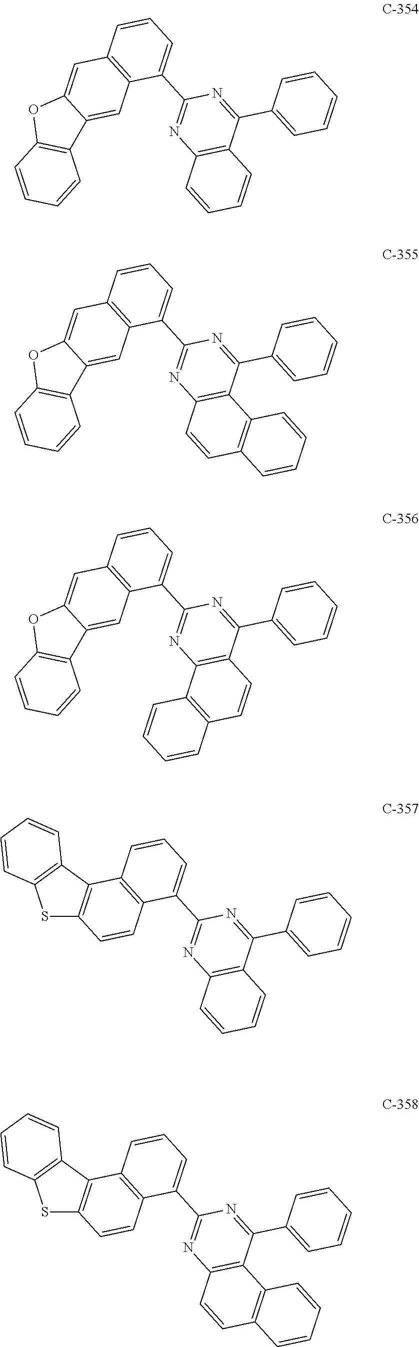 [SMILES: c1ccc(-c2nc(-c3cccc4cc5oc6ccccc6c5cc34)nc3ccccc23)cc1 ; c1ccc(-c2nc(-c3cccc4cc5oc6ccccc6c5cc34)nc3ccc4ccccc4c23)cc1 ; c1ccc(-c2nc(-c3cccc4cc5oc6ccccc6c5cc34)nc3c2ccc2ccccc23)cc1 ; c1ccc(-c2nc(-c3cccc4c3ccc3sc5ccccc5c34)nc3ccccc23)cc1 ; c1ccc(-c2nc(-c3cccc4c3ccc3sc5ccccc5c34)nc3ccc4ccccc4c23)cc1]

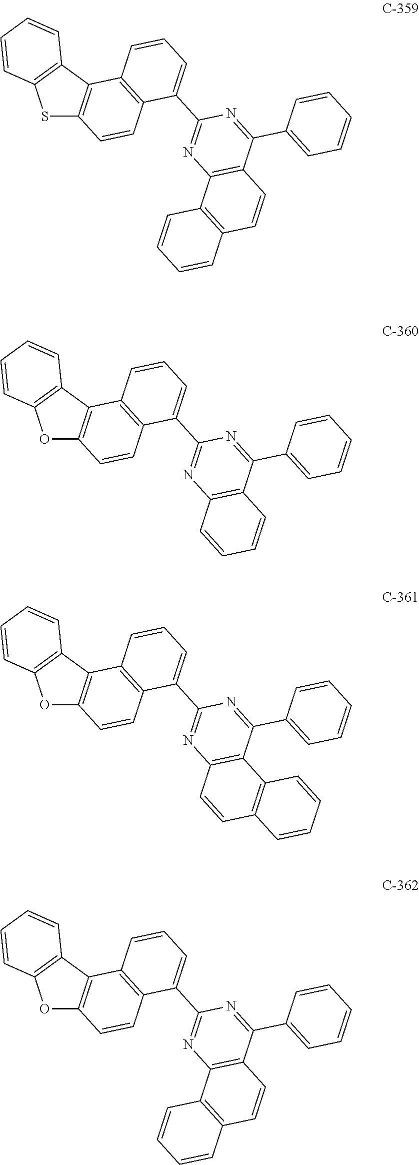 [SMILES: c1ccc(-c2nc(-c3cccc4c3ccc3sc5ccccc5c34)nc3c2ccc2ccccc23)cc1 ; c1ccc(-c2nc(-c3cccc4c3ccc3oc5ccccc5c34)nc3ccccc23)cc1 ; c1ccc(-c2nc(-c3cccc4c3ccc3oc5ccccc5c34)nc3ccc4ccccc4c23)cc1 ; c1ccc(-c2nc(-c3cccc4c3ccc3oc5ccccc5c34)nc3c2ccc2ccccc23)cc1]

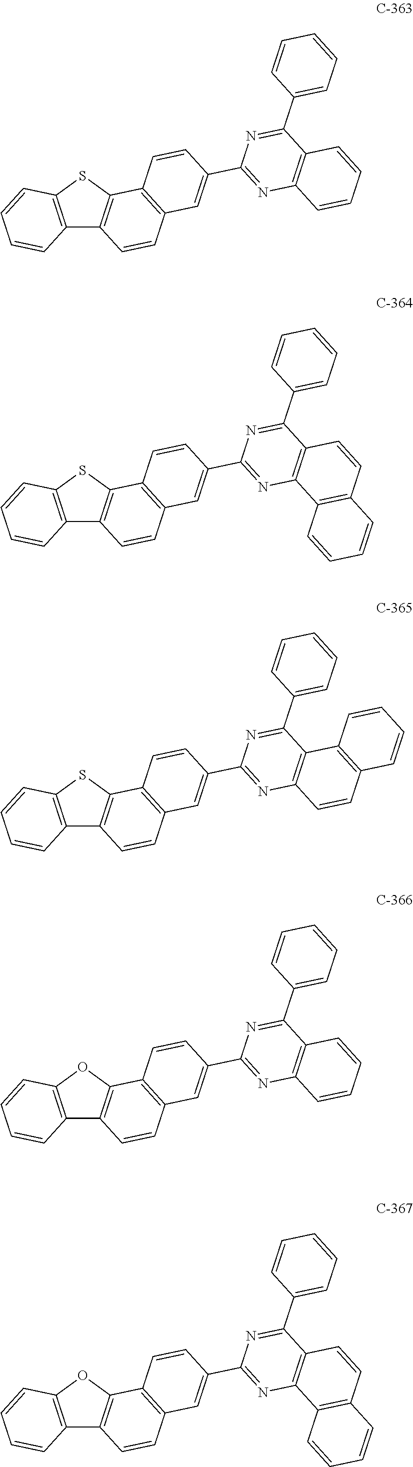 [SMILES: c1ccc(-c2nc(-c3ccc4c(ccc5c6ccccc6sc45)c3)nc3ccccc23)cc1 ; c1ccc(-c2nc(-c3ccc4c(ccc5c6ccccc6sc45)c3)nc3c2ccc2ccccc23)cc1 ; c1ccc(-c2nc(-c3ccc4c(ccc5c6ccccc6sc45)c3)nc3ccc4ccccc4c23)cc1 ; c1ccc(-c2nc(-c3ccc4c(ccc5c6ccccc6oc45)c3)nc3ccccc23)cc1 ; c1ccc(-c2nc(-c3ccc4c(ccc5c6ccccc6oc45)c3)nc3c2ccc2ccccc23)cc1]

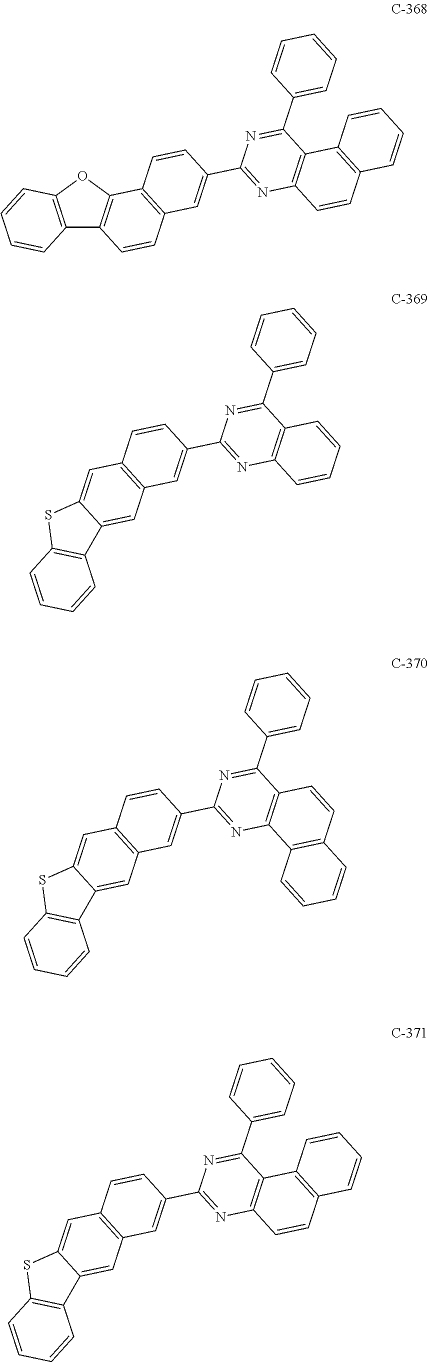 [SMILES: c1ccc(-c2nc(-c3ccc4c(ccc5c6ccccc6oc45)c3)nc3ccc4ccccc4c23)cc1 ; c1ccc(-c2nc(-c3ccc4cc5sc6ccccc6c5cc4c3)nc3ccccc23)cc1 ; c1ccc(-c2nc(-c3ccc4cc5sc6ccccc6c5cc4c3)nc3c2ccc2ccccc23)cc1 ; c1ccc(-c2nc(-c3ccc4cc5sc6ccccc6c5cc4c3)nc3ccc4ccccc4c23)cc1]

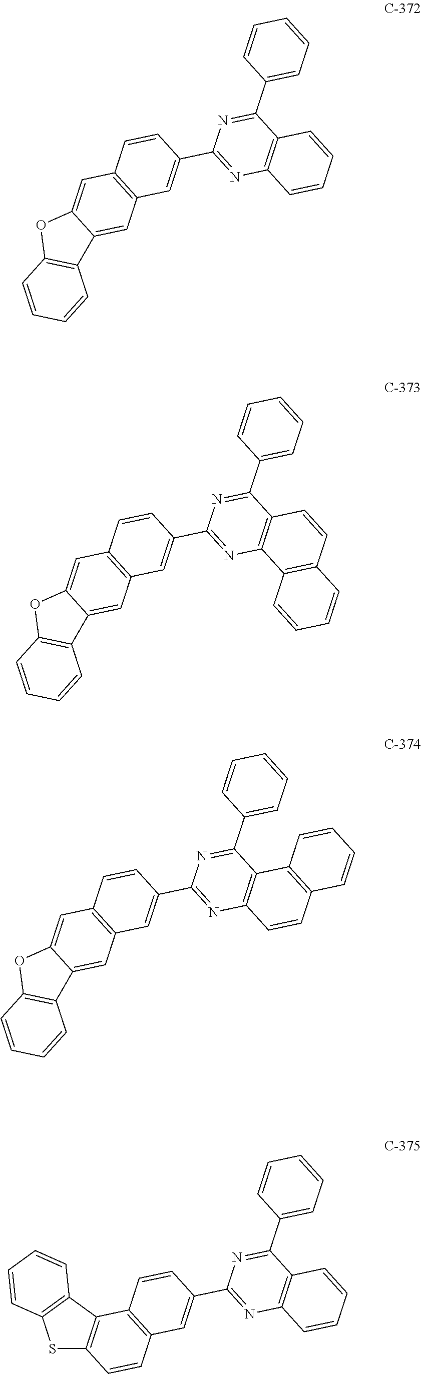 [SMILES: c1ccc(-c2nc(-c3ccc4cc5oc6ccccc6c5cc4c3)nc3ccccc23)cc1 ; c1ccc(-c2nc(-c3ccc4cc5oc6ccccc6c5cc4c3)nc3c2ccc2ccccc23)cc1 ; c1ccc(-c2nc(-c3ccc4cc5oc6ccccc6c5cc4c3)nc3ccc4ccccc4c23)cc1 ; c1ccc(-c2nc(-c3ccc4c(ccc5sc6ccccc6c54)c3)nc3ccccc23)cc1]

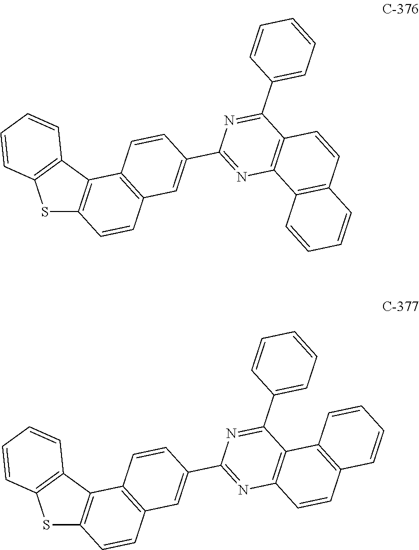 [SMILES: c1ccc(-c2nc(-c3ccc4c(ccc5sc6ccccc6c54)c3)nc3c2ccc2ccccc23)cc1 ; c1ccc(-c2nc(-c3ccc4c(ccc5sc6ccccc6c54)c3)nc3ccc4ccccc4c23)cc1]

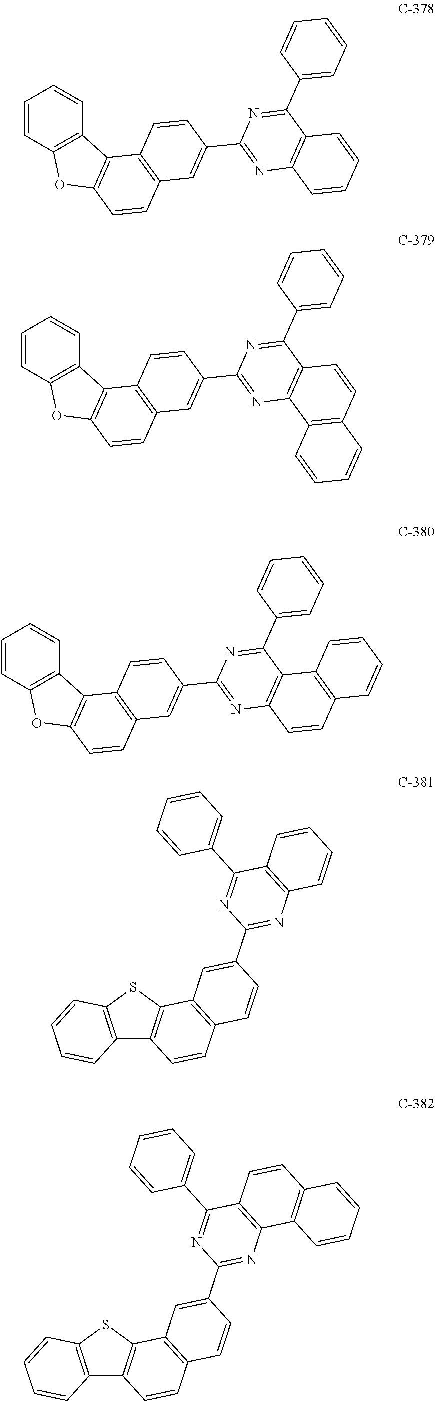 [SMILES: c1ccc(-c2nc(-c3ccc4c(ccc5oc6ccccc6c54)c3)nc3ccccc23)cc1 ; c1ccc(-c2nc(-c3ccc4c(ccc5oc6ccccc6c54)c3)nc3c2ccc2ccccc23)cc1 ; c1ccc(-c2nc(-c3ccc4c(ccc5oc6ccccc6c54)c3)nc3ccc4ccccc4c23)cc1 ; c1ccc(-c2nc(-c3ccc4ccc5c6ccccc6sc5c4c3)nc3ccccc23)cc1 ; c1ccc(-c2nc(-c3ccc4ccc5c6ccccc6sc5c4c3)nc3c2ccc2ccccc23)cc1]

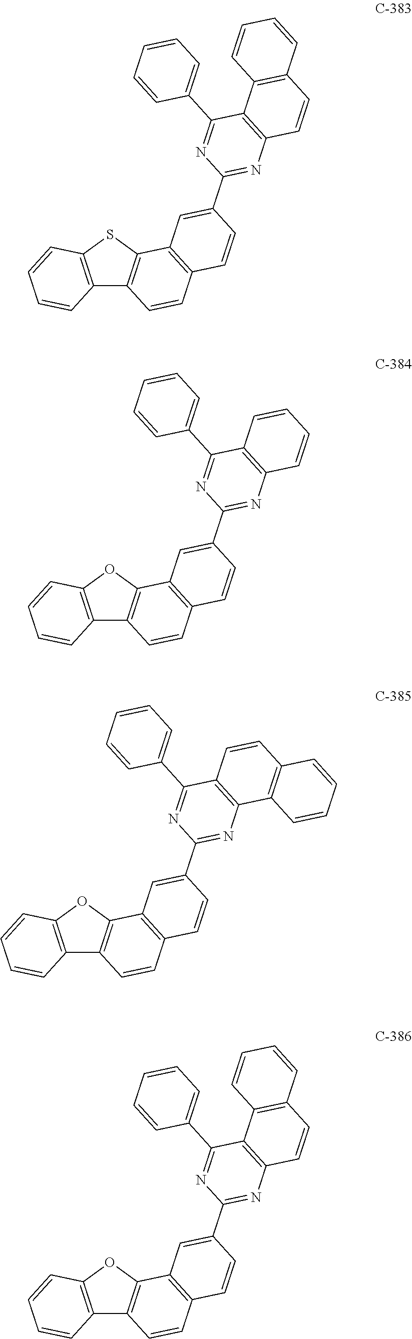 [SMILES: c1ccc(-c2nc(-c3ccc4ccc5c6ccccc6sc5c4c3)nc3ccc4ccccc4c23)cc1 ; c1ccc(-c2nc(-c3ccc4ccc5c6ccccc6oc5c4c3)nc3ccccc23)cc1 ; c1ccc(-c2nc(-c3ccc4ccc5c6ccccc6oc5c4c3)nc3c2ccc2ccccc23)cc1 ; c1ccc(-c2nc(-c3ccc4ccc5c6ccccc6oc5c4c3)nc3ccc4ccccc4c23)cc1]

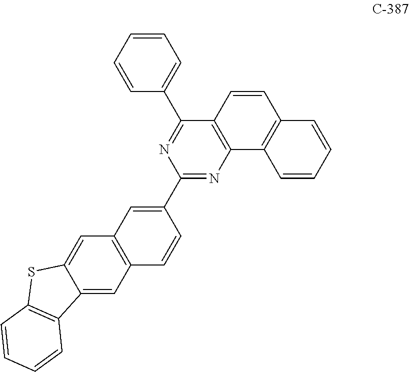 [SMILES: c1ccc(-c2nc(-c3ccc4cc5c(cc4c3)sc3ccccc35)nc3c2ccc2ccccc23)cc1]

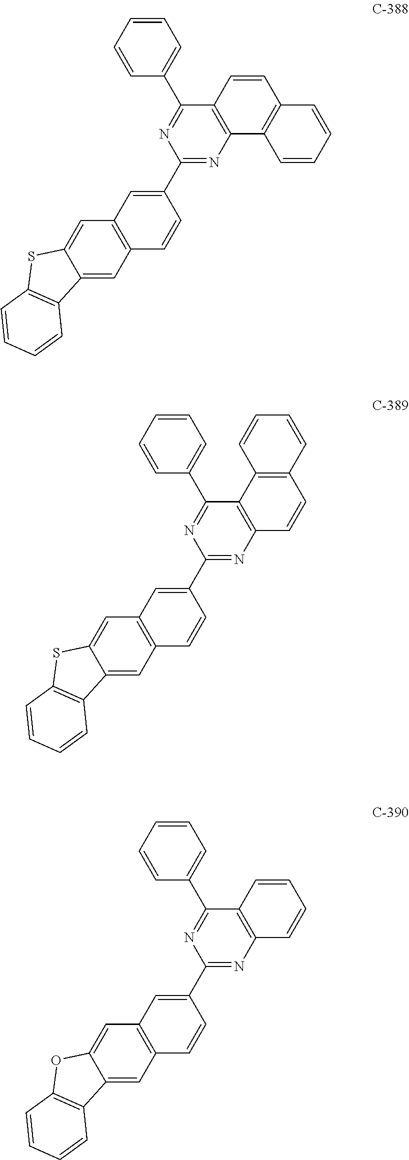 [SMILES: c1ccc(-c2nc(-c3ccc4cc5c(cc4c3)sc3ccccc35)nc3c2ccc2ccccc23)cc1 ; c1ccc(-c2nc(-c3ccc4cc5c(cc4c3)sc3ccccc35)nc3ccc4ccccc4c23)cc1 ; c1ccc(-c2nc(-c3ccc4cc5c(cc4c3)oc3ccccc35)nc3ccccc23)cc1]

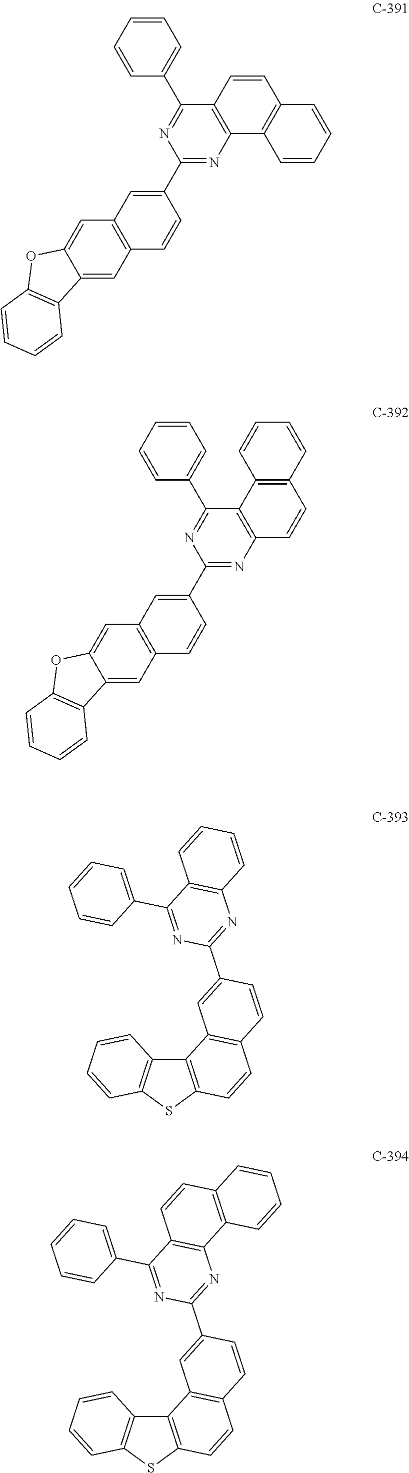 [SMILES: c1ccc(-c2nc(-c3ccc4cc5c(cc4c3)oc3ccccc35)nc3c2ccc2ccccc23)cc1 ; c1ccc(-c2nc(-c3ccc4cc5c(cc4c3)oc3ccccc35)nc3ccc4ccccc4c23)cc1 ; c1ccc(-c2nc(-c3ccc4ccc5sc6ccccc6c5c4c3)nc3ccccc23)cc1 ; c1ccc(-c2nc(-c3ccc4ccc5sc6ccccc6c5c4c3)nc3c2ccc2ccccc23)cc1]

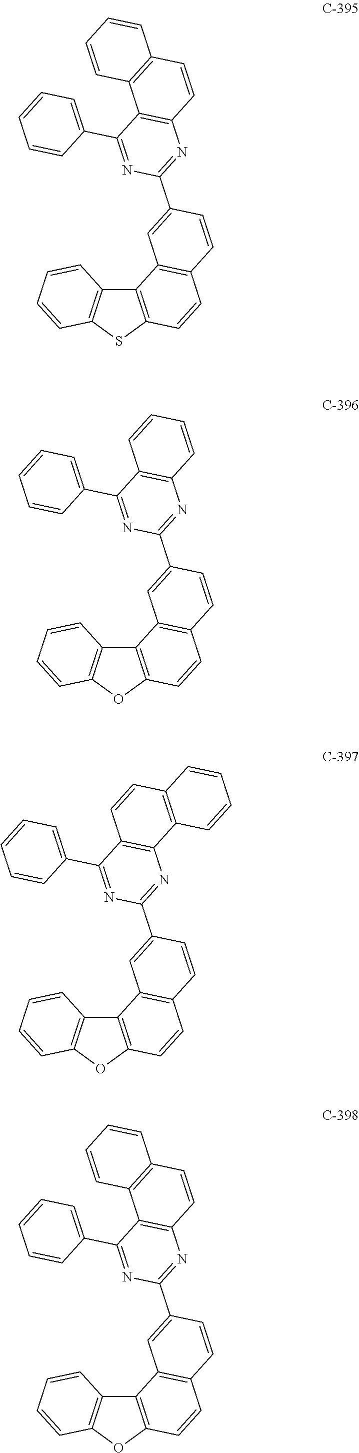 [SMILES: c1ccc(-c2nc(-c3ccc4ccc5sc6ccccc6c5c4c3)nc3ccc4ccccc4c23)cc1 ; c1ccc(-c2nc(-c3ccc4ccc5oc6ccccc6c5c4c3)nc3ccccc23)cc1 ; c1ccc(-c2nc(-c3ccc4ccc5oc6ccccc6c5c4c3)nc3c2ccc2ccccc23)cc1 ; c1ccc(-c2nc(-c3ccc4ccc5oc6ccccc6c5c4c3)nc3ccc4ccccc4c23)cc1]

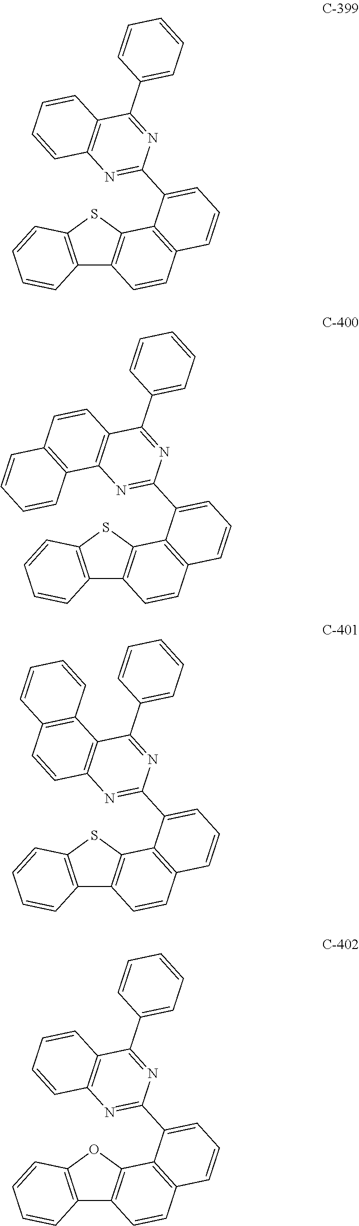

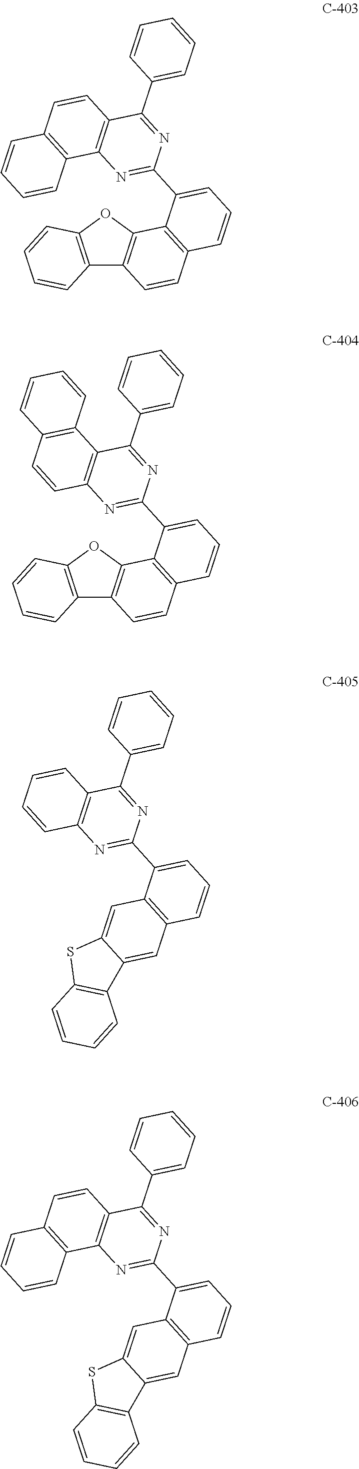 [SMILES: c1ccc(-c2nc(-c3cccc4ccc5c6ccccc6oc5c34)nc3c2ccc2ccccc23)cc1 ; c1ccc(-c2nc(-c3cccc4ccc5c6ccccc6oc5c34)nc3ccc4ccccc4c23)cc1 ; c1ccc(-c2nc(-c3cccc4cc5c(cc34)sc3ccccc35)nc3ccccc23)cc1 ; c1ccc(-c2nc(-c3cccc4cc5c(cc34)sc3ccccc35)nc3c2ccc2ccccc23)cc1]

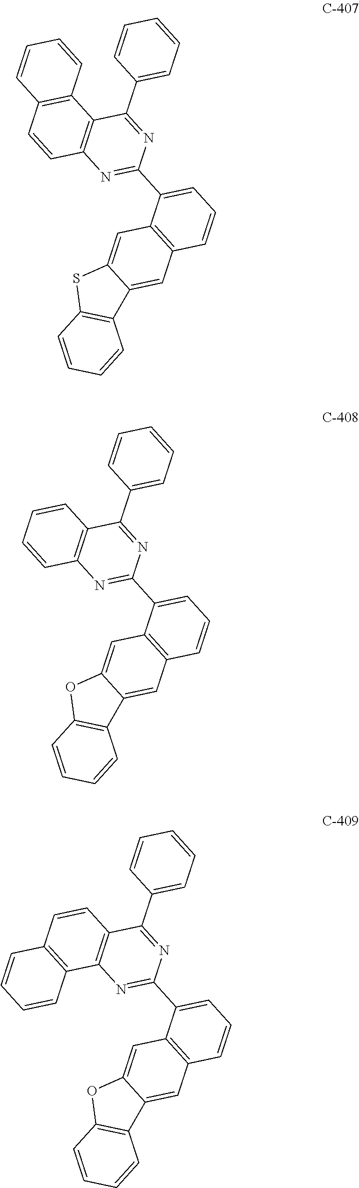 [SMILES: c1ccc(-c2nc(-c3cccc4cc5c(cc34)sc3ccccc35)nc3ccc4ccccc4c23)cc1 ; c1ccc(-c2nc(-c3cccc4cc5c(cc34)oc3ccccc35)nc3ccccc23)cc1 ; c1ccc(-c2nc(-c3cccc4cc5c(cc34)oc3ccccc35)nc3c2ccc2ccccc23)cc1]

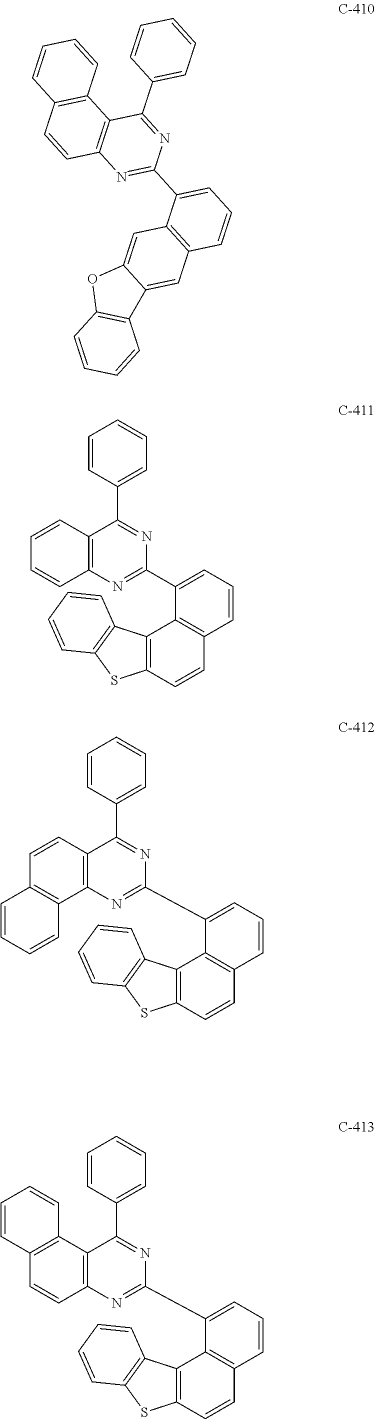

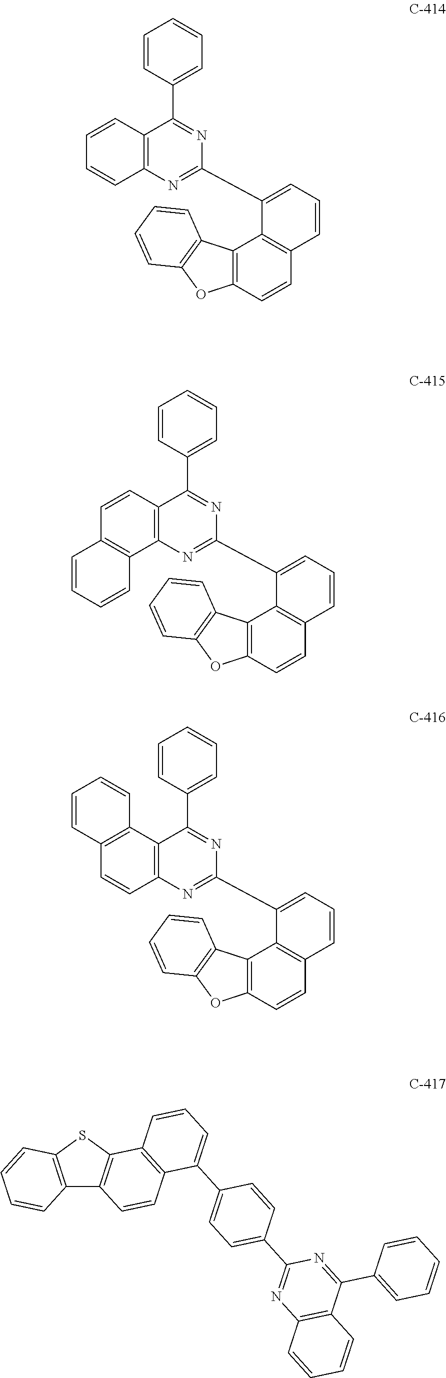 [SMILES: c1ccc(-c2nc(-c3cccc4ccc5oc6ccccc6c5c34)nc3ccccc23)cc1 ; c1ccc(-c2nc(-c3cccc4ccc5oc6ccccc6c5c34)nc3c2ccc2ccccc23)cc1 ; c1ccc(-c2nc(-c3cccc4ccc5oc6ccccc6c5c34)nc3ccc4ccccc4c23)cc1 ; c1ccc(-c2nc(-c3ccc(-c4cccc5c4ccc4c6ccccc6sc54)cc3)nc3ccccc23)cc1]

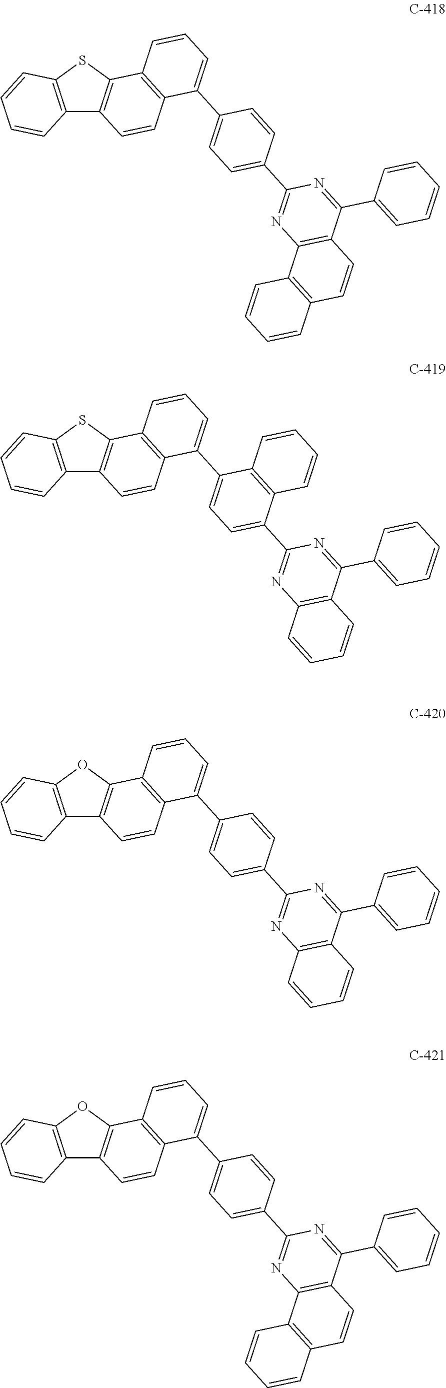 [SMILES: c1ccc(-c2nc(-c3ccc(-c4cccc5c4ccc4c6ccccc6sc54)cc3)nc3c2ccc2ccccc23)cc1 ; c1ccc(-c2nc(-c3ccc(-c4cccc5c4ccc4c6ccccc6sc54)c4ccccc34)nc3ccccc23)cc1 ; c1ccc(-c2nc(-c3ccc(-c4cccc5c4ccc4c6ccccc6oc54)cc3)nc3ccccc23)cc1 ; c1ccc(-c2nc(-c3ccc(-c4cccc5c4ccc4c6ccccc6oc54)cc3)nc3c2ccc2ccccc23)cc1]

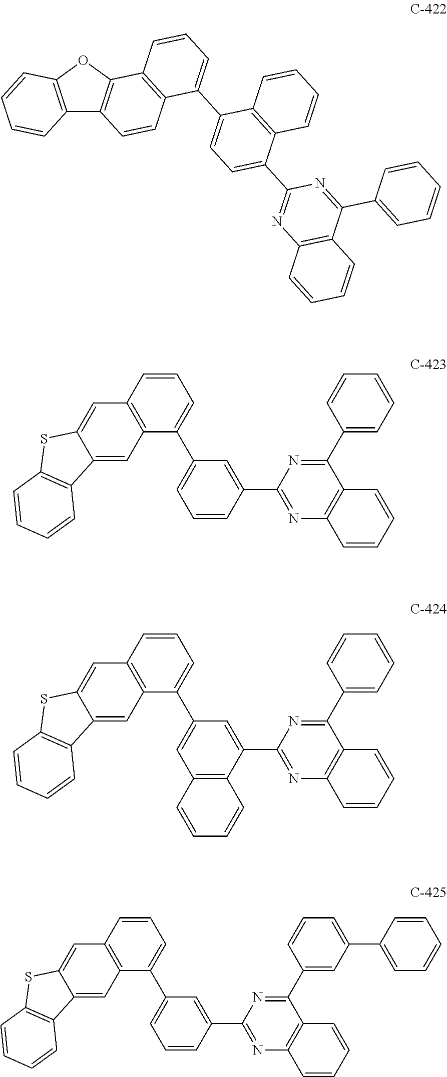 [SMILES: c1ccc(-c2nc(-c3ccc(-c4cccc5c4ccc4c6ccccc6oc54)c4ccccc34)nc3ccccc23)cc1 ; c1ccc(-c2nc(-c3cccc(-c4cccc5cc6sc7ccccc7c6cc45)c3)nc3ccccc23)cc1 ; c1ccc(-c2nc(-c3cc(-c4cccc5cc6sc7ccccc7c6cc45)cc4ccccc34)nc3ccccc23)cc1 ; c1ccc(-c2cccc(-c3nc(-c4cccc(-c5cccc6cc7sc8ccccc8c7cc56)c4)nc4ccccc34)c2)cc1]

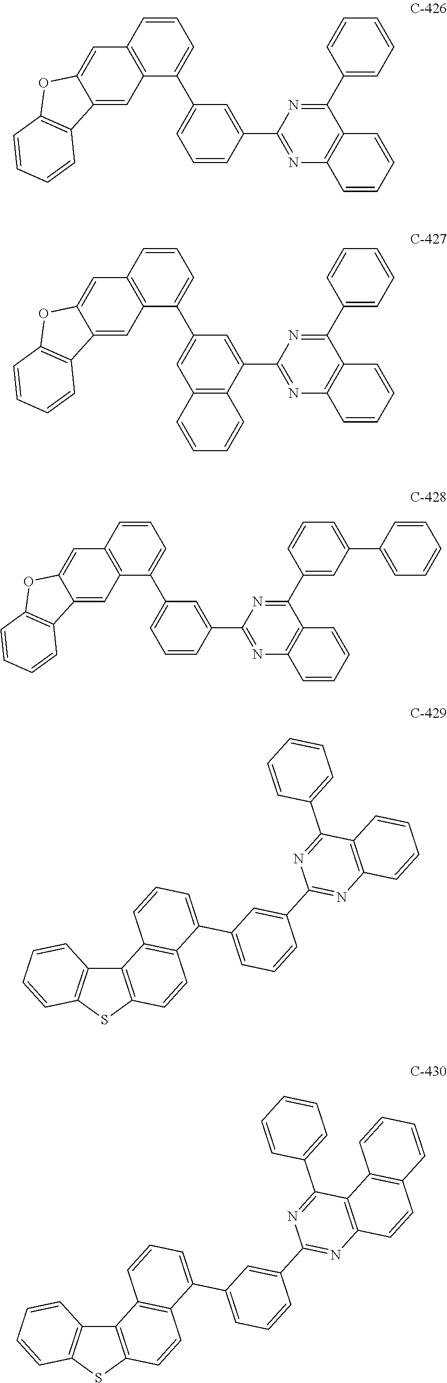 [SMILES: c1ccc(-c2nc(-c3cccc(-c4cccc5cc6oc7ccccc7c6cc45)c3)nc3ccccc23)cc1 ; c1ccc(-c2nc(-c3cc(-c4cccc5cc6oc7ccccc7c6cc45)cc4ccccc34)nc3ccccc23)cc1 ; c1ccc(-c2cccc(-c3nc(-c4cccc(-c5cccc6cc7oc8ccccc8c7cc56)c4)nc4ccccc34)c2)cc1 ; c1ccc(-c2nc(-c3cccc(-c4cccc5c4ccc4sc6ccccc6c45)c3)nc3ccccc23)cc1 ; c1ccc(-c2nc(-c3cccc(-c4cccc5c4ccc4sc6ccccc6c45)c3)nc3ccc4ccccc4c23)cc1]

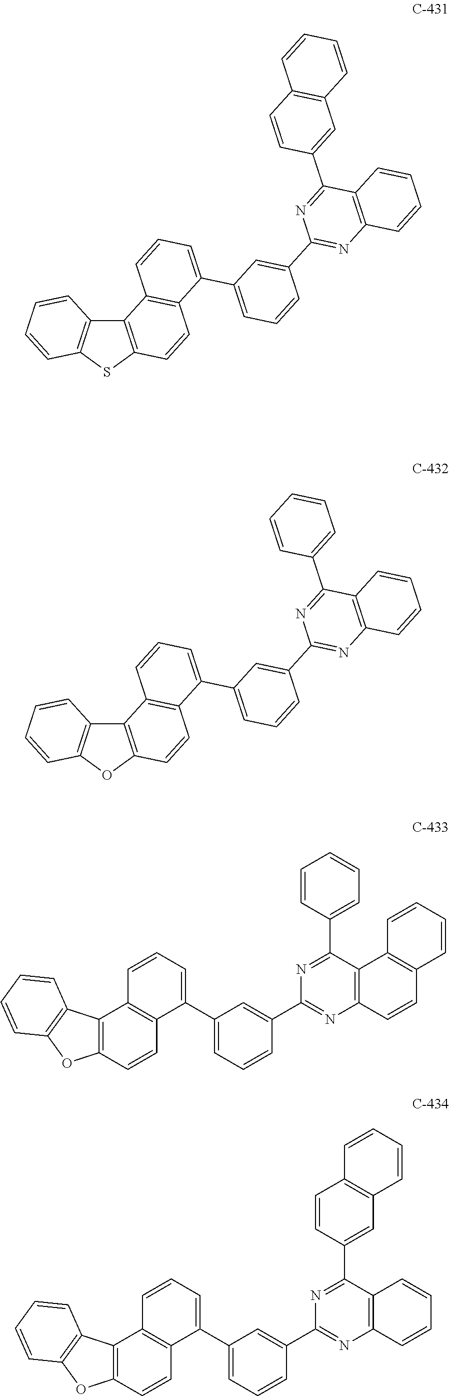 [SMILES: c1cc(-c2nc(-c3ccc4ccccc4c3)c3ccccc3n2)cc(-c2cccc3c2ccc2sc4ccccc4c23)c1 ; c1ccc(-c2nc(-c3cccc(-c4cccc5c4ccc4oc6ccccc6c45)c3)nc3ccccc23)cc1 ; c1ccc(-c2nc(-c3cccc(-c4cccc5c4ccc4oc6ccccc6c45)c3)nc3ccc4ccccc4c23)cc1 ; c1cc(-c2nc(-c3ccc4ccccc4c3)c3ccccc3n2)cc(-c2cccc3c2ccc2oc4ccccc4c23)c1]

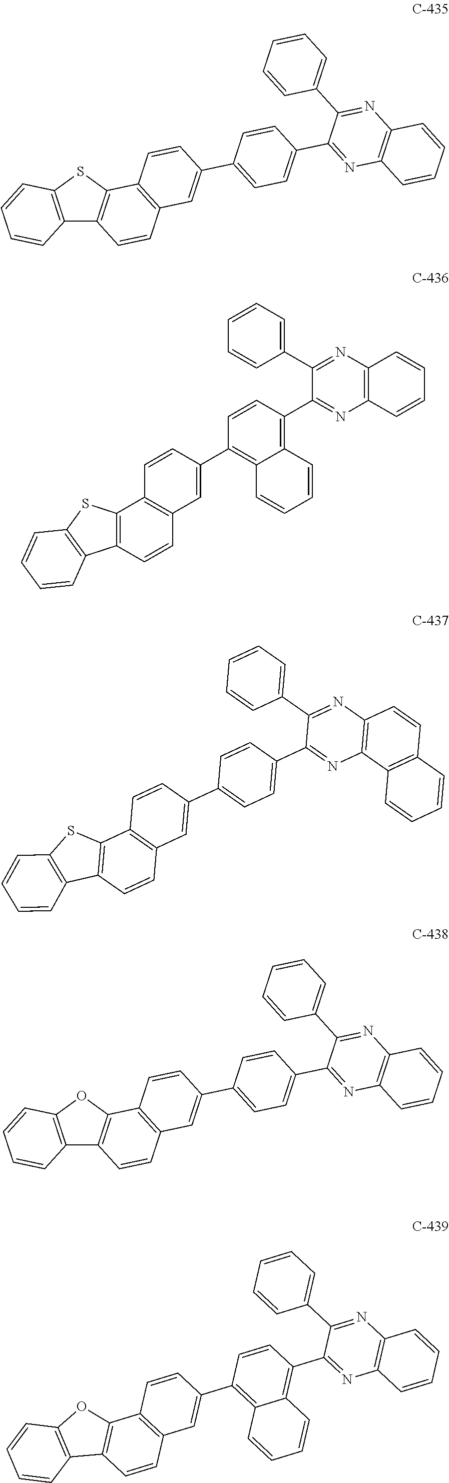 [SMILES: c1ccc(-c2nc3ccccc3nc2-c2ccc(-c3ccc4c(ccc5c6ccccc6sc45)c3)cc2)cc1 ; c1ccc(-c2nc3ccccc3nc2-c2ccc(-c3ccc4c(ccc5c6ccccc6sc45)c3)c3ccccc23)cc1 ; c1ccc(-c2nc3ccc4ccccc4c3nc2-c2ccc(-c3ccc4c(ccc5c6ccccc6sc45)c3)cc2)cc1 ; c1ccc(-c2nc3ccccc3nc2-c2ccc(-c3ccc4c(ccc5c6ccccc6oc45)c3)cc2)cc1 ; c1ccc(-c2nc3ccccc3nc2-c2ccc(-c3ccc4c(ccc5c6ccccc6oc45)c3)c3ccccc23)cc1]

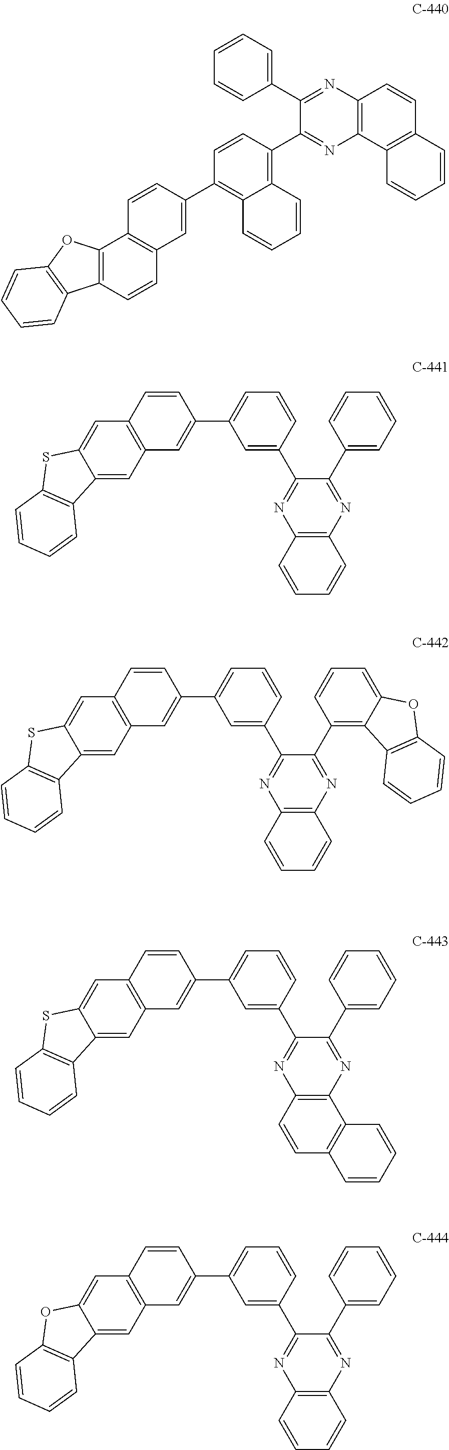 [SMILES: c1ccc(-c2nc3ccc4ccccc4c3nc2-c2ccc(-c3ccc4c(ccc5c6ccccc6oc45)c3)c3ccccc23)cc1 ; c1ccc(-c2nc3ccccc3nc2-c2cccc(-c3ccc4cc5sc6ccccc6c5cc4c3)c2)cc1 ; c1cc(-c2ccc3cc4sc5ccccc5c4cc3c2)cc(-c2nc3ccccc3nc2-c2cccc3oc4ccccc4c23)c1 ; c1ccc(-c2nc3c(ccc4ccccc43)nc2-c2cccc(-c3ccc4cc5sc6ccccc6c5cc4c3)c2)cc1 ; c1ccc(-c2nc3ccccc3nc2-c2cccc(-c3ccc4cc5oc6ccccc6c5cc4c3)c2)cc1]

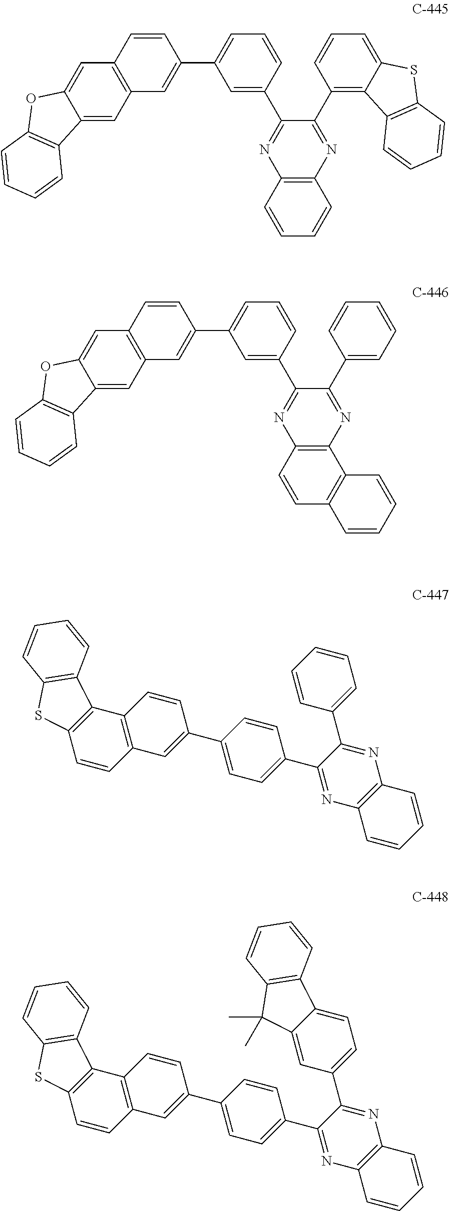 [SMILES: c1cc(-c2ccc3cc4oc5ccccc5c4cc3c2)cc(-c2nc3ccccc3nc2-c2cccc3sc4ccccc4c23)c1 ; c1ccc(-c2nc3c(ccc4ccccc43)nc2-c2cccc(-c3ccc4cc5oc6ccccc6c5cc4c3)c2)cc1 ; c1ccc(-c2nc3ccccc3nc2-c2ccc(-c3ccc4c(ccc5sc6ccccc6c54)c3)cc2)cc1 ; CC1(C)c2ccccc2-c2ccc(-c3nc4ccccc4nc3-c3ccc(-c4ccc5c(ccc6sc7ccccc7c65)c4)cc3)cc21]

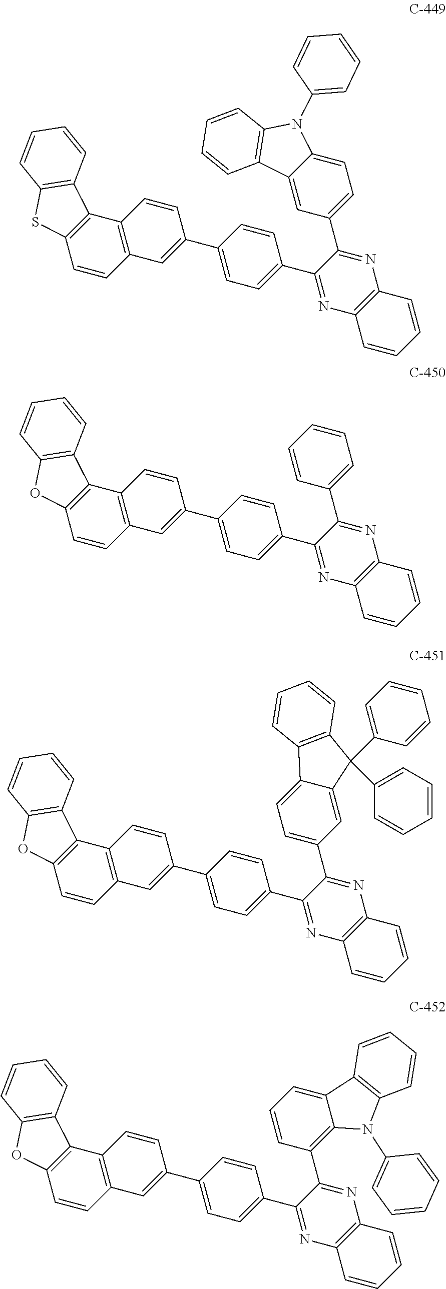 [SMILES: c1ccc(-n2c3ccccc3c3cc(-c4nc5ccccc5nc4-c4ccc(-c5ccc6c(ccc7sc8ccccc8c76)c5)cc4)ccc32)cc1 ; c1ccc(-c2nc3ccccc3nc2-c2ccc(-c3ccc4c(ccc5oc6ccccc6c54)c3)cc2)cc1 ; c1ccc(C2(c3ccccc3)c3ccccc3-c3ccc(-c4nc5ccccc5nc4-c4ccc(-c5ccc6c(ccc7oc8ccccc8c76)c5)cc4)cc32)cc1 ; c1ccc(-n2c3ccccc3c3cccc(-c4nc5ccccc5nc4-c4ccc(-c5ccc6c(ccc7oc8ccccc8c76)c5)cc4)c32)cc1]

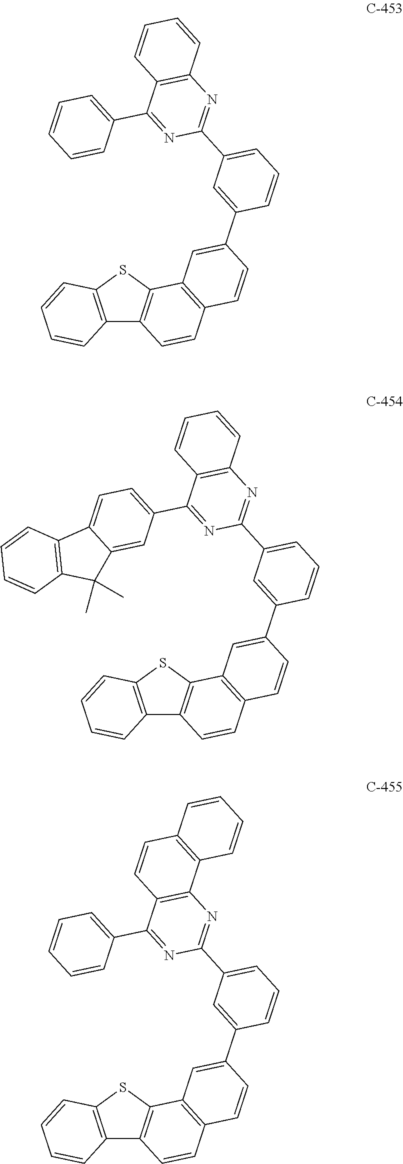 [SMILES: c1ccc(-c2nc(-c3cccc(-c4ccc5ccc6c7ccccc7sc6c5c4)c3)nc3ccccc23)cc1 ; CC1(C)c2ccccc2-c2ccc(-c3nc(-c4cccc(-c5ccc6ccc7c8ccccc8sc7c6c5)c4)nc4ccccc34)cc21 ; c1ccc(-c2nc(-c3cccc(-c4ccc5ccc6c7ccccc7sc6c5c4)c3)nc3c2ccc2ccccc23)cc1]

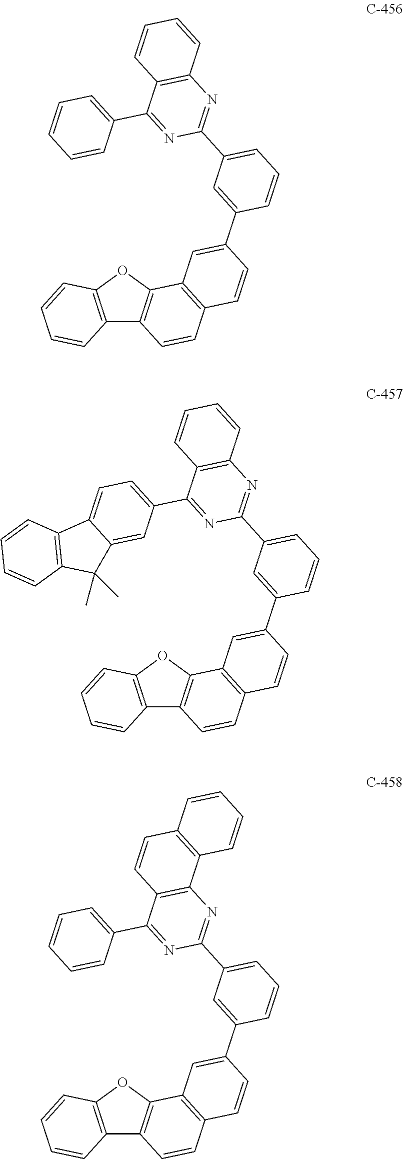 [SMILES: c1ccc(-c2nc(-c3cccc(-c4ccc5ccc6c7ccccc7oc6c5c4)c3)nc3ccccc23)cc1 ; CC1(C)c2ccccc2-c2ccc(-c3nc(-c4cccc(-c5ccc6ccc7c8ccccc8oc7c6c5)c4)nc4ccccc34)cc21 ; c1ccc(-c2nc(-c3cccc(-c4ccc5ccc6c7ccccc7oc6c5c4)c3)nc3c2ccc2ccccc23)cc1]

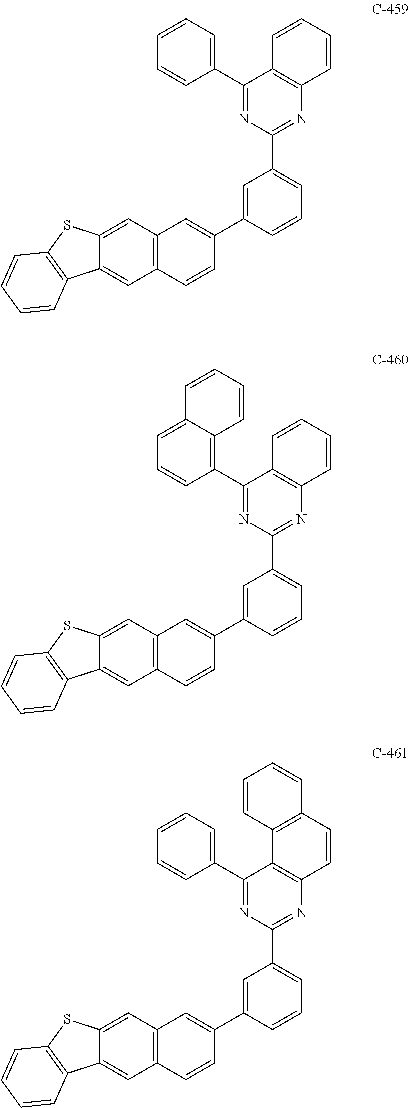 [SMILES: c1ccc(-c2nc(-c3cccc(-c4ccc5cc6c(cc5c4)sc4ccccc46)c3)nc3ccccc23)cc1 ; c1cc(-c2ccc3cc4c(cc3c2)sc2ccccc24)cc(-c2nc(-c3cccc4ccccc34)c3ccccc3n2)c1 ; c1ccc(-c2nc(-c3cccc(-c4ccc5cc6c(cc5c4)sc4ccccc46)c3)nc3ccc4ccccc4c23)cc1]

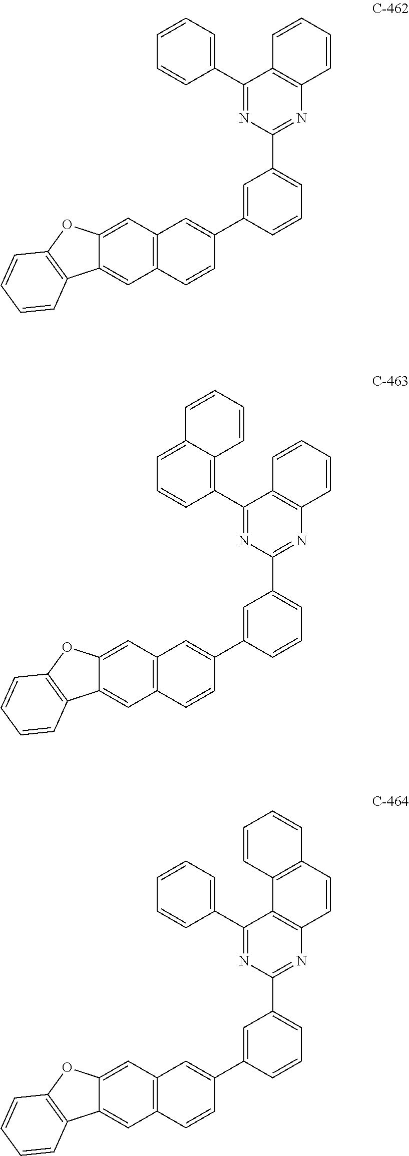 [SMILES: c1ccc(-c2nc(-c3cccc(-c4ccc5cc6c(cc5c4)oc4ccccc46)c3)nc3ccccc23)cc1 ; c1cc(-c2ccc3cc4c(cc3c2)oc2ccccc24)cc(-c2nc(-c3cccc4ccccc34)c3ccccc3n2)c1 ; c1ccc(-c2nc(-c3cccc(-c4ccc5cc6c(cc5c4)oc4ccccc46)c3)nc3ccc4ccccc4c23)cc1]

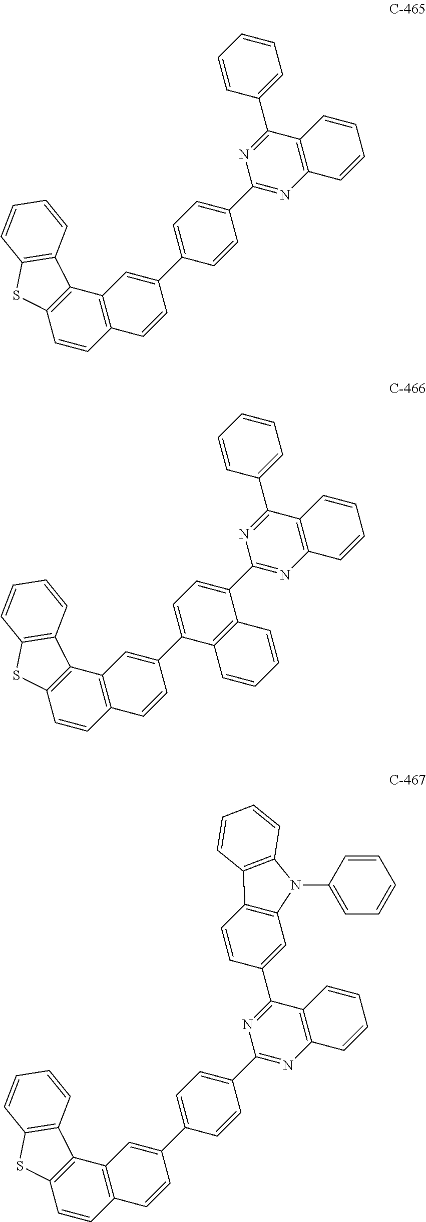 [SMILES: c1ccc(-c2nc(-c3ccc(-c4ccc5ccc6sc7ccccc7c6c5c4)cc3)nc3ccccc23)cc1 ; c1ccc(-c2nc(-c3ccc(-c4ccc5ccc6sc7ccccc7c6c5c4)c4ccccc34)nc3ccccc23)cc1 ; c1ccc(-n2c3ccccc3c3ccc(-c4nc(-c5ccc(-c6ccc7ccc8sc9ccccc9c8c7c6)cc5)nc5ccccc45)cc32)cc1]

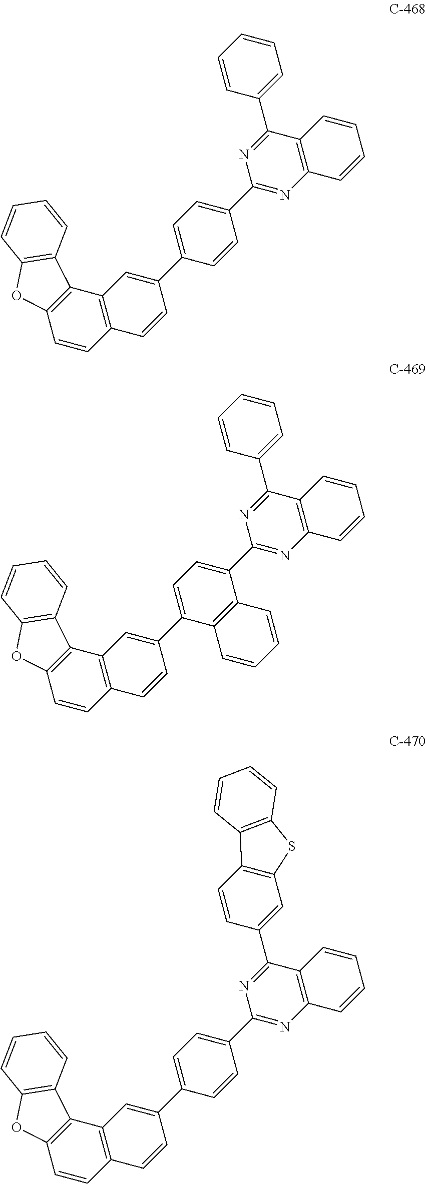 [SMILES: c1ccc(-c2nc(-c3ccc(-c4ccc5ccc6oc7ccccc7c6c5c4)cc3)nc3ccccc23)cc1 ; c1ccc(-c2nc(-c3ccc(-c4ccc5ccc6oc7ccccc7c6c5c4)c4ccccc34)nc3ccccc23)cc1 ; c1ccc2c(-c3ccc4c(c3)sc3ccccc34)nc(-c3ccc(-c4ccc5ccc6oc7ccccc7c6c5c4)cc3)nc2c1]

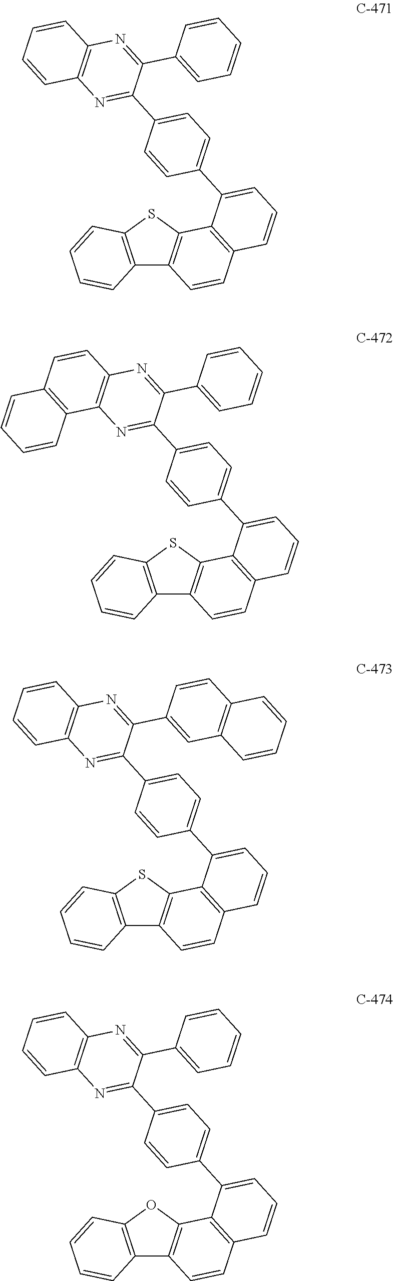 [SMILES: c1ccc(-c2nc3ccccc3nc2-c2ccc(-c3cccc4ccc5c6ccccc6sc5c34)cc2)cc1 ; c1ccc(-c2nc3ccc4ccccc4c3nc2-c2ccc(-c3cccc4ccc5c6ccccc6sc5c34)cc2)cc1 ; c1ccc2cc(-c3nc4ccccc4nc3-c3ccc(-c4cccc5ccc6c7ccccc7sc6c45)cc3)ccc2c1 ; c1ccc(-c2nc3ccccc3nc2-c2ccc(-c3cccc4ccc5c6ccccc6oc5c34)cc2)cc1]

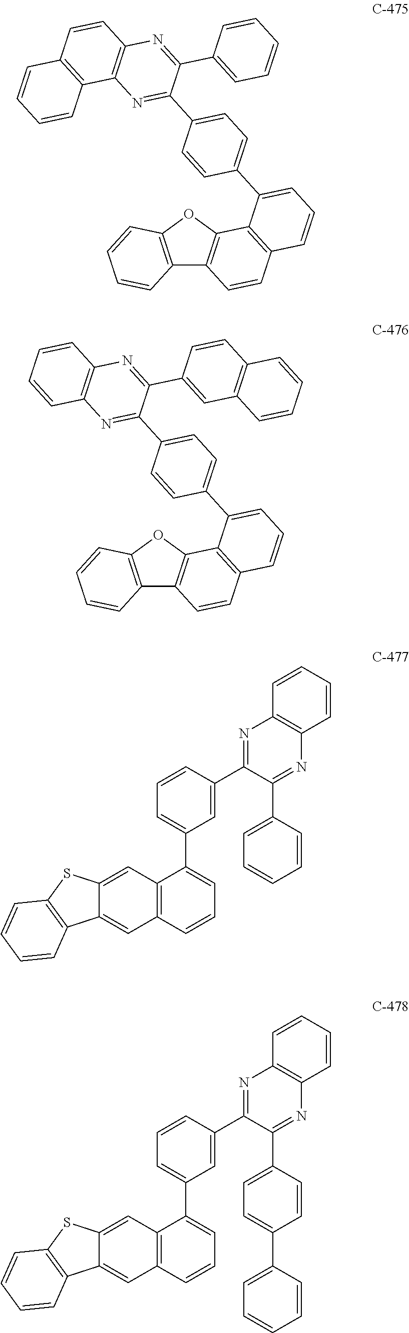 [SMILES: c1ccc(-c2nc3ccc4ccccc4c3nc2-c2ccc(-c3cccc4ccc5c6ccccc6oc5c34)cc2)cc1 ; c1ccc2cc(-c3nc4ccccc4nc3-c3ccc(-c4cccc5ccc6c7ccccc7oc6c45)cc3)ccc2c1 ; c1ccc(-c2nc3ccccc3nc2-c2cccc(-c3cccc4cc5c(cc34)sc3ccccc35)c2)cc1 ; c1ccc(-c2ccc(-c3nc4ccccc4nc3-c3cccc(-c4cccc5cc6c(cc45)sc4ccccc46)c3)cc2)cc1]

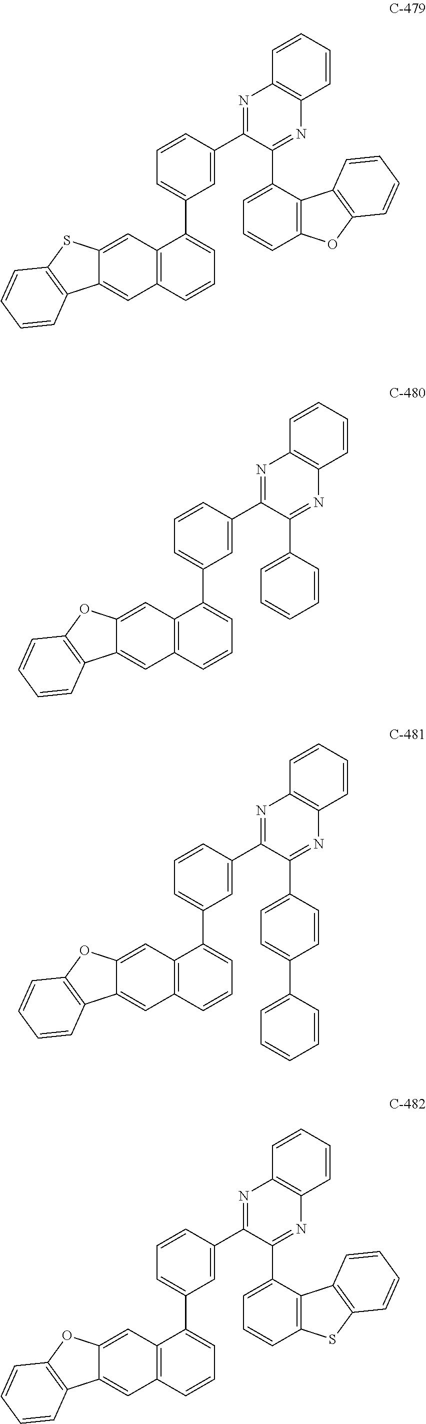 [SMILES: c1cc(-c2nc3ccccc3nc2-c2cccc3oc4ccccc4c23)cc(-c2cccc3cc4c(cc23)sc2ccccc24)c1 ; c1ccc(-c2nc3ccccc3nc2-c2cccc(-c3cccc4cc5c(cc34)oc3ccccc35)c2)cc1 ; c1ccc(-c2ccc(-c3nc4ccccc4nc3-c3cccc(-c4cccc5cc6c(cc45)oc4ccccc46)c3)cc2)cc1 ; c1cc(-c2nc3ccccc3nc2-c2cccc3sc4ccccc4c23)cc(-c2cccc3cc4c(cc23)oc2ccccc24)c1]

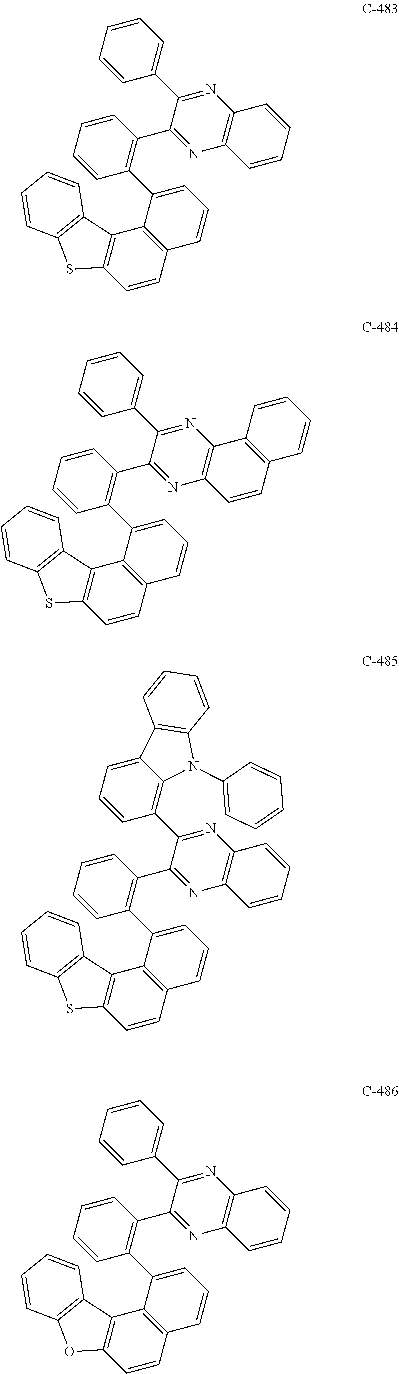 [SMILES: c1ccc(-c2nc3ccccc3nc2-c2ccccc2-c2cccc3ccc4sc5ccccc5c4c23)cc1 ; c1ccc(-c2nc3c(ccc4ccccc43)nc2-c2ccccc2-c2cccc3ccc4sc5ccccc5c4c23)cc1 ; c1ccc(-n2c3ccccc3c3cccc(-c4nc5ccccc5nc4-c4ccccc4-c4cccc5ccc6sc7ccccc7c6c45)c32)cc1 ; c1ccc(-c2nc3ccccc3nc2-c2ccccc2-c2cccc3ccc4oc5ccccc5c4c23)cc1]

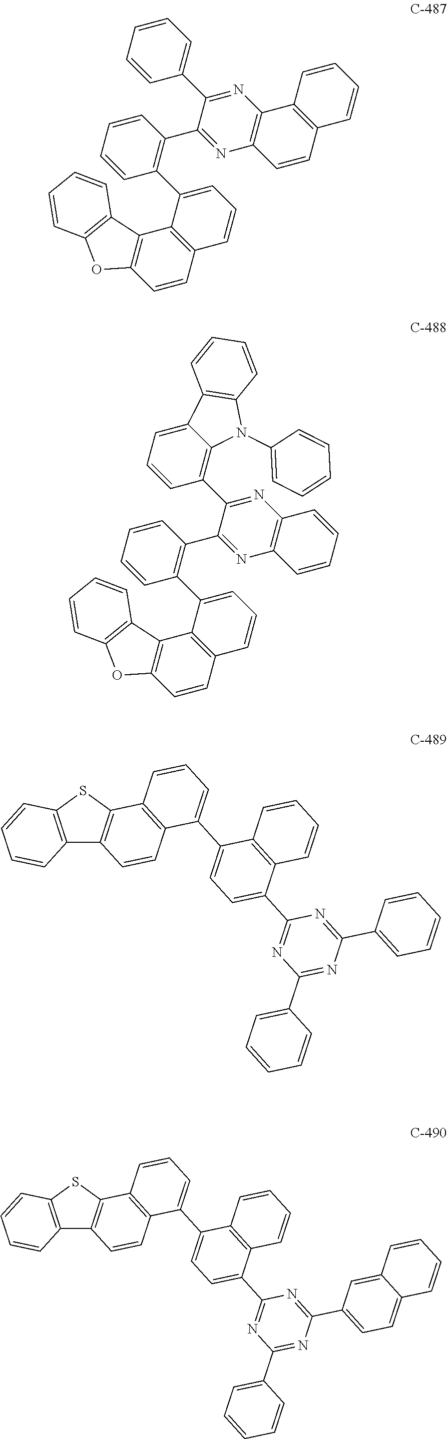 [SMILES: c1ccc(-c2nc3c(ccc4ccccc43)nc2-c2ccccc2-c2cccc3ccc4oc5ccccc5c4c23)cc1 ; c1ccc(-n2c3ccccc3c3cccc(-c4nc5ccccc5nc4-c4ccccc4-c4cccc5ccc6oc7ccccc7c6c45)c32)cc1 ; c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1]

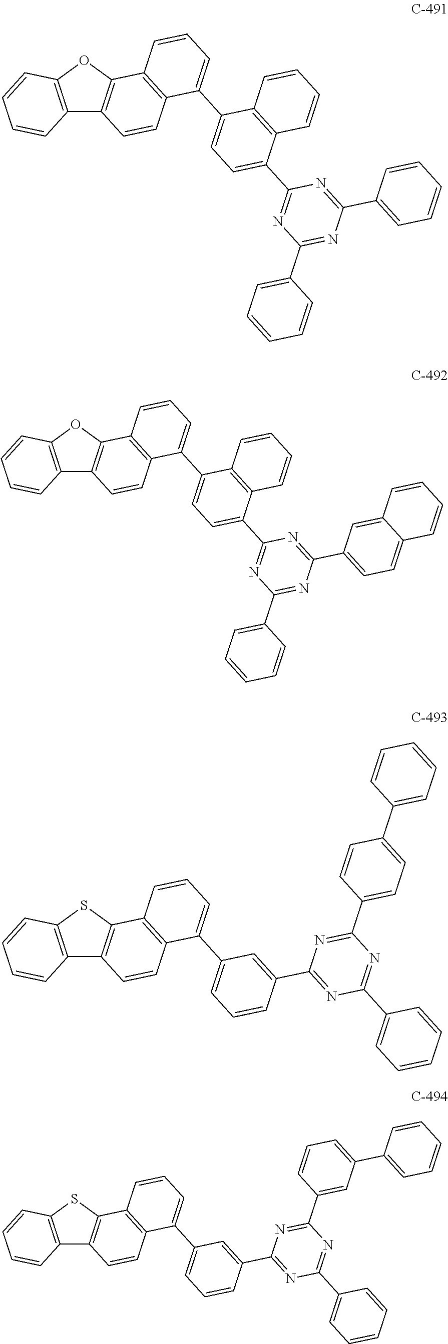 [SMILES: c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1 ; c1ccc(-c2ccc(-c3nc(-c4ccccc4)nc(-c4cccc(-c5cccc6c5ccc5c7ccccc7sc65)c4)n3)cc2)cc1 ; c1ccc(-c2cccc(-c3nc(-c4ccccc4)nc(-c4cccc(-c5cccc6c5ccc5c7ccccc7sc65)c4)n3)c2)cc1]

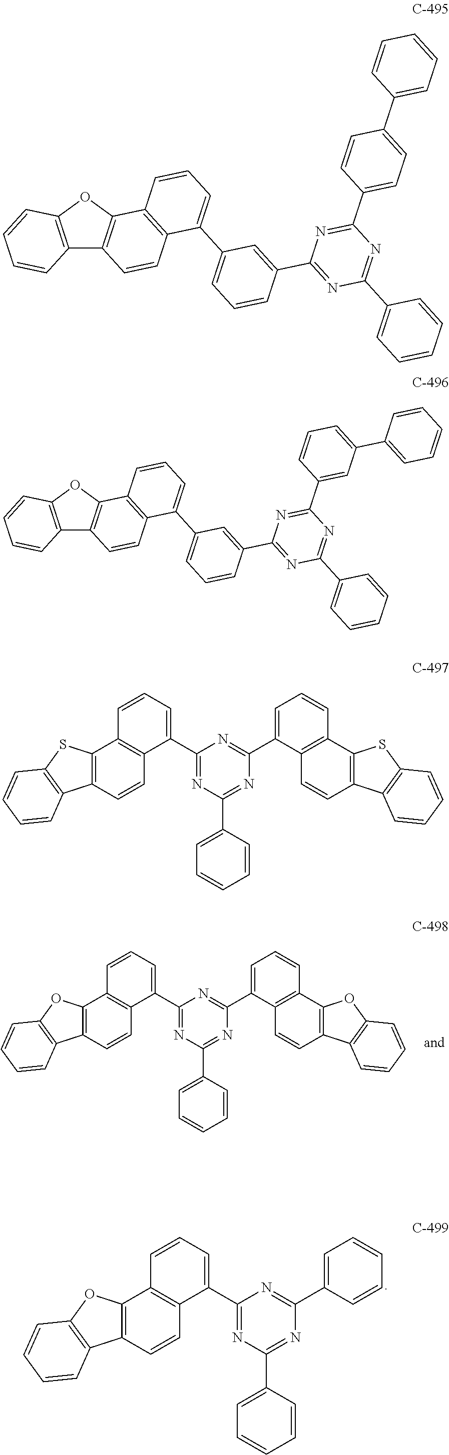 [SMILES: c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1 ; c1ccc(-c2nc(-c3cccc4c3ccc3c5ccccc5sc43)nc(-c3cccc4c3ccc3c5ccccc5sc43)n2)cc1 ; c1ccc(-c2nc(-c3cccc4c3ccc3c5ccccc5oc43)nc(-c3cccc4c3ccc3c5ccccc5oc43)n2)cc1 ; c1ccc(-c2nc(-c3ccccc3)nc(-c3cccc4c3ccc3c5ccccc5oc43)n2)cc1]

6. An organic electroluminescent material comprising the organic electroluminescent compound according to claim 1.

7. An organic electroluminescent device comprising the organic electroluminescent compound according to claim 1.

8. The organic electroluminescent device according to claim 7, wherein the organic electroluminescent compound is comprised in at least one of a light-emitting layer and an electron transport zone.
